SEQUENCE LISTING (317 sequences)

SEQ ID NO: 1 - protein, 160 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYS

SEQ ID NO: 2 - DNA, 480 bp, Artificial Sequence
  Synthetic
ggacgcggcgatagtccatattcaggtcggggagatagtccctactcaggtaggggagacagcccatatagcgggcgtggcgactctccatatagtgggagaggggactctccgtactctgggcgcggtgattcaccatattctggtcgaggtgactcaccttattctggcaggggtgatagtccgtattcaggccgtggtgattccccctatagtggaaggggagattcaccgtatagtggtagaggcgacagtccttattcgggacgaggtgattctccttatagtggccgaggggattcaccctattctggaagaggtgatagcccttattcagggaggggcgattctccctattcaggacggggtgactctccttactccggtcgcggagattctccatactcgggtcgtggagactccccatattccggcagaggtgacagtccatacagtggacgtggggatagtccttacagt

SEQ ID NO: 3 - protein, 160 aa, Artificial Sequence
  Synthetic
GRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQ

SEQ ID NO: 4 - DNA, 480 bp, Artificial Sequence
  Synthetic
gggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcag

SEQ ID NO: 5 - protein, 160 aa, Artificial Sequence
  Synthetic
GRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYT

SEQ ID NO: 6 - DNA, 480 bp, Artificial Sequence
  Synthetic
ggacgtggtgacaccccctatacgggacgtggagatacaccttacactggacgcggtgatacgccttacaccggacgtggcgatacaccatatactggtagaggtgatacaccctatacaggccgtggagacactccatacactggtcggggagacacgccatatacaggacgaggtgacacaccttatactggaagaggggacacaccatacacaggtcgcggcgatacgccgtatactgggagaggcgacactccttatacagggcgcggagataccccgtacactgggcgtggggatactccgtataccggtcgaggggatacaccgtatacaggtaggggtgacactccctatactggcaggggagatactccctacactggccggggtgatactccttacacgggtcgtggtgataccccttacacaggaaggggcgatactccatatacgggccgaggcgataccccatatacc

SEQ ID NO: 7 - protein, 160 aa, Artificial Sequence
  Synthetic
GRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYN

SEQ ID NO: 8 - DNA, 480 bp, Artificial Sequence
  Synthetic
ggtcgaggcgataacccgtataatggaaggggagataacccttataatggtagaggggataacccatataatgggcgtggcgacaacccgtacaatggcaggggcgataatccctataatggacgaggtgataacccctacaacggacgcggtgataatccatacaacgggaggggtgacaacccatacaatgggagaggggacaatccttataatggccgtggtgataatccctacaatggtcgtggggataatccatataacggtcgcggagataatccttataacggccggggtgataatccgtataacggaagaggtgacaatccctataacgggcgcggggacaacccctataatggtaggggagacaacccttacaacggcagaggcgacaatccatataatggtcggggagataatccgtacaacggtagaggtgataatccttacaatggacgtggagacaatccgtataat

SEQ ID NO: 9 - protein, 160 aa, Artificial Sequence
  Synthetic
GRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHS

SEQ ID NO: 10 - DNA, 480 bp, Artificial Sequence
  Synthetic
gggcgtggagactctccacatagcggaagaggggacagtcctcattcaggtcggggtgattcccctcattcgggtcgaggtgattctccacactcgggacggggagattctccccattctgggcgcggtgatagcccgcattcaggacgcggggactcgccacatagtgggagaggtgatagtccacacagtggaaggggagatagtccccatagtggccgaggggattcaccacattccggtaggggagacagccctcatagtggacgtggtgattcgccgcatagtggtagaggcgatagtcctcactcagggaggggcgattcaccccactccggcaggggtgactcacctcacagcggtcgtggagatagcccacattcaggccgtggggatagtccgcactctggcagaggtgacagtccacattctggtcgcggagattcacctcattctggacgaggcgactccccccacagt

SEQ ID NO: 11 - protein, 160 aa, Artificial Sequence
  Synthetic
GRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFS

SEQ ID NO: 12 - DNA, 480 bp, Artificial Sequence
  Synthetic
ggccgcggcgattctccattttctggcagaggtgacagccctttttccggtcggggtgactctccgttctcgggacgtggcgacagtccttttagtggacgaggtgactccccctttagtggccgtggggattcaccgttttctgggagaggggactctccattcagcgggcgtggtgattctcccttttcaggccgaggggatagtcccttctccggcaggggtgattcacctttttcaggacggggagatagtcctttctcagggaggggcgatagtccgtttagtggtaggggagactctcctttcagtggaaggggagattccccatttagtgggcgcggagattctcctttttctggtcgaggtgatagcccattctctggaagaggtgattcgcctttttcgggtcgtggagattcaccattttcaggtcgcggtgatagtccatttagcggtagaggcgactcaccctttagt

SEQ ID NO: 13 - protein, 160 aa, Artificial Sequence
  Synthetic
GRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYS

SEQ ID NO: 14 - DNA, 480 bp, Artificial Sequence
  Synthetic
ggacgaggtgatcagccgtattcgggtcgaggggatcagccatattcaggtcgcggggatcaaccatactcaggtagaggggaccaaccgtattccggtcgtggtgaccaaccatatagcggcaggggtgatcaaccgtatagtggtcggggtgaccaaccctattctggtaggggagatcagccctattcaggacgcggagatcaaccttactctgggagaggtgaccagccttacagtggaaggggcgaccagccatatagtggccgaggcgatcaaccgtactcgggacggggagatcaaccctatagtgggcgcggtgatcaaccctactccgggcgtggcgatcagccatacagcggacgtggtgatcaaccatattctggcagaggtgatcaaccttatagtggacgtggagatcagccttattctggaagaggcgaccaaccttattcaggccgtggggaccagccctacagt

SEQ ID NO: 15 - protein, 160 aa, Artificial Sequence
  Synthetic
GRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYS

SEQ ID NO: 16 - DNA, 480 bp, Artificial Sequence
  Synthetic
ggacggggtgataatccgtattcgggacgaggtgataatccctacagcggcagaggtgacaacccctactcagggaggggcgataatccttacagtggaaggggagacaacccttactccggtcgaggcgacaacccatatagcggacgtggcgacaatccgtatagtgggagaggcgataacccttattccgggcgtggggataatccatatagtggcaggggtgacaatccctatagtggtagaggggacaacccgtattctgggcgcggggacaatccatattctggaagaggtgataacccctattctggccgtggagacaatccttattcaggacgtggtgataatccatactctggtcgtggagataatccgtactcgggtcgcggtgataatccttatagtggacgcggagataacccatattcaggtcggggagataatccctattcaggccgaggggataacccatacagt

SEQ ID NO: 17 - protein, 160 aa, Artificial Sequence
  Synthetic
GRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYN

SEQ ID NO: 18 - DNA, 480 bp, Artificial Sequence
  Synthetic
ggtcgtggtgattctccttataatgggcgtggggattcaccttacaatggacgaggtgatagtccgtataatggaagaggtgactccccatacaacggtagaggtgatagcccatataatggacgtggcgattctccatataacggcaggggagatagtccatataatgggaggggtgattccccctataatggtaggggagactctccatacaatgggagaggtgattcgccgtataacggaagaggcgatagtccctataacgggcgcggtgattcaccatataatggtagaggggacagcccttataatggaaggggcgactctccgtacaatggtcggggtgactcaccttataatggccgtggagattctccctacaatggcagaggtgacagtccttacaacggacgcggggactcgccttataatggtcgaggggatagtccttataacggtcgcggagattcaccctataat

SEQ ID NO: 19 - protein, 160 aa, Artificial Sequence
  Synthetic
GRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQ

SEQ ID NO: 20 - DNA, 480 bp, Artificial Sequence
  Synthetic
ggacgtggtgataatccttaccagggacggggagataacccgtaccaaggacgcggagataatccctaccagggccggggtgataatccctatcagggtagaggggataacccctaccaaggccgtggggacaacccgtatcaaggtcggggcgataacccataccaagggaggggtgacaacccctatcaaggacgaggtgataacccttatcaaggcaggggagacaacccttaccaaggtcgcggggataatccttatcagggcagaggcgacaatccgtatcagggaaggggagataatccgtaccagggtcgaggcgataatccatatcagggtcgtggcgacaacccatatcaaggaagaggtgacaatccatatcaagggagaggggacaatccctatcaagggcgcggtgataatccataccaggggcgtggagacaatccttatcaaggtagaggtgataatccgtatcaa

SEQ ID NO: 21 - protein, 160 aa, Artificial Sequence
  Synthetic
GRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYN

SEQ ID NO: 22 - DNA, 480 bp, Artificial Sequence
  Synthetic
ggacgtggtgatcaaccttacaacggacgcggagatcaaccgtacaatgggcgcggggaccaaccctataatggtagaggcgaccagccctataatggcagaggtgatcagccatacaatggtcgtggcgaccaaccgtataacgggcgtggagatcagccgtataatggaagaggggaccagccttataatggtcgaggtgatcaaccgtataatggtaggggagatcaaccctataacggtcgcggtgatcaaccatacaacggtagaggggatcaaccttataatggacgaggcgatcaaccatataacggaaggggtgaccagccatataatgggagaggtgaccaaccatataatggtcggggtgatcaaccctacaacgggaggggcgatcagccctacaatggacgtggggatcagccttacaatggccgtggtgaccaaccttataacggccggggagatcagccttataac

SEQ ID NO: 23 - protein, 160 aa, Artificial Sequence
  Synthetic
GRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQ

SEQ ID NO: 24 - DNA, 480 bp, Artificial Sequence
  Synthetic
ggacgagatggaagtccttatcaaggtcgggatggctctccgtatcagggaagagacggtagtccataccaaggccgtgacggttctccttatcagggtagagacgggtcaccatatcagggccgagacggaagcccttaccagggacgtgatggttcgccatatcaagggagggacggatccccttatcaagggcgtgatgggtccccctaccaagggagagatgggagcccataccagggtcgtgatggatcaccctatcaaggaagggatggatcgccttatcaaggacgcgatggtagcccgtaccaaggtcgagatggttccccatatcaaggtagggatggttcaccgtatcaaggtagagatggcagtccgtaccagggcagggatgggagtccctatcaggggcgcgacggctcaccttatcaaggcagagatggtagtccttaccaaggaagagatggatctccatatcaa

SEQ ID NO: 25 - protein, 160 aa, Artificial Sequence
  Synthetic
GRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQ

SEQ ID NO: 26 - DNA, 480 bp, Artificial Sequence
  Synthetic
ggacgtgatggacaaccctatcagggccgagatggacaaccataccaaggtagagacggacaaccatatcaaggtagggatggtcaaccctaccaaggtcgtgatggtcaaccgtaccaggggagagatggacagccttaccaagggagggatggccaaccctatcaaggaagagacggccaaccttaccagggcagagatgggcagccttatcaaggacgagatggtcagccgtaccaaggaagggacggtcaaccatatcagggtcgggatgggcaaccgtatcaggggcgcgatggacaaccgtatcaaggtcgagacggtcagccatatcaaggcagggatggacaaccttatcagggacgcgacggacagccctaccagggaagagatggtcaaccttatcaagggcgtgacgggcaaccttaccaaggccgcgatggtcaaccataccagggtagagatggccagccataccaa

SEQ ID NO: 27 - protein, 160 aa, Artificial Sequence
  Synthetic
GRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYN

SEQ ID NO: 28 - DNA, 480 bp, Artificial Sequence
  Synthetic
ggccgtgatgggtccccgtacaatggtcgggatggctcaccgtataatggaagagatggcagtccatacaatggcagagatgggagcccatacaacggaagagacggctctccttataacgggcgcgatggatctccatataatggtcgtgatggatcaccttataatgggagggacggttcaccatataacggtcgagatggatcgccatataatgggcgtgatggttccccttacaacggtagagacgggtcaccctataacggacgagatggtagtccttacaatgggagagatggaagtccttataatggtagagatggttcgccttataatggccgagacggatctccgtataacggcagggatggttctccctataatggtagggatggatccccatataatggacgtgacggtagcccttataatggaagggatgggagtccctacaatggacgcgacggaagcccctataat

SEQ ID NO: 29 - protein, 160 aa, Artificial Sequence
  Synthetic
GRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHN

SEQ ID NO: 30 - DNA, 480 bp, Artificial Sequence
  Synthetic
ggtcgtggcgacaatcctcataacggtcgaggtgataatccacataatgggagaggtgacaatccccataatggtaggggcgataacccccacaatggcagaggggataatccacacaacggcaggggagacaatcctcacaacgggaggggtgataatccgcataacggacgcggggataaccctcataatggacgaggcgataatccccataacggtagaggggacaatccacataacggccggggtgacaaccctcataacgggcgcggagacaatccgcacaatgggcgtggagacaacccccataatggaagaggcgacaacccacataatggtagaggtgataaccctcacaatggtcggggagataacccacacaatggacgtggggacaacccgcataatggccgtggtgataatcctcataatggtcgcggtgataatccccacaacggaaggggagataatcctcacaat

SEQ ID NO: 31 - protein, 160 aa, Artificial Sequence
  Synthetic
GRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHS

SEQ ID NO: 32 - DNA, 480 bp, Artificial Sequence
  Synthetic
ggacgtggggacaaccctcattccggtcgaggggacaatcctcactcaggtagaggggataaccctcactctggtcgtggagataaccctcatagtgggagaggtgacaacccccattcaggtcggggtgacaatccccattcgggtcgcggtgataatccgcactccgggcgcggggataatccacacagtggaaggggagataatccccatagtggccgtggtgataatccacattcaggccgaggtgataatccccactctggaagaggtgataacccacactcgggacgtggcgataatcctcattctgggcgtggagacaacccgcatagtggtaggggcgacaatcctcatagcggacgcggagacaatccacatagtggacggggagacaatccgcattcaggacgaggcgacaacccacattctggcaggggtgataatcctcacagcggcagaggcgataacccccacagt

SEQ ID NO: 33 - protein, 160 aa, Artificial Sequence
  Synthetic
GRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHN

SEQ ID NO: 34 - DNA, 480 bp, Artificial Sequence
  Synthetic
ggtcgaggggattcacctcacaatggtcgcggggacagtcctcacaacggtagaggcgattcaccccataatggacgcggagattcaccacataatggtaggggagactcgccacataatgggcgtggggatagtccccataacggacgaggtgatagtccgcacaatggacgtggagatagccctcataatggtcgtggtgattcccctcataacgggcgcggcgatagtccacataatggaaggggagattcgcctcataatgggaggggcgactcacctcataatggcagaggtgattcaccgcataatggtagaggtgactcaccacacaacggaagaggggactccccccacaatggccggggtgattctccacataacggcaggggtgactctccacacaatgggagaggtgatagcccgcataacggtcggggagatagtcctcataatggaagaggtgacagcccacataat

SEQ ID NO: 35 - protein, 160 aa, Artificial Sequence
  Synthetic
GRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHN

SEQ ID NO: 36 - DNA, 480 bp, Artificial Sequence
  Synthetic
ggtcgtggtgatcaaccccataatggacgcggagatcagccgcataacgggagaggggatcaacctcataatggcagaggggaccaaccacataacggtagaggtgaccagccccataatggtcggggagatcagccccacaacggacgtggcgatcaaccccacaatggccgtggggaccagccacataatggtagaggcgaccaacctcataacggcaggggtgaccaaccccataacggaaggggagatcaaccacacaacgggcgcggggatcagccacacaatggacgtggtgaccaacctcacaacggccgaggtgatcaaccacataatggaagaggtgatcagcctcacaatgggcgtggagatcaaccgcacaatggtcgaggcgatcagcctcataacggtcgcggtgaccaaccgcataatggtaggggcgaccagcctcataatgggaggggtgatcaacctcacaat

SEQ ID NO: 37 - protein, 160 aa, Artificial Sequence
  Synthetic
GRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQGRGDNPHQ

SEQ ID NO: 38 - DNA, 480 bp, Artificial Sequence
  Synthetic
ggtcgtggtgataatccgcaccagggtagaggcgataacccccatcaaggaagaggggataatccccaccaagggcgcggggataacccgcaccaaggtcggggagataatcctcaccaaggccgtggggacaaccctcatcaaggtagaggtgacaatccgcatcagggacgcggtgataatccacatcaagggagaggtgataatcctcatcagggtaggggagataaccctcatcaggggaggggtgataatccccatcagggccggggcgacaacccccaccagggcaggggtgacaacccgcatcaaggcagaggggacaatccacatcagggtcgcggagacaatcctcatcaaggacgtggcgataatccacaccaggggcgtggagataacccacatcaaggtcgaggtgataaccctcaccagggaaggggagacaacccacaccaaggacgaggcgacaatccccatcaa

SEQ ID NO: 39 - protein, 160 aa, Artificial Sequence
  Synthetic
GRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPASGRGDSPAS

SEQ ID NO: 40 - DNA, 480 bp, Artificial Sequence
  Synthetic
ggccgcggcgattccccagcctccggaagaggtgattcgccagcatctggccgaggggattcacctgcttcggggaggggtgactctccagccagtggcagaggggatagtccggcatccggtagaggggactctcctgcctcgggccgtggagattcaccagctagtggtaggggcgatagtccagcgtcaggacgtggcgacagtcctgcgagcggtcgtggggacagtccagcttcgggtcggggtgattcccctgcaagcggacgaggtgatagtcctgcatcaggtagaggcgactcccccgcctcaggtcgcggagatagccctgcgtctgggagaggtgactcacccgctagtgggcgtggtgattcaccggcttctggtcgaggtgacagcccggcctctggaaggggagattctcccgcatcgggacgcggtgatagcccagcaagtggacggggagactcgcctgctagt

SEQ ID NO: 41 - protein, 160 aa, Artificial Sequence
  Synthetic
GRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPISGRGDSPIS

SEQ ID NO: 42 - DNA, 480 bp, Artificial Sequence
  Synthetic
ggccgtggggattctcccatctcgggtcggggtgactctcctatttcgggacggggagatagtccgatttctgggaggggtgatagtcctatatccggtcgcggagactctccaataagcgggcgtggtgattcaccaatttcaggacgcggtgattctcctatcagtggacgaggggatagtccaattagtggtcgaggtgattccccaatatctggccgaggtgacagccctatatcaggccgtggcgattcacccatttccggcagaggggactctccgatctcaggtagaggcgacagtcctattagtgggcgcggcgactcccccataagtggcaggggcgatagtcccatatcaggaaggggagattcaccgatatcagggagaggtgactcacctatatctggtaggggagacagtccaatatcaggtcgtggagattctccaatctctggaagaggtgatagcccaatcagc

SEQ ID NO: 43 - protein, 160 aa, Artificial Sequence
  Synthetic
GGRSDSYPGGRPSDYSSDRPSYGGGYRSGSPDSPDGYRSGRGYDGPSSRGSPGYDSYPGSDGSRDYRPGSGSPRGGDYSSSPGDSRYGGGRYDPSSPSDSRGGYSPYGSRDGSSPGYGRDRSDSGGYPPDRSSGGYDGYSGRSPSGSDGPRYSYGRSPGD

SEQ ID NO: 44 - DNA, 480 bp, Artificial Sequence
  Synthetic
ggccgcagtgacagctatccaggcggacgcccgagcgactattcctcggaccgtccatcttatgggggtggttaccgttctggcagccccgatagccctgacggttatcgctctggacgcgggtatgacggtccctcatcccgcggctctccaggatacgatagttatccgggctccgatggtagtcgcgactatcgtcccgggtcagggtccccccgcggcggggactactccagttcaccaggagactcgcgctacggcgggggtcgttatgatcccagctccccttcggatagtcgtggtggctacagtccctacggttctcgcgacggtagctctccagggtacggacgtgatcgttctgatagtggaggttacccgccggatcgttcaagtgggggatacgacgggtattcaggtcgctcacctagtggtagcgatggccctcgttactcatacggtcgctcgccaggggatggc

SEQ ID NO: 45 - protein, 160 aa, Artificial Sequence
  Synthetic
GRDSGYSPPGRDGYSSYSPGDSGRSRSGPDGYSRDSYGGPSDGYPRSGGDSSPGRYGPYRDGSSGPGSRDYSRGSGSYDPYPRGDSGSPYRGGDSSDSGGYSPRYDGPGRSSYGSPDSRGRDSGPGYSDPGYSRSGSDPGYGSRYSDPSRGGRSPDGYGS

SEQ ID NO: 46 - DNA, 480 bp, Artificial Sequence
  Synthetic
ggccgcgactctggttattcgccccctggtcgcgatggatattcctcatacagccccggcgatagcggacgtagtcgttctggaccagacggctactctcgcgattcgtatggcgggccaagtgatggttacccccgcagcggaggcgattcatcccccggacgctacgggccgtatcgtgatggaagcagtggcccgggttcacgcgactattcacgcggtagtgggagttatgatccgtacccgcgtggagattcggggagcccttatcgcggcggcgatagctccgattccggtggatattcccctcgctacgacggaccaggccgcagctcttatgggtcccctgatagtcgcgggcgcgatagcgggcctggctattcagatccagggtacagtcgctcggggagtgatccaggttacggcagccgctattctgatccgtcgcgtggaggtcgttcacctgacggatacgggtcc

SEQ ID NO: 47 - protein, 160 aa, Artificial Sequence
  Synthetic
GDRSPYSGGSRPGSDYGSDSRYPGGPYRGSDSSGRSYPDGDGSGPYRSSSDRPYGGSSYGGDRPGYDRSGPSGGDYSPRSGYRSPSDGSGPYDGRSYGPGSRSDSDGRSGPYSDGYPSGRDPRSGGYSRSYDGSGPYRPSDSGGPDGSRYSGSRDSGYPG

SEQ ID NO: 48 - DNA, 480 bp, Artificial Sequence
  Synthetic
ggcgatcgttcaccttacagtggaggttcacgtcctgggtcggattatggtagtgactcacgctatccaggaggcccttatcgtggatcagattctagtggtcgttcttatccagatggtgatggcagcggtccgtatcgctcgtcgtccgaccgtccatacggggggtcatcctatggcggtgaccgccccggctacgaccgcagtggacccagtggtggtgattattcacctcgttccgggtaccgcagtcccagtgatggatcagggccttatgatggccgcagttacggtccgggcagtcgtagtgacagcgatggccgctccggaccttactcggatggctacccttctggccgtgacccgcgctctggtggttattcacgcagttatgatggttcaggtccgtatcgtccatctgattctggagggcctgatggcagccgttacagcggttcgcgcgattcagggtaccctgga

SEQ ID NO: 49 - protein, 160 aa, Artificial Sequence
  Synthetic
GPYSDRGSGDSGSYPRPRSGGDSYPGSYDGRSYSGRPDSGGSDGRYPSRGGPYSDSRPSGYGSDDPYSGGSRGRGSPSDYDGSYRGPSPGDYSSGRGGSRYDPSDRSPSGYGPDGSGYRSSRDSGYGPGDGRPSYSDPGGSYSRRSSDGPGYDGSRPYGS

SEQ ID NO: 50 - DNA, 480 bp, Artificial Sequence
  Synthetic
ggcccgtatagtgaccgtggctcgggagatagcggatcgtatccgcgcccacgtagtggtggcgattcatatccaggatcttatgatgggcgctcatattccggacgtccagattcgggtgggagcgatggacgctaccctagtcgtggtggtccctacagtgattcccgtccctccggctacggcagcgacgacccatactcaggaggttcccgcggtcgcggaagcccgtccgattacgatggtagctaccgcggtccttcacccggagattactcaagcggtcgtgggggtagtcgttatgaccccagtgatcgctcacccagcggctacggaccagatgggagtggctatcgttcatcgcgcgactctggctatgggcctggggacggccgtccttcttactcagatcctggaggcagctactcacgtcgttcaagcgatggcccaggatacgacggctcacgtccttatggtagc

SEQ ID NO: 51 - protein, 160 aa, Artificial Sequence
  Synthetic
GRDSGYSPPSRGYSDGGDSYSPGRPSGRYSGDGSGPDSRYSDSGGRYPPGSYSDGRSPRSYGDGSYPDGRGSPYRSGGSDPDYGSRSGGRGSSPDYGYDSPSRGRPGSDYSGGDPYSGRSSGRPDSGYYDRSSGPGYGDSRPGSGDSGPYRSPSRSGYGD

SEQ ID NO: 52 - DNA, 480 bp, Artificial Sequence
  Synthetic
ggccgtgacagcggctactcacctccgagtcgtggctatagcgacggtggcgattcatactcaccgggccgtccttccggtcgttactctggggacggttctggaccagactctcgctactcggacagtggtggacgctacccgccgggttcgtattcagatggccgctcaccccgttcgtatggagacggatcttaccccgatggacgtggatcaccataccgctcaggaggatctgatcccgattacggcagtcgttcaggcggacgcggttctagccctgattacggctacgactccccatcacgtggtcgtcccggtagtgactactctggaggggacccgtatagcggacgctcatcagggcgtcctgactccggttattacgaccgcagttccggtccaggttatggtgattctcgccccggcagcggtgacagcggtccgtatcgttcaccctcccgtagtggatatggagac

SEQ ID NO: 53 - protein, 160 aa, Artificial Sequence
  Synthetic
GGSPDYSRPYRGSDGSDSGGYRPSGSGDSRYPSRDYSPGGYDSPGSRGRGSYDSGPPSRSYGGDDGRPGSSYSDRSYGGPGGDSYRSPYDGSSGPRRSGGYSPDGDYSGPSRGDSGYSPRRSGSGDYPYSRGDSGPDGRPSGYSYDGSRPSGSGYPGDRS

SEQ ID NO: 54 - DNA, 480 bp, Artificial Sequence
  Synthetic
ggaagcccggactatagccgcccataccgtggatctgacggttccgactccggcggataccgcccaagtggttcgggcgacagccgctatccgtcacgtgattatagtccggggggatacgattcccctggctctcgtggccgtggaagttacgatagtgggccaccgtcacgttcttacggcggcgatgatggtcgccccggctcatcgtacagcgaccgttcatatggcggaccaggaggcgattcataccgctctccatatgacggttcgtcaggcccacgtcgcagtgggggatatagtccggacggtgactactctgggcctagccgtggggatagcggatattctccacgccgtagtgggtcgggagattacccctactcgcgcggggattctggtcccgatggtcgtcccagtggctacagttacgatggttcgcgcccgagtggtagcgggtatccaggagaccgctcgggc

SEQ ID NO: 55 - protein, 160 aa, Artificial Sequence
  Synthetic
GGDSPGYYDGYRRDSSRPDGGSYPGSGRSPSSGRPSSYGDSPRDGSSGRSPSGGGYSSSDGSYYDGRPRYGPSDPRDGGYRGSGDDPSSSPSYDYYDRSRGPYPSGSSSGGRYPGDGRGGYDYGGYPRDYRDPSSGRDGGYSSPSRGGSPSSPSRSGGGD

SEQ ID NO: 56 - DNA, 480 bp, Artificial Sequence
  Synthetic
ggagattccccgggttactatgatggttatcgtcgtgatagcagccgcccagatgggggctcatatcccggctccgggcgctctccgtcgtcaggacgcccgtcgagttacggtgactctccccgcgacggctcgtccgggcgtagccctagtggggggggatactcgagttctgatggtagctattatgatggacgccctcgttacggtccttcggacccccgcgatggtggataccgtggctctggagacgacccttcttcctcacccagctacgattattacgatcgttcccgtggcccatacccaagtgggtcgtcgtccggcggtcgttatcccggtgatggtcgtggtggttatgattacggagggtatccacgcgattatcgcgatccaagctctggtcgcgatggcgggtattcgtcccccagccgtggcgggtccccctcatctccatcacgctctgggggtggagatggc

SEQ ID NO: 57 - protein, 160 aa, Artificial Sequence
  Synthetic
GRSYRGDGYGSSSYYYGGPDPSGGPSSPPSRDGSDGRRDSGGGPSSSDYPSSPSGGDYSYGPDGSRYDGSRRRDYPRSGGSRGYSDYGRPYGGSGGDGGPYSPPRDSSSYGPDRSSDSGRSRPSGSSYPYSDSGYPRPGGGPRRDSSGDGGYDYRSGGDS

SEQ ID NO: 58 - DNA, 480 bp, Artificial Sequence
  Synthetic
ggccgctcttaccgtggggacggttacgggtcctcgtcctactactatgggggtcccgatccctcgggaggtcccagctctcccccctcacgcgacggatcggacgggcgccgtgacagtggcggggggccttcttctagtgactacccgtccagtccttctggcggcgactattcttacggcccagatgggtcacgctacgacgggagtcgtcgtcgtgattacccccgctcaggaggctcacgcggttacagtgactatgggcgtccatatggcggcagcggtggagatggggggccatattctccaccccgcgactccagctcttatgggcctgaccgtagctccgattctggtcgttctcgtccctcgggtagttcttacccctattcagatagtggctatccacgtcctggcgggggacctcgccgtgacagctcaggagacggaggctacgattatcgttcggggggtgactcc

SEQ ID NO: 59 - protein, 160 aa, Artificial Sequence
  Synthetic
GDYYRGYPSPRGSPGPGSSGSDDSRYPGDSGDSRRYSSGGGYSRGSDRRYDSSSGGGPYPYDRDSRSGYDGPSSSGPGGPSDSPGGRPSGSYPDDGGGRSDSSPYRGYDSRSSPGYYGGRSPYRDSPRDYGPPSDDRGSDGGRYPSGSSGGGRSGYGSYS

SEQ ID NO: 60 - DNA, 480 bp, Artificial Sequence
  Synthetic
ggcgactattatcgcgggtacccatctccccgcgggtctcccgggcctgggtcaagcggcagcgatgattcacgttacccaggtgattctggcgattctcgtcgttacagttccggaggtggttattctcgcggatcggatcgtcgttatgattcgtcatccggaggcggtccctacccttatgaccgcgacagtcgtagcgggtatgatgggccgtccagttctggccctggcggacctagtgactcacccggtgggcgtcccagcggttcatacccggatgatggggggggacgttccgattcgagtccctatcgcggttacgatagtcgtagctcgcctgggtactatggtggacgttccccttaccgtgactcaccccgtgattacggcccaccatccgatgatcgcggatcagacggtggccgttacccctcaggttcttcgggagggggtcgctctgggtacggttcttatagc

SEQ ID NO: 61 - protein, 160 aa, Artificial Sequence
  Synthetic
GYSGSSPYRPGSSRPSDGYGPGDGGPSRDSRSYSYDDGGRGGSPDPGGSRSRRPGDGGYSYSSPYGSGYDGRYRDSSDPSDDGRPPRRGYDGPSYGGGRYGGSRPSDYSPSSDSSSGYSGGDGGSSRDGYYYDRGSSGPSYSDGPRSSGDPYRPRGPGSS

SEQ ID NO: 62 - DNA, 480 bp, Artificial Sequence
  Synthetic
ggctactcgggcagttcaccctaccgccccggctcttcccgtccatccgatggatatggtccgggcgatggtggtccaagccgcgatagtcgttcgtactcatatgacgacggggggcgcggggggagtcccgatccgggaggatcacgctcccgccgccctggagatggagggtacagctattctagtccctatggctcgggatacgacggccgctaccgcgactcgtcagatccctcggacgacggacgtccaccacgccgcgggtacgatgggccgtcttacggcggtggacgttatgggggctcccgtccttctgattactccccttcttctgatagttcgtctggctactcaggtggggacggaggaagcagccgcgatggatattactacgaccgcggttctagtggaccatcttactcagatggtccgcgttccagtggtgacccgtaccgccctcgtgggcctggttcttcg

SEQ ID NO: 63 - protein, 160 aa, Artificial Sequence
  Synthetic
GYDSSYGGDSPYPSGGSRGYRGPPDRRRGGDSSSSSPDGYRPSDGPGSDRYDSSGGSSYSRGYRSPSPGDDGYSGRGPGYDGRYYPGRSRSGSSPGSGPGSSYPRSDDGDGSPYSYDGRGSPSPSGYGPGSRGDGRDYRDGGDSSSDGPGRRPYSYSGSY

SEQ ID NO: 64 - DNA, 480 bp, Artificial Sequence
  Synthetic
ggctacgatagctcctatggtggcgattctccgtatcctagtggtggtagtcgcgggtatcgtggtcctcctgaccgccgccgtgggggagattcatcctcaagttccccagacggataccgcccgagtgatggcccagggagtgaccgctacgactctagcggcgggtcgtcttacagtcgcggatatcgtagtccatcacccggcgatgacggatactcggggcgtgggcctggttacgatggtcgttactatccaggacgttcgcgttctgggtcatcgcctggttccgggccaggttcgtcctaccctcgtagcgacgacggtgacggctccccatacagctacgacggacgtgggagcccatctccttctgggtatggtcccggtagtcgtggagacggtcgtgactatcgtgatggaggcgacagtagttctgatggaccgggtcgtcgcccgtactcgtactcgggctcttac

SEQ ID NO: 65 - protein, 160 aa, Artificial Sequence
  Synthetic
GDGSYPRSPSPSYGGGSGPSRRRDSYRGDGDGYYGDSSSPGDSGGRGSDDGYPSSSDSYSRSRGGSSPDGYPYPGPRRYGYGPGSGYYSSSDDGSRDSPRYRGGPYRGPSDGGRSGSDSPGGSYSSGRGSDSYGGGDRPRPGRDDYGPYPSPSDSYSSRG

SEQ ID NO: 66 - DNA, 480 bp, Artificial Sequence
  Synthetic
ggcgatgggagttatccacgcagtccttcgcccagctacggtggcggaagcgggccaagtcgccgtcgtgactcatatcgtggtgatggcgacggttactacggtgattcatcttcgccaggggacagtggcgggcgtgggagtgatgacggctacccttcttccagtgattcgtattcgcgttcccgtggcggttcatcgccggatggatatccttaccccggaccgcgccgttacggatatgggcctggcagcggttattattcttctagcgatgacgggtctcgcgattcgccccgttaccgtggaggaccctaccgtggaccgtctgatgggggacgttccggcagtgatagccctggcggttcgtattcatctggacgtggcagtgacagctacggcggcggagatcgtcctcgccctggtcgcgacgactatggaccgtatccatcaccctcggattcgtattcatcacgcggg

SEQ ID NO: 67 - protein, 160 aa, Artificial Sequence; identical to SEQ ID NO: 43
  Synthetic

SEQ ID NO: 68 - DNA, 480 bp, Artificial Sequence
  Synthetic
ggcggcaggtctgactcttatccaggaggtcgacctagtgattatagctccgacaggccatcttatggcggcggatatcgcagcggttcaccggattctcctgacggttatcgcagtggacgcggatacgatggcccgagttcccgcggaagcccggggtacgattcctatcctggctcagatggaagtcgcgactaccgaccaggtagtggctcacctcgaggtggcgattatagttcttcacctggagactcacgatacggcgggggtagatatgatccctcttccccatccgacagcagaggagggtattccccttatgggtcccgggatggctcttcacccggatacggaagagaccgaagtgatagcggtgggtacccccctgatcggtccagcgggggatacgatgggtacagcggacggtcacctagcggtagcgatggccccagatactcctacggaagatccccaggtgat

SEQ ID NO: 69 - protein, 160 aa, Artificial Sequence; identical to SEQ ID NO: 45
  Synthetic

SEQ ID NO: 70 - DNA, 480 bp, Artificial Sequence
  Synthetic
ggccgggattccggatacagtcccccaggcagagatgggtactcaagctatagtccaggggatagcggtcgatctcgaagcggcccagatggctattcccgcgatagttacggcggtccgagcgacggttacccccgaagcggcggtgattctagtccggggcgctacgggccatatcgggacgggtctagtggtcctggatcacgggattattctcgagggtccggttcctatgatccatatcccaggggggactccggtagtccctatagggggggcgacagttctgatagtggagggtattccccaaggtacgatggtcctggcagatcaagctatggaagtcccgacagtcgaggacgcgactcagggcctggctactctgatccgggctacagcaggagtggatctgatcctggctacggatcaagatattccgacccgtcacgcggtggtagaagtcccgacgggtatggttcc

SEQ ID NO: 71 - protein, 160 aa, Artificial Sequence
  Synthetic
GRGDSPYSGRGDSPYSGRGDSPYSGRGDQPYQGRGDSPYSGRGDSPYSGRGDSPYSGRGDQPYQGRGDSPYSGRGDSPYSGRGDSPYSGRGDQPYQGRGDSPYSGRGDSPYSGRGDSPYSGRGDQPYQGRGDSPYSGRGDSPYSGRGDSPYSGRGDQPYQ

SEQ ID NO: 72 - DNA, 480 bp, Artificial Sequence
  Synthetic
ggccgtggggattctccttatagtggtcgaggggatagtccgtactcaggacggggtgatagtccttattcgggacgcggcgatcaaccataccaaggtcgtggcgattcaccctactcgggtcggggagattctccatactctgggagaggcgactctccatatagtgggcgcggtgatcagccatatcaagggaggggtgacagcccatatagcgggcgtggtgattcaccatattcaggccgtggagatagtccctattctggcagaggggaccagccttatcagggaagaggtgatagcccttattccggtcgcggagattccccctatagtggacgaggtgattctccctacagtggccgaggtgaccaaccttaccagggtagaggtgactcaccttattcaggtaggggagacagtccttactccggcaggggcgatagtccatattctggaaggggagatcaaccgtatcaa

SEQ ID NO: 73 - protein, 160 aa, Artificial Sequence
  Synthetic
GRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQ

SEQ ID NO: 74 - DNA, 480 bp, Artificial Sequence
  Synthetic
gggcgcggcgactcaccatattccggtcggggtgaccaaccttaccaaggtcgcggtgatagcccatactctggcaggggagaccaaccataccagggtcgaggtgacagtccatatagtgggagaggggatcaaccctatcagggtagaggcgattccccctatagcgggcgtggtgatcaaccttatcagggaaggggtgatagtccctattcaggacgtggcgatcaaccatatcaagggaggggcgatagtccgtattctgggcgcggagatcagccctatcaaggtaggggagattctccttatagtggacggggagatcaaccgtatcaaggcagaggtgactctccatacagtggtcgtggggatcagccatatcagggccgtggagatagtccttactcaggccgaggggaccagccttatcaaggaagaggtgattcaccttattcgggacgaggtgatcagccgtaccaa

SEQ ID NO: 75 - protein, 160 aa, Artificial Sequence
  Synthetic
GRGDQPYQGRGDQPYQGRGDQPYQGRGDSPYSGRGDQPYQGRGDQPYQGRGDQPYQGRGDSPYSGRGDQPYQGRGDQPYQGRGDQPYQGRGDSPYSGRGDQPYQGRGDQPYQGRGDQPYQGRGDSPYSGRGDQPYQGRGDQPYQGRGDQPYQGRGDSPYS

SEQ ID NO: 76 - DNA, 480 bp, Artificial Sequence
  Synthetic
ggccggggtgaccaaccgtatcagggccgtggagaccagccataccagggtcgcggagatcaaccctaccaaggccgaggtgattctccatattccggtcgtggggaccaaccataccaaggtcggggagatcagccgtatcaaggacgcggggatcagccctatcagggaaggggagattcaccttattctgggcgcggtgatcagccttaccagggacgtggcgaccaaccctatcaaggtaggggagaccaaccttaccaagggcgtggtgactctccttatagtggacgaggggatcaaccatatcagggtagaggggaccagccttatcaagggagaggtgatcaaccttatcaggggaggggtgatagtccttactcaggtcgaggtgaccagccctaccagggcagaggcgatcagccatatcaaggcaggggcgatcaaccgtaccaaggaagaggtgacagtccatacagc

SEQ ID NO: 77 - protein, 160 aa, Artificial Sequence
  Synthetic
GRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVS

SEQ ID NO: 78 - DNA, 480 bp, Artificial Sequence
  Synthetic
ggacgaggggactcaccttatagtgggcgcggcgactctccatattccggtcggggagattcaccatatagtggccgtggcgatagtcctgtgtctggtaggggagactctccgtattcaggacgcggtgattcaccctacagtggacggggtgattctccttattcgggtcgtggggattctccagtttcaggtagaggtgatagcccttattcaggccgaggtgacagtccttactccggcaggggtgactccccatactcgggacgtggtgatagtccagtatcaggaaggggagatagtccctactctgggagaggggatagtccgtactcaggtcgaggtgattccccctattctggaagaggtgactctcctgtatctggcagaggcgacagtccatacagcgggcgtggagattctccctatagtggtcgcggagacagcccatattcagggaggggcgattcacctgttagt

SEQ ID NO: 79 - protein, 160 aa, Artificial Sequence
  Synthetic
GRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVS

SEQ ID NO: 80 - DNA, 480 bp, Artificial Sequence
  Synthetic
ggacgcggagattctccatactctggtcgaggggatagtccggtctcaggaagaggcgatagtccatattcaggacgaggtgacagtccagtatctggcagaggggactctccgtattccggcaggggagacagccctgtttcaggccgaggtgattctccttactcaggtcggggagatagtcctgtgtctggtaggggtgactccccctattctggaaggggagattcacctgtaagtgggcgtggtgattcaccctactcgggtcgtggagactctccagttagtggtcgcggtgattccccatatagcgggcgcggcgattctcccgtttctgggaggggcgacagtccttattcgggacgtggcgactctcctgtatcagggagaggtgatagtccctacagtggccgtggggattcaccagtgtcaggtagaggtgactcaccttatagtggacggggtgatagcccagtcagt

SEQ ID NO: 81 - protein, 160 aa, Artificial Sequence
  Synthetic
GRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYS

SEQ ID NO: 82 - DNA, 480 bp, Artificial Sequence
  Synthetic
ggacggggtgatagccctgtatctggaaggggagattcaccagtcagtggacgcggtgattcccctgttagtggccgaggtgatagtccttactcaggacgtggcgactctccagtatcgggacgaggggacagcccagtaagcggcaggggcgattctcctgtaagtgggagaggtgattctccatatagtggtcgcggagattctcccgtttcaggtaggggagactcgcctgtctcaggtcgaggtgacagtcctgtgtctggtcggggagatagtccctattctggtagaggggatagtccggtttcgggtcgtggagattcgccggtctctggcagaggtgactcacctgtttctgggcgtggtgattcaccttattcagggaggggtgactccccagtgtcaggccgtggggattcacccgtatcaggaagaggcgatagtccagtttccgggcgcggggactctccttatagt

SEQ ID NO: 83 - protein, 160 aa, Artificial Sequence
  Synthetic
GRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVS

SEQ ID NO: 84 - DNA, 480 bp, Artificial Sequence
  Synthetic
ggacgaggtgattcgcctgttagtggccgaggggatagtcccgtatctgggaggggcgatagtccggtctcaggtaggggagatagtcctgtatcagggagaggcgactctccagttagtggtcgaggtgacagtccagtgagcggcaggggtgatagtccagtatcaggacggggagattcccctgtgtcaggccgtggcgattctcccgtgtctggaagaggtgatagccctgtctctggtcgcggggactctcctgtgagtggacgcggtgattcaccggtatcaggtcggggtgactcgccggtttcaggaaggggagactccccagtaagcggacgtggagattctcctgtttccgggcgcggagattcaccagtttctggcagaggtgactcacctgtaagtgggcgtggggattcacccgtttcgggtcgtggtgattctccagtcagtggtagaggggacagcccagtgtcg

SEQ ID NO: 85 - protein, 160 aa, Artificial Sequence
  Synthetic
GRGDSPYSGRGDSPYSGRGDSPYSGRGDSPASGRGDSPYSGRGDSPYSGRGDSPYSGRGDSPASGRGDSPYSGRGDSPYSGRGDSPYSGRGDSPASGRGDSPYSGRGDSPYSGRGDSPYSGRGDSPASGRGDSPYSGRGDSPYSGRGDSPYSGRGDSPAS

SEQ ID NO: 86 - DNA, 480 bp, Artificial Sequence
  Synthetic
ggacggggtgactctccgtattccggtcgaggtgatagcccttattcgggtcggggagattcaccctactcgggacgcggcgattctcctgcttcaggtcgtggcgatagtccttatagtggtagaggcgactctccttactccggcagaggtgactccccatactcaggtaggggagatagtccagcttctggccgtggagacagcccatattcaggacgtggtgatagtccgtactctggtcgcggtgattcaccttattcaggccgaggggactcacctgcatctgggcgcggagattctccctattcagggaggggtgattccccctatagtggcaggggagactctccatattctggaagaggtgattctccagcatcaggaaggggcgacagtccatacagcgggcgtggggattcaccatatagtgggagaggggatagtccctacagtggacgaggtgacagtcctgcgagc

SEQ ID NO: 87 - protein, 160 aa, Artificial Sequence
  Synthetic
GRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPAS

SEQ ID NO: 88 - DNA, 480 bp, Artificial Sequence
  Synthetic
ggacggggagactctccatattcaggacgaggtgattcccctgcctcaggaagaggcgactctccgtattccggtcgtggagattctcccgcatcaggtagaggggactctccttacagtggacgtggcgatagtccggcttcaggccgtggggacagtccttatagcggacgcggtgattcaccagcatctggtagaggtgactccccctattctggcagaggggattctccagcgtcaggtcgcggcgattcaccctatagtggtcggggtgatagcccagctagtgggcgcggagatagtccctactccgggcgtggtgattctcctgcgagtggtaggggcgacagcccttactcagggaggggtgactcacctgcaagtggccgaggggatagtccatactctgggagaggtgatagtcctgcttctggaaggggagattcaccttattcgggtcgaggtgacagtccagccagt

SEQ ID NO: 89 - protein, 160 aa, Artificial Sequence
  Synthetic
GRGDSPYSGRGDSPYSGRGDSPYSGRGDSPISGRGDSPYSGRGDSPYSGRGDSPYSGRGDSPISGRGDSPYSGRGDSPYSGRGDSPYSGRGDSPISGRGDSPYSGRGDSPYSGRGDSPYSGRGDSPISGRGDSPYSGRGDSPYSGRGDSPYSGRGDSPIS

SEQ ID NO: 90 - DNA, 480 bp, Artificial Sequence
  Synthetic
ggacgtggagattcaccctactctggcagaggcgactctccatacagtggaagaggtgactccccatactcgggtcgcggcgatagtccaatatcaggtcgaggggatagtccgtattctgggagaggtgatagcccatattccggtcgtggtgattccccctatagtggtcggggagattctccaatttcagggaggggcgacagcccttattctggaaggggagatagtccttactccggcaggggagactctccttattcaggtagaggggactctccgataagtgggcgcggagacagtccatattcaggacggggtgactcaccttatagtggccgtggcgattcaccgtactcaggccgaggtgacagtcctatatctggtaggggtgattctccctattcgggacgaggtgattcaccatatagtggacgcggtgatagtccctacagcgggcgtggggattctcctattagt

SEQ ID NO: 91 - protein, 160 aa, Artificial Sequence
  Synthetic
GRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPIS

SEQ ID NO: 92 - DNA, 480 bp, Artificial Sequence
  Synthetic
ggacgcggcgattctccctattctggccgtggtgattcacctattagtgggcgcggtgatagcccttacagtggtaggggcgactcccccatctctggaaggggagatagtccttactcagggaggggtgattccccaataagtggacgtggagattctccttatagcgggcgtggcgacagtcctatatcaggccgaggggatagtccgtattccggtcgcggagactctccgatatccggcaggggagacagcccatatagtggcagaggggactctcctatttcaggacggggagattcaccctactcgggtcgtggggattcaccaatctcaggaagaggtgactcaccttattcgggacgaggtgatagtcccatttctggtagaggtgattctccatactctggtcgaggtgacagtccaatatctgggagaggcgatagtccatattcaggtcggggtgactctccaatttcc

SEQ ID NO: 93 - protein, 160 aa, Artificial Sequence
  Synthetic
GRGDSPYSGRGDSPYSGRGDSPYSGRGDSPMSGRGDSPYSGRGDSPYSGRGDSPYSGRGDSPMSGRGDSPYSGRGDSPYSGRGDSPYSGRGDSPMSGRGDSPYSGRGDSPYSGRGDSPYSGRGDSPMSGRGDSPYSGRGDSPYSGRGDSPYSGRGDSPMS

SEQ ID NO: 94 - DNA, 480 bp, Artificial Sequence
  Synthetic
ggccgcggtgattctccctactcaggtagaggggactccccatattcaggacggggtgactcaccttattctggcagaggtgacagtcctatgagtggccgaggtgattccccctatagtggacgtggtgatagtccctattcaggccgtggggattcaccatattcgggacgaggggatagtccaatgtctggtcgtggagactctccgtactccggcaggggagattcaccctacagtggaagaggtgatagcccttattcaggtcgaggtgactctccaatgagcgggcgtggcgacagtccatattctgggcgcggcgatagtccttactcgggtcgcggagattctccatatagtgggagaggcgactctcctatgtcagggaggggcgattctccttatagtggtaggggagacagcccatactctggaaggggtgattcaccgtattccggtcggggagatagtccgatgtcc

SEQ ID NO: 95 - protein, 160 aa, Artificial Sequence
  Synthetic
GRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMS

SEQ ID NO: 96 - DNA, 480 bp, Artificial Sequence
  Synthetic
ggccgtggagactctccttatagtggaagaggggatagtccaatgagcgggcgtggggattcaccctattccggtcgaggtgatagtccgatgtccggcaggggagacagtccatactcaggccgtggtgattcacctatgtctggaaggggcgattctccatatagtggccgaggtgactcccccatgagtgggcgcggagattcaccatacagtggtcgcggtgattccccaatgagtggacgcggcgacagtccttactctggtagaggtgacagccctatgtcaggacggggtgatagcccatattcagggaggggtgactctccaatgtcaggtcgtggcgactcaccttattctgggagaggcgatagtcccatgtctggcagaggtgattctccctatagtggtaggggagatagtcctatgtcgggacgaggggactctccgtattcgggtcggggagattctcctatgagt

SEQ ID NO: 97 - protein, 160 aa, Artificial Sequence
  Synthetic
GRGDSPYSGRGDSPYSGRGDSPYSGRGDSPHSGRGDSPYSGRGDSPYSGRGDSPYSGRGDSPHSGRGDSPYSGRGDSPYSGRGDSPYSGRGDSPHSGRGDSPYSGRGDSPYSGRGDSPYSGRGDSPHSGRGDSPYSGRGDSPYSGRGDSPYSGRGDSPHS

SEQ ID NO: 98 - DNA, 480 bp, Artificial Sequence
  Synthetic
ggacgtggggattcaccatattcaggtcgcggagattctccctatagtggcaggggcgactctccttattccggcagaggtgactcacctcattcaggacggggagattcaccttattcagggaggggtgattccccatattctggccgtggtgatagcccttatagtggaagaggcgacagtccacattctggacgaggggatagtccctacagtggtcgtggcgattcaccgtactccggtcggggtgactctccgtattctggtaggggagatagtccgcattcgggtcgaggtgatagtccatatagtgggagaggggactctccatacagcgggcgtggagactccccctattcaggccgaggtgacagtcctcatagtggtagaggtgattcaccctactcgggacgcggcgatagtccttactcaggaaggggagacagcccatactctgggcgcggtgattctccacactct

SEQ ID NO: 99 - protein, 160 aa, Artificial Sequence
  Synthetic
GRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHS

SEQ ID NO: 100 - DNA, 480 bp, Artificial Sequence
  Synthetic
ggacggggagattcaccctatagtggtaggggagatagtccgcactctggccgtggtgattctccttactcaggacgcggggattcacctcattcaggccgaggggatagtccatacagtggtcgaggtgacagtccacactccgggcgcggtgattccccttattcaggtcgtggagacagccctcatagcggtcggggtgatagtccctattcaggaagaggggactcgccgcattcgggacgtggggacagtccttatagtggaaggggcgattctccacattccggcagaggtgactcaccatattctggacgaggtgatagcccacatagtggcaggggagactcaccttattctgggcgtggcgatagtcctcactcgggtcgcggagattctccgtattcagggaggggtgactccccccattctggtagaggcgactctccatatagtgggagaggtgattcaccacacagt

SEQ ID NO: 101 - protein, 160 aa, Artificial Sequence
  Synthetic
GRGDSPHSGRGDSPHSGRGDSPHSGRGDSPYSGRGDSPHSGRGDSPHSGRGDSPHSGRGDSPYSGRGDSPHSGRGDSPHSGRGDSPHSGRGDSPYSGRGDSPHSGRGDSPHSGRGDSPHSGRGDSPYSGRGDSPHSGRGDSPHSGRGDSPHSGRGDSPYS

SEQ ID NO: 102 - DNA, 480 bp, Artificial Sequence
  Synthetic
ggacgaggtgattcccctcatagtggccgaggtgactccccccatagtggtaggggtgactcacctcactccgggcgcggggattctccttactcaggaagaggggacagcccacattctggcaggggagacagtccacatagtgggagaggtgacagtcctcattcgggacgtggagattctccgtatagtggaaggggagattcacctcattcaggccgtggcgattctccacattcaggacggggtgatagccctcattctgggcgtggtgattctccctattctggtagaggcgactcgccgcactctggacgcggtgattcaccacactcgggtcgtggggatagtccacacagcggtcgcggagactctccatattcagggaggggcgatagtccccattccggcagaggtgatagtcctcacagtggtcggggagatagtccgcattcaggtcgaggggattcaccctatagt

SEQ ID NO: 103 - protein, 160 aa, Artificial Sequence
  Synthetic
GRGDSPYSGRGDSPYSGRGDSPYSGKGDSPYSGRGDSPYSGRGDSPYSGRGDSPYSGKGDSPYSGRGDSPYSGRGDSPYSGRGDSPYSGKGDSPYSGRGDSPYSGRGDSPYSGRGDSPYSGKGDSPYSGRGDSPYSGRGDSPYSGRGDSPYSGKGDSPYS

SEQ ID NO: 104 - DNA, 480 bp, Artificial Sequence
  Synthetic
ggacgtggtgatagtccgtactcaggtcgaggtgatagcccatattctggaaggggagactctccgtatagtggaaaaggggactctccttatagtggccgaggtgacagtccatacagtggaagaggggattcaccctattctggcagaggtgactctccatatagtgggaagggtgattctccttactctgggcgtggagacagcccttattccggtcggggtgactcaccttattcaggccgtggcgatagtccctattcagggaaaggtgattcaccgtattcaggacggggagattcaccatattcgggacgcggagattctccctactcgggtcgtggggatagtccatattcaggtaagggcgactccccctatagtggtaggggcgattctccatactccggcaggggagatagtccttattctggtcgcggtgattccccatatagcgggaaaggcgacagtccttacagt

SEQ ID NO: 105 - protein, 160 aa, Artificial Sequence
  Synthetic
GRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYS

SEQ ID NO: 106 - DNA, 480 bp, Artificial Sequence
  Synthetic
ggccgcggagattccccgtatagcgggaagggcgattctccttattctggcagaggggatagtccttactcaggaaaaggtgactccccatattccggtcgtggagattcaccgtattcaggcaagggggacagtccttatagtggccgtggtgattcaccatattcaggtaaaggcgactcaccttattcaggacgtggggattctccctactccggcaaaggtgattctccatatagtggtcgaggtgatagcccatacagtggtaagggagatagtccgtatagtggaagaggcgatagtccatattctgggaaaggagacagtccatactcgggcaggggagactctccttacagcggtaaaggggactcgccgtactctggtagaggtgacagcccctattctggaaagggtgatagtccctatagtgggaggggtgattcgccatattcgggtaaaggagattctccgtacagt

SEQ ID NO: 107 - protein, 160 aa, Artificial Sequence
  Synthetic
GRGDSPYSGRGDSPYSGRGDSPYSGRGESPYSGRGDSPYSGRGDSPYSGRGDSPYSGRGESPYSGRGDSPYSGRGDSPYSGRGDSPYSGRGESPYSGRGDSPYSGRGDSPYSGRGDSPYSGRGESPYSGRGDSPYSGRGDSPYSGRGDSPYSGRGESPYS

SEQ ID NO: 108 - DNA, 480 bp, Artificial Sequence
  Synthetic
ggacgaggggatagtccgtattcagggcgcggagattctccgtacagcggtcgcggtgatagcccttattctgggagaggtgagagtccttatagtggcagaggtgactctccatatagtggtaggggagatagtccctattctggaaggggtgattctccatactctggtagaggcgaatctccttactccggcaggggcgattctccttattccggtagaggggacagcccgtatagtgggcgtggtgattccccctatagtggacgcggcgagtctccctactcaggtcgtggcgactcaccgtactcgggtcgaggtgattcaccctattcaggccgtggagattcaccatattctggtcggggagaaagcccatattcaggacgtggggattcaccttattcaggtagaggtgatagtccatattcgggacggggtgacagtccttacagtggaagaggtgaaagtccatacagt

SEQ ID NO: 109 - protein, 160 aa, Artificial Sequence
  Synthetic
GRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYS

SEQ ID NO: 110 - DNA, 480 bp, Artificial Sequence
  Synthetic
ggacggggtgattcaccatattcaggccgtggtgagtctccctattctggtcggggagatagtccgtactcaggtcgtggagagagtccatacagcggtagaggggacagcccatattcgggacgcggtgaatctccttacagtggaagaggtgattctccatatagtggcaggggtgaatccccctatagtggacgaggtgatagtccatattctgggcgcggcgagtcaccctattcaggacgtggggatagtccttattcagggcgtggcgaatctccatactctggtaggggcgactctccttatagtgggagaggtgaaagtccttactccggtcgcggagattctccgtatagtggtagaggtgagagcccgtattcaggtagaggcgattcaccttattctggaaggggagaaagcccttattccggcagaggtgacagtccctactcgggtcgaggggaatcaccgtacagt

SEQ ID NO: 111 - protein, 160 aa, Artificial Sequence
  Synthetic
GRPDSPYSGRGDSPYSGRGDSPYSGRGDSPYSPRGDSPYSGRGDSPYSGRGDSPYSGRGDSPYSGRPDSPYSGRGDSPYSGRGDSPYSGRGDSPYSPRGDSPYSGRGDSPYSGRGDSPYSGRGDSPYSGRPDSPYSGRGDSPYSGRGDSPYSGRGDSPYS

SEQ ID NO: 112 - DNA, 480 bp, Artificial Sequence
  Synthetic
ggcaggccagattcaccctattctgggagaggtgacagtccttacagtggtcgcggagactctccatattcagggcgtggcgactcgccgtatagtcctagaggcgattccccctattcaggaagaggtgattcaccatattccgggaggggtgattctccatacagcggtcgaggtgatagcccatattctggtaggcccgattctccttattcgggtcgtggggacagcccttattctggacgcggtgatagtccttatagtgggcgcggggattcaccgtactctccgagaggcgattctccctactccggcaggggagattctccgtattcaggccgtggtgactcaccttactcgggacgaggggatagtccatatagtggtagaccagactccccatactcaggacgtggagatagtccctatagtggaaggggcgattcaccttattcaggtagaggcgatagtccgtacagt

SEQ ID NO: 113 - protein, 160 aa, Artificial Sequence
  Synthetic
GRPDSPYSGRGDSPYSPRGDSPYSGRGDSPYSGRPDSPYSGRGDSPYSPRGDSPYSGRGDSPYSGRPDSPYSGRGDSPYSPRGDSPYSGRGDSPYSGRPDSPYSGRGDSPYSPRGDSPYSGRGDSPYSGRPDSPYSGRGDSPYSPRGDSPYSGRGDSPYS

SEQ ID NO: 114 - DNA, 480 bp, Artificial Sequence
  Synthetic
ggccgtcctgactctccttatagtggtcgcggagattctccgtatagtcctcgtggagactcaccttattcaggccgtggtgatagtccctatagtggaagaccagactccccctattcaggtagaggcgattctccctactctcccagaggtgatagcccttattcgggacgtggggatagtccttacagtggtaggccagattcaccctacagcggcaggggagatagtccgtacagtccaagaggggattcaccgtactcgggtcgaggtgattccccatattctggtagaccggattctccatactcaggaaggggtgacagtccttattctcctagaggtgactcgccgtattcagggcgtggcgatagtccatatagtggcagacctgattctccttactctggacgcggtgattcaccatattcacctaggggcgactctccatattccgggcgcggggacagcccatacagt

SEQ ID NO: 115 - protein, 160 aa, Artificial Sequence
  Synthetic
GRPDSPYSPRGDSPYSGRPDSPYSGRGDSPYSPRGDSPYSGRPDSPYSPRGDSPYSGRGDSPYSGRPDSPYSPRGDSPYSGRPDSPYSGRGDSPYSPRGDSPYSGRPDSPYSPRGDSPYSGRGDSPYSGRPDSPYSPRGDSPYSGRPDSPYSGRGDSPYS

SEQ ID NO: 116 - DNA, 480 bp, Artificial Sequence
  Synthetic
ggccgtcctgatagtccatacagtcccaggggcgactcgccgtattcaggtagaccggattcaccgtattctgggcgtggggacagcccatattcacctaggggagattcaccttattcaggaaggcccgattcaccctatagtccaaggggtgattccccttattcgggacgcggagacagtccttacagtgggagaccagattctccttatagtcctcgtggagactcaccatattctggaagacctgattctccctactcagggcgcggtgactcaccttactctccgagaggtgattcgccttattccgggaggcctgactctccatattccccccgtggcgatagtccgtacagcggacgtggtgattcaccatactctggtaggccagatagtccctattctccaagaggtgacagtccatatagtggccgtccagactccccatatagcggtcgcggggatagtccttattct

SEQ ID NO: 117 - protein, 160 aa, Artificial Sequence
  Synthetic
GRPDSPYSPRGDSPYSGRPDSPYSPRGDSPYSGRPDSPYSPRGDSPYSGRPDSPYSPRGDSPYSGRPDSPYSPRGDSPYSGRPDSPYSPRGDSPYSGRPDSPYSPRGDSPYSGRPDSPYSPRGDSPYSGRPDSPYSPRGDSPYSGRPDSPYSPRGDSPYS

SEQ ID NO: 118 - DNA, 480 bp, Artificial Sequence
  Synthetic
ggccgacctgactctccatacagtccccgtggtgatagtccatatagcggtaggcctgattcgccgtactctccgagaggcgactcaccctattcagggcgtccagatagtccctactcaccaagaggtgactcgccatattccgggaggccagattcaccatattcacctaggggtgacagtccttactcaggaaggcccgattcaccgtatagtccacgcggagactccccatatagtggtcgtcctgattcaccttattctcctagaggggattctccatattctgggagaccggatagtccttatagtcctcgtggagacagcccatactctggtagacccgatagtccgtattctccaaggggcgattctccttacagtggaagacctgatagcccttattccccccgaggtgattctccctatagtggcagaccagactctccttattcacccaggggagattctccgtacagt

SEQ ID NO: 119 - protein, 160 aa, Artificial Sequence
  Synthetic
GRGDSPYSGRGDSPVSGRGDSPYSGKGDSPYSGRGDSPYSGRGDSPVSGRGDSPYSGKGDSPYSGRGDSPYSGRGDSPVSGRGDSPYSGKGDSPYSGRGDSPYSGRGDSPVSGRGDSPYSGKGDSPYSGRGDSPYSGRGDSPVSGRGDSPYSGKGDSPYS

SEQ ID NO: 120 - DNA, 480 bp, Artificial Sequence
  Synthetic
ggccgcggagattctccatactccggtagaggggacagtccagtttcaggcagaggcgattcaccgtatagtggaaaaggagactccccctatagtggcaggggcgatagtccgtattccgggcgcggggattctcctgtaagtggacgtggagattccccatatagcggcaagggtgatagtccttactcaggtcgcggtgattcaccatatagtggtcgtggcgactcacctgtgtctggccgtggggactctccttattctgggaaaggtgattcgccgtactctggaagaggtgatagcccttattcagggcgtggtgattctcccgtttctggtaggggtgacagcccatacagtggtaaaggagattcaccttattcgggtcgaggggatagtccctattcaggacgaggtgactctccagtatcaggaaggggagatagtccatattcaggtaagggagacagtccttatagt

SEQ ID NO: 121 - protein, 160 aa, Artificial Sequence
  Synthetic
GRGDSPYSGRGDSPVSGRGDSPYSGRGDSPVSGRGDSPYSGRGDSPVSGRGDSPYSGKGDSPYSGRGDSPYSGRGDSPVSGRGDSPYSGRGDSPVSGRGDSPYSGRGDSPVSGRGDSPYSGKGDSPYSGRGDSPYSGRGDSPVSGRGDSPYSGRGDSPVS

SEQ ID NO: 122 - DNA, 480 bp, Artificial Sequence
  Synthetic
ggccgcggagattctccatactctggtcgaggggatagtccggtctcaggaagaggcgatagtccatattcaggacgaggtgacagtccagtatctggcagaggggactctccgtattccggcaggggagacagccctgtttcaggccgaggtgattctccttactcaggtaagggagatagtccttattctggtaggggtgactccccctattctggaaggggagattcacctgtaagtgggcgtggtgattcaccctactcgggtcgtggagactctccagttagtggtcgcggtgattccccatatagcgggcgcggcgattctcccgtttctgggaggggcgacagtccttattcgggaaaaggcgactctccttattcagggagaggtgatagtccctacagtggccgtggggattcaccagtgtcaggtagaggtgactcaccttatagtggacggggtgatagcccagtcagt

SEQ ID NO: 123 - protein, 160 aa, Artificial Sequence
  Synthetic
GRGDSPYSGKGDSPYSGRGDSPYSGKGDSPYSGRGDSPYSGKGDSPYSGRGDSPYSGRGDSPVSGRGDSPYSGKGDSPYSGRGDSPYSGKGDSPYSGRGDSPYSGKGDSPYSGRGDSPYSGRGDSPVSGRGDSPYSGKGDSPYSGRGDSPYSGKGDSPYS

SEQ ID NO: 124 - DNA, 480 bp, Artificial Sequence
  Synthetic
ggccgcggagattccccgtatagcgggaagggcgattctccttattctggcagaggggatagtccttactcaggaaaaggtgactccccatattccggtcgtggagattcaccgtattcaggcaagggggacagtccttatagtggccgtggtgattcaccatattcaggtcgcggcgactcacctgtctcaggacgtggggattctccctactccggcaaaggtgattctccatatagtggtcgaggtgatagcccatacagtggtaagggagatagtccgtatagtggaagaggcgatagtccatattctgggaaaggagacagtccatactcgggcaggggagactctccttacagcggtcgtggggactcgccggtgtctggtagaggtgacagcccctattctggaaagggtgatagtccctatagtgggaggggtgattcgccatattcgggtaaaggagattctccgtacagt

SEQ ID NO: 125 - protein, 160 aa, Artificial Sequence
  Synthetic
GRGDSPYSGRGDSPYSGRGDSPYSGRGDSPLSGRGDSPYSGRGDSPYSGRGDSPYSGRGDSPLSGRGDSPYSGRGDSPYSGRGDSPYSGRGDSPLSGRGDSPYSGRGDSPYSGRGDSPYSGRGDSPLSGRGDSPYSGRGDSPYSGRGDSPYSGRGDSPLS

SEQ ID NO: 126 - DNA, 480 bp, Artificial Sequence
  Synthetic
ggacgtggcgacagcccttattcaggtagaggcgactctccttattcgggacggggtgactccccatattctggacgcggtgattctcctttgtcagggaggggtgattccccctatagtggaaggggagactcaccctacagtgggagaggtgattcaccgtactcgggtcggggagatagtccgttatcaggacgaggtgatagcccatacagcgggcgtggtgatagtccctattcaggccgtggggatagtccttactcaggtcgaggggactctccattgtctggccgaggtgactctccgtattccggcaggggagattcaccttatagtggcagaggtgacagtccatatagtggtcgtggagacagtcctctatctgggcgcggagattctccctactccggtcgcggcgattcaccatattcaggaagaggggattctccatactctggtaggggcgatagtccactttct

SEQ ID NO: 127 - protein, 160 aa, Artificial Sequence
  Synthetic
GRGDSPYSGRGDSPLSGRGDSPYSGRGDSPLSGRGDSPYSGRGDSPLSGRGDSPYSGRGDSPLSGRGDSPYSGRGDSPLSGRGDSPYSGRGDSPLSGRGDSPYSGRGDSPLSGRGDSPYSGRGDSPLSGRGDSPYSGRGDSPLSGRGDSPYSGRGDSPLS

SEQ ID NO: 128 - DNA, 480 bp, Artificial Sequence
  Synthetic
ggacgtggtgattcaccttattctggtagaggtgattctccattaagtgggcgcggtgattccccctattcgggtcgtggggattcaccattgagtggcaggggagactctccgtattccggtcgcggcgattctcctttatcaggccgtggcgactctccatatagcgggcgtggagacagtccactttcaggacgaggggactctccttatagtggaaggggtgatagtcccttgtctggccgaggtgatagcccatattcagggaggggcgatagtccgttatctggacggggtgactcaccatacagtggtcgaggtgacagccctttgtcaggtcggggagattctccctactctgggagaggtgactccccattatccggcagaggggatagtccatactcaggtaggggagatagtcctctctcaggaagaggcgacagtccttactcgggacgcggagattcacccttaagc

SEQ ID NO: 129 - protein, 160 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGDGDSPYSGRGDSPYSGRGDSPYSGRGDSPYSGRGDSPYSGRGDSPYSGRGDSPYSGRGDSPYSGRGDSPYSGRGDSPYS

SEQ ID NO: 130 - DNA, 480 bp, Artificial Sequence
  Synthetic
ggccggggtgattcgccgtactccggtcgcggagattcaccctattctggaaggggcgatagtccttattctggtcgaggtgatagtccatattcgggtaggggagattctccctatagtggacgcggtgatagcccttattccgggcgcggggattcaccgtatagtgggagaggtgatagtccgtattctgggcgtggtgattcaccttattcaggtcgtggcgattctccatactctggtgatggagattccccatatagcggcaggggtgactctccttacagtggaagaggcgactctccatatagtggccgtggagacagcccatattcaggccgaggggatagtccctactcgggacgaggtgattctccttatagtggtagaggggactcaccatattctggccgtggagactccccctattcaggacgtggggacagtccttactcaggtagaggtgacagtccatacagt

SEQ ID NO: 131 - protein, 160 aa, Artificial Sequence
  Synthetic
GRGDSPYSGRGDSPYSGRGDSPYSGRGDSPYSGRGDSPYSGRGDSPYSGDGDSPYSGRGDSPYSGRGDSPYSGRGDSPYSGRGDSPYSGRGDSPYSGRGDSPYSGDGDSPYSGRGDSPYSGRGDSPYSGRGDSPYSGRGDSPYSGRGDSPYSGRGDSPYS

SEQ ID NO: 132 - DNA, 480 bp, Artificial Sequence
  Synthetic
ggccggggtgattcgccgtactccggtcgcggagattcaccctattctggaaggggcgatagtccttattctggtcgaggtgatagtccatattcgggtaggggagattctccctatagtggacgcggtgatagcccttattccggggacggggattcaccgtatagtgggagaggtgatagtccgtattctgggcgtggtgattcaccttattcaggtcgtggcgattctccatactctggtaggggagattccccatatagcggcaggggtgactctccttacagtggaagaggcgactctccatatagtggcgatggagacagcccatattcaggccgaggggatagtccctactcgggacgaggtgattctccttatagtggtagaggggactcaccatattctggccgtggagactccccctattcaggacgtggggacagtccttactcaggtagaggtgacagtccatacagt

SEQ ID NO: 133 - protein, 160 aa, Artificial Sequence
  Synthetic
GRGRSPYSGRGDSPYSGRGDSPYSGRGDSPYSGRGRSPYSGRGDSPYSGRGDSPYSGRGDSPYSGRGRSPYSGRGDSPYSGRGDSPYSGRGDSPYSGRGRSPYSGRGDSPYSGRGDSPYSGRGDSPYSGRGRSPYSGRGDSPYSGRGDSPYSGRGDSPYS

SEQ ID NO: 134 - DNA, 480 bp, Artificial Sequence
  Synthetic
ggacgagggaggtcaccctatagtggaagaggcgatagtccatatagtggccgtggagattccccctattctggacgcggtgactctccttattctggtagaggaaggtctccatattccggcagaggtgattcaccatattctgggcgcggagactcgccgtactcaggtcgcggggacagtccttatagtgggagaggtaggagcccatattcaggacgtggcgattctccctactccggtcggggtgattctccttactctggcaggggagatagtccctattcaggccgaggaagatcaccgtatagtggtaggggcgactcaccttattcagggcgtggtgatagtccttacagtggtcgaggtgatagcccttattcgggaaggggtagaagtccatacagcggtagaggggattctccatactcgggtcgtggggatagtccgtattcaggtagaggtgacagcccgtacagt

SEQ ID NO: 135 - protein, 160 aa, Artificial Sequence
  Synthetic
GRGRSPYSGRGDSPYSGRGRSPYSGRGDSPYSGRGRSPYSGRGDSPYSGRGRSPYSGRGDSPYSGRGRSPYSGRGDSPYSGRGRSPYSGRGDSPYSGRGRSPYSGRGDSPYSGRGRSPYSGRGDSPYSGRGRSPYSGRGDSPYSGRGRSPYSGRGDSPYS

SEQ ID NO: 136 - DNA, 480 bp, Artificial Sequence
  Synthetic
ggtagaggaaggtcaccatatagcggtcggggtgacagtccatatagtggaaggggcagatcaccgtattctgggagaggtgattcaccttattctggtaggggtagatccccctattcaggccgaggtgactctccatattctggcagaggacgcagtccttatagtggccgtggcgatagtccctactcaggtagaggtaggagtccttactctggtcgtggtgatagtccatactcgggtcgagggaggtctccttattcaggacggggagatagcccttattcgggacgaggaagatctccttacagtggacgtggagactcaccctattctggaagaggtcgttctccctatagtggtagaggggattcgccttattccggtagaggcagaagtccgtatagtgggcgtggggacagcccgtactccggcaggggaagaagcccatattcaggtcgcggagattctccatacagt

SEQ ID NO: 137 - protein, 160 aa, Artificial Sequence
  Synthetic
GDGDSPYSGRGDSPYSGRGDSPYSGRGDSPYSGDGDSPYSGRGDSPYSGRGDSPYSGRGDSPYSGDGDSPYSGRGDSPYSGRGDSPYSGRGDSPYSGDGDSPYSGRGDSPYSGRGDSPYSGRGDSPYSGDGDSPYSGRGDSPYSGRGDSPYSGRGDSPYS

SEQ ID NO: 138 - DNA, 480 bp, Artificial Sequence
  Synthetic
ggggatggagactctccttatagtggaagaggggattctccctactctggaaggggcgactcaccttattccggtagaggcgattcaccctattcaggtgatggagatagtccttattcaggcagaggtgactccccctatagtggtcgcggtgacagtccatatagcggtcgtggggatagtccctattctggagatggtgatagtccatacagtggacgtggcgatagtccgtattctgggaggggtgattcgccgtatagtggcaggggagattccccatattcaggagacggggacagcccatatagtgggagaggtgattcaccgtactccgggcgtggagattctccttactcgggacgaggtgatagcccttattctggtgacggagattcaccatattctggccgtggtgattctccatattcgggtcgaggggactctccatactcaggacgcggagacagtccttacagt

SEQ ID NO: 139 - protein, 159 aa, Artificial Sequence
  Synthetic
DGDSPYSGRGDSPYSGDGDSPYSGRGDSPYSGDGDSPYSGRGDSPYSGDGDSPYSGRGDSPYSGDGDSPYSGRGDSPYSGDGDSPYSGRGDSPYSGDGDSPYSGRGDSPYSGDGDSPYSGRGDSPYSGDGDSPYSGRGDSPYSGDGDSPYSGRGDSPYS

SEQ ID NO: 140 - DNA, 477 bp, Artificial Sequence
  Synthetic
gacggagacagtccttatagtgggaggggtgatagtccgtattcaggagatggcgatagtccctacagcggtcgaggtgattccccttattcgggagatggggatagtccatatagcggaagaggcgactctccatacagtggagacggtgattctccttactcaggacgtggggattctccatattctggagatggagactctccttattcaggcagaggtgactccccctattcaggtgatggtgattcaccatatagtggacgcggagattcgccctactcgggtgacggcgattcaccctattctggtagaggggactcaccttactctggtgatggagattctccctatagtggtcgtggtgatagcccatattcaggggatggtgacagtccatactccggtaggggagattcaccttattctggggacggggacagcccgtatagtggccgtggagatagtccttacagt

SEQ ID NO: 141 - protein, 160 aa, Artificial Sequence
  Synthetic
GRGDSPYSGRGDSPYSGRGDSPYSGRGDNPYNGRGDSPYSGRGDSPYSGRGDSPYSGRGDNPYNGRGDSPYSGRGDSPYSGRGDSPYSGRGDNPYNGRGDSPYSGRGDSPYSGRGDSPYSGRGDNPYNGRGDSPYSGRGDSPYSGRGDSPYSGRGDNPYN

SEQ ID NO: 142 - DNA, 480 bp, Artificial Sequence
  Synthetic
ggacgcggagattcaccatattctggacgaggggacagtccttattcaggtcggggagattctccttatagtggtcgtggagataatccttataatggccgaggtgactctccatattcaggccgtggggattcaccctacagtgggagaggcgactccccatactcaggacggggtgacaacccttacaatggaagaggggatagtccctatagtggaaggggagatagtccgtattcagggaggggtgatagcccttattcgggtcgaggtgataatccatacaacggtcgcggcgattctccctactctggcagaggtgattccccctattctggtagaggtgacagcccatattccggcaggggcgacaatccatataatggtaggggagactcaccttattctgggcgcggtgatagtccatatagcgggcgtggcgatagtccttactcgggacgtggtgataacccatataac

SEQ ID NO: 143 - protein, 160 aa, Artificial Sequence
  Synthetic
GRGDSPYSGRGDNPYNGRGDSPYSGRGDNPYNGRGDSPYSGRGDNPYNGRGDSPYSGRGDNPYNGRGDSPYSGRGDNPYNGRGDSPYSGRGDNPYNGRGDSPYSGRGDNPYNGRGDSPYSGRGDNPYNGRGDSPYSGRGDNPYNGRGDSPYSGRGDNPYN

SEQ ID NO: 144 - DNA, 480 bp, Artificial Sequence
  Synthetic
ggtcggggagattccccctatagtggtaggggagacaatccttataatggaaggggtgacagtccctattcaggtcgaggggacaacccatataatggccgtggtgatagcccttacagtggaagaggtgacaatccatacaatgggcgtggcgattcaccctactccggcaggggagataacccgtacaatggacgcggcgactctccttactctgggaggggcgataatccctataatggtcgtggagatagtccatattctggcagaggcgacaatccctacaatggtagaggtgattctccatactcaggccgaggtgataatccttacaacggacgaggtgactcaccatatagcgggcgcggagataatccgtataatgggagaggggattcaccttattcgggacgtggggataatccatataacggtcgcggtgatagtccttattcaggacggggtgataacccttataac

SEQ ID NO: 145 - protein, 160 aa, Artificial Sequence
  Synthetic
GRGDNPYNGRGDNPYNGRGDNPYNGRGDSPYSGRGDNPYNGRGDNPYNGRGDNPYNGRGDSPYSGRGDNPYNGRGDNPYNGRGDNPYNGRGDSPYSGRGDNPYNGRGDNPYNGRGDNPYNGRGDSPYSGRGDNPYNGRGDNPYNGRGDNPYNGRGDSPYS

SEQ ID NO: 146 - DNA, 480 bp, Artificial Sequence
  Synthetic
ggacgaggtgactcaccatattctggacgcggagataatccgtacaatggcagaggggataatccttataacggcaggggagacaatccctataatgggaggggtgatagcccttattccgggagaggcgacaatccatacaacggacgtggagacaacccttacaatgggcgtggggataacccatacaatggaagaggtgattcaccttatagtggtcgcggggacaacgccaactatggtcgaagttatcacccgtattatggacggcgagatcacccttattatggttggggagattctccttattcaggccgaggggacaatccgtataacggtagaggtgacaatccttacaacggtcgtggtgataatccatataacgggcgcggtgatagtccttactcaggtcggggtgacaacccatataatggccgtggcgataacccctataacggaaggggcgataatccctacaac

SEQ ID NO: 147 - protein, 160 aa, Artificial Sequence
  Synthetic
GRGDQPYQGRGDQPYQGRGDQPYQGRGDQPHQGRGDQPYQGRGDQPYQGRGDQPYQGRGDQPHQGRGDQPYQGRGDQPYQGRGDQPYQGRGDQPHQGRGDQPYQGRGDQPYQGRGDQPYQGRGDQPHQGRGDQPYQGRGDQPYQGRGDQPYQGRGDQPHQ

SEQ ID NO: 148 - DNA, 480 bp, Artificial Sequence
  Synthetic
gggcgtggagaccagccgtatcaagggagaggtgaccagccttatcaaggtcggggagatcaaccttatcaaggcaggggcgatcaacctcatcagggtagaggggaccaaccctaccaaggccggggtgatcaaccgtaccaaggacgtggtgatcaaccatatcagggccgtggagatcaaccccatcaaggaaggggagatcagccataccagggaagaggcgaccaaccgtatcagggacgaggtgatcaaccctatcagggtcgcggggatcaaccacaccagggtcgtggggaccagccatatcaaggtagaggtgatcagccctatcaaggacgcggtgatcaaccttaccagggcagaggggatcagccgcaccaaggtaggggagaccaaccttatcaggggaggggtgaccaaccataccaaggtcgaggcgatcagccttaccaagggcgcggagatcaaccgcatcaa

SEQ ID NO: 149 - protein, 160 aa, Artificial Sequence
  Synthetic
GRGDQPYQGRGDQPHQGRGDQPYQGRGDQPHQGRGDQPYQGRGDQPHQGRGDQPYQGRGDQPHQGRGDQPYQGRGDQPHQGRGDQPYQGRGDQPHQGRGDQPYQGRGDQPHQGRGDQPYQGRGDQPHQGRGDQPYQGRGDQPHQGRGDQPYQGRGDQPHQ

SEQ ID NO: 150 - DNA, 480 bp, Artificial Sequence
  Synthetic
gggcgcggtgatcaaccctatcaaggacgtggagatcaacctcatcagggaagaggtgaccagccatatcaagggagaggggaccagccccatcaaggaaggggtgatcaaccttaccaaggacgcggagatcaaccccaccaagggaggggcgatcagccctatcagggcaggggagatcaaccgcatcaagggcgtggtgatcaaccatatcagggacgaggcgatcaaccacatcaaggccgtggggatcaaccataccaaggtaggggagaccaaccacaccagggccggggtgaccaaccgtatcaaggtcgcggggaccaacctcaccaaggtcgtggagaccagccttatcagggtagaggggatcagccacatcagggtcgaggtgatcaaccgtaccagggtcggggagatcagcctcatcaaggtagaggcgaccaaccctaccaaggcagaggtgatcagccgcaccaa

SEQ ID NO: 151 - protein, 160 aa, Artificial Sequence
  Synthetic
GRGDQPHQGRGDQPHQGRGDQPHQGRGDQPYQGRGDQPHQGRGDQPHQGRGDQPHQGRGDQPYQGRGDQPHQGRGDQPHQGRGDQPHQGRGDQPYQGRGDQPHQGRGDQPHQGRGDQPHQGRGDQPYQGRGDQPHQGRGDQPHQGRGDQPHQGRGDQPYQ

SEQ ID NO: 152 - DNA, 480 bp, Artificial Sequence
  Synthetic
gggcgtggggaccaaccgcaccaagggagaggggatcagccgcatcaagggcgcggggatcaacctcatcaggggaggggcgatcaaccttatcaaggacgaggtgatcaacctcaccaaggacgtggtgatcaaccgcatcagggccgtggagaccaaccccaccaaggcaggggtgatcaaccataccaaggtaggggagaccagccacatcaaggtagaggcgaccaaccacaccaaggtcgaggcgatcagcctcaccagggaaggggagatcaaccctatcagggtcggggagatcagccccatcagggtagaggggaccagcctcatcaaggaagaggtgatcagccacaccagggtcgtggagatcaaccgtaccagggcagaggtgaccaacctcatcaaggtcgcggagatcaaccacatcagggacgcggtgatcaaccccatcaaggccggggtgaccagccctatcaa

SEQ ID NO: 153 - protein, 160 aa, Artificial Sequence; identical to SEQ ID NO: 23
  Synthetic

SEQ ID NO: 154 - DNA, 480 bp, Artificial Sequence; identical to SEQ ID NO: 24
  Synthetic

SEQ ID NO: 155 - protein, 160 aa, Artificial Sequence; identical to SEQ ID NO: 25
  Synthetic

SEQ ID NO: 156 - DNA, 480 bp, Artificial Sequence; identical to SEQ ID NO: 26
  Synthetic

SEQ ID NO: 157 - protein, 160 aa, Artificial Sequence; identical to SEQ ID NO: 27
  Synthetic

SEQ ID NO: 158 - DNA, 480 bp, Artificial Sequence; identical to SEQ ID NO: 28
  Synthetic

SEQ ID NO: 159 - protein, 58 aa, Artificial Sequence [sequence omitted]

SEQ ID NO: 160 - DNA, 174 bp, Artificial Sequence
  Synthetic
gtggacaataagttcaataaggagcagcagaatgccttttacgagattctccacctgcccaacctcaacgaagagcagagaaatgcgtttatccagagtctgaaagacgacccaagccagtccgcaaacctgctggctgaggccaagaagcttaacgacgcacaggccccaaag

SEQ ID NO: 161 - protein, 37 aa, Artificial Sequence
  Synthetic
LLGDFFRKSKEKIGKEFKRIVQRIKDFLRNLVPRTES

SEQ ID NO: 162 - DNA, 111 bp, Artificial Sequence
  Synthetic
cttctgggggatttttttcgcaaatctaaggaaaaaatcgggaaggaatttaaacgcatcgttcaacgcattaaagacttccttcgtaaccttgtccctcgtacagagagt

SEQ ID NO: 163 - protein, 20 aa, Artificial Sequence
  Synthetic
RWWRMMRRGPGRGMMRRGWE

SEQ ID NO: 164 - DNA, 60 bp, Artificial Sequence
  Synthetic
cgctggtggcgtatgatgcgtcgcggccctggccgcggtatgatgcgccgcggctgggaa

SEQ ID NO: 165 - protein, 20 aa, Artificial Sequence
  Synthetic
RWWRMMRRGPRWGMMRRGWE

SEQ ID NO: 166 - DNA, 60 bp, Artificial Sequence
  Synthetic
cgctggtggcgtatgatgcgtcgcggccctcgctggggtatgatgcgccgcggctgggaa

SEQ ID NO: 167 - protein, 20 aa, Artificial Sequence
  Synthetic
WWRRMMRRGPRWGMMRRGWE

SEQ ID NO: 168 - DNA, 60 bp, Artificial Sequence
  Synthetic
tggtggcgccgtatgatgcgtcgcggccctcgctggggtatgatgcgccgtggctgggaa

SEQ ID NO: 169 - protein, 26 aa, Artificial Sequence
  Synthetic
RVKRVWPLVIRTVIAGYNLYRAIKKK

SEQ ID NO: 170 - DNA, 78 bp, Artificial Sequence
  Synthetic
cgcgtgaaacgcgtgtggccgctggtgattcgcaccgtgattgcgggctataacctgtatcgcgcgataaaaaagaaa

SEQ ID NO: 171 - protein, 24 aa, Artificial Sequence
  Synthetic
GRIGSILGRLAKGLPTLKSWIKNR

SEQ ID NO: 172 - DNA, 72 bp, Artificial Sequence
  Synthetic
ggcagaataggctctatccttggaagacttgccaaggggctgcctactctaaagagctggattaagaaccgc

SEQ ID NO: 173 - protein, 190 aa, Artificial Sequence
  Synthetic
GTESLVLSPAPAKPKRVKASRRSASHPTYSEMIAAAIRAEKSRGGSSRQSIQKYIKSHYKVGHNADLQIKLSIRRLLAAGVLKQTKGVGASGSFRLAKSDKAKRSPGKKKKAVRRSTSPKKAARPRKARSPAKKPKATARKARKKSRASPKKAKKPKTVKAKSRKASKAKKVKRSKPRAKSGARKSPKKK

SEQ ID NO: 174 - DNA, 573 bp, Artificial Sequence
  Synthetic
atgggcaccgaaagccttgttttatctccagctccagccaaacccaaacgcgttaaagcaagccgccgcagtgccagccatccgacctacagcgaaatgattgccgcggcgattcgtgcggaaaaatcacgcggcggcagctcccgccagtcgattcagaaatatattaaaagccactataaagtgggtcataacgccgatctgcaaatcaaactgagcattcgccgtctgctggcagcgggcgtgctgaaacagaccaaaggcgttggcgcgagcggcagtttccgcctggcgaaatccgataaagcgaaacggagtccgggcaaaaaaaaaaaggcggtgcgtcgcagcaccagcccgaaaaaagcggcacgtccgcgtaaagcgcgttctccggcaaaaaagccgaaggcaacggcgcgcaaagcacgaaaaaaaagtcgcgcctcgccgaaaaaagcgaagaaaccgaaaaccgtgaaagctaaaagccgtaaggctagcaaagccaaaaaagtcaaacgttcaaaaccgcgcgcaaagagcggtgcccgtaaatcgccaaaaaagaag

SEQ ID NO: 175 - protein, 31 aa, Artificial Sequence
  Synthetic
GVGHNADLQIKLSIRRLLAAGVLKQTKGVGA

SEQ ID NO: 176 - DNA, 93 bp, Artificial Sequence
  Synthetic
ggcgtcggacataatgcggatctgcaaatcaaactgagcattcgccgtctgctggcggcgggcgttctgaaacagaccaaaggcgtgggcgca

SEQ ID NO: 177 - protein, 32 aa, Artificial Sequence
  Synthetic
AAHGEGTFTSDVSSYLEEQAAKEFIAWLVKGA

SEQ ID NO: 178 - DNA, 96 bp, Artificial Sequence
  Synthetic
gctgcccatggggaggggacattcacctcggacgtaagcagttacttagaagaacaggctgcaaaagagtttattgcgtggcttgtgaaaggagca

SEQ ID NO: 179 - protein, 235 aa, Artificial Sequence
  Synthetic
VSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 180 - DNA, 705 bp, Artificial Sequence
  Synthetic
gtgagcaagggcgaggaagataacatggcctctctcccagcgacacatgagttacacatctttggctccatcaacggtgtggactttgacatggtgggtcagggcaccggcaatccaaatgatggttatgaagagttaaacctcaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctaaagaaccagccgatgtacgtgttccgtaagacggagctcaagcactccaagaccgagctcaacttcaaggagtggcaaaaggcctttaccgatgtgatgggcatggacgagctgtacaag

SEQ ID NO: 181 - protein, 236 aa, Artificial Sequence
  Synthetic
VSKGEELIKENMRMKVVMEGSVNGHQFKCTGEGEGRPYEGVQTMRIKVIEGGPLPFAFDILATSFMYGSRTFIKYPADIPDFFKQSFPEGFTWERVTRYEDGGVVTVTQDTSLEDGELVYNVKVRGVNFPSNGPVMQKKTKGWEPNTEMMYPADGGLRGYTDIALKVDGGGHLHCNFVTTYRSKKTVGNIKMPGVHAVDHRLERIEESDNETYVVQREVAVAKYSNLGGGMDELYK

SEQ ID NO: 182 - DNA, 708 bp, Artificial Sequence
  Synthetic
gtgtctaagggcgaagagctgatcaaggaaaatatgcgtatgaaggtggtcatggaaggttcggtcaacggccaccaattcaaatgcacaggtgaaggagaaggcagaccgtacgagggagtgcaaaccatgaggatcaaagtcatcgagggaggacccctgccatttgcctttgacattcttgccacgtcgttcatgtatggcagccgtacctttatcaagtacccggccgacatccctgatttctttaaacagtcctttcctgagggttttacttgggaaagagttacgagatacgaagatggtggagtcgtcaccgtcacgcaggacaccagccttgaggatggcgagctcgtctacaacgtcaaggtcagaggggtaaactttccctccaatggtcccgtgatgcagaagaagaccaagggttgggagcctaatacagagatgatgtatccagcagatggtggtctgagaggatacactgacatcgcactgaaagttgatggtggtggccatctgcactgcaacttcgtgacaacttacaggtcaaaaaagaccgtcgggaacatcaagatgcccggtgtccatgccgttgatcaccgcctggaaaggatcgaagagagtgacaatgaaacctacgtagtgcaaagagaagtggcagttgccaaatacagcaaccttggtggtggcatggacgagctgtacaag

SEQ ID NO: 183 - protein, 347 aa, Artificial Sequence
  Synthetic
RSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDL

SEQ ID NO: 184 - DNA, 1041 bp, Artificial Sequence
  Synthetic
cgcagtcgtttattagaggatttccgtaacaaccgttaccctaatttgcaactgcgtgagattgctggccacattatggaattttcacaagaccagcacggatcgcgtttcattcaacttaaattagaacgcgctacgccagccgaacgccagttagttttcaacgaaattttacaagcggcataccaactgatggtggacgtttttgggaattatgttatccagaagttcttcgagtttggaagcctggagcagaaacttgccctggcggaacgcattcgtggtcacgtcttaagtttggcactgcagatgtatgggtgccgtgttatccaaaaagctcttgagtttatcccatctgatcagcagaacgaaatggtacgcgagctggacggccatgttcttaaatgcgtaaaggaccagaacggtaatcatgttgtacagaaatgcatcgaatgcgtccagccacagtctctgcagttcatcattgatgctttcaaaggccaagtttttgcccttagcacgcatccctacgggtgtcgtgtcatccaacgcattcttgagcattgcttaccagatcaaaccttgccaatcctggaggaattgcaccaacacactgaacagctggtacaagatcaatatggtaactatgtaattcagcacgttctggaacatggacgccctgaggacaagtctaaaattgtcgcggaaatccgtggaaacgtattggtgttgtcccaacacaaatttgccagtaatgtcgtggagaaatgcgtaacccatgctagccgtaccgaacgcgcagtcttaatcgacgaagtttgcacaatgaatgacggcccgcattcggctttatatacaatgatgaaggaccagtacgctaattatgtcgtgcagaaaatgattgatgtagcggagcctggtcagcgcaagattgtgatgcacaagatccgcccgcacattgcgacattgcgtaagtatacgtacggcaaacacatcttggcgaaacttgaaaagtactacatgaaaaatggcgtggacctt

SEQ ID NO: 185 - protein, 182 aa, Artificial Sequence
  Synthetic
KHPLQNRWALWFFKNDKSKTWQANLRLISKFDTVEDFWALYNHIQLSSNLMPGCDYSLFKDGIEPMWEDEKNKRGGRWLITLNKQQRRSDLDRFWLETLLCLIGESFDDYSDDVCGAVVNVRAKGDKIAIWTTECENREAVTHIGRVYKERLGLPPKIVIGYQSHADTATKSGSTTKNRFVV

SEQ ID NO: 186 - DNA, 546 bp, Artificial Sequence
  Synthetic
aaacatcctcttcagaatcgttgggcgctttggttttttaagaatgacaaatctaaaacatggcaagcgaatcttcgtctgatttcaaagtttgacacagtggaagatttttgggccctttataatcacatccaattaagctctaatctgatgccagggtgcgactatagcttgtttaaggacggtattgaacccatgtgggaagatgaaaagaacaaacgcggtggacgttggcttattactttgaataaacagcagcgtcgtagtgatttagatcgtttctggcttgaaacactgctgtgccttatcggtgaatcctttgacgactactctgacgacgtatgtggcgcagtagtaaacgtgcgtgccaaaggggacaagatcgcaatctggacaacggagtgcgagaatcgtgaagccgttactcacatcggtcgtgtctacaaggaacgcttggggttgccgccaaagattgttatcggctatcagagccacgctgatacagcaaccaaaagcggatcgaccacgaagaatcgttttgtagtg

SEQ ID NO: 187 - protein, 173 aa, Artificial Sequence
  Synthetic
SYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKER

SEQ ID NO: 188 - DNA, 519 bp, Artificial Sequence
  Synthetic
agctacccaatggcgagcctgtatgtcggagaccttcaccctgatgttaccgaggccatgctgtatgagaaattcagcccagctggtccgatcctttccattcgcgtgtgccgcgatatgatcacacgtcgttcccttggttacgcctatgtaaattttcaacaaccagccgatgcagaacgcgccttggacactatgaatttcgatgtgattaagggtaaaccggttcgcattatgtggtcccaacgtgatccttcgcttcgcaagtctggggtagggaacatctttattaaaaatctggacaagtccattgataacaaagcattatatgataccttctcagcgttcgggaacattttatcctgtaaggttgtgtgcgacgaaaatggaagcaaaggctatggctttgtacattttgagacacaggaggcggcagagcgtgccattgaaaagatgaatggaatgttacttaatgaccgtaaggtgttcgtcggtcgttttaaatcgcgcaaggaacgc

SEQ ID NO: 189 - protein, 70 aa, Artificial Sequence
  Synthetic
STRYKTELCRPFEESGTCKYGEKCQFAHGFHELRSLTRHPKYKTELCRTFHTIGFCPYGPRCHFIHNADE

SEQ ID NO: 190 - DNA, 210 bp, Artificial Sequence
  Synthetic
agcacccgctataaaaccgaactgtgccgcccgtttgaagaaagcggcacgtgcaaatacggcgagaaatgccagttcgcccatggcttccatgaactgcgcagtctgacgcgccatccgaagtacaaaaccgagctgtgccgcaccttccataccatcggcttttgtccgtacggcccgcgctgccacttcatccataatgcggatgaa

SEQ ID NO: 191 - protein, 26 aa, Artificial Sequence
  Synthetic
GPRRGDGRRRGGGGRGQGGRGRGGGF

SEQ ID NO: 192 - DNA, 78 bp, Artificial Sequence
  Synthetic
gggcctcgtcgtggtgatggccgacgtcgtggcggtggaggacgtggccaaggtggacgtggacgtggtggaggtttc

SEQ ID NO: 193 - protein, 95 aa, Artificial Sequence
  Synthetic
NRPPSAGESVQITLPKNEVQLNAYVLQEPPKGETYTYDWQLITHPRDYSGEMEGKHSQILKLSKLTPGLYEFKVIVEGQNAHGEGYVNVTVKPEP

SEQ ID NO: 194 - DNA, 285 bp, Artificial Sequence
  Synthetic
aatcgtccgcccagcgcgggcgaaagcgtgcagatcacgctgccgaaaaacgaagtgcagctgaacgcctacgtgctgcaagaaccgccgaaaggcgaaacctacacctacgattggcagctgatcacccatccgcgcgattacagcggcgaaatggaaggcaagcacagccagattctgaaactgagcaagctgaccccgggtctgtacgagttcaaggtgatcgtggaaggccagaatgcccatggcgaaggctacgttaatgtgacggtgaagccggaaccg

SEQ ID NO: 195 - protein, 98 aa, Artificial Sequence
  Synthetic
NRPPIAIVSPQFQEISLPTTSTVIDGSQSTDDDKIVQYHWEELKGPLREEKISEDTAILKLSKLVPGNYTFSLTVVDSDGATNSTTANLTVNKAVDYP

SEQ ID NO: 196 - DNA, 294 bp, Artificial Sequence
  Synthetic
aatcgtccgcccatcgctattgtcagccctcaatttcaggagatcagcttgccgacaacctccactgtaattgatggttcccagtcgaccgacgacgataaaatcgtacaataccactgggaggaactcaaggggccgctgcgtgaggaaaagatcagcgaggataccgcaattttgaaacttagtaaactggttcccggcaactacacattttctttaacggttgtagactcggatggtgccaccaactctacgaccgctaatcttacagtgaacaaagcggtcgattatccg

SEQ ID NO: 197 - protein, 166 aa, Artificial Sequence
  Synthetic
SKGPGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGY

SEQ ID NO: 198 - protein, 326 aa, Artificial Sequence
  Synthetic
SKGPGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGY

SEQ ID NO: 199 - protein, 486 aa, Artificial Sequence
  Synthetic
SKGPGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGY

SEQ ID NO: 200 - protein, 646 aa, Artificial Sequence
  Synthetic
SKGPGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGY

SEQ ID NO: 201 - protein, 166 aa, Artificial Sequence
  Synthetic
SKGPGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGY

SEQ ID NO: 202 - protein, 326 aa, Artificial Sequence
  Synthetic
SKGPGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGY

SEQ ID NO: 203 - protein, 326 aa, Artificial Sequence
  Synthetic
SKGPGRGDSPYSGRGDSPYSGRGDSPYSGRGDQPYQGRGDSPYSGRGDSPYSGRGDSPYSGRGDQPYQGRGDSPYSGRGDSPYSGRGDSPYSGRGDQPYQGRGDSPYSGRGDSPYSGRGDSPYSGRGDQPYQGRGDSPYSGRGDSPYSGRGDSPYSGRGDQPYQGRGDSPYSGRGDSPYSGRGDSPYSGRGDQPYQGRGDSPYSGRGDSPYSGRGDSPYSGRGDQPYQGRGDSPYSGRGDSPYSGRGDSPYSGRGDQPYQGRGDSPYSGRGDSPYSGRGDSPYSGRGDQPYQGRGDSPYSGRGDSPYSGRGDSPYSGRGDQPYQGY

SEQ ID NO: 204 - protein, 326 aa, Artificial Sequence
  Synthetic
SKGPGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGRGDSPYSGRGDQPYQGY

SEQ ID NO: 205 - protein, 326 aa, Artificial Sequence
  Synthetic
SKGPGRGDQPYQGRGDQPYQGRGDQPYQGRGDSPYSGRGDQPYQGRGDQPYQGRGDQPYQGRGDSPYSGRGDQPYQGRGDQPYQGRGDQPYQGRGDSPYSGRGDQPYQGRGDQPYQGRGDQPYQGRGDSPYSGRGDQPYQGRGDQPYQGRGDQPYQGRGDSPYSGRGDQPYQGRGDQPYQGRGDQPYQGRGDSPYSGRGDQPYQGRGDQPYQGRGDQPYQGRGDSPYSGRGDQPYQGRGDQPYQGRGDQPYQGRGDSPYSGRGDQPYQGRGDQPYQGRGDQPYQGRGDSPYSGRGDQPYQGRGDQPYQGRGDQPYQGRGDSPYSGY

SEQ ID NO: 206 - protein, 326 aa, Artificial Sequence
  Synthetic
SKGPGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGY

SEQ ID NO: 207 - protein, 326 aa, Artificial Sequence
  Synthetic
SKGPGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGY

SEQ ID NO: 208 - protein, 326 aa, Artificial Sequence
  Synthetic
SKGPGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGRGDSPVSGY

SEQ ID NO: 209 - protein, 326 aa, Artificial Sequence
  Synthetic
SKGPGRGDSPYSGRGDSPYSGRGDSPYSGKGDSPYSGRGDSPYSGRGDSPYSGRGDSPYSGKGDSPYSGRGDSPYSGRGDSPYSGRGDSPYSGKGDSPYSGRGDSPYSGRGDSPYSGRGDSPYSGKGDSPYSGRGDSPYSGRGDSPYSGRGDSPYSGKGDSPYSGRGDSPYSGRGDSPYSGRGDSPYSGKGDSPYSGRGDSPYSGRGDSPYSGRGDSPYSGKGDSPYSGRGDSPYSGRGDSPYSGRGDSPYSGKGDSPYSGRGDSPYSGRGDSPYSGRGDSPYSGKGDSPYSGRGDSPYSGRGDSPYSGRGDSPYSGKGDSPYSGY

SEQ ID NO: 210 - protein, 326 aa, Artificial Sequence
  Synthetic
SKGPGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGRGDSPYSGKGDSPYSGY

SEQ ID NO: 211 - protein, 486 aa, Artificial Sequence
  Synthetic
SKGPGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGY

SEQ ID NO: 212 - protein, 646 aa, Artificial Sequence
  Synthetic
SKGPGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGY

SEQ ID NO: 213 - protein, 326 aa, Artificial Sequence
  Synthetic
SKFPGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGRGDTPYTGY

SEQ ID NO: 214 - protein, 166 aa, Artificial Sequence
  Synthetic
SKGPGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGY

SEQ ID NO: 215 - protein, 326 aa, Artificial Sequence
  Synthetic
SKGPGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGY

SEQ ID NO: 216 - protein, 486 aa, Artificial Sequence
  Synthetic
SKGPGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGRGDNPYNGY

SEQ ID NO: 217 - protein, 166 aa, Artificial Sequence
  Synthetic
SKGPGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGY

SEQ ID NO: 218 - protein, 326 aa, Artificial Sequence
  Synthetic
SKGPGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGY

SEQ ID NO: 219 - protein, 486 aa, Artificial Sequence
  Synthetic
SKGPGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGY

SEQ ID NO: 220 - protein, 646 aa, Artificial Sequence
  Synthetic
SKGPGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGRGDSPHSGY

SEQ ID NO: 221 - protein, 166 aa, Artificial Sequence
  Synthetic
SKGPGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGY

SEQ ID NO: 222 - protein, 326 aa, Artificial Sequence
  Synthetic
SKGPGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGRGDSPFSGY

SEQ ID NO: 223 - protein, 166 aa, Artificial Sequence
  Synthetic
SKGPGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGY

SEQ ID NO: 224 - protein, 326 aa, Artificial Sequence
  Synthetic
SKGPGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGY

SEQ ID NO: 225 - protein, 486 aa, Artificial Sequence
  Synthetic
SKGPGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGY

SEQ ID NO: 226 - protein, 646 aa, Artificial Sequence
  Synthetic
SKGPGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGRGDSPYQGY

SEQ ID NO: 227 - protein, 166 aa, Artificial Sequence
  Synthetic
SKGPGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGY

SEQ ID NO: 228 - protein, 326 aa, Artificial Sequence
  Synthetic
SKGPGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGY

SEQ ID NO: 229 - protein, 486 aa, Artificial Sequence
  Synthetic
SKGPGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGY

SEQ ID NO: 230 - protein, 646 aa, Artificial Sequence
  Synthetic
SKGPGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGRGDQPYSGY

SEQ ID NO: 231 - protein, 166 aa, Artificial Sequence
  Synthetic
SKGPGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGY

SEQ ID NO: 232 - protein, 326 aa, Artificial Sequence
  Synthetic
SKGPGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGY

SEQ ID NO: 233 - protein, 484 aa, Artificial Sequence
  Synthetic
SKGPGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYS

SEQ ID NO: 234 - protein, 645 aa, Artificial Sequence
  Synthetic
SKGPGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSGRGDNPYSG

SEQ ID NO: 235 - protein, 166 aa, Artificial Sequence
  Synthetic
SKGPGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGY

SEQ ID NO: 236 - protein, 326 aa, Artificial Sequence
  Synthetic
SKGPGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGY

SEQ ID NO: 237 - protein, 486 aa, Artificial Sequence
  Synthetic
SKGPGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGRGDSPYNGY

SEQ ID NO: 238 - protein, 166 aa, Artificial Sequence
  Synthetic
SKGPGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQCY

SEQ ID NO: 239 - protein, 326 aa, Artificial Sequence
  Synthetic
SKGPGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGY

SEQ ID NO: 240 - protein, 486 aa, Artificial Sequence
  Synthetic
SKGPGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGY

SEQ ID NO: 241 - protein, 646 aa, Artificial Sequence
  Synthetic
SKGPGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGRGDNPYQGY

SEQ ID NO: 242 - protein, 166 aa, Artificial Sequence
  Synthetic
SKGPGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGY

SEQ ID NO: 243 - protein, 326 aa, Artificial Sequence
  Synthetic
SKGPGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGY

SEQ ID NO: 244 - protein, 486 aa, Artificial Sequence
  Synthetic
SKGPGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGRGDQPYNGY

SEQ ID NO: 245 - protein, 410 aa, Artificial Sequence
  Synthetic
SKGPGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSVLSKDPNEKRDHMVLLEFVTAAGITHGMDELYKELHHHHHHG

SEQ ID NO: 246 - protein, 570 aa, Artificial Sequence
  Synthetic
SKGPGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSVLSKDPNEKRDHMVLLEFVTAAGITHGMDELYKELHHHHHHG

SEQ ID NO: 247 - protein, 166 aa, Artificial Sequence
  Synthetic
SKGPGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGRDGSPYQGY

SEQ ID NO: 248 - protein, 166 aa, Artificial Sequence
  Synthetic
SKGPGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGRDGQPYQGY

SEQ ID NO: 249 - protein, 166 aa, Artificial Sequence
  Synthetic
SKGPGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGRDGSPYNGY

SEQ ID NO: 250 - protein, 166 aa, Artificial Sequence
  Synthetic
SKGPGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGRGDNPHNGY

SEQ ID NO: 251 - protein, 166 aa, Artificial Sequence
  Synthetic
SKGPGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGRGDNPHSGY

SEQ ID NO: 252 - protein, 166 aa, Artificial Sequence
  Synthetic
SKGPGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGRGDSPHNGY

SEQ ID NO: 253 - protein, 166 aa, Artificial Sequence
  Synthetic
SKGPGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGRGDQPHNGY

SEQ ID NO: 254 - protein, 326 aa, Artificial Sequence; identical to SEQ ID NO: 203
  Synthetic

SEQ ID NO: 255 - protein, 326 aa, Artificial Sequence; identical to SEQ ID NO: 204
  Synthetic

SEQ ID NO: 256 - protein, 326 aa, Artificial Sequence; identical to SEQ ID NO: 205
  Synthetic

SEQ ID NO: 257 - protein, 326 aa, Artificial Sequence; identical to SEQ ID NO: 206
  Synthetic

SEQ ID NO: 258 - protein, 326 aa, Artificial Sequence
  Synthetic
SKFPGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGY

SEQ ID NO: 259 - protein, 326 aa, Artificial Sequence
  Synthetic
SKGPGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGY

SEQ ID NO: 260 - protein, 326 aa, Artificial Sequence; identical to SEQ ID NO: 208
  Synthetic

SEQ ID NO: 261 - protein, 326 aa, Artificial Sequence
  Synthetic
SKGPGRGDSPYSGRGDSPYSGRGDSPYSGRGDSPASGRGDSPYSGRGDSPYSGRGDSPYSGRGDSPASGRGDSPYSGRGDSPYSGRGDSPYSGRGDSPASGRGDSPYSGRGDSPYSGRGDSPYSGRGDSPASGRGDSPYSGRGDSPYSGRGDSPYSGRGDSPASGRGDSPYSGRGDSPYSGRGDSPYSGRGDSPASGRGDSPYSGRGDSPYSGRGDSPYSGRGDSPASGRGDSPYSGRGDSPYSGRGDSPYSGRGDSPASGRGDSPYSGRGDSPYSGRGDSPYSGRGDSPASGRGDSPYSGRGDSPYSGRGDSPYSGRGDSPASGY

SEQ ID NO: 262 - protein, 326 aa, Artificial Sequence
  Synthetic
SKGPGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGRGDSPYSGRGDSPASGY

SEQ ID NO: 263 - protein, 326 aa, Artificial Sequence
  Synthetic
SKGPGRGDSPYSGRGDSPYSGRGDSPYSGRGDSPISGRGDSPYSGRGDSPYSGRGDSPYSGRGDSPISGRGDSPYSGRGDSPYSGRGDSPYSGRGDSPISGRGDSPYSGRGDSPYSGRGDSPYSGRGDSPISGRGDSPYSGRGDSPYSGRGDSPYSGRGDSPISGRGDSPYSGRGDSPYSGRGDSPYSGRGDSPISGRGDSPYSGRGDSPYSGRGDSPYSGRGDSPISGRGDSPYSGRGDSPYSGRGDSPYSGRGDSPISGRGDSPYSGRGDSPYSGRGDSPYSGRGDSPISGRGDSPYSGRGDSPYSGRGDSPYSGRGDSPISGY

SEQ ID NO: 264 - protein, 358 aa, Artificial Sequence
  Synthetic
SKGPGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGRGDSPYSGRGDSPISGY

SEQ ID NO: 265 - protein, 326 aa, Artificial Sequence
  Synthetic
SKGPGRGDSPYSGRGDSPYSGRGDSPYSGRGDSPMSGRGDSPYSGRGDSPYSGRGDSPYSGRGDSPMSGRGDSPYSGRGDSPYSGRGDSPYSGRGDSPMSGRGDSPYSGRGDSPYSGRGDSPYSGRGDSPMSGRGDSPYSGRGDSPYSGRGDSPYSGRGDSPMSGRGDSPYSGRGDSPYSGRGDSPYSGRGDSPMSGRGDSPYSGRGDSPYSGRGDSPYSGRGDSPMSGRGDSPYSGRGDSPYSGRGDSPYSGRGDSPMSGRGDSPYSGRGDSPYSGRGDSPYSGRGDSPMSGRGDSPYSGRGDSPYSGRGDSPYSGRGDSPMSGY

SEQ ID NO: 266 - protein, 326 aa, Artificial Sequence
  Synthetic
SKGPGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGRGDSPYSGRGDSPMSGY

SEQ ID NO: 267 - protein, 326 aa, Artificial Sequence
  Synthetic
SKGPGRGDSPYSGRGDSPYSGRGDSPYSGRGDSPHSGRGDSPYSGRGDSPYSGRGDSPYSGRGDSPHSGRGDSPYSGRGDSPYSGRGDSPYSGRGDSPHSGRGDSPYSGRGDSPYSGRGDSPYSGRGDSPHSGRGDSPYSGRGDSPYSGRGDSPYSGRGDSPHSGRGDSPYSGRGDSPYSGRGDSPYSGRGDSPHSGRGDSPYSGRGDSPYSGRGDSPYSGRGDSPHSGRGDSPYSGRGDSPYSGRGDSPYSGRGDSPHSGRGDSPYSGRGDSPYSGRGDSPYSGRGDSPHSGRGDSPYSGRGDSPYSGRGDSPYSGRGDSPHSGY

SEQ ID NO: 268 - protein, 326 aa, Artificial Sequence
  Synthetic
SKGPGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGRGDSPYSGRGDSPHSGY

SEQ ID NO: 269 - protein, 326 aa, Artificial Sequence
  Synthetic
SKGPGRGDSPHSGRGDSPHSGRGDSPHSGRGDSPYSGRGDSPHSGRGDSPHSGRGDSPHSGRGDSPYSGRGDSPHSGRGDSPHSGRGDSPHSGRGDSPYSGRGDSPHSGRGDSPHSGRGDSPHSGRGDSPYSGRGDSPHSGRGDSPHSGRGDSPHSGRGDSPYSGRGDSPHSGRGDSPHSGRGDSPHSGRGDSPYSGRGDSPHSGRGDSPHSGRGDSPHSGRGDSPYSGRGDSPHSGRGDSPHSGRGDSPHSGRGDSPYSGRGDSPHSGRGDSPHSGRGDSPHSGRGDSPYSGRGDSPHSGRGDSPHSGRGDSPHSGRGDSPYSGY

SEQ ID NO: 270 - protein, 326 aa, Artificial Sequence; identical to SEQ ID NO: 209
  Synthetic

SEQ ID NO: 271 - protein, 326 aa, Artificial Sequence; identical to SEQ ID NO: 210
  Synthetic

SEQ ID NO: 272 - protein, 326 aa, Artificial Sequence
  Synthetic
SKGPGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGRGDSPYSGRGESPYSGY

SEQ ID NO: 273 - protein, 326 aa, Artificial Sequence
  Synthetic
SKGPGRGDSPYSGRGDSPYSGRGDSPYSGRGDSPWSGRGDSPYSGRGDSPYSGRGDSPYSGRGDSPWSGRGDSPYSGRGDSPYSGRGDSPYSGRGDSPWSGRGDSPYSGRGDSPYSGRGDSPYSGRGDSPWSGRGDSPYSGRGDSPYSGRGDSPYSGRGDSPWSGRGDSPYSGRGDSPYSGRGDSPYSGRGDSPWSGRGDSPYSGRGDSPYSGRGDSPYSGRGDSPWSGRGDSPYSGRGDSPYSGRGDSPYSGRGDSPWSGRGDSPYSGRGDSPYSGRGDSPYSGRGDSPWSGRGDSPYSGRGDSPYSGRGDSPYSGRGDSPWSGY

SEQ ID NO: 274 - protein, 326 aa, Artificial Sequence
  Synthetic
SKGPGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGRGDSPYSGRGDSPWSGY

SEQ ID NO: 275 - protein, 326 aa, Artificial Sequence
  Synthetic
SKGPGRPDSPYSGRGDSPYSGRGDSPYSGRGDSPYSPRGDSPYSGRGDSPYSGRGDSPYSGRGDSPYSGRPDSPYSGRGDSPYSGRGDSPYSGRGDSPYSPRGDSPYSGRGDSPYSGRGDSPYSGRGDSPYSGRPDSPYSGRGDSPYSGRGDSPYSGRGDSPYSGRPDSPYSGRGDSPYSGRGDSPYSGRGDSPYSPRGDSPYSGRGDSPYSGRGDSPYSGRGDSPYSGRPDSPYSGRGDSPYSGRGDSPYSGRGDSPYSPRGDSPYSGRGDSPYSGRGDSPYSGRGDSPYSGRPDSPYSGRGDSPYSGRGDSPYSGRGDSPYSGY

SEQ ID NO: 276 - protein, 326 aa, Artificial Sequence
  Synthetic
SKGPGRPDSPYSGRGDSPYSPRGDSPYSGRGDSPYSGRPDSPYSGRGDSPYSPRGDSPYSGRGDSPYSGRPDSPYSGRGDSPYSPRGDSPYSGRGDSPYSGRPDSPYSGRGDSPYSPRGDSPYSGRGDSPYSGRPDSPYSGRGDSPYSPRGDSPYSGRGDSPYSGRPDSPYSGRGDSPYSPRGDSPYSGRGDSPYSGRPDSPYSGRGDSPYSPRGDSPYSGRGDSPYSGRPDSPYSGRGDSPYSPRGDSPYSGRGDSPYSGRPDSPYSGRGDSPYSPRGDSPYSGRGDSPYSGRPDSPYSGRGDSPYSPRGDSPYSGRGDSPYSGY

SEQ ID NO: 277 - protein, 326 aa, Artificial Sequence
  Synthetic
SKGPGRPDSPYSPRGDSPYSGRPDSPYSGRGDSPYSPRGDSPYSGRPDSPYSPRGDSPYSGRGDSPYSGRPDSPYSPRGDSPYSGRPDSPYSGRGDSPYSPRGDSPYSGRPDSPYSPRGDSPYSGRGDSPYSGRPDSPYSPRGDSPYSGRPDSPYSGRGDSPYSGRPDSPYSPRGDSPYSGRPDSPYSGRGDSPYSPRGDSPYSGRPDSPYSPRGDSPYSGRGDSPYSGRPDSPYSPRGDSPYSGRPDSPYSGRGDSPYSPRGDSPYSGRPDSPYSPRGDSPYSGRGDSPYSGRPDSPYSPRGDSPYSGRPDSPYSGRGDSPYSGY

SEQ ID NO: 278 - protein, 570 aa, Artificial Sequence
  Synthetic
SKGPGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSVLSKDPNEKRDHMVLLEFVTAAGITHGMDELYKELHHHHHHG

SEQ ID NO: 279 - protein, 323 aa, Artificial Sequence
  Synthetic
  MISC_FEATURE (1)..(1): X = azido-phenylalanine  (AzF)
  MISC_FEATURE (162)..(162): X = azido-phenylalanine  (AzF)
  MISC_FEATURE (323)..(323): X = azido-phenylalanine  (AzF)
XGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSXGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSX

SEQ ID NO: 280 - protein, 246 aa, Artificial Sequence
  Synthetic
GKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSVLSKDPNEKRDHMVLLEFVTAAGITHGMDELYKELHHHHHHG

SEQ ID NO: 281 - protein, 237 aa, Artificial Sequence
  Synthetic
GVSKGEELIKENMRMKVVMEGSVNGHQFKCTGEGEGRPYEGVQTMRIKVIEGGPLPFAFDILATSFMYGSRTFIKYPADIPDFFKQSFPEGFTWERVTRYEDGGVVTVTQDTSLEDGELVYNVKVRGVNFPSNGPVMQKKTKGWEPNTEMMYPADGGLRGYTDIALKVDGGGHLHCNFVTTYRSKKTVGNIKMPGVHAVDHRLERIEESDNETYVVQREVAVAKYSNLGGGMDELYK

SEQ ID NO: 282 - DNA, 60 bp, Artificial Sequence
  Synthetic
ctcactatagggagacccaagctggctagcatgagcaaagggccgggacgcggcgatagt

SEQ ID NO: 283 - DNA, 60 bp, Artificial Sequence
  Synthetic
ttagccgtgatggtgatggtgatggagctcgttgattgtcgagggccctctagactcgag

SEQ ID NO: 284 - protein, 378 aa, Artificial Sequence
  Synthetic
GRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQVDNKFNKEQQNAFYEILHLPNLNEEQRNAFIQSLKDDPSQSANLLAEAKKLNDAQAPK

SEQ ID NO: 285 - DNA, 1134 bp, Artificial Sequence
  Synthetic
gggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcaggggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcaggtggacaataagttcaataaggagcagcagaatgccttttacgagattctccacctgcccaacctcaacgaagagcagagaaatgcgtttatccagagtctgaaagacgacccaagccagtccgcaaacctgctggctgaggccaagaagcttaacgacgcacaggccccaaag

SEQ ID NO: 286 - protein, 218 aa, Artificial Sequence
  Synthetic
GRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQVDNKFNKEQQNAFYEILHLPNLNEEQRNAFIQSLKDDPSQSANLLAEAKKLNDAQAPK

SEQ ID NO: 287 - DNA, 654 bp, Artificial Sequence
  Synthetic
gggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcaggtggacaataagttcaataaggagcagcagaatgccttttacgagattctccacctgcccaacctcaacgaagagcagagaaatgcgtttatccagagtctgaaagacgacccaagccagtccgcaaacctgctggctgaggccaagaagcttaacgacgcacaggccccaaag

SEQ ID NO: 288 - protein, 418 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSNRPPIAIVSPQFQEISLPTTSTVIDGSQSTDDDKIVQYHWEELKGPLREEKISEDTAILKLSKLVPGNYTFSLTVVDSDGATNSTTANLTVNKAVDYP

SEQ ID NO: 289 - DNA, 1254 bp, Artificial Sequence
  Synthetic
ggacgcggcgatagtccatattcaggtcggggagatagtccctactcaggtaggggagacagcccatatagcgggcgtggcgactctccatatagtgggagaggggactctccgtactctgggcgcggtgattcaccatattctggtcgaggtgactcaccttattctggcaggggtgatagtccgtattcaggccgtggtgattccccctatagtggaaggggagattcaccgtatagtggtagaggcgacagtccttattcgggacgaggtgattctccttatagtggccgaggggattcaccctattctggaagaggtgatagcccttattcagggaggggcgattctccctattcaggacggggtgactctccttactccggtcgcggagattctccatactcgggtcgtggagactccccatattccggcagaggtgacagtccatacagtggacgtggggatagtccttacagtggacgcggcgatagtccatattcaggtcggggagatagtccctactcaggtaggggagacagcccatatagcgggcgtggcgactctccatatagtgggagaggggactctccgtactctgggcgcggtgattcaccatattctggtcgaggtgactcaccttattctggcaggggtgatagtccgtattcaggccgtggtgattccccctatagtggaaggggagattcaccgtatagtggtagaggcgacagtccttattcgggacgaggtgattctccttatagtggccgaggggattcaccctattctggaagaggtgatagcccttattcagggaggggcgattctccctattcaggacggggtgactctccttactccggtcgcggagattctccatactcgggtcgtggagactccccatattccggcagaggtgacagtccatacagtggacgtggggatagtccttacagtaatcgtccgcccatcgctattgtcagccctcaatttcaggagatcagcttgccgacaacctccactgtaattgatggttcccagtcgaccgacgacgataaaatcgtacaataccactgggaggaactcaaggggccgctgcgtgaggaaaagatcagcgaggataccgcaattttgaaacttagtaaactggttcccggcaactacacattttctttaacggttgtagactcggatggtgccaccaactctacgaccgctaatcttacagtgaacaaagcggtcgattatccg

SEQ ID NO: 290 - protein, 555 aa, Artificial Sequence
  Synthetic
GRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 291 - DNA, 1665 bp, Artificial Sequence
  Synthetic
ggacgcggcgatagtccatattcaggtcggggagatagtccctactcaggtaggggagacagcccatatagcgggcgtggcgactctccatatagtgggagaggggactctccgtactctgggcgcggtgattcaccatattctggtcgaggtgactcaccttattctggcaggggtgatagtccgtattcaggccgtggtgattccccctatagtggaaggggagattcaccgtatagtggtagaggcgacagtccttattcgggacgaggtgattctccttatagtggccgaggggattcaccctattctggaagaggtgatagcccttattcagggaggggcgattctccctattcaggacggggtgactctccttactccggtcgcggagattctccatactcgggtcgtggagactccccatattccggcagaggtgacagtccatacagtggacgtggggatagtccttacagtggacgcggcgatagtccatattcaggtcggggagatagtccctactcaggtaggggagacagcccatatagcgggcgtggcgactctccatatagtgggagaggggactctccgtactctgggcgcggtgattcaccatattctggtcgaggtgactcaccttattctggcaggggtgatagtccgtattcaggccgtggtgattccccctatagtggaaggggagattcaccgtatagtggtagaggcgacagtccttattcgggacgaggtgattctccttatagtggccgaggggattcaccctattctggaagaggtgatagcccttattcagggaggggcgattctccctattcaggacggggtgactctccttactccggtcgcggagattctccatactcgggtcgtggagactccccatattccggcagaggtgacagtccatacagtggacgtggggatagtccttacagtgtgagcaagggcgaggaagataacatggcctctctcccagcgacacatgagttacacatctttggctccatcaacggtgtggactttgacatggtgggtcagggcaccggcaatccaaatgatggttatgaagagttaaacctcaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctaaagaaccagccgatgtacgtgttccgtaagacggagctcaagcactccaagaccgagctcaacttcaaggagtggcaaaaggcctttaccgatgtgatgggcatggacgagctgtacaag

SEQ ID NO: 292 - protein, 192 aa, Artificial Sequence
  Synthetic
AAHGEGTFTSDVSSYLEEQAAKEFIAWLVKGAGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVS

SEQ ID NO: 293 - DNA, 576 bp, Artificial Sequence
  Synthetic
gctgcccatggggaggggacattcacctcggacgtaagcagttacttagaagaacaggctgcaaaagagtttattgcgtggcttgtgaaaggagcaggacgaggggactcaccttatagtgggcgcggcgactctccatattccggtcggggagattcaccatatagtggccgtggcgatagtcctgtgtctggtaggggagactctccgtattcaggacgcggtgattcaccctacagtggacggggtgattctccttattcgggtcgtggggattctccagtttcaggtagaggtgatagcccttattcaggccgaggtgacagtccttactccggcaggggtgactccccatactcgggacgtggtgatagtccagtatcaggaaggggagatagtccctactctgggagaggggatagtccgtactcaggtcgaggtgattccccctattctggaagaggtgactctcctgtatctggcagaggcgacagtccatacagcgggcgtggagattctccctatagtggtcgcggagacagcccatattcagggaggggcgattcacctgttagt

SEQ ID NO: 294 - protein, 192 aa, Artificial Sequence
  Synthetic
AAHGEGTFTSDVSSYLEEQAAKEFIAWLVKGAGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVS

SEQ ID NO: 295 - DNA, 576 bp, Artificial Sequence
  Synthetic
gctgcccatggggaggggacattcacctcggacgtaagcagttacttagaagaacaggctgcaaaagagtttattgcgtggcttgtgaaaggagcaggacgcggagattctccatactctggtcgaggggatagtccggtctcaggaagaggcgatagtccatattcaggacgaggtgacagtccagtatctggcagaggggactctccgtattccggcaggggagacagccctgtttcaggccgaggtgattctccttactcaggtcggggagatagtcctgtgtctggtaggggtgactccccctattctggaaggggagattcacctgtaagtgggcgtggtgattcaccctactcgggtcgtggagactctccagttagtggtcgcggtgattccccatatagcgggcgcggcgattctcccgtttctgggaggggcgacagtccttattcgggacgtggcgactctcctgtatcagggagaggtgatagtccctacagtggccgtggggattcaccagtgtcaggtagaggtgactcaccttatagtggacggggtgatagcccagtcagt

SEQ ID NO: 296 - protein, 192 aa, Artificial Sequence
  Synthetic
AAHGEGTFTSDVSSYLEEQAAKEFIAWLVKGAGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYS

SEQ ID NO: 297 - DNA, 576 bp, Artificial Sequence
  Synthetic
gctgcccatggggaggggacattcacctcggacgtaagcagttacttagaagaacaggctgcaaaagagtttattgcgtggcttgtgaaaggagcaggacggggtgatagccctgtatctggaaggggagattcaccagtcagtggacgcggtgattcccctgttagtggccgaggtgatagtccttactcaggacgtggcgactctccagtatcgggacgaggggacagcccagtaagcggcaggggcgattctcctgtaagtgggagaggtgattctccatatagtggtcgcggagattctcccgtttcaggtaggggagactcgcctgtctcaggtcgaggtgacagtcctgtgtctggtcggggagatagtccctattctggtagaggggatagtccggtttcgggtcgtggagattcgccggtctctggcagaggtgactcacctgtttctgggcgtggtgattcaccttattcagggaggggtgactccccagtgtcaggccgtggggattcacccgtatcaggaagaggcgatagtccagtttccgggcgcggggactctccttatagt

SEQ ID NO: 298 - protein, 352 aa, Artificial Sequence
  Synthetic
AAHGEGTFTSDVSSYLEEQAAKEFIAWLVKGAGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYS

SEQ ID NO: 299 - DNA, 1056 bp, Artificial Sequence
  Synthetic
gctgcccatggggaggggacattcacctcggacgtaagcagttacttagaagaacaggctgcaaaagagtttattgcgtggcttgtgaaaggagcaggacgcggcgatagtccatattcaggtcggggagatagtccctactcaggtaggggagacagcccatatagcgggcgtggcgactctccatatagtgggagaggggactctccgtactctgggcgcggtgattcaccatattctggtcgaggtgactcaccttattctggcaggggtgatagtccgtattcaggccgtggtgattccccctatagtggaaggggagattcaccgtatagtggtagaggcgacagtccttattcgggacgaggtgattctccttatagtggccgaggggattcaccctattctggaagaggtgatagcccttattcagggaggggcgattctccctattcaggacggggtgactctccttactccggtcgcggagattctccatactcgggtcgtggagactccccatattccggcagaggtgacagtccatacagtggacgtggggatagtccttacagtggacgcggcgatagtccatattcaggtcggggagatagtccctactcaggtaggggagacagcccatatagcgggcgtggcgactctccatatagtgggagaggggactctccgtactctgggcgcggtgattcaccatattctggtcgaggtgactcaccttattctggcaggggtgatagtccgtattcaggccgtggtgattccccctatagtggaaggggagattcaccgtatagtggtagaggcgacagtccttattcgggacgaggtgattctccttatagtggccgaggggattcaccctattctggaagaggtgatagcccttattcagggaggggcgattctccctattcaggacggggtgactctccttactccggtcgcggagattctccatactcgggtcgtggagactccccatattccggcagaggtgacagtccatacagtggacgtggggatagtccttacagt

SEQ ID NO: 300 - protein, 192 aa, Artificial Sequence
  Synthetic
AAHGEGTFTSDVSSYLEEQAAKEFIAWLVKGAGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYSGRGDSPYS

SEQ ID NO: 301 - DNA, 576 bp, Artificial Sequence
  Synthetic
gctgcccatggggaggggacattcacctcggacgtaagcagttacttagaagaacaggctgcaaaagagtttattgcgtggcttgtgaaaggagcaggacgcggcgatagtccatattcaggtcggggagatagtccctactcaggtaggggagacagcccatatagcgggcgtggcgactctccatatagtgggagaggggactctccgtactctgggcgcggtgattcaccatattctggtcgaggtgactcaccttattctggcaggggtgatagtccgtattcaggccgtggtgattccccctatagtggaaggggagattcaccgtatagtggtagaggcgacagtccttattcgggacgaggtgattctccttatagtggccgaggggattcaccctattctggaagaggtgatagcccttattcagggaggggcgattctccctattcaggacggggtgactctccttactccggtcgcggagattctccatactcgggtcgtggagactccccatattccggcagaggtgacagtccatacagtggacgtggggatagtccttacagt

SEQ ID NO: 302 - protein, 352 aa, Artificial Sequence
  Synthetic
AAHGEGTFTSDVSSYLEEQAAKEFIAWLVKGAGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVS

SEQ ID NO: 303 - DNA, 1056 bp, Artificial Sequence
  Synthetic
gctgcccatggggaggggacattcacctcggacgtaagcagttacttagaagaacaggctgcaaaagagtttattgcgtggcttgtgaaaggagcaggacgaggggactcaccttatagtgggcgcggcgactctccatattccggtcggggagattcaccatatagtggccgtggcgatagtcctgtgtctggtaggggagactctccgtattcaggacgcggtgattcaccctacagtggacggggtgattctccttattcgggtcgtggggattctccagtttcaggtagaggtgatagcccttattcaggccgaggtgacagtccttactccggcaggggtgactccccatactcgggacgtggtgatagtccagtatcaggaaggggagatagtccctactctgggagaggggatagtccgtactcaggtcgaggtgattccccctattctggaagaggtgactctcctgtatctggcagaggcgacagtccatacagcgggcgtggagattctccctatagtggtcgcggagacagcccatattcagggaggggcgattcacctgttagtggacgaggggactcaccttatagtgggcgcggcgactctccatattccggtcggggagattcaccatatagtggccgtggcgatagtcctgtgtctggtaggggagactctccgtattcaggacgcggtgattcaccctacagtggacggggtgattctccttattcgggtcgtggggattctccagtttcaggtagaggtgatagcccttattcaggccgaggtgacagtccttactccggcaggggtgactccccatactcgggacgtggtgatagtccagtatcaggaaggggagatagtccctactctgggagaggggatagtccgtactcaggtcgaggtgattccccctattctggaagaggtgactctcctgtatctggcagaggcgacagtccatacagcgggcgtggagattctccctatagtggtcgcggagacagcccatattcagggaggggcgattcacctgttagt

SEQ ID NO: 304 - protein, 352 aa, Artificial Sequence
  Synthetic
AAHGEGTFTSDVSSYLEEQAAKEFIAWLVKGAGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVSGRGDSPYSGRGDSPVS

SEQ ID NO: 305 - DNA, 1056 bp, Artificial Sequence
  Synthetic
gctgcccatggggaggggacattcacctcggacgtaagcagttacttagaagaacaggctgcaaaagagtttattgcgtggcttgtgaaaggagcaggacgcggagattctccatactctggtcgaggggatagtccggtctcaggaagaggcgatagtccatattcaggacgaggtgacagtccagtatctggcagaggggactctccgtattccggcaggggagacagccctgtttcaggccgaggtgattctccttactcaggtcggggagatagtcctgtgtctggtaggggtgactccccctattctggaaggggagattcacctgtaagtgggcgtggtgattcaccctactcgggtcgtggagactctccagttagtggtcgcggtgattccccatatagcgggcgcggcgattctcccgtttctgggaggggcgacagtccttattcgggacgtggcgactctcctgtatcagggagaggtgatagtccctacagtggccgtggggattcaccagtgtcaggtagaggtgactcaccttatagtggacggggtgatagcccagtcagtggacgcggagattctccatactctggtcgaggggatagtccggtctcaggaagaggcgatagtccatattcaggacgaggtgacagtccagtatctggcagaggggactctccgtattccggcaggggagacagccctgtttcaggccgaggtgattctccttactcaggtcggggagatagtcctgtgtctggtaggggtgactccccctattctggaaggggagattcacctgtaagtgggcgtggtgattcaccctactcgggtcgtggagactctccagttagtggtcgcggtgattccccatatagcgggcgcggcgattctcccgtttctgggaggggcgacagtccttattcgggacgtggcgactctcctgtatcagggagaggtgatagtccctacagtggccgtggggattcaccagtgtcaggtagaggtgactcaccttatagtggacggggtgatagcccagtcagt

SEQ ID NO: 306 - protein, 352 aa, Artificial Sequence
  Synthetic
AAHGEGTFTSDVSSYLEEQAAKEFIAWLVKGAGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYSGRGDSPVSGRGDSPVSGRGDSPVSGRGDSPYS

SEQ ID NO: 307 - DNA, 1056 bp, Artificial Sequence
  Synthetic
gctgcccatggggaggggacattcacctcggacgtaagcagttacttagaagaacaggctgcaaaagagtttattgcgtggcttgtgaaaggagcaggacggggtgatagccctgtatctggaaggggagattcaccagtcagtggacgcggtgattcccctgttagtggccgaggtgatagtccttactcaggacgtggcgactctccagtatcgggacgaggggacagcccagtaagcggcaggggcgattctcctgtaagtgggagaggtgattctccatatagtggtcgcggagattctcccgtttcaggtaggggagactcgcctgtctcaggtcgaggtgacagtcctgtgtctggtcggggagatagtccctattctggtagaggggatagtccggtttcgggtcgtggagattcgccggtctctggcagaggtgactcacctgtttctgggcgtggtgattcaccttattcagggaggggtgactccccagtgtcaggccgtggggattcacccgtatcaggaagaggcgatagtccagtttccgggcgcggggactctccttatagtggacggggtgatagccctgtatctggaaggggagattcaccagtcagtggacgcggtgattcccctgttagtggccgaggtgatagtccttactcaggacgtggcgactctccagtatcgggacgaggggacagcccagtaagcggcaggggcgattctcctgtaagtgggagaggtgattctccatatagtggtcgcggagattctcccgtttcaggtaggggagactcgcctgtctcaggtcgaggtgacagtcctgtgtctggtcggggagatagtccctattctggtagaggggatagtccggtttcgggtcgtggagattcgccggtctctggcagaggtgactcacctgtttctgggcgtggtgattcaccttattcagggaggggtgactccccagtgtcaggccgtggggattcacccgtatcaggaagaggcgatagtccagtttccgggcgcggggactctccttatagt

SEQ ID NO: 308 - protein, 344 aa, Artificial Sequence
  Synthetic
GRIGSILGRLAKGLPTLKSWIKNRGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQ

SEQ ID NO: 309 - DNA, 1032 bp, Artificial Sequence
  Synthetic
ggcagaataggctctatccttggaagacttgccaaggggctgcctactctaaagagctggattaagaaccgcgggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcaggggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcag

SEQ ID NO: 310 - protein, 351 aa, Artificial Sequence
  Synthetic
GVGHNADLQIKLSIRRLLAAGVLKQTKGVGAGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQ

SEQ ID NO: 311 - DNA, 1053 bp, Artificial Sequence
  Synthetic
ggcgtcggacataatgcggatctgcaaatcaaactgagcattcgccgtctgctggcggcgggcgttctgaaacagaccaaaggcgtgggcgcagggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcaggggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcag

SEQ ID NO: 312 - protein, 357 aa, Artificial Sequence
  Synthetic
LLGDFFRKSKEKIGKEFKRIVQRIKDFLRNLVPRTESGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQ

SEQ ID NO: 313 - DNA, 1071 bp, Artificial Sequence
  Synthetic
cttctgggggatttttttcgcaaatctaaggaaaaaatcgggaaggaatttaaacgcatcgttcaacgcattaaagacttccttcgtaaccttgtccctcgtacagagagtgggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcaggggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcag

SEQ ID NO: 314 - protein, 667 aa, Artificial Sequence
  Synthetic
GRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSGRGDSPYSGRGDSPYSGRGDSPYSGRGDSPVSRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDL

SEQ ID NO: 315 - DNA, 2001 bp, Artificial Sequence
  Synthetic
ggacgaggggactcaccttatagtgggcgcggcgactctccatattccggtcggggagattcaccatatagtggccgtggcgatagtcctgtgtctggtaggggagactctccgtattcaggacgcggtgattcaccctacagtggacggggtgattctccttattcgggtcgtggggattctccagtttcaggtagaggtgatagcccttattcaggccgaggtgacagtccttactccggcaggggtgactccccatactcgggacgtggtgatagtccagtatcaggaaggggagatagtccctactctgggagaggggatagtccgtactcaggtcgaggtgattccccctattctggaagaggtgactctcctgtatctggcagaggcgacagtccatacagcgggcgtggagattctccctatagtggtcgcggagacagcccatattcagggaggggcgattcacctgttagtggacgaggggactcaccttatagtgggcgcggcgactctccatattccggtcggggagattcaccatatagtggccgtggcgatagtcctgtgtctggtaggggagactctccgtattcaggacgcggtgattcaccctacagtggacggggtgattctccttattcgggtcgtggggattctccagtttcaggtagaggtgatagcccttattcaggccgaggtgacagtccttactccggcaggggtgactccccatactcgggacgtggtgatagtccagtatcaggaaggggagatagtccctactctgggagaggggatagtccgtactcaggtcgaggtgattccccctattctggaagaggtgactctcctgtatctggcagaggcgacagtccatacagcgggcgtggagattctccctatagtggtcgcggagacagcccatattcagggaggggcgattcacctgttagtcgcagtcgtttattagaggatttccgtaacaaccgttaccctaatttgcaactgcgtgagattgctggccacattatggaattttcacaagaccagcacggatcgcgtttcattcaacttaaattagaacgcgctacgccagccgaacgccagttagttttcaacgaaattttacaagcggcataccaactgatggtggacgtttttgggaattatgttatccagaagttcttcgagtttggaagcctggagcagaaacttgccctggcggaacgcattcgtggtcacgtcttaagtttggcactgcagatgtatgggtgccgtgttatccaaaaagctcttgagtttatcccatctgatcagcagaacgaaatggtacgcgagctggacggccatgttcttaaatgcgtaaaggaccagaacggtaatcatgttgtacagaaatgcatcgaatgcgtccagccacagtctctgcagttcatcattgatgctttcaaaggccaagtttttgcccttagcacgcatccctacgggtgtcgtgtcatccaacgcattcttgagcattgcttaccagatcaaaccttgccaatcctggaggaattgcaccaacacactgaacagctggtacaagatcaatatggtaactatgtaattcagcacgttctggaacatggacgccctgaggacaagtctaaaattgtcgcggaaatccgtggaaacgtattggtgttgtcccaacacaaatttgccagtaatgtcgtggagaaatgcgtaacccatgctagccgtaccgaacgcgcagtcttaatcgacgaagtttgcacaatgaatgacggcccgcattcggctttatatacaatgatgaaggaccagtacgctaattatgtcgtgcagaaaatgattgatgtagcggagcctggtcagcgcaagattgtgatgcacaagatccgcccgcacattgcgacattgcgtaagtatacgtacggcaaacacatcttggcgaaacttgaaaagtactacatgaaaaatggcgtggacctt

SEQ ID NO: 316 - protein, 667 aa, Artificial Sequence
  Synthetic
GRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQGRGDQPYQRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDL

SEQ ID NO: 317 - DNA, 2001 bp, Artificial Sequence
  Synthetic
gggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcaggggcgcggggaccagccatatcaaggaagaggtgaccaaccttatcagggtagaggcgatcagccttatcaaggtcgcggtgatcaaccgtatcaaggtagaggggaccaaccatatcagggaaggggagatcaaccttaccaaggtcggggagatcagccctaccaaggcagaggggatcaaccgtaccaggggaggggtgatcaaccataccagggtcgaggcgaccaaccctatcagggccgaggtgatcagccgtaccaagggcgtggcgaccagccctatcaaggacgtggggatcagccataccaaggacgcggagatcaaccatatcaagggagaggtgatcaaccctatcaaggtaggggtgaccaaccgtatcagggtcgtggagatcagccttaccagggcaggggcgatcaaccctaccagggacgtggtgatcaaccttatcaaggccgtggtgaccagccttatcagcgcagtcgtttattagaggatttccgtaacaaccgttaccctaatttgcaactgcgtgagattgctggccacattatggaattttcacaagaccagcacggatcgcgtttcattcaacttaaattagaacgcgctacgccagccgaacgccagttagttttcaacgaaattttacaagcggcataccaactgatggtggacgtttttgggaattatgttatccagaagttcttcgagtttggaagcctggagcagaaacttgccctggcggaacgcattcgtggtcacgtcttaagtttggcactgcagatgtatgggtgccgtgttatccaaaaagctcttgagtttatcccatctgatcagcagaacgaaatggtacgcgagctggacggccatgttcttaaatgcgtaaaggaccagaacggtaatcatgttgtacagaaatgcatcgaatgcgtccagccacagtctctgcagttcatcattgatgctttcaaaggccaagtttttgcccttagcacgcatccctacgggtgtcgtgtcatccaacgcattcttgagcattgcttaccagatcaaaccttgccaatcctggaggaattgcaccaacacactgaacagctggtacaagatcaatatggtaactatgtaattcagcacgttctggaacatggacgccctgaggacaagtctaaaattgtcgcggaaatccgtggaaacgtattggtgttgtcccaacacaaatttgccagtaatgtcgtggagaaatgcgtaacccatgctagccgtaccgaacgcgcagtcttaatcgacgaagtttgcacaatgaatgacggcccgcattcggctttatatacaatgatgaaggaccagtacgctaattatgtcgtgcagaaaatgattgatgtagcggagcctggtcagcgcaagattgtgatgcacaagatccgcccgcacattgcgacattgcgtaagtatacgtacggcaaacacatcttggcgaaacttgaaaagtactacatgaaaaatggcgtggacctt

(19) United States
(12) Patent Application Publication
(10) Pub. No.: US 2023/0086188 A1
(43) Pub. Date: Mar. 23, 2023

(54) PROTEINS WITH PREDICTABLE LIQUID-LIQUID PHASE SEPARATION

(71) Applicant: Duke University, Durham, NC (US)

(72) Inventors: Michael Dzuricky, Durham, NC (US); Ashutosh Chilkoti, Durham, NC (US)

(21) Appl. No.: 17/908,427

(22) PCT Filed: Mar. 3, 2021
(86) PCT No.: PCT/US2021/020591

§ 371 Date: Aug. 31, 2022

Related U.S. Application Data
(60) Provisional application No. 62/985,179, filed on Mar. 4, 2020.

(51) Int. Cl. C07K 14/435

(52) U.S. Cl. CPC C07K 14/435; C07K 2319/35

(57) ABSTRACT

Described herein are peptide biopolymers that exhibit controlled phase separation based on their amino acid sequence, aromatic:aliphatic ratio, hydrophobicity, temperature, molecular weight, and concentration.

### CROSS REFERENCE TO RELATED APPLICATIONS

This application claims priority to U.S. Provisional Patent Application No. 62/985,179 filed on Mar. 4, 2020, which is incorporated by reference herein in its entirety.

### FEDERALLY SPONSORED RESEARCH

This invention was made with United States government support under National Institutes of Health grant number R35GM127042 and the National Science Foundation grant number DMR-17-29671. The United States government has certain rights in the invention.

### SEQUENCE LISTING

This application is filed with a Computer Readable Form of a Sequence Listing in accord with 37 C.F.R. § 1.821(c). The text file submitted by EFS, “028193-9340-WO01_sequence_listing_2-MAR-2021_ST25.txt,” was created on Mar. 2, 2021, contains 317 sequences, has a file size of 527 Kbytes, and is hereby incorporated by reference in its entirety.

### TECHNICAL FIELD

Described herein are peptide biopolymers that exhibit controlled phase separation based on their amino acid sequence, aromatic:aliphatic ratio, hydrophobicity, temperature, molecular weight, and concentration.

### BACKGROUND

Intrinsically disordered proteins (IDPs) are receiving significant recognition for their role in various biological (dys)functions. A subset of IDPs, termed biological condensates, physically separate themselves from the cytoplasm to control the accessibility of a variety of macromolecules. While our ability to detect protein disorder has advanced rapidly thanks to sophisticated statistical methods, the ability to predict phase separation has lagged behind. The prediction of phase separation is non-trivial, as numerous variables influence phase separation. Broadly, they involve: (1) amino acid composition and amino acid patterning of the primary protein sequence; (2) heterotypic interactions with RNA or other macromolecules; and (3) solvent quality. There are many studies that note the challenge of predicting IDP phase behavior, but few studies that have directly tackled this problem. Given the recognition of its importance to cellular function, this is now an area of active research and many efforts are ongoing using computational and experimental approaches. To date, however, most experimental methods to develop a sequence level understanding of IDP phase behavior have relied on mutational strategies of native IDPs with sweeping residue level or domain level mutations.

What is needed are peptide biopolymers comprising intrinsically disordered proteins that exhibit controlled phase separation based on their amino acid sequence, aromatic:aliphatic ratio, hydrophobicity, temperature, molecular weight, and concentration.

### SUMMARY

One embodiment described herein is a polypeptide with controlled reversible phase separation comprising ten or more repeats of an amino acid sequence comprising: (X-Z₁-X-Z₂-Z₃-X-Z₄-Z₃)ₙ, where: X is proline (P) or glycine (G) and the ratio of P:G is any number, Z₁ is arginine (R), aspartic acid (D), or lysine (K) and the ratio of R:D is any number and the ratio of K:R can be any number; Z₂ is Asp (D), Arg (R), Glu (E), where the ratio of R:D can be any number and D:E can be any number; Z₃ is asparagine (N), glutamine (Q), serine (S), or threonine (T) were the ratio among N:Q:S:T can be any number, and Z₄ is tyrosine (Y), histidine (H), tryptophan (W), phenylalanine (F), methionine (M), valine (V), isoleucine (I), alanine (A), or leucine (L) and the ratio among Y:H:W:F:M:V:I:A:L can be any number. In one aspect, X is proline (P) or glycine (G) and the ratio of P:G is between 1:3 and 3:1. In another aspect, Z₁ is arginine (R), aspartic acid (D), or lysine (K) and the ratio of R:D does not exceed 1:5 and the ratio of K:R can be any number. In another aspect, the phase separation is dependent on temperature, molecular weight, hydrophobicity, aromatic:aliphatic ratio, and concentration. In another aspect, n is 10 to 200. In another aspect, molecular weight is at least 5 kDa to 500 kDa. In another aspect, the molecular weight is about 5 kDa to about 100 kDa. In another aspect, the phase separation temperature is 0 to 100° C. In another aspect, the phase separation temperature is 4 to 25° C.; ˜25° C.; 25 to 37° C.; ˜37° C.; 35 to 38° C.; or >38° C. In another aspect, the polypeptide comprises modified amino acids, a reporter protein, or an enzyme. In another aspect, the sequence comprises: (G-R-G-D-S-P-Y-S)ₘ, where m is 20 to 80. In another aspect, the polypeptide comprises a sequence selected from one or more of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, or 197-279, or combinations thereof.

Another embodiment described herein is a pharmaceutically acceptable composition comprising a polypeptide with controlled reversible phase separation comprising ten or more repeats of an amino acid sequence comprising: (X-Z₁-X-Z₂-Z₃-X-Z₄-Z₃)ₙ, where: X is proline (P) or glycine (G) and the ratio of P:G is any number; Z₁ is arginine (R), aspartic acid (D), or lysine (K) and the ratio of R:D is any number and the ratio of K:R can be any number; Z₂ is Asp (D), Arg (R), Glu (E), where the ratio of R:D can be any number and D:E can be any number; Z₃ is asparagine (N), glutamine (Q), serine (S), or threonine (T) were the ratio among N:Q:S:T can be any number; and Z₄ is tyrosine (Y), histidine (H), tryptophan (W), phenylalanine (F), methionine (M), valine (V), isoleucine (I), alanine (A), or leucine (L) and the ratio among Y:H:W:F:M:V:I:A:L can be any number. In one aspect, X is proline (P) or glycine (G) and the ratio of P:G is between 1:3 and 3:1. In another aspect, Z₁ is arginine (R), aspartic acid (D), or lysine (K) and the ratio of R:D does not exceed 1:5 and the ratio of K:R can be any number. In another aspect, the composition further comprises an attached molecule comprising one or more of an antibody binding domain derived from Staphylococcus protein A (ZD) (SEQ ID NO:159), an antimicrobial peptide selected from LL37 (SEQ ID NO: 161), Ib-M1 (SEQ ID NO: 163), Ib-M2 (SEQ ID NO: 165), Ib-M5 (SEQ ID NO: 167), Cathelecidin-1 (SEQ ID NO: 169), A(A1R, A8R, I17K) (SEQ ID NO: 171), H5 (SEQ ID NO: 173), H5-61-90 (SEQ ID NO: 175); RGD peptide (RGDSPAS, SEQ ID NO: 39); protein drugs, GLP-1 (SEQ ID NO: 177); fluorescent reporters (sfGFP (SEQ ID NO: 179), mRuby3 (SEQ ID NO: 181); RNA binding proteins (PUM-HD (SEQ ID NO: 183), eIF4E (SEQ ID NO: 185), PABP (SEQ ID NO: 187), Tis11D (SEQ ID NO: 189)); KH domains (Yifan or FMRP (SEQ ID NO: 191)); or AAV binding peptides PKD1 (SEQ ID NO: 193) or PKD2 (SEQ ID NO: 195). In another aspect, the composition enhances bioavailability of the attached molecule as compared to the free form of the attached molecule. In another aspect, the composition enhances expression of the attached molecule as compared to the free form of the attached molecule. In another aspect, the composition enhances the stability of the attached molecule as compared to the free form of the attached molecule. In another aspect, the composition enhances stability of the attached molecule during prokaryotic and eukaryotic expression as compared to the free form of the attached molecule. In another aspect, the enhanced stability includes resistance to denaturation during freezing, thawing, or lyophilization. In another aspect, the composition modulates enzymatic, metabolic, or physiological functions within cells or organisms. In another aspect, the modulation reduces bioavailability of the attached molecules. In another aspect, the attached molecules comprise therapeutic or cytotoxic proteins or peptides.

Another embodiment described herein is a method for enhancing the bioavailability or stability of a protein, the method comprising creating a fusion protein of one or more proteins and a polypeptide with controlled reversible phase separation comprising ten or more repeats of an amino acid sequence comprising: (X-Z₁-X-Z₂-Z₃-X-Z₄-Z₃)ₙ, where: X is proline (P) or glycine (G) and the ratio of P:G is any number; Z₁ is arginine (R), aspartic acid (D), or lysine (K) and the ratio of R:D is any number and the ratio of K:R can be any number; Z₂ is Asp (D), Arg (R), Glu (E), where the ratio of R:D can be any number and D:E can be any number Z₃ is asparagine (N), glutamine (Q), serine (S), or threonine (T) were the ratio among N:Q:S:T can be any number; and Z₄ is tyrosine (Y), histidine (H), tryptophan (W), phenylalanine (F), methionine (M), valine (V), isoleucine (I), alanine (A), or leucine (L) and the ratio among Y:H:W:F:M:V:I:A:L can be any number. In one aspect, X is proline (P) or glycine (G) and the ratio of P:G is between 1:3 and 3:1. In another aspect, Z₁ is arginine (R), aspartic acid (D), or lysine (K) and the ratio of R:D does not exceed 1:5 and the ratio of K:R can be any number. In one aspect, X is proline (P) or glycine (G) and the ratio of P:G is between 1:3 and 3:1. In another aspect, Z₁ is arginine (R), aspartic acid (D), or lysine (K) and the ratio of R:D does not exceed 1:5 and the ratio of K:R can be any number. In another aspect, the protein comprises one or more of an antibody binding domain derived from Staphylococcus protein A (ZD) (SEQ ID NO:159), an antimicrobial peptide selected from LL37 (SEQ ID NO: 161), Ib-M1 (SEQ ID NO: 163), Ib-M2 (SEQ ID NO: 165), Ib-M5 (SEQ ID NO: 167), Cathelecidin-1 (SEQ ID NO: 169), A(A1R, A8R, I17K) (SEQ ID NO: 171), H5 (SEQ ID NO: 173), H5-61-90 (SEQ ID NO: 175); RGD peptide (RGDSPAS, SEQ ID NO: 39); protein drugs, GLP-1 (SEQ ID NO: 177); fluorescent reporters (sfGFP (SEQ ID NO: 179), mRuby3 (SEQ ID NO: 181); RNA binding proteins (PUM-HD (SEQ ID NO: 183), eIF4E (SEQ ID NO: 185), PABP (SEQ ID NO: 187), Tis11D (SEQ ID NO: 189)); KH domains (Yifan or FMRP (SEQ ID NO: 191)); or AAV binding peptides PKD1 (SEQ ID NO: 193) or PKD2 (SEQ ID NO: 195). In another aspect, the enhanced bioavailability of the fusion protein can be used for isolation or separation of a biologic molecule. In another aspect, the biologic molecule comprises one or more of a lipid, a cell, a protein, a nucleic acid, a carbohydrate, or a viral particle. In another aspect, the nucleic acid is single stranded or double stranded DNA or RNA. In another aspect, the viral particle is an adenovirus particle, an adeno-associated virus particle, a lentivirus particle, a retrovirus particle, a poxvirus particle, a measle virus particle, or herpesvirus particle. In another aspect, the protein comprises albumin, monoclonal IgG antibodies, or Fc fusion antibodies. In another aspect, the isolation or separation is accomplished via reversible phase separation.

### BRIEF DESCRIPTION OF THE DRAWINGS

This patent or application contains at least one drawing executed in color. Copies of this patent or patent application publication with color drawing(s) will be provided by the Office upon request and payment of the necessary fee.

FIG. 1A-G shows artificial intrinsically disordered polypeptides (A-IDPs) inspired from native idps exhibit reversible UCST phase behavior. FIG. 1A shows proteomic analysis of native IDPs that form biomolecular condensates reveal that they have an abundance of G/P, charged and uncharged polar residues, yet exhibit a balance of overall charge. FIG. 1B shows an example of a dense, exclusionary phase formed by an UCST exhibiting A-IDP even in the complex medium of bacterial cell lysate. The coacervate shows observes almost complete separation from all other cellular proteins and debris present in the cell lysate after centrifugation, facilitating purification from the insoluble cell lysate fraction without affinity tags. FIG. 1C shows an example SDS-PAGE gel of a set of A-IDPs-[Q5,8]-20 to [Q5,8]-80—with conserved sequence but increasing MW that show the high purity of the A-IDPs that is obtained by exploiting their UCST phase behavior without need for any chromatographic purification. FIG. 1D shows a visualization of UCST phase separation of [Q5,8]-20 in water-in-oil droplets with fluorescence microscopy. Upon cooling, phase separation in a droplet is initiated at multiple sites, the puncta that grow from each site slowly coalesce with one another into a single dense phase. Upon reheating, equilibrium with the surrounding dilute phase is constantly re-established, leading to a higher concentration dilute phase and smaller volume occupied by the dense phase. ϕ=0.0018, scale bar=50 μm. FIG. 1E shows a schematic UCST phase diagram for a cooling-heating cycle of a UCST polypeptide in a water-in-oil droplet. FIG. 1F shows dynamic light scattering data of [Q5,8]-20 demonstrating the change in hydrodynamic radius upon cooling. Upon reaching the cloud point, [Q5,8]-20 transitions from soluble unimeric polypeptides with a radius of hydration of 5-6 nm to micron-sized aggregates. Data collected at ϕ=0.0043 in 140 mM PBS, pH 7.4. FIG. 1G shows UCST cloud points are affected by polypeptide volume fraction in solution. This behavior follows a natural log dependence in the dilute regime (R²=0.98).

FIG. 2 [WT]-20-sfGFP exhibits phase separation memory upon multiple cycles of heating and cooling. Upon multiple heating and cooling cycles, [WT]-20-sfGFP form puncta in the same location as the first cooling cycle. Given the importance of memory, it is critical to note that the observed transition temperature was below room temperature (˜15° C.), suggesting that these cells are naïve to phase separation as they were incubated at 37° C. and processed at room temperature. Scale bar indicated 5 μm. Cooling and heating rate were set to a constant 5° C. min⁻¹.

FIG. 3A-B. additional proteomic analysis. FIG. 3A shows a graph of the difference in amino acid composition between ordered and disordered regions within the same protein. A disordered region was defined as being scored with a >0.5 value using PONDR VSL2, ordered regions with a score of <0.5. Values were calculated by subtracting the percentage of chain composition in disordered regime from ordered regime. Bars indicate 25^{th}-75^{th} percentiles and whiskers indicate 10^{th}-90^{th} percentiles. Middle line indicates the median of the data set. N=63, *P<0.01 in students t-test between ordered and disordered regions of all proteins sampled. FIG. 3B shows a histogram plotting the length of the disordered regions analyzed in this study. Bars indicate 25^{th}-75^{th} percentiles and whiskers indicate 10^{th}-90^{th} percentiles.

FIG. 4. SDS-PAGE gels of purified proteins used in this study relevant to FIG. 1-5. Lane labels for each protein purified in this study are listed in Table 2 and Table 3.

FIG. 5. wide-field fluorescent microscope images of fluorescently labeled [Q5,8]-20 Inside water-in-oil compartments. [Q5,8]-20 was labeled with AlexaFluor 350 via NHS chemistry and resuspended in 140 mM PBS at pH 7.4 to a final ϕ=0.003. Water-in-oil mixture was transferred to glass slide and cooled from 50° C. to 10° C. Scale bar=20 μm.

FIG. 6. additional dynamic light scattering data. Data collected on 20 nm filtered samples at volume fractions that were predicted to exhibit liquid-liquid phase separation at 40° C. Data collected in 140 mM PBS, pH 7.4.

FIG. 7. cyclic cooling and heating cycles exhibit minimal hysteric behavior. Optical turbidity measured at 350 nm of repeated cooling and heating curves of [Q5,8]-20@ϕ=0.0025 between 40° C. and 30° C.

FIG. 8A-F show control of UCST cloud point using main chain amino acid composition.

FIG. 8A shows a schematic describing the methodology for doping repeat unit b into a homopolymer of a. The WT A-IDP with a high UCST cloud point consisting of 40 repeats of a—GRGDSPYS—is doped with increasing fractions of repeat b—GRGDQPYQ—to probe “loss of function” of UCST phase behavior of polymers of a. The doping of b into a is designed to ensure mixing of the two repeats along the polypeptide chain and minimize blocky behavior. FIG. 8B shows doping of b into a results in mutant IDPs; the UCST cloud point temperature (Tₜ) of each mutant IDP is a linear function of volume fraction (ϕ) of the A-IDP. FIG. 8C shows the effect of composition—degree of doping—is a linear function of the degree of substitution of b into a at constant volume fraction of 10⁻³ (R²=0.97). FIG. 8D shows substitution of aromatic Y residues with aliphatic V dramatically reduces Tₜ. FIG. 8E shows substitution of R with K dramatically reduces Tₜ. A 50% substitution of K for R lowers the Tₜ by more than 40° C. FIG. 8F shows the chemical composition can affect the saturation concentration by two orders of magnitude at constant molecular weight (Csat@37° C.=1-800 μM). This can be visualized by normalizing to the saturation concentration of [WT]-40 which is conveniently ˜1 μM and is shown by the dashed horizontal line.

FIG. 9A-E show effects of single amino acid substitutions on UCST cloud point and new relative UCST propensity scale. FIG. 9A shows partial binodal phase boundary of well-mixed, di-block polypeptides with varied ratio of aromatic:aliphatic residues. FIG. 9B shows partial binodal phase boundary of well-mixed, di-block polypeptides with varied ratio of polar non-charged residues. FIG. 9C shows partial binodal phase boundary of well-mixed, di-block polypeptides with varied identity of positively charged and negatively residues. FIG. 9D shows partial binodal phase boundary of well-mixed, di-block polypeptides with varied amount of. Data collected under physiologic solution conditions (140 mM PBS, pH 7.4) at ϕ=10⁻³. All polypeptides are 326 amino acids in length. FIG. 8E shows a relatively scale for UCST propensity based on substitutions made to the [WT] repeating motif. The transition temperatures listed are the UCST cloud point at ϕ=10⁻³ if the left amino acid was replaced with the amino acid to the right of the arrow and the total number of amino acids was 326.

FIG. 10 shows analysis of secondary structure with circular dichroism (CD) spectroscopy. CD spectra of various A-IDPs lack a defined secondary structure curve shape, characteristic of other IDP and other repetitive protein polymers. Data collected at 50° C. (soluble chains) at 5 μM in 5 mM PBS, pH 7.4. Error bars indicate standard deviation of three sequential runs.

FIG. 11A-C show control of UCST cloud point by molecular weight of A-IDP. FIG. 11A shows the molecular weight of the polypeptide affects the Tₜ. FIG. 11B shows the Tₜ directly scales with the natural log of MW. FIG. 11C shows at constant chemical composition, it is possible to modulate Cₛₐₜ by over five orders of magnitude simply by changing the MW of the A-IDP (Cₛₐₜ at 37° C.=1 nM-400 μM). [WT]-40 has a Cₛₐₜ of ˜1 μM.

FIG. 12A-D show minor effects on UCST cloud point in protein polypeptides. FIG. 12A shows partial binodal phase diagram of sequence syntax permutations focused around the Pro residue. Mutations reveal that amino acid mutation site affects the UCST binodal, particularly at the fifth position, but do not eliminate phase behavior. Data collected under physiologic conditions (140 mM PBS, pH 7.4). FIG. 12B shows partial binodal phase boundaries of agnostically non-repetitive but compositionally identical versions of [WT]-20. FIG. 12C shows turbidity curves of [H7]-60 in different pH solutions. Decreasing the pH and protonation of the His residues increases and broadens observed UCST phase behavior. This effect centers at ˜pH 7, very close to the predicted pKa of the imidazole group in H. In contrast to, [WT]-60's UCST cloud point does not change as a function of pH (black dots, graph insert). FIG. 12D shows turbidity curves of [Q5,8]-40 in solutions with different concentrations of NaCl. In pure water, [Q5,8]-40 exhibits a broad transition at higher temperatures. Increasing the concentration of NaCl between 0-140 mM reduces and sharpens the UCST cloud point, finally reaching a minimum at ˜500 mM. From this point, the protein exhibits a salting-out effect and the transition temperature begins to rise again.

FIG. 13A-C. mapping phase diagrams using a temperature gradient device. FIG. 13A shows representative dark-field image of [Q5,8]-20 solutions on a temperature gradient device. The transition temperatures of the reference solutions (red and blue lines) and the 20 mg·mL⁻¹ [Q5,8]-20 solution (green line) are indicated by the horizontal colored lines. The dashed vertical magenta line along the 20 mg·mL⁻¹ capillary tube illustrated the region of the image used to measure the line scan. FIG. 13B shows line scan of normalized light scattering intensity versus temperature for the 20 mg·mL⁻¹ [Q5,8]-20 capillary shown in FIG. 13A. The dashed black lines represent tangent lines for the high temperature baseline and increase in light scattering at lower temperatures. These two lines intersect at Tph, as indicated by the vertical green line. FIG. 13C shows final binodal phase lines of [WT]-20 and [Q5,8]-20 using multiple data points from temperature gradient device. A three-piece fit was utilized to fit three regimes that roughly correspond to the dilute, overlap, and semi-dilute regimes of the polypeptide phase diagram. The observed data and subsequent fits demonstrate that polypeptide sequence not only affects UCST cloud point in the dilute regime but over the entire concentration range measured (ϕ≤0.5) FIG. 14A-B show quantification of dextran uptake during phase separation of A-IDPs.

FIG. 14A shows fluorescent microscopy images of phase separated droplets in the presence of dextran molecules of different molecular weight (10/40 kDa) labeled with Alexa488 (green) fluorophore. Inside the phase separated space (dark circles), there is very little sequestration of the dextran molecules as a function of dextran molecular weight or A-IDP sequence. Scale bar is 20 μm. FIG. 14B shows quantification of fluorescent signal between the area inside of phase separated droplets and outside.

FIG. 15A-G show A-IDPs exhibit tunable intracellular droplet formation based on molecular weight and ratio of aromatic:aliphatic content. All scale bars are 5 μm. FIG. 15A shows a schematic describing the use of two key parameters—ratio of aromatic:aliphatic content and molecular weight—to control intracellular droplet formation by modulating Cₛₐₜ. FIG. 15B shows partial in vitro binodal of A-IDP-sfGFP fusions in the dilute regime in 140 mM PBS, pH 7.4. Similar to A-IDPs, A-IDP-GFP fusion proteins exhibit molecular weight and aromatic content dependent phase behavior. FIG. 15C shows [WT]-20-sfGFP fusion phase separates in eukaryotic cells (HEK293 cells, Day 5). Instead of forming a single droplet as seen in vitro in protocells (see FIG. 1C), many distinct droplets are formed indicating either diffusion-limited or arrest-limited coalescence. FIG. 15D shows confocal fluorescence images of A-IDP-sfGFP as a function of induction time and molecular weight in E. coli. A higher intracellular concentration is required for [WT]-20 versus [WT]-40 to form intracellular droplets. It is noticeable that [WT]-40 has a lower ϕ′-A-IDP poor-soluble phase outside the dense droplet phase compared with [WT]-20. FIG. 15E shows reducing the aromatic content increases the Cₛₐₜ in a dose-dependent manner. FIG. 15F shows A-IDP-sfGFP fusions exhibit a one order of magnitude shift in their Cₛₐₜ as determined by their molecular weight and ratio of aromatic:aliphatic content. FIG. 15G shows the size of intracellular droplets (ϕ″ or dense phase) grow with induction time. As concentration of the A-IDP-sfGFP increases inside the cell, the soluble concentration outside the droplet does not change (FIG. 19) but the size of the intracellular droplets grows relative to the total cell area. Images are individual cells from [3Y7:V7]-40-sfGFP cultures at various time points. Error bars represent standard error of the mean.

FIG. 16A-C show a comparison of partial binodal phase diagrams of A-IDP and A-IDP-sfGFP fusions. FIG. 16A shows partial binodal phase boundaries of [WT]-40 and [WT]-40-sfGFP. FIG. 16B shows partial binodal phase boundaries of [3Y7:V7]-40 and [3Y7:V7]-40-sfGFP. FIG. 15C shows partial binodal phase boundaries of [WT]-20 and [WT]-20-sfGFP. The sfGFP fusion lowers the UCST binodal line for all A-IDPs. These data suggest that the larger molecular weight polypeptides are less affected by sfGFP fusion as the observed difference in [WT]-40 and [3Y7:V7]-40 is only −10° C. instead of ˜20° C. for [WT]-20.

FIG. 17 show confocal microscopy images of HEK293 cells with transiently transfected [WT]-20-sfGFP. Confocal fluorescence image slices throughout the cell demonstrate that phase separated droplets are formed throughout the cytoplasm without obvious colocalization with other cellular structures. Images taken 24 hours after transfection with 3 μg of pCDNA plasmid that encode [WT]-20-sfGFP. Scale bar=5 μm.

FIG. 18 shows measurement of total cellular fluorescence as a function of time post induction. E. coli cultures were spun down and resuspended in 140 mM PBS, pH 7.4. The optical turbidity and fluorescence intensity of sfGFP were measured and plotted as a function of time. Data collected at 22° C.

FIG. 19 shows measurement of the cellular fluorescence at different locations within the cell. Digital partitions were made between the dense phase separated area of the cell and soluble cytoplasmic space using ImageJ. The mean of the total cell fluorescence intensity (solid line) and cytoplasmic fluorescence intensity (dotted line) are plotted as a function of time post-IPTG induction. [WT]-20-sfGFP does not exhibit intracellular droplets until the 6 hr mark. At this point the cytoplasmic fluorescence intensity remains constant but the total fluorescent increases from 6 hr onward. [WT]-40-sfGFP phase transitions prior to the 2-hr timepoint.

FIG. 20A-D show A-IDPs exhibit reversible coacervation in E. coli determined by their molecular weight and aromatic:aliphatic ratio. All scale bars are 5 μm. FIG. 20A shows intracellular droplets comprised of [WT]-20-sfGFP can be formed and dissolved reversibly via alternating cooling and heating cycles. This process is completely reversible over four rounds of cooling and heating. Cooling rate=5° C. min⁻¹, induction time of 4 hr. FIG. 20B shows Tₜ normalized to the intracellular fluorescence of sfGFP in each individual cell (n=30) does not change significantly over four heating (red bars) and cooling (blue bars) cycles. Boxes indicate 25^{th}-75^{th} percentile. FIG. 20C shows the intracellular Tₜ—similar to in vitro—is a function of A-IDP molecular weight and aromatic content. Cooling ramp=60° C.→10° C. Cooling rate=5° C. min 1, A-IDP gene induction time of 8 hr. Whiskers indicate 10^{th}-90^{th} percentile. FIG. 20D shows intracellular binodal lines of various A-IDP-sfGFP fusions. Tₜ increases as a function of cellular fluorescence, a surrogate of A-IDP concentration, and aromatic content of the A-IDP. Data analyzed at 2, 4, 8 hr for [WT]-40-sfGFP and [3Y7:V7]-10 and 4, 8, 24 hr for [Y7:V7]-40 (n=30). Error bars indicate standard error of the mean. FIG. 20D shows upon reconstitution of sfGFP in the dense phase, the solubility of the reconstituted GFP-A-IDP complex can be modulated with temperature. Data was collected for 36 hr post-IPTG induction and 12 hours post-arabinose induction.

FIG. 21A-C show image analysis of the number of puncta formed in each cell. 100 cells at random were tabulated for each histogram. FIG. 21A shows a number of intracellular puncta formed in each cell containing [WT]-20-sfGFP during a cooling ramp from 60° C.→10° C. (green) and imaged isothermally at 22° C. Isothermal analysis performed at 6 hours post induction, the first timepoint where intracellular puncta were observed. Cooling ramp performed at 4 hours post induction, where transition temperature (Tₜ) was between 22° C. and 37° C. FIG. 21B shows a number of intracellular puncta formed in each cell containing [3Y:V]-40-sfGFP during a cooling ramp from 60° C.→10° C. (green) and imaged isothermally at 22° C. Isothermal analysis performed at 4 hours post induction, the first timepoint where intracellular puncta were observed. Cooling ramp performed at 4 hours post induction, where Tₜ was between 22° C. and 37° C. FIG. 21C shows a number of intracellular puncta formed in each cell containing [WT]-40-sfGFP during a cooling ramp from 60° C.→10° C. (green) and imaged isothermally at 22° C. Isothermal analysis performed at 4 hours post induction, the first timepoint where intracellular puncta were observed. Cooling ramp performed at 4 hours post induction, but the transition observed was >37° C. indicating the possibility of memory.

FIG. 22A-E show engineered intracellular droplets with programmable functions. FIG. 22A shows site specific labeling of droplets with a small molecule fluorescent dye. E. coli cells containing condensates formed by a [3Y7:V7]-40 variant with azido-phenylalanine (AzF) residues that presents a biorthogonal azide which can be labeled in situ with a dibenzocyclooctyne-dye conjugate (DBCO-Alexa488). DBCO-Alexa488 mixture can diffuse into cells and into the A-IDP condensates within the cell, labeling the azide groups within 10 min of incubation with live E. coli. FIG. 22B shows reconstitution of function GFP in condensates by recruitment of a partner from the cytoplasm using a split GFP system. A GFP-11-[3Y7:V7]-40 fusion protein is able to recruit GFP-1-10 from the surrounding cytoplasm into intracellular droplets. Upon formation of intracellular condensates after 24 hr of IPTG induction of GFP-11-[3Y7:V7]-40 (left panel), subsequent induction GFP-1-10 by arabinose induction enables recruitment of GFP-10 into the condensates and reconstitution of functional sfGFP within existing intracellular condensates within 12 hr of GFP-1-10 induction (right panel). FIG. 22C shows a schematic of enzyme-condensate experiment. The α-peptide (αp) of LacZ is fused to a fluorescent reporter protein (mRuby3) and expressed from an IPTG-inducible gene from a plasmid in the E. coli strain KRX that has a deletion mutant of the LacZ gene that produces a truncated, catalytically inactive enzyme lacking the ap. Complementation of LacΔM15 by a αp-A-IDP-mRuby3 fusion creates an active enzyme that converts FDG into fluorescein that is then rapidly exported from the intracellular space into the surrounding medium. FIG. 22D shows confocal microscopy images showing the fluorescent conversion of fluorescein Di-β-D-galactopyranoside (FDG). Note that the puncta-like structures of αp-mRuby3 in the top panel are due to a fraction of the fusion forming inclusion bodies in cells. When the αp is fused to [WT]-20-mRuby3 the fluorescence is first observed at the sites of intracellular phase transition in coacervate droplets, and the fluorescein then diffuses into the cytosol and then out of the cell into the extracellular space. Increasing the molecular weight of the A-IDP leads to increased FDG conversion at earlier timepoints and higher overall conversion after 20 min. Rebalanced images of αp-[WT]-40-mRuby3 and αp-[WT]-80-mRuby3 can be found in FIG. 25 for improved visualization of the colocalization of intracellular droplets and converted FDG. FIG. 22E shows intracellular concentration of fluorescein produced by catalytic conversion of FDG, normalized to the mRuby3 fluorescence of each individual cell (n≈300). The catalytic efficiency increases with A-IDP MW, as seen by the greater ratio of green fluorescence resulting from FDG conversion to fluorescein normalized to the red fluorescence of mRuby3 on a molar basis. Both αp-[WT]-40-mRuby3 and αp-[WT]-80-mRuby3 exhibit statistically significant differences from the control (αp-mRuby3). Error bars indicate standard error of the mean. FIG. 22F shows all αp-A-IDP-mRuby3 fusions exhibit a higher ratio of green fluorescence inside the cell, indicating a greater persistence of fluorescent FDG inside the intracellular space compared to the αp-mRuby3 control. Error bars indicate standard error of the mean. All scale bars are 5 μm.

FIG. 23A-B show confocal microscope images of split GFP recruitment into intracellular droplets. FIG. 23A shows GFP-11-[3Y7:V7]-40-mRuby3 co-expressed in the presence of GFP-1-10 creates fluorescently active GFP only in the interior of the droplet. FIG. 23B shows in the absence of GFP-1-10 induction, there is little green fluorescent inside the intracellular droplets. Data taken at 22° C. Scale bar=5 μm.

FIG. 24 shows A-IDPs can modulate the solubility of an endogenously bound molecule 2. Upon recruitment of sfGFP into the dense phase, the solubility of the entire complex can be modulated with temperature. Data was collected for 36 hours post-IPTG induction and 12 hours post-arabinose induction. Scale bar=5 μm.

FIG. 25A-B show color balanced confocal microscopy images of αp-[WT]-40-mRuby3 and αp-[WT]-80-mRuby3. All scale bars are 5 μm. FIG. 25A shows color re-balanced images from FIG. 22B for improved visualization of the intracellular droplets formed by αp-A-IDP-mRuby3 fusions. FIG. 25B shows split channel images of αp-[WT]-40-mRuby3 and αp-[WT]-80-mRuby3.

FIG. 26. Mander's colocalization score between converted FDG and fluorescent reporter. Data analyzed 30 min after FDG addition. Background threshold was set automatically.

FIG. 27A-B shows Lineweaver-Burk plots for determining Kₘ and Vₘₐₓ. Lineweaver-Burk plots created with variable starting concentrations of FDG for FIG. 27A, αp-mRuby3; FIG. 27B, αp-[WT]-20-mRuby3; FIG. 27C, αp-[WT]-40-mRuby3; and FIG. 27D, αp-[WT]-80-mRuby3. Slopes (Vₒ) were determined from fluorescent generation over the course of 20 minutes. Intercepts and slope were used in the calculation of Kₘ and Vₘₐₓ.

FIG. 28A-C show enzymatic droplets formed with variable ratios of aromatic to aliphatic residues. All scale bars are 5 μm. FIG. 28A shows confocal microscopy images observing the fluorescent conversion of Fluorescein Di-β-D-Galactopyranoside (FDG) of αp-mRuby3, αp-[WT]-40-mRuby3, αp-[3Y7:V7]-40-mRuby3 and αp-[Y7:V7]-40-mRuby3. Decreasing the aromatic:aliphatic ratio does not increase FDG conversion over time but does change the dynamics of uptake with lower aromatic:aliphatic ratio polypeptides observing higher uptake at earlier timepoints after FDG addition. FIG. 28B shows quantified amount of converted FDG intracellularly, normalized to the amount of mRuby3 fluorescence. There is little difference between A-IDPs with different ratios of aromatic:aliphatic content. Error bars indicate standard error of the mean. FIG. 28C shows all αp-A-IDP-mRuby3 fusions exhibit a higher ratio of FDG fluorescence inside the cell, indicating a greater persistence of fluorescent FDG inside the intracellular space compared to the αp-mRuby3 control. There is little difference between A-IDPs with different levels of aromatic content. Error bars indicate standard error of the mean.

FIG. 29A-B shows enzymatic Activity of αp-[V7]-40-mRuby3. All scale bars are 5 μm. FIG. 29A shows confocal microscopy images showing the fluorescent conversion of fluorescein Di-β-D-galactopyranoside (FDG) attached to soluble αp-[V7]-40-mRuby3. FIG. 29B shows intracellular concentration of fluorescein produced by catalytic conversion of FDG by αp-[V7]-40-mRuby3, normalized to the mRuby3 fluorescence of each individual cell (n≈300). The soluble fusion exhibits a lower level of enzymatic activity than all puncta-forming αp-A-IDP fusions. αp-mRuby3 data from FIG. 24 redrawn to show scale. Error bars indicate standard error of the mean.

FIG. 30A-D show examples of various fusion proteins that express at low levels in prokaryotic expression systems that when fused to disordered biopolymers rescue expression levels and using the phase separation behavior of the biopolymers allow for recovery into soluble fractions. This can be performed with mAb binding proteins that have a nanobody folded structure that bind to mAb (ZD, FIG. 30A); fluorescent fusion proteins that have beta-barrel structures (sfGFP, FIG. 30B); therapeutic protein peptides (GLP-1, FIG. 30C) with strong alpha-helical tendencies, RNA binding proteins (PUMHD, FIG. 30D) that have tandem repeat structures and antimicrobial peptides that exhibit cytotoxic tendencies in E. coli.

FIG. 31 shows the incubation of mAb with a phase separating biopolymer fused to a domain from protein A that binds mAbs. The biopolymer is bound to the mAb and centrifuged to capture the mAb heavy (HC) and light chain (LC). The supernatant of this step is run in lanes 2 and 5. Then, the supernatant is removed, and the pellet is resuspended in an elution buffer that is a lower pH which causes dissociation between the biopolymer-ZD fusion and the mAb. The solution is spun again creating an elution supernatant (lanes 3, 6) contains pure mAb HC and LC and other protein contaminants. The elution pellet contains the biopolymer and no mAb (lanes 4, 7).

FIG. 32 shows microscopic images of fluorescently labeled mAb (red/white in grey-scale) visualized in the presence of phase separated protein ((GRGDQPYQ)₄₀, SEQ ID NO: 3 with m=40) fused to the Z-domain of Protein A (ZD). The colocalization of the droplets with a fluorescent signal is observable in the first image. When the buffer pH is dropped at t=0, there is an inversion in the fluorescent signal suggesting the mAb has completely dissociated from the phase separated protein-ZD fusion protein (white arrow) and entered the surrounding solution (red). These biopolymer-fusion proteins retain their liquid-like behavior as droplet fusion occurs between 60-240 sec (arrows).

FIG. 33 shows fusion proteins containing various AMPs were expressed fused to nothing (left side) and various biopolymer (right side). When fusion protein is not expressed (top), cell growth proceeds as normal measured by an increasing absorbance at OD₆₀₀. When the AMP alone is expressed (bottom-left), cell growth is stunted (growth curves shifted to later times). When AMP-biopolymer fusion protein is expressed, normal growth is recovered, suggesting reduced availability of the AMP.

FIG. 34 shows injection strategies for forming subcutaneous depots in vivo. Injection with solubilizing agent such as urea allows for injection under ambient conditions. As the solubilizing agent diffuses faster than the polypeptide, the solvent becomes a poor solvent and the polypeptide will phase separate. Alternatively, a dehydrated coacervate can be implanted in the subcutaneous space which will slowly rehydrate indirectly into a two-phase regime.

FIG. 35 shows fluorescence molecular tomography of (GRGDSPYQ)₄₀ labeled with a near infrared fluorescent dye after injection in the presence of 2 M urea+PBS. Injection concentration of 175 μM, corresponding to 1.2 mg of protein total.

FIG. 36 shows fluorescence molecular tomography of (GRGDSPYQ)₄₀ labeled with a near infrared fluorescent dye after injection in dehydrated state. Injection mass equal to 1.2 mg of protein total.

FIG. 37 shows binodal phase boundary of GLP-1-RIDPs of lower molecular weight. Data collected in 140 mM PBS. Dotted lines are fit lines for y=m*ln(x)+b.

FIG. 38 shows blood glucose of GLP-1-RIDP fusion proteins of ˜20 kDa in size and variable Ca. Data collected from C57Bl/6J mice that have been fed 60% fat diet. Error bars are standard error of the mean (n=5).

FIG. 39: Body weight change of mice with sub-cutaneous GLP-1-RIDP depots of ˜20 kDa in size and variable Cₛₐₜ. Data collected from C57Bl/6J mice that have been fed 60% fat diet. Error bars are standard deviation of the mean (n=5).

FIG. 40 shows binodal phase boundary of GLP-1-RIDPs of higher molecular weight. Data collected in 140 mM PBS. Dotted lines are fit lines for y=m*ln(x)+b.

FIG. 41 shows blood glucose of GLP-1-RIDP fusion proteins of ˜35 kDa in size and variable Cₛₐₜ. Data collected from C57Bl/6J mice that have been fed 60% fat diet. Error bars are standard error of the mean (n=5).

FIG. 42 shows blood glucose of GLP-1-RIDP fusion proteins of different molecular weights but similar Cₛₐₜ. Data collected from C57Bl/6J mice that have been fed 60% fat diet. Error bars are standard error of the mean (n=5).

FIG. 43 shows body weight change of mice with sub-cutaneous GLP-1-RIDP depots of ˜35 kDa in size and variable Cₛₐₜ. Data collected from C57Bl/6J mice that have been fed 60% fat diet. Error bars are standard error of the mean (n=5).

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the specification of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Affinity” refers to the binding strength of a binding polypeptide to its target (i.e., binding partner).

“Agonist” refers to an entity that binds to a receptor and activates the receptor to produce a biological response. An “antagonist” blocks or inhibits the action or signaling of the agonist. An “inverse agonist” causes an action opposite to that of the agonist. The activities of agonists, antagonists, and inverse agonists may be determined in vitro, in situ, in vivo, or a combination thereof.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

As used herein, the term “biomarker” refers to a naturally occurring biological molecule present in a subject at varying concentrations that is useful in identifying and/or classifying a disease or a condition. The biomarker can include genes, proteins, polynucleotides, nucleic acids, ribonucleic acids, polypeptides, or other biological molecules used as an indicator or marker for disease. In some embodiments, the biomarker comprises a disease marker. For example, the biomarker can be a gene that is upregulated or downregulated in a subject that has a disease. As another example, the biomarker can be a polypeptide whose level is increased or decreased in a subject that has a disease or risk of developing a disease. In some embodiments, the biomarker comprises a small molecule. In some embodiments, the biomarker comprises a polypeptide.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25^{th}-75^{th} percentile range, preferably a value that corresponds to the 25^{th} percentile, the 50^{th} percentile or the 75^{th} percentile, and more preferably the 75^{th} percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for a protein activity may be defined in accordance with standard practice.

The term “expression vector” indicates a plasmid, a virus, or another medium, known in the art, into which a nucleic acid sequence for encoding a desired protein can be inserted or introduced.

The term “host cell” is a cell that is susceptible to transformation, transfection, transduction, conjugation, and the like with a nucleic acid construct or expression vector. Host cells can be derived from plants, bacteria, yeast, fungi, insects, animals, etc. In some embodiments, the host cell includes Escherichia coli.

“Polymer” as used herein is intended to encompass a homopolymer, heteropolymer, block polymer, co-polymer, ter-polymer, etc., and blends, combinations, and mixtures thereof. Examples of polymers include, but are not limited to, functionalized polymers, such as a polymer comprising 5-vinyltetrazole monomer units and having a molecular weight distribution less than 2.0. The polymer may be or contain one or more of a star block copolymer, a linear polymer, a branched polymer, a hyperbranched polymer, a dendritic polymer, a comb polymer, a graft polymer, a brush polymer, a bottle-brush copolymer and a crosslinked structure, such as a block copolymer comprising a block of 5-vinyltetrazole monomer units. Polymers include, without limitation, polyesters, poly(meth)acrylamides, poly(meth)acrylates, polyethers, polystyrenes, polynorbonenes and monomers that have unsaturated bonds. For example, amphiphilic comb polymers are described in U.S. Patent Application Publication No. US 2007/0087114 and in U.S. Pat. No. 6,207,749 to Mayes et al., the disclosure of each of which is herein incorporated by reference in its entirety. The amphiphilic comb-type polymers may be present in the form of copolymers, containing a backbone formed of a hydrophobic, water-insoluble polymer and side chains formed of short, hydrophilic non-cell binding polymers. Examples of other polymers include, but are not limited to, polyalkylenes such as polyethylene and polypropylene; polychloroprene; polyvinyl ethers; such as polyvinyl acetate); polyvinyl halides such as polyvinyl chloride); polysiloxanes; polystyrenes; polyurethanes; polyacrylates; such as poly(methyl (meth)acrylate), poly(ethyl (meth)acrylate), poly(n-butyl(meth)acrylate), poly(isobutyl (meth)acrylate), poly(tert-butyl (meth)acrylate), poly(hexyl(meth)acrylate), poly(isodecyl (meth)acrylate), poly(lauryl (meth)acrylate), poly(phenyl (meth)acrylate), poly(methyl acrylate), poly(isopropyl acrylate), poly(isobutyl acrylate), and poly(octadecyl acrylate); polyacrylamides such as poly(acrylamide), poly(methacrylamide), poly(ethyl acrylamide), poly(ethyl methacrylamide), poly(N-isopropyl acrylamide), poly(n, iso, and tert-butyl acrylamide); and copolymers and mixtures thereof. These polymers may include useful derivatives, including polymers having substitutions, additions of chemical groups, for example, alkyl groups, alkylene groups, hydroxylations, oxidations, and other modifications routinely made by those skilled in the art. The polymers may include zwitterionic polymers such as, for example, polyphosphorycholine, polycarboxybetaine, and polysulfobetaine. The polymers may have side chains of betaine, carboxybetaine, sulfobetaine, oligoethylene glycol (OEG), sarcosine, or polyethyleneglycol (PEG). For example, poly(oligoethyleneglycol methacrylate) (poly(OEGMA)) may be used. Poly(OEGMA) may be hydrophilic, water-soluble, non-fouling, non-toxic and non-immunogenic due to the OEG side chains.

“Polynucleotide” as used herein can be single stranded or double stranded or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide,” “protein,” and “peptide” are used interchangeably herein. “Primary structure” refers to the amino acid sequence of a particular peptide. “Secondary structure” refers to locally ordered, three-dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units.

“Reporter,” “reporter group,” “label,” and “detectable label” are used interchangeably herein. The reporter is capable of generating a detectable signal. The label can produce a signal that is detectable by visual or instrumental means. A variety of reporter groups can be used, differing in the physical nature of signal transduction (e.g., fluorescence, electrochemical, nuclear magnetic resonance (NMR), and electron paramagnetic resonance (EPR)) and in the chemical nature of the reporter group. Various reporters include signal-producing substances, such as chromagens, fluorescent compounds, chemiluminescent compounds, radioactive compounds, and the like. In some embodiments, the reporter comprises a radiolabel. Reporters may include moieties that produce light, e.g., acridinium compounds, and moieties that produce fluorescence, e.g., fluorescein. In some embodiments, the signal from the reporter is a fluorescent signal. The reporter may comprise a fluorophore. Examples of fluorophores include, but are not limited to, acrylodan (6-acryloyl-2-dimethylaminonaphthalene), badan (6-bromo-acetyl-2-dimethylamino-naphthalene), rhodamine, naphthalene, danzyl aziridine, 4-[N-[(2-iodoacetoxy)ethyl]-N-methylamino]-7-nitrobenz-2-oxa-1,3-diazole ester (IANBDE), 4-[N-[(2-iodoacetoxy)ethyl]-N-methylamino-7-nitrobenz-2-oxa-1,3-diazole (IANBDA), fluorescein, dipyrrometheneboron difluoride (BODIPY), 4-nitrobenzo[c][1,2,5]oxadiazole (NBD), Alexa fluorescent dyes, and derivatives thereof. Fluorescein derivatives may include, for example, 5-fluorescein, 6-carboxyfluorescein, 3′6-carboxyfluorescein, 5(6)-carboxyfluorescein, 6-hexachlorofluorescein, 6-tetrachlorofluorescein, fluorescein, and isothiocyanate.

“Sample” or “test sample” as used herein can mean any sample in which the presence and/or level of a target is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

The term “sensitivity” as used herein refers to the number of true positives divided by the number of true positives plus the number of false negatives, where sensitivity (“sens”) may be within the range of 0<sens<1. Ideally, method embodiments herein have the number of false negatives equaling zero or close to equaling zero, so that no subject is wrongly identified as not having a disease when they indeed have the disease. Conversely, an assessment often is made of the ability of a prediction algorithm to classify negatives correctly, a complementary measurement to sensitivity.

The term “specificity” as used herein refers to the number of true negatives divided by the number of true negatives plus the number of false positives, where specificity (“spec”) may be within the range of 0<spec<1. Ideally, the methods described herein have the number of false positives equaling zero or close to equaling zero, so that no subject is wrongly identified as having a disease when they do not in fact have disease. Hence, a method that has both sensitivity and specificity equaling one, or 100%, is preferred.

By “specifically binds,” it is generally meant that a polypeptide binds to a target when it binds to that target more readily than it would bind to a random, unrelated target.

“Subject” as used herein can mean a mammal that wants or is in need of the herein described peptide biopolymers comprising one or more fusion proteins. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant.

“Transition” or “phase transition” refers to the aggregation of the thermally responsive polypeptides. Phase transition occurs sharply and reversibly at a specific temperature called the lower critical solution temperature (LCST) or the inverse transition temperature T^{A}. Below the transition temperature, the thermally responsive polypeptide (or a polypeptide comprising a thermally responsive polypeptide) is highly soluble. Upon heating past the transition temperature, the thermally responsive polypeptides hydrophobically collapse and aggregate, forming a separate, gel-like phase. “Inverse transition cycling” refers to a protein purification method for thermally responsive polypeptides (or a polypeptide comprising a thermally responsive polypeptide). The protein purification method may involve the use of thermally responsive polypeptide's reversible phase transition behavior to cycle the solution through soluble and insoluble phases, thereby removing contaminants.

“Treatment” or “treating,” when referring to protection of a subject from a disease, means preventing, suppressing, repressing, ameliorating, or eliminating the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or greater number of amino acids.

“Valency” as used herein refers to the potential binding units or binding sites. The term “multivalent” refers to multiple potential binding units. The terms “multimeric” and “multivalent” are used interchangeably herein.

“Variant” used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequences substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids. See Kyte et al., J. Mol. Biol. 1982, 757, 105-132. The hydropathic index of an amino acid is based on a consideration of its hydrophobicity and charge. It is known in the art that amino acids of similar hydropathic indexes can be substituted and retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a polynucleotide sequence that is substantially identical over the full length of the full gene sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

### Fusion Protein

The term “fusion protein” as described herein at least one intrinsically disordered polypeptide and at least one other polypeptide. The fusion protein may optionally include at least one linker. In one aspect, the intrinsically disordered polypeptide has controlled reversible phase separation.

In some embodiments, the fusion protein includes more than one polypeptide with controlled reversible phase separation. The polypeptide with controlled reversible phase separation can include multiple repeats of a peptide motif. The fusion protein may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, and least 40, at least 60, at least 80, at least 120, at least 160, or at least 200 polypeptides with controlled reversible phase separation or repeats of a peptide motif with controlled reversible phase separation. The fusion protein may include less than 30, less than 25, or less than 20 polypeptides with controlled reversible phase separation or repeats of a peptide motif. The fusion protein may include between 1 and 160, between 1 and 80, between 1 and 60, between 1 and 40, between 1 and 20, or between 1 and 10 polypeptides with controlled reversible phase separation or repeats of a peptide motif. In such embodiments, the polypeptides with controlled reversible phase separation may be the same or different from one another. In some embodiments, the fusion protein includes more than one polypeptide with controlled reversible phase separation positioned in tandem to one another (e.g., repeats of a peptide motif).

In some embodiments, the fusion protein includes one or more binding polypeptide. The fusion protein may include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 binding polypeptides. The fusion protein may include less than 30, less than 25, less than 20, less than 10, or less than 5 binding polypeptides. The fusion protein may include between 1 and 30, between 1 and 20, or between 1 and 10 binding polypeptides. In such embodiments, the binding polypeptides may be the same or different from one another. In some embodiments, the fusion protein includes more than one binding polypeptide positioned in tandem to one another. In some embodiments, the fusion protein includes 2 to 6 binding polypeptides. In some embodiments, the fusion protein includes two binding polypeptides. In some embodiments, the fusion protein includes three binding polypeptides. In some embodiments, the fusion protein includes four binding polypeptides. In some embodiments, the fusion protein includes five binding polypeptides. In some embodiments, the fusion protein includes six binding polypeptides.

The fusion protein may be expressed recombinantly in a host cell according to one of ordinary skill in the art. The fusion protein may be purified by any means known to one of skill in the art. For example, the fusion protein may be purified using chromatography, such as liquid chromatography, size exclusion chromatography, or affinity chromatography, or a combination thereof. In some embodiments, the fusion protein is purified without chromatography. In some embodiments, the fusion protein is purified using inverse transition cycling.

Polypeptides with Controlled Reversible Phase Separation

The polypeptides with controlled reversible phase separation may comprise any polypeptide that has minimal or no secondary structure as observed by CD, being soluble at a temperature below its lower critical solution temperature (LCST) and/or at a temperature above its upper critical solution temperature (UCST), and comprising a repeated amino acid sequence. LCST is the temperature below which the polypeptide is miscible. UCST is the temperature above which the polypeptide is miscible. In some embodiments, the polypeptide with controlled reversible phase separation has only UCST behavior. In some embodiments, the polypeptide with controlled reversible phase separation has only LCST behavior. In some embodiments, the polypeptide with controlled reversible phase separation has both UCST and LCST behavior. The polypeptide with controlled reversible phase separation may comprise a repeated sequence of amino acids. The polypeptides with controlled reversible phase separation may have a LCST between about 0° C. and about 100° C., between about 10° C. and about 50° C., or between about 20° C. and about 42° C. The polypeptide with controlled reversible phase separation may have a UCST between about 0° C. and about 100° C., between about 10° C. and about 50° C., or between about 20° C. and about 42° C. In some embodiments, the polypeptide with controlled reversible phase separation has a transition temperature between room temperature (about 25° C.) and body temperature (about 37° C.). In some embodiments, a fusion protein comprising one or more thermally responsive polypeptides has a transition temperature between room temperature (about 25° C.) and body temperature (about 37° C.). In some embodiments, the polypeptide with controlled reversible phase separation has no LCST or UCST behavior. The polypeptide with controlled reversible phase separation may have its LCST or UCST below body temperature or above body temperature at the concentration at which the peptide biopolymer comprising one or more fusion proteins is administered to a subject.

In some embodiments, the polypeptide with controlled reversible phase separation comprises one or more thermally responsive polypeptides. Thermally responsive polypeptides may include, for example, elastin-like polypeptides (ELP) and resilin-like protein (RLP).

In some embodiment, the polypeptide with controlled reversible phase separation comprises a plurality of polypeptides with controlled reversible phase separation. In one aspect, the polypeptide with controlled reversible phase separation a di-block of two or more polypeptides with controlled reversible phase separation. In one aspect, the polypeptides with controlled reversible phase separation comprise a di-block of a resilin-like protein (RLP) and an elastin-like polypeptide (ELP).

In one embodiment, the polypeptide with controlled reversible phase separation comprises one or more core polypeptides. In one aspect, the core polypeptide is a resilin-like polypeptide (RLP). RLPs are derived from arthropod Rec1-resilin. Rec1-resilin is environmentally responsive and exhibits a dual phase transition behavior. The thermally responsive RLPs can have LCST and UCST. Additional examples of suitable thermally responsive polypeptides are described in U.S. Patent Application Publication Nos. US 2012/0121709, and US 2015/0112022, each of which is incorporated herein by reference. In one embodiment, the RLP polypeptide comprises the sequence (GRGDSPYS)ₙ (SEQ ID NO: 1). The polypeptide with controlled reversible phase separation may comprise an amino acid sequence comprising (G₁-R₂-G₃-D₄-S₅-P₆-Y₇-S₈))ₙ, where n is 20-200. In some embodiments, n is 20, 30, 40, 50, 60, 70, 80, 90, 100, 110, 120, 130, 140, 150, 160, 170, 180, 190, 200, 210, 220, 230, 240, 250, 260, 270, 280, 290, or 300. In some embodiments, n may be less than 500, less than 400, less than 300, less than 200, or less than 100. In some embodiments, n may be between 1 and 500, between 1 and 400, between 1 and 300, or between 1 and 200. In some embodiments, n is 20, 40, 60, 80, 100, 120, 160, 180, or 200. In one aspect, n is 20 to 200 repeats. In one aspect, n is 20 to 60 repeats.

Thermally responsive polypeptides may have a phase transition. The thermally responsive polypeptide may impart a phase transition characteristic to an unstructured polypeptide or fusion protein. “Phase transition” or “transition” may refer to the aggregation of the thermally responsive polypeptide, which occurs sharply and reversibly at a specific temperature called the lower critical solution temperature (LCST) or the inverse transition temperature (T₁). Below the transition temperature (LCST or T₁), the thermally responsive polypeptides, (or polypeptides comprising a thermally responsive polypeptide) may be highly soluble. Upon heating above the transition temperature, thermally responsive polypeptides hydrophobically may collapse and aggregate, forming a separate, gel-like phase.

The thermally responsive polypeptides can phase transition at a variety of temperatures and concentrations. Thermally responsive polypeptides, for example, may not affect the binding or potency of the binding polypeptides. Thermally responsive polypeptides may allow the fusion protein to be tuned by a user to any number of desired transition temperatures, molecular weights, and formats.

Thermally responsive polypeptides may exhibit inverse phase transition behavior and thus, the fusion protein comprising the thermally responsive polypeptide may exhibit inverse phase transition behavior. Inverse phase transition behavior may be used to form drug depots within a tissue of a subject for controlled (slow) release of the fusion protein. Inverse phase transition behavior may also enable purification of the fusion protein using inverse transition cycling, thereby eliminating the need for chromatography.

One embodiment described herein is a polypeptide with controlled reversible phase separation comprising ten or more repeats of an amino acid sequence comprising:

(X-Z₁-X-Z₂-Z₃-X-Z₄-Z₃)ₙ,

where:
X is proline (P) or glycine (G) and the ratio of P:G is any number;
Z₁ is arginine (R), aspartic acid (D), or lysine (K) and the ratio of R:D is any number and the ratio of K:R can be any number;
Z₂ is Asp (D), Arg (R), Glu (E), where the ratio of R:D can be any number and D:E can be any number;
Z₃ is asparagine (N), glutamine (Q), serine (S), or threonine (T) were the ratio among N:Q:S:T can be any number; and
Z₄ is tyrosine (Y), histidine (H), tryptophan (W), phenylalanine (F), methionine (M), valine (V), isoleucine (I), alanine (A), or leucine (L) and the ratio among Y:H:W:F:M:V:I:A:L can be any number.
   In one aspect, X is proline (P) or glycine (G) and the ratio of P:G is between 1:3 and 3:1. In another aspect, Z₁ is arginine (R), aspartic acid (D), or lysine (K) and the ratio of R:D does not exceed 1:5 and the ratio of K:R can be any number. In another aspect, the phase separation is dependent on temperature, molecular weight, hydrophobicity, aromatic:aliphatic ratio, and concentration. In another aspect, n is 10 to 200. In another aspect, molecular weight is at least 5 kDa to 500 kDa. In another aspect, the molecular weight is about 5 kDa to about 100 kDa. In another aspect, the phase separation temperature is 0 to 100° C. In another aspect, the phase separation temperature is 4 to 25° C.; ˜25° C.; 25 to 37° C.; ˜37° C.; 35 to 38° C.; or >38° C. In another aspect, the polypeptide comprises modified amino acids, a reporter protein, or an enzyme. In another aspect, the sequence comprises: (G-R-G-D-S-P-Y-S)ₘ, where m is 20 to 80. In another aspect, the polypeptide comprises a sequence selected from one or more of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, or 197-279, or combinations thereof.

| | SEQ |
|---|---|
| | ID |
| NAME | NO |
| SKGP-[GRGDSPYS]20-GY | 1, |
| SKGPGRGDSPYSGRGDSPYSGRGDSPYSGRGDSPYSGRGDSPYSGRGDSPYSGRGDSPYSGRGDSPYSGR | 2 |
| GDSPYSGRGDSPYSGRGDSPYSGRGDSPYSGRGDSPYSGRGDSPYSGRGDSPYSGRGDSPYSGRGDSPYS | |
| GRGDSPYSGRGDSPYSGRGDSPYSGY | |
| SKGP-[GRGDQPYQ]20-GY | 3, |
| SKGPGRGDQPYQGRGDQPYOGRGDQPYQGRGDQPYQGRGDQPYQGRGDQPYQGRGDQPYQGRGDQPYQGR | 4 |
| GDQPYQGRGDQPYQGRGDQPYQGRGDQPYQGRGDQPYQGRGDQPYQGRGDQPYQGRGDQPYQGRGDQPYQ | |
| GRGDQPYQGRGDQPYQGRGDQPYQGY | |
| SKGP-[GRGDTPYT]20-GY | |
| GYSKGPGRGDTPYTGRGDTPYTGRGDTPYTGRGDTPYTGRGDTPYTGRGDTPYTGRGDTPYTGRGDTPYT | 5, |
| GRGDTPYTGRGDTPYTGRGDTPYTGRGDTPYTGRGDTPYTGRGDTPYTGRGDTPYTGRGDTPYTGRGDTP | 6 |
| YTGRGDTPYTGRGDTPYTGRGDTPYTGY | |
| SKGP-[GRGDNPYN]20-GY | 7, |
| GYSKGPGRGDNPYNGRGDNPYNGRGDNPYNGRGDNPYNGRGDNPYNGRGDNPYNGRGDNPYNGRGDNPYN | 8 |
| GRGDNPYNGRGDNPYNGRGDNPYNGRGDNPYNGRGDNPYNGRGDNPYNGRGDNPYNGRGDNPYNGRGDNP | |
| YNGRGDNPYNGRGDNPYNGRGDNPYNGY | |
| SKGP-[GRGDSPHS]20-GY | 9, |
| GYSKGPGRGDSPHSGRGDSPHSGRGDSPHSGRGDSPHSGRGDSPHSGRGDSPHSGRGDSPHSGRGDSPHS | 10 |
| GRGDSPHSGRGDSPHSGRGDSPHSGRGDSPHSGRGDSPHSGRGDSPHSGRGDSPHSGRGDSPHSGRGDSP | |
| HSGRGDSPHSGRGDSPHSGRGDSPHSGY | |
| SKGP-[GRGDSPFS]20-GY | 11, |
| SKGPGRGDSPFSGRGDSPFSGRGDSPFSGRGDSPFSGRGDSPFSGRGDSPFSGRGDSPFSGRGDSPFSGR | 12 |
| GDSPFSGRGDSPFSGRGDSPFSGRGDSPFSGRGDSPFSGRGDSPFSGRGDSPFSGRGDSPFSGRGDSPFS | |
| GRGDSPFSGRGDSPFSGRGDSPFSGY | |
| SKGP-[GRGDQPYS]20-GY | 13, |
| SKGPGRGDQPYSGRGDQPYSGRGDQPYSGRGDQPYSGRGDQPYSGRGDQPYSGRGDQPYSGRGDQPYSGR | 14 |
| GDQPYSGRGDQPYSGRGDQPYSGRGDQPYSGRGDQPYSGRGDQPYSGRGDQPYSGRGDQPYSGRGDQPYS | |
| GRGDQPYSGRGDQPYSGRGDQPYSGY | |
| SKGP-[GRGDNPYS]20-GY | 15, |
| SKGPGRGDNPYSGRGDNPYSGRGDNPYSGRGDNPYSGRGDNPYSGRGDNPYSGRGDNPYSGRGDNPYSGR | 16 |
| GDNPYSGRGDNPYSGRGDNPYSGRGDNPYSGRGDNPYSGRGDNPYSGRGDNPYSGRGDNPYSGRGDNPYS | |
| GRGDNPYSGRGDNPYSGRGDNPYSGY | |
| SKGP-[GRGDSPYN]20-GY | 17, |
| SKGPGRGDSPYNGRGDSPYNGRGDSPYNGRGDSPYNGRGDSPYNGRGDSPYNGRGDSPYNGRGDSPYNGR | 18 |
| GDSPYNGRGDSPYNGRGDSPYNGRGDSPYNGRGDSPYNGRGDSPYNGRGDSPYNGRGDSPYNGRGDSPYN | |
| GRGDSPYNGRGDSPYNGRGDSPYNGY | |
| SKGP-[GRGDNPYQ]40-GY | 19, |
| SKGPGRGDNPYQGRGDNPYQGRGDNPYQGRGDNPYQGRGDNPYQGRGDNPYQGRGDNPYQGRGDNPYQGR | 20 |
| GDNPYQGRGDNPYQGRGDNPYQGRGDNPYQGRGDNFYQGRGDNPYQGRGDNPYQGRGDNPYQGRGDNPYQ | |
| GRGDNPYQGRGDNPYQGRGDNPYQGY | |
| SKGP-[GRGDQPYN]20-GY | 21, |
| SKGPGRGDQPYNGRGDQPYNGRGDQPYNGRGDQPYNGRGDQPYNGRGDQPYNGRGDQPYNGRGDQPYNGR | 22 |
| GDQPYNGRGDQPYNGRGDQPYNGRGDQPYNGRGDQPYNGRGDQPYNGRGDQPYNGRGDQPYNGRGDQPYN | |
| GRGDQPYNGRGDQPYNGRGDQPYNGY | |
| SKGP-[GRDGSPYQ]20-GY | 23, |
| SKGPGRDGSPYQGRDGSPYQGRDGSPYQGRDGSPYQGRDGSPYQGRDGSPYQGRDGSPYQGRDGSPYQGR | 24 |
| DGSPYQGRDGSPYQGRDGSPYQGRDGSPYQGRDGSPYQGRDGSPYQGRDGSPYQGRDGSPYQGRDGSPYQ | |
| GRDGSPYQGRDGSPYQGRDGSPYQGY | |
| SKGP-[GRDGQPYQ]20-GY | 25, |
| SKGPGRDGQPYQGRDGQPYQGRDGQPYQGRDGQPYQGRDGQPYQGRDGQPYQGRDGQPYQGRDGQPYQGR | 26 |
| DGQPYQGRDGQPYQGRDGQPYQGRDGQPYQGRDGQPYQGRDGQPYQGRDGQPYQGRDGQPYQGRDGQPYQ | |
| GRDGQPYQGRDGQPYQGRDGQPYQGY | |
| SKGP-[GRDGSPYN]20-GY | 27, |
| SKGPGRDGSPYNGRDGSPYNGRDGSPYNGRDGSPYNGRDGSPYNGRDGSPYNGRDGSPYNGRDGSPYNGR | 28 |
| DGSPYNGRDGSPYNGRDGSPYNGRDGSPYNGRDGSPYNGRDGSPYNGRDGSPYNGRDGSPYNGRDGSPYN | |
| GRDGSPYNGRDGSPYNGRDGSPYNGY | |
| SKGP-[GRGDNPHN]20-GY | 29, |
| SKGPGRGDNPHNGRGDNPHNGRGDNPHNGRGDNPHNGRGDNPHNGRGDNPHNGRGDNPHNGRGDNPHNGR | 30 |
| GDNPHNGRGDNPHNGRGDNPHNGRGDNPHNGRGDNPHNGRGDNPHNGRGDNPHNGRGDNPHNGRGDNPHN | |
| GRGDNPHNGRGDNPHNGRGDNPHNGY | |
| SKGP-[GRGDNPHS]20-GY | 31, |
| SKGPGRGDNPHSGRGDNPHSGRGDNPHSGRGDNPHSGRGDNPHSGRGDNPHSGRGDNPHSGRGDNPHSGR | 32 |
| GDNPHSGRGDNPHSGRGDNPHSGRGDNPHSGRGDNPHSGRGDNPHSGRGDNPHSGRGDNPHSGRGDNPHS | |
| GRGDNPHSGRGDNPHSGRGDNPHSGY | |
| SKGP-[GRGDSPHN]20-GY | 33, |
| SKGPGRGDSPHNGRGDSPHNGRGDSPHNGRGDSPHNGRGDSPHNGRGDSPHNGRGDSPHNGRGDSPHNGR | 34 |
| GDSPHNGRGDSPHNGRGDSPHNGRGDSPHNGRGDSPHNGRGDSPHNGRGDSPHNGRGDSPHNGRGDSPHN | |
| GRGDSPHNGRGDSPHNGRGDSPHNGY | |
| SKGP-[GRGDQPHN]20-GY | 35, |
| SKGPGRGDQPHNGRGDQPHNGRGDQPHNGRGDQPHNGRGDQPHNGRGDQPHNGRGDQPHNGRGDQPHNGR | 36 |
| GDQPHNGRGDQPHNGRGDQPHNGRGDQPHNGRGDQPHNGRGDQPHNGRGDQPHNGRGDQPHNGRGDQPHN | |
| GRGDQPHNGRGDQPHNGRGDQPHNGY | |
| (GRGDNPHQ)20-GY | 37, |
| GRGDNPHQGRGDNPHQGRGDNPHQGRGDNPHQGRGDNPHQGRGDNPHQGRGDNPHQGRGDNPHQGRGDNP | 38 |
| HQGRGDNPHQGRGDNPHQGRGDNPHQGRGDNPHQGRGDNPHQGRGDNPHQGRGDNPHQGRGDNPHQGRGD | |
| NPHQGRGDNPHQGRGDNPHQGY | |
| GRGDSPAS | 39, |
| GRGDSPASGRGDSPASGRGDSPASGRGDSPASGRGDSPASGRGDSPASGRGDSPASGRGDSPASGRGDSP | 40 |
| ASGRGDSPASGRGDSPASGRGDSPASGRGDSPASGRGDSPASGRGDSPASGRGDSPASGRGDSPASGRGD | |
| SPASGRGDSPASGRGDSPASGY | |
| GRGDSPIS | 41, |
| GRGDSPISGRGDSPISGRGDSPISGRGDSPISGRGDSPISGRGDSPISGRGDSPISGRGDSPISGRGDSP | 42 |
| ISGRGDSPISGRGDSPISGRGDSPISGRGDSPISGRGDSPISGRGDSPISGRGDSPISGRGDSPISGRGD | |
| SPISGRGDSPISGRGDSPIS | |
| NR1 | 43, |
| GGRSDSYPGGRPSDYSSDRPSYGGGYRSGSPDSPDGYRSGRGYDGPSSRGSPGYDSYPGSDGSRDYRPGS | 44 |
| GSPRGGDYSSSPGDSRYGGGRYDPSSPSDSRGGYSPYGSRDGSSPGYGRDRSDSGGYPPDRSSGGYDGYS | |
| GRSPSGSDGPRYSYGRSPGD | |
| NR2 | 45, |
| GRDSGYSPPGRDGYSSYSPGDSGRSRSGPDGYSRDSYGGPSDGYPRSGGDSSPGRYGPYRDGSSGPGSRD | 46 |
| YSRGSGSYDPYPRGDSGSPYRGGDSSDSGGYSPRYDGPGRSSYGSPDSRGRDSGPGYSDPGYSRSGSDPG | |
| YGSRYSDPSRGGRSPDGYGS | |
| NR3 | 47, |
| GDRSPYSGGSRPGSDYGSDSRYPGGPYRGSDSSGRSYPDGDGSGPYRSSSDRPYGGSSYGGDRPGYDRSG | 48 |
| PSGGDYSPRSGYRSPSDGSGPYDGRSYGPGSRSDSDGRSGPYSDGYPSGRDPRSGGYSRSYDGSGPYRPS | |
| DSGGPDGSRYSGSRDSGYPG | |
| NR4 | 49, |
| GPYSDRGSGDSGSYPRPRSGGDSYPGSYDGRSYSGRPDSGGSDGRYPSRGGPYSDSRPSGYGSDDPYSGG | 50 |
| SRGRGSPSDYDGSYRGPSPGDYSSGRGGSRYDPSDRSPSGYGPDGSGYRSSRDSGYGPGDGRPSYSDPGG | |
| SYSRRSSDGPGYDGSRPYGS | |
| NR5 | 51, |
| GRDSGYSPPSRGYSDGGDSYSPGRPSGRYSGDGSGPDSRYSDSGGRYPPGSYSDGRSPRSYGDGSYPDGR | 52 |
| GSPYRSGGSDPDYGSRSGGRGSSPDYGYDSPSRGRPGSDYSGGDPYSGRSSGRPDSGYYDRSSGPGYGDS | |
| RPGSGDSGPYRSPSRSGYGD | |
| NR6 | 53, |
| GGSPDYSRPYRGSDGSDSGGYRPSGSGDSRYPSRDYSPGGYDSPGSRGRGSYDSGPPSRSYGGDDGRPGS | 54 |
| SYSDRSYGGPGGDSYRSPYDGSSGPRRSGGYSPDGDYSGPSRGDSGYSPRRSGSGDYPYSRGDSGPDGRP | |
| SGYSYDGSRPSGSGYPGDRS | |
| NR7 | 55, |
| GGDSPGYYDGYRRDSSRPDGGSYPGSGRSPSSGRPSSYGDSPRDGSSGRSPSGGGYSSSDGSYYDGRPRY | 56 |
| GPSDPRDGGYRGSGDDPSSSPSYDYYDRSRGPYPSGSSSGGRYPGDGRGGYDYGGYPRDYRDPSSGRDGG | |
| YSSPSRGGSPSSPSRSGGGD | |

NR8

| | |
|---|---|
| GRSYRGDGYGSSSYYYGGPDPSGGPSSPPSRDGSDGRRDSGGGPSSSDYPSSPSGGDYSYGPDGSRYDGS | 57, |
| RRRDYPRSGGSRGYSDYGRPYGGSGGDGGPYSPPRDSSSYGPDRSSDSGRSRPSGSSYPYSDSGYPRPGG | 58 |
| sERRDSSDGGYDYRSGGDS | |
| NR9 | |
| GDYYRGYPSPRGSPGPGSSGSDDSRYPGDSGDSRRYSSGGGYSRGSDRRYDSSSGGGPYPYDRDSRSGYD | 59, |
| GPSSSGPGGPSDSPGGRPSGSYPDDGGGRSDSSPYRGYDSRSSPGYYGGRSPYRDSPRDYGPPSDDRGSD | 60 |
| GGRYPSGSSGGGRSGYGSYS | |
| NR10 | 61, |
| GYSGSSPYRPGSSRPSDGYGPGDGGPSRDSRSYSYDDGGRGGSPDPGGSRSRRPGDGGYSYSSPYGSGYD | 62 |
| GKYKDSSDPSDDGRPPKKGYDGPSYGGGKYGGSRPSDYSPSSDSSSGYSGGDGGSSRDFYYYKKGSSGPS | |
| YSDGPRSSGDPYRPRGPGSS | |
| NR11 | |
| GDGSSYGGDSPYPSGGSRGYRGPPDRRRGGDSSSSSPDGYRPSDGPGSDRYDSSGGSSYSRGYRSPSPGD | 63, |
| DGYSGRGPGYDGRYYPGRSRSGSSPGSGPGSSYPRSDDGDGSPYSYDGRGSPSPSGYGPGSRGDGRDYRD | 64 |
| GGDSSSDGPGRRPYSYSGSY | |
| NR12 | 65, |
| GDGSYPRSPSPSYGGGSGPSRRRDSYRGDGDGYYGDSSSPGDSGGRGSDDGYPSSSDSYSRSRGGSSPDG | 66 |
| YPYPGPRRYGYGPGSGYYSSSDDGSRDSPRYRGGPYRGPSDGGRSGSDSPGGSYSSGRGSDSYGGGDRPR | |
| PGRDDYGPYPSPSDSYSSRG | |
| NR1-eukOpt | 67, |
| GGRSDSYPGGRPSDYSSDRPSYGGGYRSGSPDSPDGYRSGRGYDGPSSRGSPGYDSYPGSDGSRDYRPGS | 68 |
| GSPRGGDYSSSPGDSRYGGGRYDPSSPSDSRGGYSPYGSRDGSSPGYGRDRSDSGGYPPDRSSGGYDGYS | |
| GRSPSGSDGPRYSYGRSPGD | |
| NR2-eukOpt | 69, |
| GRDSGYSPPGRDGYSSYSPGDSGRSRSGPDGYSRDSYGGPSDGYPRSGGDSSPGRYGPYRDGSSGPGSRD | 70 |
| YSRGSGSYDPYPRGDSGSPYRGGDSSDSGGYSPRYDGPGRSSYGSPDSRGRDSGPGYSDPGYSRSGSDPG | |
| YGSRYSDPSRGGRSPDGYGS | |
| SKGP-[GRGDSPYSGRGDSPYSGRGDSPYSGRGDQPYQ]5-GY | 71, |
| SKGPGRGDSPYSGRGDSPYSGRGDSPYSGRGDQPYQGRGDSPYSGRGDSPYSGRGDSPYSGRGDQPYQGR | 72 |
| GDSPYSGRGDSPYSGRGDSPYSGRGDQPYQGRGDSFYSGRGDSPYSGRGDSPYSGRGDQPYQGRGDSPYS | |
| GRGDSPYSGRGDSPYSGRGDQPYQGY | |
| SKGP-[GRGDSPYSGRGDQPYQ]10-GY | 73, |
| SKGPGRGDSPYSGRGDQPYQGRGDSPYSGRGDQPYQGRGDSPYSGRGDQPYQGRGDSPYSGRGDQPYQGR | 74 |
| GDSPYSGRGDQPYQGRGDSPYSGRGDQPYQGRGDSPYSGRGDQPYQGRGDSPYSGRGDQPYQGRGDSPYS | |
| GRGDQPYQGRGDSPYSGRGDQPYQGY | |
| SKGP-[GRGDQPYQGRGDQPYQGRGDQPYQGRGDSPYS]5-GY | 75, |
| SKGPGRGDQPYQGRGDQPYQGRGDQPYQGRGDSPYSGRGDQPYQGRGDQPYQGRGDQPYQGRGDSPYSGR | 76 |
| GDQPYQGRGDQPYQGRGDQPYQGRGDSPYSGRGDQPYQGRGDQPYQGRGDQPYQGRGDSPYSGRGDQPYQ | |
| GRGDQPYQGRGDQPYQGRGDSPYSGY | |
| SKGP-[GRGDSPYSGRGDSPYSGRGDSPYSGRGDSPVS]5-GY | 77, |
| SKGPGRGDSPYSGRGDSPYSGRGDSPYSGRGDSPVSGRGDSPYSGRGDSPYSGRGDSPYSGRGDSPVSGR | 78 |
| GDSPYSGRGDSPYSGRGDSPYSGRGDSPVSGRGDSPYSGRGDSPYSGRGDSPYSGRGDSPVSGRGDSPYS | |
| GRGDSPYSGRGDSPYSGRGDSPVSGY | |
| SKGP-[GRGDSPYSGRGDSPVS]10-GY | 79, |
| SKGPGRGDSPYSGRGDSPVSGRGDSPYSGRGDSPVSGRGDSPYSGRGDSPVSGRGDSPYSGRGDSPVSGR | 80 |
| GDSPYSGRGDSPVSGRGDSPYSGRGDSPVSGRGDSFYSGRGDSPVSGRGDSPYSGRGDSPVSGRGDSPYS | |
| GRGDSPVSGRGDSPYSGRGDSPVSGY | |
| SKGP-[GRGDSPVSGRGDSPVSGRGDSPVSGRGDSPYS]5-GY | 81, |
| SKGPGRGDSPVSGRGDSPVSGRGDSPVSGRGDSPYSGRGDSPVSGRGDSPVSGRGDSPVSGRGDSPYSGR | 82 |
| GDSPVSGRGDSPVSGRGDSPVSGRGDSPYSGRGDSPVSGRGDSPVSGRGDSPVSGRGDSPYSGRGDSPVS | |
| GRGDSPVSGRGDSPVSGRGDSPYSGY | |
| SKGP-[GRGDSPVS]20-GY | 83, |
| SKGPGRGDSPVSGRGDSPVSGRGDSPVSGRGDSPVSGRGDSPVSGRGDSPVSGRGDSPVSGRGDSPVSGR | 74 |
| GDSPVSGRGDSPVSGRGDSPVSGRGDSPVSGRGDSPVSGRGDSPVSGRGDSPVSGRGDSPVSGRGDSPVS | |
| GRGDSPVSGRGDSPVSGRGDSPVSGY | |
| SKGP-[GRGDSPYSGRGDSPYSGRGDSPYSGRGDSPAS]5-GY | 85, |
| SKGPGRGDSPYSGRGDSPYSGRGDSPYSGRGDSPASGRGDSPYSGRGDSPYSGRGDSPYSGRGDSPASGR | 86 |
| GDSPYSGRGDSPYSGRGDSPYSGRGDSPASGRGDSPYSGRGDSPYSGRGDSPYSGRGDSPASGRGDSPYS | |
| GRGDSPYSGRGDSPYSGRGDSPASGY | |
| SKGP-[GRGDSPYSGRGDSPAS]10-GY | 87, |
| SKGPGRGDSPYSGRGDSPASGRGDSPYSGRGDSPASGRGDSPYSGRGDSPASGRGDSPYSGRGDSPASGR | 88 |
| GDSPYSGRGDSPASGRGDSPYSGRGDSPASGRGDSPYSGRGDSPASGRGDSPYSGRGDSPASGRGDSPYS | |
| GRGDSPASGRGDSPYSGRGDSPASGY | |
| SKGP-[GRGDSPYSGRGDSPYSGRGDSPYSGRGDSPIS]5-GY | 89, |
| SKGPGRGDSPYSGRGDSPYSGRGDSPYSGRGDSPISGRGDSPYSGRGDSPYSGRGDSPYSGRGDSPISGR | 90 |
| GDSPYSGRGDSPYSGRGDSPYSGRGDSPISGRGDSPYSGRGDSPYSGRGDSPYSGRGDSPISGRGDSPYS | |
| GRGDSPYSGRGDSPYSGRGDSPISGY | |
| SKGP-[GRGDSPYSGRGDSPIS]W-GY | 91, |
| SKGPGRGDSPYSGRGDSPISGRGDSPYSGRGDSPISGRGDSPYSGRGDSPISGRGDSPYSGRGDSPISGR | 92 |
| GDSPYSGRGDSPISGRGDSPYSGRGDSPISGRGDSPYSGRGDSPISGRGDSPYSGRGDSPISGRGDSPYS | |
| GRGDSPISGRGDSPYSGRGDSPISGY | |
| SKGP-[GRGDSPYSGRGDSPYSGRGDSPYSGRGDSPMS]5-GY | 93, |
| SKGPGRGDSPYSGRGDSPYSGRGDSPYSGRGDSPMSGRGDSPYSGRGDSPYSGRGDSPYSGRGDSPMSGR | 94 |
| GDSPYSGRGDSPYSGRGDSPYSGRGDSPMSGRGDSPYSGRGDSPYSGRGDSPYSGRGDSPMSGRGDSPYS | |
| GRGDSPYSGRGDSPYSGRGDSPMSGY | |
| SKGP-[GRGDSPYSGRGDSPMS]10-GY | 95, |
| SKGPGRGDSPYSGRGDSPMSGRGDSPYSGRGDSPMSGRGDSPYSGRGDSPMSGRGDSPYSGRGDSPMSGR | 96 |
| GDSPYSGRGDSPMSGRGDSPYSGRGDSPMSGRGDSPYSGRGDSPMSGRGDSPYSGRGDSPMSGRGDSPYS | |
| GRGDSPMSGRGDSPYSGRGDSPMSGY | |
| SKGP-[GRGDSPYSGRGDSPYSGRGDSPYSGRGDSPHS]5-GY | |
| SKGPGRGDSPYSGRGDSPYSGRGDSPYSGRGDSPHSGRGDSPYSGRGDSPYSGRGDSPYSGRGDSPHSGR | 97, |
| GDSPYSGRGDSPYSGRGDSPYSGRGDSPHSGRGDSPYSGRGDSPYSGRGDSPYSGRGDSPHSGRGDSPYS | 98 |
| GRGDSPYSGRGDSPYSGRGDSPMSGY | |
| SKGP-[GRGDSPYSGRGDSPHS]10-GY | 99, |
| SKGPGRGDSPYSGRGDSPHSGRGDSPYSGRGDSPHSGRGDSPYSGRGDSPMSGRGDSPYSGRGDSPHSGR | 100 |
| GDSPYSGRGDSPHSGRGDSPYSGRGDSPHSGRGDSPYSGRGDSPHSGRGDSPYSGRGDSPHSGRGDSPYS | |
| GRGDSPHSGRGDSPYSGRGDSPHSGY | |
| SKGP-[GRGDSPHSGRGDSPHSGRGDSPHSGRGDSPYS]5-GY | 101, |
| SKGPGRGDSPMSGRGDSPHSGRGDSPMSGRGDSPYSGRGDSPHSGRGDSPMSGRGDSPHSGRGDSPYSGR | 102 |
| GDSPHSGRGDSPHSGRGDSPHSGRGDSPYSGRGDSPMSGRGDSPHSGRGDSPHSGRGDSPYSGRGDSPHS | |
| GRGDSPHSGRGDSPHSGRGDSPYSGY | |
| SKGP-[GRGDSPYSGRGDSPYSGRGDSPYSGKGDSPYS]5-GY | 103, |
| SKGPGRGDSPYSGRGDSPYSGRGDSPYSGRGDSPYSGRGDSPYSGRGDSPYSGRGDSPYSGKGDSPYSGR | 104 |
| GDSPYSGRGDSPYSGRGDSPYSGKGDSPYSGRGDSPYSGRGDSPYSGRGDSPYSGKGDSPYSGRGDSPYS | |
| GRGDSPYSGRGDSPYSGKGDSPYSGY | |
| SKGP-[GRGDSPYSGKGDSPYS]10-GY | 105, |
| SKGPGRGDSPYSGKGDSPYSGRGDSPYSGKGDSPYSGRGDSPYSGKGDSPYSGRGDSPYSGKGDSPYSGR | 106 |
| GDSPYSGKGDSPYSGRGDSPYSGKGDSPYSGRGDSPYSGKGDSPYSGRGDSPYSGKGDSPYSGRGDSPYS | |
| GKGDSPYSGRGDSPYSGKGDSPYSGY | |
| GRGDSPYSGRGDSPYSGRGDSPYSGRGESPYS | 107, |
| GRGDSPYSGRGDSPYSGRGDSPYSGRGESPYSGRGDSPYSGRGDSPYSGRGDSPYSGRGESPYSGRGDSP | 108 |
| YSGRGDSPYSGRGDSPYSGRGESPYSGRGDSPYSGRGDSPYSGRGDSPYSGRGESPYSGRGDSPYSGRGD | |
| SPYSGRGDSPYSGRGESPYS | |
| SKGP-[GRGDSPYSGRGESPYS]10-GY | 109, |
| SKGPGRGDSPYSGRGESPYSGRGDSPYSGRGESPYSGRGDSPYSGRGESPYSGRGDSPYSGRGESPYSGR | 110 |
| GDSPYSGRGESPYSGRGDSPYSGRGESPYSGRGDSPYSGRGESPYSGRGDSPYSGRGESPYSGRGDSPYS | |
| GRGESPYSGRGDSPYSGRGESPYSGY | |
| [(GRPDSPYSGRGDSPYSGRGDSPYSGRGDSPYS)- | 111, |
| (PRGDSPYSGRGDSPYSGRGDSPYSGRGDSPYS)-2.5 | 112 |
| GRPDSPYSGRGDSPYSGRGDSPYSGRGDSPYSPRGDSPYSGRGDSPYSGRGDSPYSGRGDSPYSGRPDSP | |
| YSGRGDSPYSGRGDSPYSGRGDSPYSPRGDSPYSGRGDSPYSGRGDSPYSGRGDSPYSGRPDSPYSGRGD | |
| SPYSGRGDSPYSGRGDSPYS | |
| GRGDSPYSGRPDSPYSGRGDSPYSGRGDSPYSGRGDSPYS- | 113, |
| [(GRPDSPYSGRGDSPYSPRGDSPYSGRGDSPYS)-5 | 114 |
| GRPDSPYSGRGDSPYSPRGDSPYSGRGDSPYSGRPDSPYSGRGDSPYSPRGDSPYSGRGDSPYSGRPDSP | |
| YSGRGDSPYSPRGDSPYSGRGDSPYSGRPDSPYSGRGDSPYSPRGDSPYSGRGDSPYSGRPDSPYSGRGD | |
| SPYSPRGDSPYSGRGDSPYS | |
| GRPDSPYSPRGDSPYSGRPDSPYSGRGDSPYSPRGDSPYSGRPDSPYSPRGDSPYSGRGD | 116, |
| SPYS-2.5 | 116 |
| GRPDSPYSGRGDSPYSPRGDSPYSGRGDSPYSGRPDSPYSGRGDSPYSPRGDSPYSGRGDSPYSGRPDSP | |
| YSGRGDSFYSPRGDSPYSGRGDSPYSGRPDSPYSGRGDSPYSPRGDSPYSGRGDSPYSGRPDSPYSGRGD | |
| SPYSPRGDSPYSGRGDSPYS | |
| (GRPDSPYSPRGDSPYS)-10 | 117, |
| GRPDSPYSPRGDSPYSGRPDSPYSPRGDSPYSGRPDSPYSPRGDSPYSGRPDSPYSPRGDSPYSGRPDSP | 118 |
| YSPRGDSPYSGRPDSPYSPRGDSPYSGRPDSPYSPRGDSPYSGRPDSPYSPRGDSPYSGRPDSPYSPRGD | |
| SPYSGRPDSPYSPRGDSPYS | |
| SKGP-[GRGDSPYSGRGDSPVSGRGDSPYSGRGDSPVSGRGDSPYSGRGDSPVSGRGDSPY | 119, |
| SGKGDSPYS]2GRGDSPYSGRGDSPVSGRGDSPYSGRGDSPVSGY | 120 |
| SKGPGRGDSPYSGRGDSPVSGRGDSPYSGKGDSPYSGRGDSPYSGRGDSPVSGRGDSPYSGKGDSPYSGR | |
| GDSPYSGRGDSPVSGRGDSPYSGKGDSPYSGRGDSPYSGRGDSPVSGRGDSPYSGKGDSPYSGRGDSPYS | |
| GRGDSPVSGRGDSPYSGKGDSPYSGY | |
| SKGP-[GRGDSPYSGRGDSPVSGRGDSPYSGKGDSPYS]10-GY | 121, |
| SKGPGRGDSPYSGRGDSPVSGRGDSPYSGRGDSPVSGRGDSPYSGRGDSPVSGRGDSPYSGKGDSPYSGR | 122 |
| GDSPYSGRGDSPVSGRGDSPYSGRGDSPVSGRGDSPYSGRGDSPVSGRGDSPYSGKGDSPYSGRGDSPYS | |
| GRGDSPVSGRGDSPYSGRGDSPVSGY | |
| SKGP-[GRGDSPYSGKGDSPYSGRGDSPYSGKGDSPYSGRGDSPYSGKGDSPYSGRGDSPYSGRG | 123, |
| DSPVS]2-GRGDSPYSGKGDSPYSGRGDSPYSGKGDSPYSGY | 124 |
| SKGPGRGDSPYSGKGDSPYSGRGDSPYSGKGDSPYSGRGDSPYSGKGDSPYSGRGDSPYSGRGDSPVSGR | |
| GDSPYSGKGDSPYSGRGDSPYSGKGDSPYSGRGDSPYSGKGDSPYSGRGDSPYSGRGDSPVSGRGDSPYS | |
| GKGDSPYSGRGDSPYSGKGDSPYSGY | |
| [(GRGDSPYS)-3-(GRGDSPLS)]-5 | 125, |
| GRGDSPYSGRGDSPYSGRGDSPYSGRGDSPLSGRGDSPYSGRGDSPYSGRGDSPYSGRGDSPLSGRGDSP | 126 |
| YSGRGDSPYSGRGDSPYSGRGDSPLSGRGDSPYSGRGDSPYSGRGDSPYSGRGDSPLSGRGDSPYSGRGD | |
| SPYSGRGDSPYSGRGDSPLS | |
| [(GRGDSPYS)-(GRGDSPLS)]-10 | 127, |
| GRGDSPYSGRGDSPLSGRGDSPYSGRGDSPLSGRGDSPYSGRGDSPLSGRGDSPYSGRGDSPLSGRGDSP | 128 |
| YSGRGDSPLSGRGDSPYSGRGDSPLSGRGDSPYSGRGDSPLSGRGDSPYSGRGDSPLSGRGDSPYSGRGD | |
| SPLSGRGDSPYSGRGDSPLS | |
| R/D 19/21 | |
| GRGDSPYSGRGDSPYSGRGDSPYSGRGDSPYSGRGDSPYSGRGDSPYSGRGDSPYSGRGDSPYSGRGDSP | 129, |
| YSGRGDSPYSGDGDSPYSGRGDSPYSGRGDSPYSGRGDSPYSGRGDSPYSGRGDSPYSGRGDSPYSGRGD | 130 |
| SPYSGRGDSPYSGRGDSPYS | |
| R/D 18/22 | 131, |
| GRGDSPYSGRGDSPYSGRGDSPYSGRGDSPYSGRGDSPYSGRGDSPYSGDGDSPYSGRGDSPYSGRGDSP | 132 |
| YSGRGDSPYSGRGDSPYSGRGDSPYSGRGDSPYSGDGDSPYSGRGDSPYSGRGDSPYSGRGDSPYSGRGD | |
| SPYSGRGDSPYSGRGDSPYS | |

GRGRSPYSGRGDSPYSGRGDSPYSGRGDSPYS

| | |
|---|---|
| GRGRSPYSGRGDSPYSGRGDSPYSGRGDSPYSGRGRSPYSGRGDSPYSGRGDSPYSGRGDSPYSGRGRSP | 133, |
| YSGRGDSPYSGRGDSPYSGRGDSPYSGRGRSPYSGRGDSPYSGRGDSPYSGRGDSPYSGRGRSPYSGRGD | 134 |
| SPYSGRGDSPYSGRGDSPYS | |
| GRGRSPYSGRGDSPYS | 135, |
| GRGRSPYSGRGDSPYSGRGRSPYSGRGDSPYSGRGRSPYSGRGDSPYSGRGRSPYSGRGDSPYSGRGRSP | 136 |
| YSGRGDSPYSGRGRSPYSGRGDSPYSGRGRSPYSGRGDSPYSGRGRSPYSGRGDSPYSGRGRSPYSGRGD | |
| SPYSGRGRSPYSGRGDSPYS | |
| GDGDSPYSGRGDSPYSGRGDSPYSGRGDSPYS | 137, |
| GDGDSPYSGRGDSPYSGRGDSPYSGRGDSPYSGDGDSPYSGRGDSPYSGRGDSPYSGRGDSPYSGDGDSP | 138 |
| YSGRGDSPYSGRGDSPYSGRGDSPYSGDGDSPYSGRGDSPYSGRGDSPYSGRGDSPYSGDGDSPYSGRGD | |
| SPYSGRGDSPYSGRGDSPYS | |
| GDGDSPYSGRGDSPYS | 139, |
| GDGDSPYSGRGDSPYSGDGDSPYSGRGDSPYSGDGDSPYSGRGDSPYSGDGDSPYSGRGDSPYSGDGDSP | 140 |
| YSGRGDSPYSGDGDSPYSGRGDSPYSGDGDSPYSGRGDSPYSGDGDSPYSGRGDSPYSGDGDSPYSGRGD | |
| SPYSGDGDSPYSGRGDSPYS | |
| [(GRGDSPYS)-3-(GRGDNPYN)]-5 | 141, |
| GRGDSPYSGRGDSPYSGRGDSPYSGRGDNPYNGRGDSPYSGRGDSPYSGRGDSPYSGRGDNPYNGRGDSP | 142 |
| YSGRGDSPYSGRGDSPYSGRGDNPYNGRGDSPYSGRGDSPYSGRGDSPYSGRGDNPYNGRGDSPYSGRGD | |
| SPYSGRGDSPYSGRGDNPYN | |
| ((GRGDSPYS)-(GRGDNPYN)-10 | 143, |
| GRGDSPYSGRGDNPYNGRGDSPYSGRGDNPYNGRGDSPYSGRGDNPYNGRGDSPYSGRGDNPYNGRGDSP | 144 |
| YSGRGDNPYNGRGDSPYSGRGDNPYNGRGDSPYSGRGDNPYNGRGDSPYSGRGDNPYNGRGDSPYSGRGD | |
| NPYNGRGDSPYSGRGDNPYN | |
| [(GRGDNPYN)-3-(GRGDSPYS)]-5 | 145, |
| GRGDNPYNGRGDNPYNGRGDNPYNGRGDSPYSGRGDNPYNGRGDNPYNGRGDNPYNGRGDSPYSGRGDNP | 146 |
| YNGRGDNPYNGRGDNPYNGRGDSPYSGRGDNPYNGRGDNPYNGRGDNPYNGRGDSPYSGRGDNPYNGRGD | |
| NPYNGRGDNPYNGRGDSPYS | |
| [(GRGDQPYQ)-3-(GRGDQPHQ)]-5 | 147, |
| GRGDQPYQGRGDQPYQGRGDQPYQGRGDQPHQGRGDQPYQGRGDQPYQGRGDQPYQGRGDQPHQGRGDQP | 148 |
| YQGRGDQFYQGRGDQPYQGRGDQPHQGRGDQPYQGRGDQPYQGRGDQPYQGRGDQPHQGRGDQPYQGRGD | |
| QPYQGRGDQPYQGRGDQPHQ | |
| [(GRGDQPYQ)-(GRGDQPHQ)]-10 | |
| GRGDQPYQGRGDQPHQGRGDQPYQGRGDQPHQGRGDQPYQGRGDQPHQGRGDQPYQGRGDQPHQGRGDQP | 149, |
| YQGRGDQPHQGRGDQPYQGRGDQPHQGRGDQPYQGRGDQPHQGRGDQPYQGRGDQPHQGRGDQPYQGRGD | 150 |
| QPHQGRGDQPYQGRGDQPHQ | |
| [(GRGDQPHQ)-3-(GRGDQPYQ)]-5 | 151, |
| GRGDQPHQGRGDQPHQGRGDQPHQGRGDQPYQGRGDQPHQGRGDQPHQGRGDQPHQGRGDQPYQGRGDQP | 152 |
| HQGRGDQPHQGRGDQPHQGRGDQPYQGRGDQPHQGRGDQPHQGRGDQPHQGRGDQPYQGRGDQPHQGRGD | |
| QPHQGRGDQPHQGRGDQPYQ | |
| GRDGSPYQ | |
| GRDGSPYQGRDGSPYQGRDGSPYQGRDGSPYQGRDGSPYQGRDGSPYQGRDGSPYQGRDGSPYQGRDGSP | 153, |
| YQGRDGSPYQGRDGSPYQGRDGSPYQGRDGSPYQGRDGSPYQGRDGSPYQGRDGSPYQGRDGSPYQGRDG | 154 |
| SPYQGRDGSPYQGRDGSPYQ | |
| GRDGQPYQ | 155, |
| GRDGQPYQGRDGQPYQGRDGQPYQGRDGQPYQGRDGQPYQGRDGQPYQGRDGQPYQGRDGQPYQGRDGQP | 156 |
| YQGRDGQPYQGRDGQPYQGRDGQPYQGRDGQPYQGRDGQPYQGRDGQPYQGRDGQPYQGRDGQPYQGRDG | |
| QPYQGRDGQPYQGRDGQPYQ | |
| GRDGSPYN | |
| GRDGSPYNGRDGSPYNGRDGSPYNGRDGSPYNGRDGSPYNGRDGSPYNGRDGSPYNGRDGSPYNGRDGSP | 157, |
| YNGRDGSPYNGRDGSPYNGRDGSPYNGRDGSPYNGRDGSPYNGRDGSPYNGRDGSPYNGRDGSPYNGRDG | 158 |
| SPYNGRDGSPYNGRDGSPYN | |

SKGP-[GRGDSPYS]20-GY

| | |
|---|---|
| SKGPGRGDSPYSGRGDSPYSGRGDSPYSGRGDSPYSGRGDSPYSGRGDSPYSGRGDSPYSGRGDSPYSGR | 197 |
| GDSPYSGRGDSPYSGRGDSPYSGRGDSPYSGRGDSPYSGRGDSPYSGRGDSPYSGRGDSPYSGRGDSPYS | |
| GRGDSPYSGRGDSPYSGRGDSPYSGY | |
| SKGP-[GRGDSPYS]40-GY | 198 |
| SKGPGRGDSPYSGRGDSPYSGRGDSPYSGRGDSPYSGRGDSPYSGRGDSPYSGRGDSPYSGRGDSPYSGR | |
| GDSPYSGRGDSPYSGRGDSPYSGRGDSPYSGRGDSPYSGRGDSPYSGRGDSPYSGRGDSPYSGRGDSPYS | |
| GRGDSPYSGRGDSPYSGRGDSPYSGRGDSPYSGRGDSPYSGRGDSPYSGRGDSPYSGRGDSPYSGRGDSP | |
| YSGRGDSPYSGRGDSPYSGRGDSPYSGRGDSPYSGRGDSPYSGRGDSPYSGRGDSPYSGRGDSPYSGRGD | |
| SPYSGRGDSPYSGRGDSPYSGRGDSPYSGRGDSPYSGRGDSPYSGY | |
| SKGP-[GRGDSPYS]60-GY | 199 |
| SKGPGRGDSFYSGRGDSPYSGRGDSPYSGRGDSPYSGRGDSPYSGRGDSPYSGRGDSPYSGRGDSPYSGR | |
| GDSPYSGRGDSPYSGRGDSPYSGRGDSPYSGRGDSPYSGRGDSPYSGRGDSPYSGRGDSPYSGRGDSPYS | |
| GRGDSPYSGRGDSPYSGRGDSPYSGRGDSPYSGRGDSPYSGRGDSPYSGRGDSPYSGRGDSPYSGRGDSP | |
| YSGRGDSPYSGRGDSPYSGRGDSPYSGRGDSPYSGRGDSPYSGRGDSPYSGRGDSPYSGRGDSPYSGRGD | |
| SPYSGRGDSPYSGRGDSPYSGRGDSPYSGRGDSPYSGRGDSPYSGRGDSPYSGRGDSPYSGRGDSPYSGR | |
| GDSPYSGRGDSPYSGRGDSPYSGRGDSPYSGRGDSPYSGRGDSPYSGRGDSPYSGRGDSPYSGRGDSPYS | |
| GRGDSPYSGRGDSPYSGRGDSPYSGRGDSPYSGRGDSPYSGRGDSPYSGRGDSPYSGRGDSPYSGY | |
| SKGP-[GRGDSPYS]80-GY | 200 |
| SKGPGRGDSPYSGRGDSPYSGRGDSPYSGRGDSPYSGRGDSPYSGRGDSPYSGRGDSPYSGRGDSPYSGR | |
| GDSPYSGRGDSPYSGRGDSPYSGRGDSPYSGRGDSPYSGRGDSPYSGRGDSPYSGRGDSPYSGRGDSPYS | |
| GRGDSPYSGRGDSPYSGRGDSPYSGRGDSPYSGRGDSPYSGRGDSPYSGRGDSPYSGRGDSPYSGRGDSP | |
| YSGRGDSPYSGRGDSPYSGRGDSPYSGRGDSPYSGRGDSPYSGRGDSPYSGRGDSPYSGRGDSPYSGRGD | |
| SPYSGRGDSPYSGRGDSPYSGRGDSPYSGRGDSPYSGRGDSPYSGRGDSPYSGRGDSPYSGRGDSPYSGR | |
| GDSPYSGRGDSPYSGRGDSPYSGRGDSPYSGRGDSPYSGRGDSPYSGRGDSPYSGRGDSPYSGRGDSPYS | |
| GRGDSPYSGRGDSPYSGRGDSPYSGRGDSPYSGRGDSPYSGRGDSPYSGRGDSPYSGRGDSPYSGRGDSP | |
| YSGRGDSPYSGRGDSPYSGRGDSPYSGRGDSPYSGRGDSPYSGRGDSPYSGRGDSPYSGRGDSPYSGRGD | |
| SPYSGRGDSPYSGRGDSPYSGRGDSPYSGRGDSPYSGRGDSPYSGRGDSFYSGRGDSPYSGRGDSPYSGR | |
| GDSPYSGRGDSPYSGY | |
| SKGP-[GRGDQPYQ]20-GY | 201 |
| SKGPGRGDQPYQGRGDQPYQGRGDQPYQGRGDQPYQGRGDQPYQGRGDQPYQGRGDQPYQGRGDQPYQGR | |
| GDQPYQGRGDQPYQGRGDQPYQGRGDQPYQGRGDQPYQGRGDQPYOGRGDQPYQGRGDQPYQGRGDQPYQ | |
| GRGDQPYQGRGDQPYQGRGDQPYQGY | |
| SKGP-[GRGDQPYQ]40-GY | 202 |
| SKGPGRGDQPYQGRGDQPYQGRGDQPYQGRGDQPYQGRGDQPYQGRGDQPYQGRGDQPYQGRGDQPYQGR | |
| GDQPYQGRGDQPYQGRGDQPYQGRGDQPYQGRGDQPYQGRGDQPYQGRGDQPYQGRGDQPYQGRGDQPYQ | |
| GRGDQPYQGRGDQPYQGRGDQPYQGRGDQPYQGRGDQPYQGRGDQPYQGRGDQPYQGRGDQPYQGRGDQP | |
| YQGRGDQPYQGRGDQPYQGRGDQPYQGRGDQPYQGRGDQPYQGRGDQPYQGRGDQPYQGRGDQPYQGRGD | |
| QPYQGRGDQPYQGRGDQPYQGRGDQPYQGRGDQPYQGRGDQPYQGY | |
| SKGP-[GRGDSPYSGRGDSPYSGRGDSPYSGRGDQPYQ]10-GY | 203 |
| SKGPGRGDSPYSGRGDSPYSGRGDSPYSGRGDQPYQGRGDSPYSGRGDSPYSGRGDSPYSGRGDQPYQGR | |
| GDSPYSGRGDSPYSGRGDSPYSGRGDQPYQGRGDSPYSGRGDSPYSGRGDSPYSGRGDQPYQGRGDSPYS | |
| GRGDSPYSGRGDSPYSGRGDQPYQGRGDSPYSGRGDSPYSGRGDSPYSGRGDQPYQGRGDSPYSGRGDSP | |
| YSGRGDSPYSGRGDQPYQGRGDSPYSGRGDSPYSGRGDSPYSGRGDQPYQGRGDSPYSGRGDSPYSGRGD | |
| SPYSGRGDQPYQGRGDSPYSGRGDSPYSGRGDSPYSGRGDQPYQGY | |
| SKGP-[GRGDSPYSGRGDQPYQ]20-GY | 204 |
| SKGPGRGDSPYSGRGDQPYQGRGDSPYSGRGDQPYQGRGDSPYSGRGDQPYQGRGDSPYSGRGDQPYQGR | |
| GDSPYSGRGDQPYQGRGDSPYSGRGDQPYQGRGDSPYSGRGDQPYQGRGDSPYSGRGDQPYQGRGDSPYS | |
| GRGDQPYQGRGDSPYSGRGDQPYQGRGDSPYSGRGDQPYQGRGDSPYSGRGDQPYQGRGDSPYSGRGDQP | |
| YQGRGDSPYSGRGDQPYQGRGDSPYSGRGDQPYQGRGDSPYSGRGDQPYQGRGDSPYSGRGDQPYQGRGD | |
| SPYSGRGDQPYQGRGDSPYSGRGDQPYQGRGDSPYSGRGDQPYQGY | |
| SKGP-[GRGDQPYQGRGDQPYQGRGDQPYQGRGDSPYS]10-GY | 205 |
| SKGPGRGDQPYQGRGDQPYQGRGDQPYQGRGDSPYSGRGDQPYQGRGDQPYQGRGDQPYQGRGDSPYSGR | |
| GDQPYQGRGDQPYQGRGDQPYQGRGDSPYSGRGDQPYQGRGDQPYQGRGDQPYQGRGDSPYSGRGDQPYQ | |
| GRGDQPYQGRGDQPYQGRGDSPYSGRGDQPYQGRGDOPYQGRGDQPYQGRGDSPYSGRGDQPYQGRGDQP | |
| YQGRGDQPYQGRGDSPYSGRGDQPYQGRGDQPYQGRGDQPYQGRGDSPYSGRGDQPYQGRGDQPYQGRGD | |
| QPYOGRGDSPYSGRGDQPYQGRGDQPYQGRGDQPYQGRGDSPYSGY | |
| SKGP-[GRGDSPYSGRGDSPYSGRGDSPYSGRGDSPVS]10-GY | 206 |
| SKGPGRGDSFYSGRGDSPYSGRGDSPYSGRGDSPVSGRGDSPYSGRGDSPYSGRGDSPYSGRGDSPVSGR | |
| GDSPYSGRGDSPYSGRGDSPYSGRGDSPVSGRGDSPYSGRGDSPYSGRGDSPYSGRGDSPVSGRGDSPYS | |
| GRGDSPYSGRGDSPYSGRGDSPVSGRGDSPYSGRGDSPYSGRGDSPYSGRGDSPVSGRGDSPYSGRGDSP | |
| YSGRGDSPYSGRGDSPVSGRGDSPYSGRGDSPYSGRGDSPYSGRGDSPVSGRGDSPYSGRGDSPYSGRGD | |
| SPYSGRGDSPVSGRGDSPYSGRGDSPYSGRGDSPYSGRGDSPVSGY | |
| SKGP-[GRGDSPYSGRGDSPVS]20-GY | 207 |
| SKGPGRGDSPYSGRGDSPVSGRGDSPYSGRGDSPVSGRGDSPYSGRGDSPVSGRGDSPYSGRGDSPVSGR | |
| GDSPYSGRGDSPVSGRGDSPYSGRGDSPVSGRGDSFYSGRGDSPVSGRGDSPYSGRGDSPVSGRGDSPYS | |
| GRGDSPVSGRGDSPYSGRGDSPVSGRGDSPYSGRGDSPVSGRGDSPYSGRGDSPVSGRGDSPYSGRGDSP | |
| VSGRGDSPYSGRGDSPVSGRGDSPYSGRGDSPVSGRGDSPYSGRGDSPVSGRGDSPYSGRGDSPVSGRGD | |
| SPYSGRGDSPVSGRGDSPYSGRGDSPVSGRGDSPYSGRGDSPVSGY | |
| SKGP-[GRGDSPVS]40-GY | 208 |
| SKGPGRGDSPVSGRGDSPVSGRGDSPVSGRGDSPVSGRGDSPVSGRGDSPVSGRGDSPVSGRGDSPVSGR | |
| GDSPVSGRGDSPVSGRGDSPVSGRGDSPVSGRGDSPVSGRGDSPVSGRGDSPVSGRGDSPVSGRGDSPVS | |
| GRGDSPVSGRGDSPVSGRGDSPVSGRGDSPVSGRGDSPVSGRGDSPVSGRGDSPVSGRGDSPVSGRGDSP | |
| VSGRGDSPVSGRGDSPVSGRGDSPVSGRGDSPVSGRGDSPVSGRGDSPVSGRGDSPVSGRGDSPVSGRGD | |
| SPVSGRGDSPVSGRGDSPVSGRGDSPVSGRGDSPVSGRGDSPVSGY | |
| SKGP-[GRGDSPYSGRGDSPYSGRGDSPYSGKGDSPYS]10-GY | 209 |
| SKGPGRGDSPYSGRGDSPYSGRGDSPYSGKGDSPYSGRGDSPYSGRGDSPYSGRGDSPYSGKGDSPYSGR | |
| GDSPYSGRGDSPYSGRGDSPYSGKGDSPYSGRGDSPYSGRGDSPYSGRGDSPYSGKGDSPYSGRGDSPYS | |
| GRGDSPYSGRGDSPYSGKGDSPYSGRGDSPYSGRGDSPYSGRGDSPYSGKGDSPYSGRGDSPYSGRGDSP | |
| YSGRGDSPYSGKGDSPYSGRGDSPYSGRGDSPYSGRGDSPYSGKGDSPYSGRGDSPYSGRGDSPYSGRGD | |
| SPYSGKGDSPYSGRGDSPYSGRGDSPYSGRGDSPYSGKGDSPYSGY | |
| SKGP-[GRGDSPYSGKGDSPYS]20-GY | 210 |
| SKGPGRGDSPYSGKGDSPYSGRGDSPYSGKGDSPYSGRGDSPYSGKGDSPYSGRGDSPYSGKGDSPYSGR | |
| GDSPYSGKGDSPYSGRGDSPYSGKGDSPYSGRGDSPYSGKGDSPYSGRGDSPYSGKGDSPYSGRGDSPYS | |
| GKGDSPYSGRGDSPYSGKGDSPYSGRGDSPYSGKGDSPYSGRGDSPYSGKGDSPYSGRGDSPYSGKGDSP | |
| YSGRGDSPYSGKGDSPYSGRGDSPYSGKGDSPYSGRGDSPYSGKGDSPYSGRGDSPYSGKGDSPYSGRGD | |
| SPYSGKGDSPYSGRGDSPYSGKGDSPYSGRGDSPYSGKGDSPYSGY | |
| SKGP-[GRGDQPYQ]60-GY | 211 |
| SKGPGRGDQPYQGRGDQPYQGRGDQPYQGRGDQPYQGRGDQPYQGRGDQPYQGRGDQPYQGRGDQPYQGR | |
| GDQPYQGRGDQPYQGRGDQPYQGRGDQPYQGRGDQPYQGRGDQPYQGRGDQPYQGRGDQPYQGRGDQPYQ | |
| GRGDQPYQGRGDQPYQGRGDQPYQGRGDQPYQGRGDQPYQGRGDQPYQGRGDQPYQGRGDQPYQGRGDQP | |
| YQGRGDQPYQGRGDQPYQGRGDQPYQGRGDQPYQGRGDQPYQGRGDQPYQGRGDQPYQGRGDQPYQGRGD | |
| QPYQGRGDQPYQGRGDQPYQGRGDQPYQGRGDQPYQGRGDQPYQGRGDQPYQGRGDQPYQGRGDQPYQGR | |
| GDQPYQGRGDQPYQGRGDQPYQGRGDQPYQGRGDQPYQGRGDQPYQGRGDQPYQGRGDQPYQGRGDQPYQ | |
| GRGDQPYOGRGDQPYQGRGDQPYQGRGDQPYQGRGDOPYQGRGDQPYQGRGDQPYQGRGDQPYOGY | |
| SKGP-[GRGDQPYQ]80-GY | 212 |
| SKGPGRGDQPYQGRGDQPYQGRGDQPYQGRGDQPYQGRGDQPYQGRGDQPYQGRGDQPYQGRGDQPYQGR | |
| GDQPYQGRGDQPYQGRGDQPYQGRGDQPYQGRGDQPYQGRGDQPYQGRGDQPYQGRGDQPYQGRGDQPYQ | |
| GRGDQPYQGRGDQPYQGRGDQPYQGRGDQPYQGRGDQPYQGRGDQPYQGRGDQPYQGRGDQPYQGRGDQP | |
| YQGRGDQFYQGRGDQPYQGRGDQPYQGRGDQPYQGRGDQPYQGRGDQPYQGRGDQPYQGRGDQPYQGRGD | |
| QPYQGRGDQPYQGRGDQPYQGRGDQPYQGRGDQPYQGRGDQPYQGRGDQPYQGRGDQPYQGRGDQPYQGR | |
| GDQPYQGRGDQPYQGRGDQPYQGRGDQPYQGRGDQPYQGRGDQPYQGRGDQPYQGRGDQPYQGRGDQPYQ | |
| GRGDQPYQGRGDQPYQGRGDQPYQGRGDQPYQGRGDQPYQGRGDQPYQGRGDQPYQGRGDQPYQGRGDQP | |
| YQGRGDQFYQGRGDQPYQGRGDQPYQGRGDQPYQGRGDQPYQGRGDQPYQGRGDQPYQGRGDQPYQGRGD | |
| QPYQGRGDQPYQGRGDQPYQGRGDQPYQGRGDQPYQGRGDQPYQGRGDQPYQGRGDQPYQGRGDQPYQGR | |
| GDQPYQGRGDQPYQGY | |
| SKGP-[GRGDTPYT]40-GY | 213 |
| SKFPGRGDTPYTGRGDTPYTGRGDTPYTGRGDTPYTGRGDTPYTGRGDTPYTGRGDTPYTGRGDTPYTGR | |
| GDTPYTGRGDTPYTGRGDTPYTGRGDTPYTGRGDTPYTGRGDTPYTGRGDTPYTGRGDTPYTGRGDTPYT | |
| GRGDTPYTGRGDTPYTGRGDTPYTGRGDTPYTGRGDTPYTGRGDTPYTGRGDTPYTGRGDTPYTGRGDTP | |
| YTGRGDTPYTGRGDTPYTGRGDTPYTGRGDTPYTGRGDTPYTGRGDTPYTGRGDTPYTGRGDTPYTGRGD | |
| TPYTGRGDTPYTGRGDTPYTGRGDTPYTGRGDTPYTGRGDTPYTGY | |
| SKGP-[GRGDNPYN]20-GY | |
| SKGPGRGDNPYNGRGDNPYNGRGDNPYNGRGDNPYNGRGDNPYNGRGDNPYNGRGDNPYNGRGDNPYNGR | |
| GDNPYNGRGDNPYNGRGDNPYNGRGDNPYNGRGDNPYNGRGDNPYNGRGDNPYNGRGDNPYNGRGDNPYN | |
| GRGDNPYNGRGDNPYNGRGDNPYNGY | |
| SKGP-[GRGDNPYN]40-GY | 215 |
| SKGPGRGDNPYNGRGDNPYNGRGDNPYNGRGDNPYNGRGDNPYNGRGDNFYNGRGDNPYNGRGDNPYNGR | |
| GDNPYNGRGDNPYNGRGDNPYNGRGDNPYNGRGDNPYNGRGDNPYNGRGDNPYNGRGDNPYNGRGDNPYN | |
| GRGDNPYNGRGDNPYNGRGDNPYNGRGDNPYNGRGDNPYNGRGDNPYNGRGDNPYNGRGDNPYNGRGDNP | |
| YNGRGDNPYNGRGDNPYNGRGDNPYNGRGDNPYNGRGDNPYNGRGDNPYNGRGDNPYNGRGDNPYNGRGD | |
| NPYNGRGDNPYNGRGDNPYNGRGDNPYNGRGDNPYNGRGDNPYNGY | |
| SKGP-[GRGDNPYN]60-GY | 216 |
| SKGPGRGDNPYNGRGDNPYNGRGDNPYNGRGDNPYNGRGDNPYNGRGDNPYNGRGDNPYNGRGDNPYNGR | |
| GDNPYNGRGDNPYNGRGDNPYNGRGDNPYNGRGDNPYNGRGDNPYNGRGDNPYNGRGDNPYNGRGDNPYN | |
| GRGDNPYNGRGDNPYNGRGDNPYNGRGDNPYNGRGDNPYNGRGDNPYNGRGDNPYNGRGDNPYNGRGDNP | |
| YNGRGDNPYNGRGDNPYNGRGDNPYNGRGDNPYNGRGDNPYNGRGDNPYNGRGDNPYNGRGDNPYNGRGD | |
| NPYNGRGDNPYNGRGDNPYNGRGDNPYNGRGDNPYNGRGDNPYNGRGDNPYNGRGDNPYNGRGDNPYNGR | |
| GDNPYNGRGDNPYNGRGDNPYNGRGDNPYNGRGDNPYNGRGDNPYNGRGDNPYNGRGDNPYNGRGDNPYN | |
| GRGDNPYNGRGDNPYNGRGDNPYNGRGDNPYNGRGDNPYNGRGDNPYNGRGDNPYNGRGDNPYNGY | |
| SKGP-[GRGDSPHS]20-GY | 217 |
| SKGPGRGDSPHSGRGDSPHSGRGDSPHSGRGDSPHSGRGDSPHSGRGDSPHSGRGDSPHSGRGDSPHSGR | |
| GDSPHSGRGDSPHSGRGDSPHSGRGDSPHSGRGDSPHSGRGDSPHSGRGDSPHSGRGDSPHSGRGDSPHS | |
| GRGDSPHSGRGDSPHSGRGDSPHSGY | |
| SKGP-[GRGDSPHS]40-GY | 218 |
| SKGPGRGDSPHSGRGDSPHSGRGDSPHSGRGDSPHSGRGDSPHSGRGDSPHSGRGDSPHSGRGDSPHSGR | |
| GBSPHSGRGDSPHSGRGDSPHSGRGDSPHSGRGDSPHSGRGDSPHSGRGDSPHSGRGDSPHSGRGDSPHS | |
| GRGDSPHSGRGDSPHSGRGDSPHSGRGDSPHSGRGDSPHSGRGDSPHSGRGDSPHSGRGDSPHSGRGDSP | |
| HSGRGDSPHSGRGDSPHSGRGDSPHSGRGDSPHSGRGDSPHSGRGDSPHSGRGDSPHSGRGDSPHSGRGD | |
| SPHSGRGDSPHSGRGDSPHSGRGDSPHSGRGDSPHSGRGDSPHSGY | |
| SKGP-[GRGDSPHS]60-GY | 219 |
| SKGPGRGDSPHSGRGDSPHSGRGDSPHSGRGDSPHSGRGDSPHSGRGDSPHSGRGDSPHSGRGDSPHSGR | |
| GDSPHSGRGDSPHSGRGDSPHSGRGDSPHSGRGDSPHSGRGDSPHSGRGDSPHSGRGDSPHSGRGDSPHS | |
| GRGBSPHSGRGDSPHSGRGDSPHSGRGDSPHSGRGDSPHSGRGDSPHSGRGDSPHSGRGDSPHSGRGDSP | |
| HSGRGDSPHSGRGDSPHSGRGDSPHSGRGDSPHSGRGDSPHSGRGDSPHSGRGDSPHSGRGDSPHSGRGD | |
| SPHSGRGDSPHSGRGDSPHSGRGDSPHSGRGDSPHSGRGDSPHSGRGDSPHSGRGDSPHSGRGDSPHSGR | |
| GDSPHSGRGDSPHSGRGDSPHSGRGDSPHSGRGDSPHSGRGDSPHSGRGDSPHSGRGDSPHSGRGDSPHS | |
| GRGDSPHSGRGDSPHSGRGDSPHSGRGDSPHSGRGDSPHSGRGDSPHSGRGDSPHSGRGDSPHSGY | |
| SKGP-[GRGDSPHS]80-GY | 220 |
| SKGPGRGDSPHSGRGDSPHSGRGDSPHSGRGDSPHSGRGDSPHSGRGDSPHSGRGDSPHSGRGDSPHSGR | |
| GDSPHSGRGDSPHSGRGDSPHSGRGDSPHSGRGDSPHSGRGDSPHSGRGDSPHSGRGDSPHSGRGDSPHS | |
| GRGDSPHSGRGDSPHSGRGDSPHSGRGDSPHSGRGDSPHSGRGDSPHSGRGDSPHSGRGDSPHSGRGDSP | |
| HSGRGDSPHSGRGDSPHSGRGDSPHSGRGDSPHSGRGDSPHSGRGDSPHSGRGDSPHSGRGDSPHSGRGD | |
| SPHSGRGDSPHSGRGDSPHSGRGDSPHSGRGDSPHSGRGDSPHSGRGDSPHSGRGDSPHSGRGDSPHSGR | |
| GDSPHSGRGDSPHSGRGDSPHSGRGDSPHSGRGDSPHSGRGDSPHSGRGDSPHSGRGDSPHSGRGDSPHS | |
| GRGDSPHSGRGDSPHSGRGDSPHSGRGDSPHSGRGDSPHSGRGDSPHSGRGDSPHSGRGDSPHSGRGDSP | |
| HSGRGDSPHSGRGDSPHSGRGDSPHSGRGDSPHSGRGDSPHSGRGDSPHSGRGDSPHSGRGDSPHSGRGD | |
| SPHSGRGDSPHSGRGDSPHSGRGDSPHSGRGDSPHSGRGDSPHSGRGDSPHSGRGDSPHSGRGDSPHSGR | |
| GDSPHSGRGDSPHSGY | |
| SKGP-[GRGDSPFS]20-GY | 221 |
| SKGPGRGDSPFSGRGDSPFSGRGDSPFSGRGDSPFSGRGDSPFSGRGDSPFSGRGDSPFSGRGDSPFSGR | |
| GDSPFSGRGDSPFSGRGDSPFSGRGDSPFSGRGDSPFSGRGDSPFSGRGDSPFSGRGDSPFSGRGDSPFS | |
| GRGDSPFSGRGDSPFSGRGDSPFSGY | |
| SKGP-[GRGDSPFS]40-GY | 222 |
| SKGPGRGDSPFSGRGDSPFSGRGDSPFSGRGDSPFSGRGDSPFSGRGDSPFSGRGDSPFSGRGDSPFSGR | |
| GDSPFSGRGDSPFSGRGDSPFSGRGDSPFSGRGDSPFSGRGDSPFSGRGDSPFSGRGDSPFSGRGDSPFS | |
| GRGDSPFSGRGDSPFSGRGDSPFSGRGDSPFSGRGDSPFSGRGDSPFSGRGDSPFSGRGDSPFSGRGDSP | |
| FSGRGDSPFSGRGDSPFSGRGDSPFSGRGDSPFSGRGDSPFSGRGDSPFSGRGDSPFSGRGDSPFSGRGD | |
| SPFSGRGDSPFSGRGDSPFSGRGDSPFSGRGDSPFSGRGDSPFSGY | |
| SKGP-[GRGDSPYQ]20-GY | 223 |
| SKGPGRGDSPYQGRGDSPYQGRGDSPYQGRGDSPYQGRGDSPYQGRGDSPYQGRGDSPYQGRGDSPYQGR | |
| GDSPYQGRGDSPYQGRGDSPYQGRGDSPYQGRGDSPYQGRGDSPYQGRGDSPYQGRGDSPYQGRGDSPYQ | |
| GRGDSPYQGRGDSPYQGRGDSPYQGY | |
| SKGP-[GRGDSPYQ]40-GY | 224 |
| SKGPGRGDSPYQGRGDSPYQGRGDSPYQGRGDSPYQGRGDSPYQGRGDSPYQGRGDSPYQGRGDSPYQGR | |
| GDSPYQGRGDSPYQGRGDSPYQGRGDSPYQGRGDSPYQGRGDSPYQGRGDSPYQGRGDSPYQGRGDSPYQ | |
| GRGDSPYQGRGDSPYQGRGDSPYQGRGDSPYQGRGDSPYQGRGDSPYQGRGDSPYQGRGDSPYQGRGDSP | |
| YQGRGDSPYQGRGDSPYQGRGDSPYQGRGDSPYQGRGDSPYQGRGDSPYQGRGDSPYQGRGDSPYQGRGD | |
| SPYQGRGDSPYQGRGDSPYQGRGDSPYQGRGDSPYQGRGDSPYQGY | |
| SKGP-[GRGDSPYQ]60-GY | 225 |
| SKGPGRGDSPYQGRGDSPYQGRGDSPYQGRGDSPYQGRGDSPYQGRGDSPYQGRGDSPYQGRGDSPYQGR | |
| GDSPYQGRGDSPYQGRGDSPYQGRGDSPYQGRGDSPYQGRGDSPYQGRGDSPYQGRGDSPYQGRGDSPYQ | |
| GRGDSPYQGRGDSPYQGRGDSPYQGRGDSPYQGRGDSPYQGRGDSPYQGRGDSPYQGRGDSPYQGRGDSP | |
| YQGRGDSPYQGRGDSPYQGRGDSPYQGRGDSPYQGRGDSPYQGRGDSPYQGRGDSPYQGRGDSPYQGRGD | |
| SPYQGRGDSPYQGRGDSPYQGRGDSPYQGRGDSPYQGRGDSPYQGRGDSPYQGRGDSPYQGRGDSPYQGR | |
| GDSPYQGRGDSPYQGRGDSPYQGRGDSPYQGRGDSPYQGRGDSPYQGRGDSPYQGRGDSPYQGRGDSPYQ | |
| GRGDSPYQGRGDSPYQGRGDSPYQGRGDSPYQGRGDSPYQGRGDSPYQGRGDSPYQGRGDSPYQGY | |
| SKGP-[GRGDSPYQ]80-GY | 226 |
| SKGPGRGDSPYQGRGDSPYQGRGDSPYQGRGDSPYQGRGDSPYQGRGDSPYQGRGDSPYQGRGDSPYQGR | |
| GDSPYQGRGDSPYQGRGDSPYQGRGDSPYQGRGDSPYQGRGDSPYQGRGDSPYQGRGDSPYQGRGDSPYQ | |
| GRGDSPYQGRGDSPYQGRGDSPYQGRGDSPYQGRGDSPYQGRGDSPYQGRGDSPYQGRGDSPYQGRGDSP | |
| YQGRGDSPYQGRGDSPYQGRGDSPYQGRGDSPYQGRGDSPYQGRGDSPYQGRGDSPYQGRGDSPYQGRGD | |
| SPYQGRGDSPYQGRGDSPYQGRGDSPYQGRGDSPYQGRGDSPYQGRGDSPYQGRGDSPYQGRGDSPYQGR | |
| GDSPYQGRGDSPYQGRGDSPYQGRGDSPYQGRGDSPYQGRGDSPYQGRGDSPYQGRGDSPYQGRGDSPYQ | |
| GRGDSPYQGRGDSPYQGRGDSPYQGRGDSPYQGRGDSPYQGRGDSPYQGRGDSPYQGRGDSPYQGRGDSP | |
| YQGRGDSPYQGRGDSPYQGRGDSPYQGRGDSPYQGRGDSPYQGRGDSPYQGRGDSPYQGRGDSPYQGRGD | |
| SPYQGRGDSPYQGRGDSPYQGRGDSPYQGRGDSPYQGRGDSPYQGRGDSPYQGRGDSPYQGRGDSPYQGR | |
| GDSPYQGRGDSPYQGY | |
| SKGP-[GRGDQPYS]20-GY | 227 |
| SKGPGRGDQPYSGRGDQPYSGRGDQPYSGRGDQPYSGRGDQPYSGRGDQPYSGRGDQPYSGRGDQPYSGR | |
| GDQPYSGRGDQPYSGRGDQPYSGRGDQPYSGRGDQPYSGRGDQPYSGRGDQPYSGRGDQPYSGRGDQPYS | |
| GRGDQPYSGRGDQPYSGRGDQPYSGY | |
| SKGP-[GRGDQPYS]40-GY | 228 |
| SKGPGRGDQPYSGRGDQPYSGRGDQPYSGRGDQPYSGRGDQPYSGRGDQPYSGRGDQPYSGRGDQPYSGR | |
| GDQPYSGRGDQPYSGRGDQPYSGRGDQPYSGRGDQPYSGRGDQPYSGRGDQPYSGRGDQPYSGRGDQPYS | |
| GRGDQPYSGRGDQPYSGRGDQPYSGRGDQPYSGRGDQPYSGRGDQPYSGRGDQPYSGRGDQPYSGRGDQP | |
| YSGRGDQPYSGRGDQPYSGRGDQPYSGRGDQPYSGRGDQPYSGRGDQPYSGRGDQPYSGRGDQPYSGRGD | |
| QPYSGRGDQPYSGRGDQPYSGRGDQPYSGRGDQPYSGRGDQPYSGY | |
| SKGP-[GRGDQPYS]60-GY | 229 |
| SKGPGRGDQPYSGRGDQPYSGRGDQPYSGRGDQPYSGRGDQPYSGRGDQPYSGRGDQPYSGRGDQPYSGR | |
| GDQPYSGRGDQPYSGRGDQPYSGRGDQPYSGRGDQFYSGRGDQPYSGRGDQPYSGRGDQPYSGRGDQPYS | |
| GRGDQPYSGRGDQPYSGRGDQPYSGRGDQPYSGRGDQPYSGRGDQPYSGRGDQPYSGRGDQPYSGRGDQP | |
| YSGRGDQPYSGRGDQPYSGRGDQPYSGRGDQPYSGRGDQPYSGRGDQPYSGRGDQPYSGRGDQPYSGRGD | |
| QPYSGRGDQPYSGRGDQPYSGRGDQPYSGRGDQPYSGRGDQPYSGRGDQPYSGRGDQPYSGRGDQPYSGR | |
| GDQPYSGRGDQPYSGRGDQPYSGRGDQPYSGRGDQFYSGRGDQPYSGRGDQPYSGRGDQPYSGRGDQPYS | |
| GRGDQPYSGRGDQPYSGRGDQPYSGRGDQPYSGRGDQPYSGRGDQPYSGRGDQPYSGRGDQPYSGY | |

SKGP-[GRGDQPYS]80-GY

| | |
|---|---|
| SKGPGRGDQPYSGRGDQPYSGRGDQPYSGRGDQPYSGRGDQPYSGRGDQPYSGRGDQPYSGRGDQPYSGR | 230 |
| GDQPYSGRGDQPYSGRGDQPYSGRGDQPYSGRGDQPYSGRGDQPYSGRGDQPYSGRGDQPYSGRGDQPYS | |
| GRGDQPYSGRGDQPYSGRGDQPYSGRGDQPYSGRGDQPYSGRGDQPYSGRGDQPYSGRGDQPYSGRGDQP | |
| YSGRGDQPYSGRGDQPYSGRGDQPYSGRGDQPYSGRGDQPYSGRGDQPYSGRGDQPYSGRGDQPYSGRGD | |
| QPYSGRGDQPYSGRGDQPYSGRGDQPYSGRGDQPYSGRGDQPYSGRGDQPYSGRGDQPYSGRGDQPYSGR | |
| GDQPYSGRGDQPYSGRGDQPYSGRGDQPYSGRGDQPYSGRGDQPYSGRGDQPYSGRGDQPYSGRGDQPYS | |
| GRGDQPYSGRGDQPYSGRGDQPYSGRGDQPYSGRGDQPYSGRGDQPYSGRGDQPYSGRGDQPYSGRGDQP | |
| YSGRGDQPYSGRGDQPYSGRGDQPYSGRGDQPYSGRGDQPYSGRGDQPYSGRGDQPYSGRGDQPYSGRGD | |
| QPYSGRGDQPYSGRGDQPYSGRGDQPYSGRGDQPYSGRGDQPYSGRGDQPYSGRGDQPYSGRGDQPYSGR | |
| GDQPYSGRGDQPYSGY | |
| SKGP-[GRGDNPYS]20-GY | 231 |
| SKGPGRGDNPYSGRGDNPYSGRGDNPYSGRGDNPYSGRGDNPYSGRGDNPYSGRGDNPYSGRGDNPYSGR | |
| GDNPYSGRGDNPYSGRGDNPYSGRGDNPYSGRGDNPYSGRGDNPYSGRGDNPYSGRGDNPYSGRGDNPYS | |
| GRGDNPYSGRGDNPYSGRGDNPYSGY | |
| SKGP-[GRGDNPYS]40-GY | 232 |
| SKGPGRGDNPYSGRGDNPYSGRGDNPYSGRGDNPYSGRGDNPYSGRGDNPYSGRGDNPYSGRGDNPYSGR | |
| GDNPYSGRGDNPYSGRGDNPYSGRGDNPYSGRGDNPYSGRGDNPYSGRGDNPYSGRGDNPYSGRGDNPYS | |
| GRGDNPYSGRGDNPYSGRGDNPYSGRGDNPYSGRGDNPYSGRGDNPYSGRGDNPYSGRGDNPYSGRGDNP | |
| YSGRGDNPYSGRGDNPYSGRGDNPYSGRGDNPYSGRGDNPYSGRGDNPYSGRGDNPYSGRGDNPYSGRGD | |
| NPYSGRGDNPYSGRGDNPYSGRGDNPYSGRGDNPYSGRGDNPYSGY | |
| SKGP-[GRGDNPYS]60-GY | 233 |
| SKGPGRGDNPYSGRGDNPYSGRGDNPYSGRGDNPYSGRGDNPYSGRGDNPYSGRGDNPYSGRGDNPYSGR | |
| GDNPYSGRGDNPYSGRGDNPYSGRGDNPYSGRGDNPYSGRGDNPYSGRGDNPYSGRGDNPYSGRGDNPYS | |
| GRGDNPYSGRGDNPYSGRGDNPYSGRGDNPYSGRGDNPYSGRGDNPYSGRGDNPYSGRGDNPYSGRGDNP | |
| YSGRGDNPYSGRGDNPYSGRGDNPYSGRGDNPYSGRGDNPYSGRGDNPYSGRGDNPYSGRGDNPYSGRGD | |
| NPYSGRGDNPYSGRGDNPYSGRGDNPYSGRGDNPYSGRGDNPYSGRGDNPYSGRGDNPYSGRGDNPYSGR | |
| GDNPYSGRGDNPYSGRGDNPYSGRGDNPYSGRGDNPYSGRGDNPYSGRGDNPYSGRGDNPYSGRGDNPYS | |
| GRGDNPYSGRGDNPYSGRGDNPYSGRGDNPYSGRGDNPYSGRGDNPYSGRGDNPYSGRGDNPYS | |
| SKGP-[GRGDNPYS]80-GY | 234 |
| SKGPGRGDNPYSGRGDNPYSGRGDNPYSGRGDNPYSGRGDNPYSGRGDNPYSGRGDNPYSGRGDNPYSGR | |
| GDNPYSGRGDNPYSGRGDNPYSGRGDNPYSGRGDNPYSGRGDNPYSGRGDNPYSGRGDNPYSGRGDNPYS | |
| GRGDNPYSGRGDNPYSGRGDNPYSGRGDNPYSGRGDNPYSGRGDNPYSGRGDNPYSGRGDNPYSGRGDNP | |
| YSGRGDNPYSGRGDNPYSGRGDNPYSGRGDNPYSGRGDNPYSGRGDNPYSGRGDNPYSGRGDNPYSGRGD | |
| NPYSGRGDNPYSGRGDNPYSGRGDNPYSGRGDNPYSGRGDNPYSGRGDNPYSGRGDNPYSGRGDNPYSGR | |
| GDNPYSGRGDNPYSGRGDNPYSGRGDNPYSGRGDNPYSGRGDNPYSGRGDNPYSGRGDNPYSGRGDNPYS | |
| GRGDNPYSGRGDNPYSGRGDNPYSGRGDNPYSGRGDNPYSGRGDNPYSGRGDNPYSGRGDNPYSGRGDNP | |
| YSGRGDNPYSGRGDNPYSGRGDNPYSGRGDNPYSGRGDNPYSGRGDNPYSGRGDNPYSGRGDNPYSGRGD | |
| NPYSGRGDNPYSGRGDNPYSGRGDNPYSGRGDNPYSGRGDNPYSGRGDNPYSGRGDNPYSGRGDNPYSGR | |
| GDNPYSGRGDNPYSG | |
| SKGP-[GRGDSPYN]20-GY | 235 |
| SKGPGRGDSPYNGRGDSPYNGRGDSPYNGRGDSPYNGRGDSPYNGRGDSPYNGRGDSPYNGRGDSPYNGR | |
| GDSPYNGRGDSPYNGRGDSPYNGRGDSPYNGRGDSPYNGRGDSPYNGRGDSPYNGRGDSPYNGRGDSPYN | |
| GRGDSPYNGRGDSPYNGRGDSPYNGY | |
| SKGP-[GRGDSPYN]4G-GY | 236 |
| SKGPGRGDSPYNGRGDSPYNGRGDSPYNGRGDSPYNGRGDSPYNGRGDSPYNGRGDSPYNGRGDSPYNGR | |
| GDSPYNGRGDSPYNGRGDSPYNGRGDSPYNGRGDSPYNGRGDSPYNGRGDSPYNGRGDSPYNGRGDSPYN | |
| GRGDSPYNGRGDSPYNGRGDSPYNGRGDSPYNGRGDSPYNGRGDSPYNGRGDSPYNGRGDSPYNGRGDSP | |
| YNGRGDSPYNGRGDSPYNGRGDSPYNGRGDSPYNGRGDSPYNGRGDSPYNGRGDSPYNGRGDSPYNGRGD | |
| SPYNGRGDSPYNGRGDSPYNGRGDSPYNGRGDSPYNGRGDSPYNGY | |
| SKGP-[GRGDSPYN]60-GY | 237 |
| SKGPGRGDSPYNGRGDSPYNGRGDSPYNGRGDSPYNGRGDSPYNGRGDSPYNGRGDSPYNGRGDSPYNGR | |
| GDSPYNGRGDSPYNGRGDSPYNGRGDSPYNGRGDSPYNGRGDSPYNGRGDSPYNGRGDSPYNGRGDSPYN | |
| GRGDSPYNGRGDSPYNGRGDSPYNGRGDSPYNGRGDSPYNGRGDSPYNGRGDSPYNGRGDSPYNGRGDSP | |
| YNGRGDSPYNGRGDSPYNGRGDSPYNGRGDSPYNGRGDSPYNGRGDSPYNGRGDSPYNGRGDSPYNGRGD | |
| SPYNGRGDSPYNGRGDSPYNGRGDSPYNGRGDSPYNGRGDSPYNGRGDSPYNGRGDSPYNGRGDSPYNGR | |
| GDSPYNGRGDSPYNGRGDSPYNGRGDSPYNGRGDSPYNGRGDSPYNGRGDSPYNGRGDSPYNGRGDSPYN | |
| GRGDSPYNGRGDSPYNGRGDSPYNGRGDSPYNGRGDSPYNGRGDSPYNGRGDSPYNGRGDSPYNGY | |
| SKGP-[GRGDNPYQ]20-GY | 238 |
| SKGPGRGDNPYQGRGDNPYQGRGDNPYQGRGDNPYQGRGDNPYQGRGDNPYQGRGDNPYQGRGDNPYQGR | |
| GDNPYQGRGDNPYQGRGDNPYQGRGDNPYQGRGDNPYQGRGDNPYQGRGDNPYQGRGDNPYQGRGDNPYQ | |
| GRGDNPYQGRGDNPYQGRGDNPYQCY | |
| SKGP-[GRGDNPYQ]40-GY | 239 |
| SKGPGRGDNPYQGRGDNPYQGRGDNPYQGRGDNPYQGRGDNPYQGRGDNPYQGRGDNPYQGRGDNPYQGR | |
| GDNPYQGRGDNPYQGRGDNPYQGRGDNPYQGRGDNPYQGRGDNPYQGRGDNPYQGRGDNPYQGRGDNPYQ | |
| GRGDNPYQGRGDNPYQGRGDNPYQGRGDNPYQGRGDNPYQGRGDNPYQGRGDNPYQGRGDNPYQGRGDNP | |
| YQGRGDNPYQGRGDNPYQGRGDNPYQGRGDNPYQGRGDNPYQGRGDNPYQGRGDNPYQGRGDNPYQGRGD | |
| NPYQGRGDNPYQGRGDNPYQGRGDNPYQGRGDNPYQGRGDNPYQGY | |
| SKGP-[GRGDNPYQ]60-GY | 240 |
| SKGPGRGDNPYQGRGDNPYQGRGDNPYQGRGDNPYQGRGDNPYQGRGDNPYQGRGDNPYQGRGDNPYQGR | |
| GDNPYQGRGDNPYQGRGDNPYQGRGDNPYQGRGDNPYQGRGDNPYQGRGDNPYQGRGDNPYQGRGDNPYQ | |
| GRGDNPYQGRGDNPYQGRGDNPYQGRGDNPYQGRGDNPYQGRGDNPYQGRGDNPYQGRGDNPYQGRGDNP | |
| YQGRGDNPYQGRGDNPYQGRGDNPYQGRGDNPYQGRGDNPYQGRGDNPYQGRGDNPYQGRGDNPYQGRGD | |
| NPYQGRGDNPYQGRGDNPYQGRGDNPYQGRGDNPYQGRGDNPYQGRGDNPYQGRGDNPYQGRGDNPYQGR | |
| GDNPYQGRGDNPYQGRGDNPYQGRGDNPYQGRGDNPYQGRGDNPYQGRGDNPYQGRGDNPYQGRGDNPYQ | |
| GRGDNPYQGRGDNPYQGRGDNPYQGRGDNPYQGRGDNPYQGRGDNPYQGRGDNPYQGRGDNPYQGY | |
| SKGP-[GRGDNPYQ]80-GY | 241 |
| SKGPGRGDNPYQGRGDNPYQGRGDNPYQGRGDNPYQGRGDNPYQGRGDNPYQGRGDNPYQGRGDNPYQGR | |
| GDNPYQGRGDNPYQGRGDNPYQGRGDNPYQGRGDNPYQGRGDNPYQGRGDNPYQGRGDNPYQGRGDNPYQ | |
| GRGDNPYQGRGDNPYQGRGDNPYQGRGDNPYQGRGDNPYQGRGDNPYQGRGDNPYQGRGDNPYQGRGDNP | |
| YQGRGDNPYQGRGDNPYQGRGDNPYQGRGDNPYQGRGDNPYQGRGDNPYQGRGDNPYQGRGDNPYQGRGD | |
| NPYQGRGDNPYQGRGDNPYQGRGDNPYQGRGDNPYQGRGDNPYQGRGDNPYQGRGDNPYQGRGDNPYQGR | |
| GDNPYQGRGDNPYQGRGDNPYQGRGDNPYQGRGDNPYQGRGDNPYQGRGDNPYQGRGDNPYQGRGDNPYQ | |
| GRGDNPYQGRGDNPYQGRGDNPYQGRGDNPYQGRGDNPYQGRGDNPYQGRGDNPYQGRGDNPYQGRGDNP | |
| YQGRGDNPYQGRGDNPYQGRGDNPYQGRGDNPYQGRGDNPYQGRGDNPYQGRGDNPYQGRGDNPYQGRGD | |
| NPYQGRGDNPYQGRGDNPYQGRGDNPYQGRGDNPYQGRGDNPYQGRGDNPYQGRGDNPYQGRGDNPYQGR | |
| GDNPYQGRGDNPYQGY | |
| SKGP-[GRGDQPYN]20-GY | 242 |
| SKGPGRGDQPYNGRGDQPYNGRGDQPYNGRGDQPYNGRGDQPYNGRGDQPYNGRGDQPYNGRGDQPYNGR | |
| GDQPYNGRGDQPYNGRGDQPYNGRGDQPYNGRGDQFYNGRGDQPYNGRGDQPYNGRGDQPYNGRGDQPYN | |
| GRGDQPYNGRGDQPYNGRGDQPYNGY | |
| SKGP-[GRGDQPYN]40-GY | 243 |
| SKGPGRGDQPYNGRGDQPYNGRGDQPYNGRGDQPYNGRGDQPYNGRGDQPYNGRGDQPYNGRGDQPYNGR | |
| GDQPYNGRGDQPYNGRGDQPYNGRGDQPYNGRGDQPYNGRGDQPYNGRGDQPYNGRGDQPYNGRGDQPYN | |
| GRGDQPYNGRGDQPYNGRGDQPYNGRGDQPYNGRGDQPYNGRGDQPYNGRGDQPYNGRGDQPYNGRGDQP | |
| YNGRGDQPYNGRGDQPYNGRGDQPYNGRGDQPYNGRGDQPYNGRGDQPYNGRGDQPYNGRGDQPYNGRGD | |
| QPYNGRGDQPYNGRGDQPYNGRGDQPYNGRGDQPYNGRGDQPYNGY | |
| SKGP-[GRGDQPYN]60-GY | 244 |
| SKGPGRGDQPYNGRGDQPYNGRGDQPYNGRGDQPYNGRGDQPYNGRGDQPYNGRGDQPYNGRGDQPYNGR | |
| GDQPYNGRGDQPYNGRGDQPYNGRGDQPYNGRGDQPYNGRGDQPYNGRGDQPYNGRGDQPYNGRGDQPYN | |
| GRGDQPYNGRGDQPYNGRGDQPYNGRGDQPYNGRGDQPYNGRGDQPYNGRGDQPYNGRGDQPYNGRGDQP | |
| YNGRGDQPYNGRGDQPYNGRGDQPYNGRGDQPYNGRGDQPYNGRGDQPYNGRGDQPYNGRGDQPYNGRGD | |
| QPYNGRGDQPYNGRGDQPYNGRGDQPYNGRGDQPYNGRGDQPYNGRGDQPYNGRGDQPYNGRGDQPYNGR | |
| GDQPYNGRGDQPYNGRGDQPYNGRGDQPYNGRGDQFYNGRGDQPYNGRGDQPYNGRGDQPYNGRGDQPYN | |
| GRGDQPYNGRGDQPYNGRGDQPYNGRGDQPYNGRGDQPYNGRGDQPYNGRGDQPYNGRGDQPYNGY | |
| SKGP-[GRGDSPYS]20-sfGFP | 245 |
| SKGPGRGDSPYSGRGDSPYSGRGDSPYSGRGDSPYSGRGDSPYSGRGDSPYSGRGDSPYSGRGDSPYSGR | |
| GDSPYSGRGDSPYSGRGDSPYSGRGDSPYSGRGDSPYSGRGDSPYSGRGDSPYSGRGDSPYSGRGDSPYS | |
| GRGDSPYSGRGDSPYSGRGDSPYSGKGEELFTGVVPILVELDGDVNGHKFSVRGEGEGDATNGKLTLKFI | |
| CTTGKLPVPWPTLVTTLTYGVQCFSRYPDHMKRHDFFKSAMPEGYVQERTISFKDDGNYKTRAEVKFEGD | |
| TLVNRIELKGIDFKEDGNILGHKLEYNYNSHNVYITADRQKNGIKANFKIRHNIEDGSVQLADHYQQNTP | |
| IGDGPVLLPDNHYLSTQSVLSKDPNEKRDHMVLLEFVTAAGITHGMDELYKELHHHHHHG | |
| SKGP-[GRGDSPYS]-40-sfGFP | 246 |
| SKGPGRGDSPYSGRGDSPYSGRGDSPYSGRGDSPYSGRGDSPYSGRGDSPYSGRGDSPYSGRGDSPYSGR | |
| GDSPYSGRGDSPYSGRGDSPYSGRGDSPYSGRGDSPYSGRGDSPYSGRGDSPYSGRGDSPYSGRGDSPYS | |
| GRGDSPYSGRGDSPYSGRGDSPYSGRGDSPYSGRGDSPYSGRGDSPYSGRGDSPYSGRGDSPYSGRGDSP | |
| YSGRGDSPYSGRGDSPYSGRGDSPYSGRGDSPYSGRGDSPYSGRGDSPYSGRGDSPYSGRGDSPYSGRGD | |
| SPYSGRGDSPYSGRGDSPYSGRGDSPYSGRGDSPYSGRGDSPYSGKGEELFTGVVPILVELDGDVNGHKF | |
| SVRGEGEGDATNGKLTLKFICTTGKLPVPWPTLVTTLTYGVQCFSRYPDHMKRHDFFKSAMPEGYVQERT | |
| ISFKDDGNYKTRAEVKFEGDTLVNRIELKGIDFKEDGNILGHKLEYNYNSHNVYITADKQKNGIKANFKI | |
| RHNIEDGSVQLADHYQQNTPIGDGPVLLPDNHYLSTQSVLSKDPNEKRDHMVLLEFVTAAGITHGMDELY | |
| KELHHHHHHG | |
| SKGP-[GRDGSPYQ]20-GY | 247 |
| SKGPGRDGSPYQGRDGSPYQGRDGSPYQGRDGSPYQGRDGSPYQGRDGSPYQGRDGSPYQGRDGSPYQGR | |
| DGSPYQGRDGSPYQGRDGSPYQGRDGSPYQGRDGSPYQGRDGSPYQGRDGSPYQGRDGSPYQGRDGSPYQ | |
| GRDGSPYQGRDGSPYQGRDGSPYQGY | |
| SKGP-[GRDGQPYQ]20-GY | |
| SKGPGRDGQPYQGRDGQPYQGRDGQPYQGRDGQPYQGRDGQPYQGRDGQPYQGRDGQPYQGRDGQPYQGR | 248 |
| DGQPYQGRDGQPYQGRDGQPYQGRDGQPYQGRDGQPYQGRDGQPYQGRDGQPYQGRDGQPYQGRDGQPYQ | |
| GRDGQPYQGRDGQPYQGRDGQPYQGY | |
| SKGP-[GRDGSPYN]20-GY | 249 |
| SKGPGRDGSPYNGRDGSPYNGRDGSPYNGRDGSPYNGRDGSPYNGRDGSPYNGRDGSPYNGRDGSPYNGR | |
| DGSPYNGRDGSPYNGRDGSPYNGRDGSPYNGRDGSPYNGRDGSPYNGRDGSPYNGRDGSPYNGRDGSPYN | |
| GRDGSPYNGRDGSPYNGRDGSPYNGY | |
| SKGP-[GRGDNPHN]20-GY | 250 |
| SKGPGRGDNPHNGRGDNPHNGRGDNPHNGRGDNPHNGRGDNPHNGRGDNPHNGRGDNPHNGRGDNPHNGR | |
| GDNPHNGRGDNPHNGRGDNPHNGRGBNPHNGRGDNPHNGRGDNPHNGRGDNPHNGRGDNPHNGRGDNPHN | |
| GRGDNPHNGRGDNPHNGRGDNPHNGY | |
| SKGP-[GRGDNPHS]20-GY | 251 |
| SKGPGRGDNPHSGRGDNPHSGRGDNPHSGRGDNPHSGRGDNPHSGRGDNPHSGRGDNPHSGRGDNPHSGR | |
| GDNPHSGRGDNPHSGRGDNPHSGRGDNPHSGRGDNPHSGRGDNPHSGRGDNPHSGRGDNPHSGRGDNPHS | |
| GRGDNPHSGRGDNPHSGRGDNPHSGY | |
| SKGP-[GRGDSPHN]20-GY | 252 |
| SKGPGRGDSPHNGRGDSPHNGRGDSPHNGRGDSPHNGRGDSPHNGRGDSPHNGRGDSPHNGRGDSPHNGR | |
| GDSPHNGRGDSPHNGRGDSPHNGRGDSPHNGRGDSPHNGRGDSPHNGRGDSPHNGRGDSPHNGRGDSPHN | |
| GRGDSPHNGRGDSPHNGRGDSPHNGY | |
| SKGP-[GRGDQPHN]20-GY | 253 |
| SKGPGRGDOPHNGRGDQPHNGRGDQPHNGRGDQPHNGRGDQPHNGRGDQPHNGRGDQPHNGRGDQPHNGR | |
| GDQPHNGRGDQPHNGRGDQPHNGRGDQPHNGRGDQPHNGRGDQPHNGRGDQPHNGRGDQPHNGRGDQPHN | |
| GRGDQPHNGRGDQPHNGRGDQPHNGY | |
| SKGP-[GRGDSPYSGRGDSPYSGRGDSPYSGRGDQPYQ]10-GY | 254 |
| SKGPGRGDSPYSGRGDSPYSGRGDSPYSGRGBQPYQGRGDSPYSGRGDSPYSGRGDSPYSGRGDQPYQGR | |
| GDSPYSGRGDSPYSGRGDSPYSGRGDQPYQGRGDSPYSGRGDSPYSGRGDSPYSGRGDQPYQGRGDSPYS | |
| GRGDSPYSGRGDSPYSGRGBQPYQGRGDSPYSGRGDSPYSGRGDSPYSGRGDQPYQGRGDSPYSGRGDSP | |
| YSGRGDSPYSGRGDQPYQGRGDSPYSGRGDSPYSGRGDSPYSGRGDQPYQGRGDSPYSGRGDSPYSGRGD | |
| SPYSGRGDQPYQGRGDSPYSGRGDSPYSGRGDSPYSGRGDQPYQGY | |
| SKGP-[GRGDSPYSGRGDQPYQ]20-GY | 255 |
| SKGPGRGDSPYSGRGDQPYQGRGDSPYSGRGDQPYQGRGDSPYSGRGDQPYQGRGDSPYSGRGDQPYQGR | |
| GDSPYSGRGDQPYQGRGDSPYSGRGDQPYQGRGBSPYSGRGDQPYQGRGDSPYSGRGBQPYQGRGDSPYS | |
| GRGDQPYQGRGDSPYSGRGDQPYQGRGDSPYSGRGDQPYQGRGDSPYSGRGDQPYQGRGDSPYSGRGDQP | |
| YQGRGDSPYSGRGDQPYQGRGDSPYSGRGDQPYQGRGDSPYSGRGBQPYQGRGDSPYSGRGDQPYQGRGD | |
| SPYSGRGDQPYQGRGDSPYSGRGDQPYQGRGDSPYSGRGDQPYQGY | |
| SKGP-[GRGDQPYQGRGDQPYQGRGDQPYQGRGDSPYS]10-GY | 256 |
| SKGPGRGDQPYQGRGDQPYQGRGDQPYQGRGDSPYSGRGDQPYQGRGDQPYQGRGDQPYQGRGDSPYSGR | |
| GDQPYQGRGDQPYQGRGDQPYQGRGDSPYSGRGDQPYQGRGDQPYOGRGDQPYQGRGDSPYSGRGDQPYQ | |
| GRGDQPYQGRGDQPYQGRGDSPYSGRGDQPYQGRGDQPYQGRGDQPYQGRGDSPYSGRGDQPYQGRGDQP | |
| YQGRGDQPYQGRGDSPYSGRGDQPYQGRGDQPYQGRGDQPYQGRGDSPYSGRGDQPYQGRGDQPYQGRGD | |
| QPYQGRGDSPYSGRGDQPYQGRGDQPYQGRGDQPYQGRGDSPYSGY | |
| SKGP-[GRGDSPYSGRGDSPYSGRGDSPYSGRGDSPVS]10-GY | 257 |
| SKGPGRGDSPYSGRGDSPYSGRGDSPYSGRGDSPVSGRGDSPYSGRGDSPYSGRGDSPYSGRGDSPVSGR | |
| GDSPYSGRGDSPYSGRGDSPYSGRGDSPVSGRGDSPYSGRGDSPYSGRGDSPYSGRGDSPVSGRGDSPYS | |
| GRGDSPYSGRGDSPYSGRGDSPVSGRGDSPYSGRGDSPYSGRGDSPYSGRGDSPVSGRGDSPYSGRGDSP | |
| YSGRGDSFYSGRGDSPVSGRGDSPYSGRGDSPYSGRGDSPYSGRGDSPVSGRGDSPYSGRGDSPYSGRGD | |
| SPYSGRGDSPVSGRGDSPYSGRGDSPYSGRGDSPYSGRGDSPVSGY | |
| SKGP-[GRGDSPYSGRGDSPVS]20-GY | 258 |
| SKFPGRGDSFYSGRGDSPVSGRGDSPYSGRGDSPVSGRGDSPYSGRGDSPVSGRGDSPYSGRGDSPVSGR | |
| GDSPYSGRGDSPVSGRGDSPYSGRGDSPVSGRGDSPYSGRGDSPVSGRGDSPYSGRGDSPVSGRGDSPYS | |
| GRGDSPVSGRGDSPYSGRGDSPVSGRGDSPYSGRGDSPVSGRGDSPYSGRGDSPVSGRGDSPYSGRGDSP | |
| VSGRGDSPYSGRGDSPVSGRGDSPYSGRGDSPVSGRGDSPYSGRGDSPVSGRGDSPYSGRGDSPVSGRGD | |
| SPYSGRGDSPVSGRGDSPYSGRGDSPVSGRGDSPYSGRGDSPVSGY | |
| SKGP-[GRGDSPVSGRGDSPVSGRGDSPVSGRGDSPYS]10-GY | 259 |
| SKGPGRGDSPVSGRGDSPVSGRGDSPVSGRGDSPYSGRGDSPVSGRGDSPVSGRGDSPVSGRGDSPYSGR | |
| GDSPVSGRGDSPVSGRGDSPVSGRGDSPYSGRGDSPVSGRGDSPVSGRGDSPVSGRGDSPYSGRGDSPVS | |
| GRGDSPVSGRGDSPVSGRGDSPYSGRGDSPVSGRGDSPVSGRGDSPVSGRGDSPYSGRGDSPVSGRGDSP | |
| VSGRGDSPVSGRGDSPYSGRGDSPVSGRGDSPVSGRGDSPVSGRGDSPYSGRGDSPVSGRGDSPVSGRGD | |
| SPVSGRGDSPYSGRGDSPVSGRGDSPVSGRGDSPVSGRGDSPYSGY | |
| SKGP-[GRGDSPVS]40-GY | 260 |
| SKGPGRGDSPVSGRGDSPVSGRGDSPVSGRGDSPVSGRGDSPVSGRGDSPVSGRGDSPVSGRGDSPVSGR | |
| GDSPVSGRGDSPVSGRGDSPVSGRGDSPVSGRGDSPVSGRGDSPVSGRGDSPVSGRGDSPVSGRGDSPVS | |
| GRGDSPVSGRGDSPVSGRGDSPVSGRGDSPVSGRGDSPVSGRGDSFVSGRGDSPVSGRGDSPVSGRGDSP | |
| VSGRGDSPVSGRGDSPVSGRGDSPVSGRGDSPVSGRGDSPVSGRGDSPVSGRGDSPVSGRGDSPVSGRGD | |
| SPVSGRGDSPVSGRGDSPVSGRGDSPVSGRGDSFVSGRGDSPVSGY | |
| SKGP-[GRGDSPYSGRGDSPYSGRGDSPYSGRGDSPAS]10-GY | 261 |
| SKGPGRGDSPYSGRGDSPYSGRGDSPYSGRGDSPASGRGDSPYSGRGDSPYSGRGDSPYSGRGDSPASGR | |
| GDSPYSGRGDSPYSGRGDSPYSGRGDSPASGRGDSPYSGRGDSPYSGRGDSPYSGRGDSPASGRGDSPYS | |
| GRGDSPYSGRGDSPYSGRGDSPASGRGDSPYSGRGDSPYSGRGDSPYSGRGDSPASGRGDSPYSGRGDSP | |
| YSGRGDSPYSGRGDSPASGRGDSPYSGRGDSPYSGRGDSPYSGRGDSPASGRGDSPYSGRGDSPYSGRGD | |
| SPYSGRGDSPASGRGDSPYSGRGDSPYSGRGDSPYSGRGDSPASGY | |
| SKGP-[GRGDSPYSGRGDSPAS]20-GY | 262 |
| SKGPGRGDSPYSGRGDSPASGRGDSPYSGRGDSPASGRGDSPYSGRGDSPASGRGDSPYSGRGDSPASGR | |
| GDSPYSGRGDSPASGRGDSPYSGRGDSPASGRGDSPYSGRGDSPASGRGDSPYSGRGDSPASGRGDSPYS | |
| GRGDSPASGRGDSPYSGRGDSPASGRGDSPYSGRGDSPASGRGDSPYSGRGDSPASGRGDSPYSGRGDSP | |
| ASGRGDSPYSGRGDSPASGRGDSPYSGRGDSPASGRGDSPYSGRGDSPASGRGDSPYSGRGDSPASGRGD | |
| SPYSGRGDSPASGRGDSPYSGRGDSPASGRGDSPYSGRGDSPASGY | |
| SKGP-[GRGDSPYSGRGDSPYSGRGDSPYSGRGDSPIS]W-GY | 263 |
| SKGPGRGDSPYSGRGDSPYSGRGDSPYSGRGDSPISGRGDSPYSGRGDSPYSGRGDSPYSGRGDSPISGR | |
| GDSPYSGRGDSPYSGRGDSPYSGRGDSPISGRGDSPYSGRGDSPYSGRGDSPYSGRGDSPISGRGDSPYS | |
| GRGDSPYSGRGDSPYSGRGDSPISGRGDSPYSGRGDSPYSGRGDSPYSGRGDSPISGRGDSPYSGRGDSP | |
| YSGRGDSPYSGRGDSPISGRGDSPYSGRGDSPYSGRGDSPYSGRGDSPISGRGDSPYSGRGDSPYSGRGD | |
| SPYSGRGDSPISGRGDSPYSGRGDSPYSGRGDSPYSGRGDSPISGY | |
| SKGP-[GRGDSPYSGRGDSPIS]20-GY | 264 |
| SKGPGRGDSPYSGRGDSPISGRGDSPYSGRGDSFISGRGDSPYSGRGDSPISGRGDSPYSGRGDSPISGR | |
| GDSPYSGRGDSPISGRGDSPYSGRGDSPISGRGDSPYSGRGDSPiSGRGDSPYSGRGDSPISGRGDSPYS | |
| GRGDSPISGRGDSPYSGRGDSPISGRGDSPYSGRGDSPISGRGDSPYSGRGDSPISGRGDSPYSGRGDSP | |
| ISGRGDSPYSGRGDSPISGRGDSPYSGRGDSFISGRGDSPYSGRGDSPISGRGDSPYSGRGDSFISGRGD | |

SPYSGRGDSPISGRGDSPYSGRGDSPISGRGDSPYSGRGDSPISGRGDSPYSGRGDSPISGRGDSPYSGR
GDSPISGY

| | |
|---|---|
| SKGP-[GRGDSPYSGRGDSPYSGRGDSPYSGRGDSPMS]10-GY | 265 |
| SKGPGRGDSFYSGRGDSPYSGRGDSPYSGRGDSPMSGRGDSPYSGRGDSPYSGRGDSPYSGRGDSPMSGR | |
| GDSPYSGRGDSPYSGRGDSPYSGRGDSPMSGRGDSPYSGRGDSPYSGRGDSPYSGRGDSPMSGRGDSPYS | |
| GRGDSPYSGRGDSPYSGRGDSPMSGRGDSPYSGRGDSPYSGRGDSPYSGRGDSPMSGRGDSPYSGRGDSP | |
| YSGRGDSPYSGRGDSPMSGRGDSPYSGRGDSPYSGRGDSPYSGRGDSPMSGRGDSPYSGRGDSPYSGRGD | |
| SPYSGRGDSPMSGRGDSPYSGRGDSPYSGRGDSPYSGRGDSPMSGY | |
| SKGP-[GRGDSPYSGRGDSPMS]20-GY | 266 |
| SKGPGRGDSPYSGRGDSPMSGRGDSPYSGRGDSPMSGRGDSPYSGRGDSPMSGRGDSPYSGRGDSPMSGR | |
| GDSPYSGRGDSPMSGRGDSPYSGRGDSPMSGRGDSFYSGRGDSPMSGRGDSPYSGRGDSPMSGRGDSPYS | |
| GRGDSPMSGRGDSPYSGRGDSPMSGRGDSPYSGRGDSPMSGRGDSPYSGRGDSPMSGRGDSPYSGRGDSP | |
| MSGRGDSPYSGRGDSPMSGRGDSPYSGRGDSPMSGRGDSPYSGRGDSPMSGRGDSPYSGRGDSPMSGRGD | |
| SPYSGRGDSPMSGRGDSPYSGRGDSPMSGRGDSPYSGRGDSPMSGY | |
| SKGP-[GRGDSPYSGRGDSPYSGRGDSPYSGRGDSPHS]10-GY | 267 |
| SKGPGRGDSPYSGRGDSPYSGRGDSPYSGRGDSPHSGRGDSPYSGRGDSPYSGRGDSPYSGRGDSPHSGR | |
| GDSPYSGRGDSPYSGRGDSPYSGRGDSPHSGRGDSPYSGRGDSPYSGRGDSPYSGRGDSPHSGRGDSPYS | |
| GRGDSPYSGRGDSPYSGRGDSPHSGRGDSPYSGRGDSPYSGRGDSPYSGRGDSPHSGRGDSPYSGRGDSP | |
| YSGRGDSPYSGRGDSPHSGRGDSPYSGRGDSPYSGRGDSPYSGRGDSPHSGRGDSPYSGRGDSPYSGRGD | |
| SPYSGRGDSPHSGRGDSPYSGRGDSPYSGRGDSPYSGRGDSPHSGY | |
| SKGP-[GRGDSPYSGRGDSPHS]20-GY | 268 |
| SKGPGRGDSPYSGRGDSPHSGRGDSPYSGRGDSPHSGRGDSPYSGRGDSPHSGRGDSPYSGRGDSPHSGR | |
| GDSPYSGRGDSPHSGRGDSPYSGRGDSPHSGRGDSPYSGRGDSPHSGRGDSPYSGRGDSPHSGRGDSPYS | |
| GRGDSPHSGRGDSPYSGRGDSPHSGRGDSPYSGRGDSPHSGRGDSPYSGRGDSPHSGRGDSPYSGRGDSP | |
| HSGRGDSPYSGRGDSPHSGRGDSPYSGRGDSPHSGRGDSPYSGRGDSPHSGRGDSPYSGRGDSPHSGRGD | |
| SPYSGRGDSPHSGRGDSPYSGRGDSPHSGRGDSPYSGRGDSPHSGY | |
| SKGP-[GRGDSPHSGRGDSPHSGRGDSPHSGRGDSPYS]10-GY | 269 |
| SKGPGRGDSPHSGRGDSPHSGRGDSPHSGRGDSPYSGRGDSPHSGRGDSPHSGRGDSPHSGRGDSPYSGR | |
| GDSPHSGRGDSPHSGRGDSPHSGRGDSPYSGRGDSPHSGRGDSPHSGRGDSPHSGRGDSPYSGRGDSPHS | |
| GRGDSPHSGRGDSPHSGRGDSPYSGRGDSPHSGRGDSPHSGRGDSPHSGRGDSPYSGRGDSPHSGRGDSP | |
| HSGRGDSPHSGRGDSPYSGRGDSPHSGRGDSPHSGRGDSPHSGRGDSPYSGRGDSPHSGRGDSPHSGRGD | |
| SPHSGRGDSPYSGRGDSPHSGRGDSPHSGRGDSPHSGRGDSPYSGY | |
| SKGP-[GRGDSPYSGRGDSPYSGRGDSPYSGKGDSPYS]10-GY | 270 |
| SKGPGRGDSPYSGRGDSPYSGRGDSPYSGKGDSPYSGRGDSPYSGRGDSPYSGRGDSPYSGKGDSPYSGR | |
| GDSPYSGRGDSPYSGRGDSPYSGKGDSPYSGRGDSPYSGRGDSPYSGRGDSPYSGKGDSPYSGRGDSPYS | |
| GRGDSPYSGRGDSPYSGKGDSPYSGRGDSPYSGRGDSPYSGRGDSPYSGKGDSPYSGRGDSPYSGRGDSP | |
| YSGRGDSPYSGKGDSPYSGRGDSPYSGRGDSPYSGRGDSPYSGKGDSPYSGRGDSPYSGRGDSPYSGRGD | |
| SPYSGKGDSPYSGRGDSPYSGRGDSPYSGRGDSPYSGKGDSPYSGY | |
| SKGP-[GRGDSPYSGKGDSPYS]20-GY | 271 |
| SKGPGRGDSPYSGKGDSPYSGRGDSPYSGKGDSPYSGRGDSPYSGKGDSPYSGRGDSPYSGKGDSPYSGR | |
| GDSPYSGKGDSPYSGRGDSPYSGKGDSPYSGRGDSPYSGKGDSPYSGRGDSPYSGKGDSPYSGRGDSPYS | |
| GKGDSPYSGRGDSPYSGKGDSPYSGRGDSPYSGKGDSPYSGRGDSPYSGKGDSPYSGRGDSPYSGKGDSP | |
| YSGRGDSPYSGKGDSPYSGRGDSPYSGKGDSPYSGRGDSPYSGKGDSPYSGRGDSPYSGKGDSPYSGRGD | |
| SPYSGKGDSPYSGRGDSPYSGKGDSPYSGRGDSPYSGKGDSPYSGY | |
| SKGP-[GRGDSPYSGRGESPYS]20-GY | 272 |
| SKGPGRGDSPYSGRGESPYSGRGDSPYSGRGESPYSGRGDSPYSGRGESPYSGRGDSPYSGRGESPYSGR | |
| GDSPYSGRGESPYSGRGDSPYSGRGESPYSGRGDSPYSGRGESPYSGRGDSPYSGRGESPYSGRGDSPYS | |
| GRGESPYSGRGDSPYSGRGESPYSGRGDSPYSGRGESPYSGRGDSPYSGRGESPYSGRGDSPYSGRGESP | |
| YSGRGDSPYSGRGESPYSGRGDSPYSGRGESPYSGRGDSPYSGRGESPYSGRGDSPYSGRGESPYSGRGD | |
| SPYSGRGESPYSGRGDSPYSGRGESPYSGRGDSPYSGRGESPYSGY | |
| SKGP-[GRGDSPYSGRGDSPYSGRGDSPYSGRGDSPWS]20-GY | 273 |
| SKGPGRGDSPYSGRGDSPYSGRGDSPYSGRGDSPWSGRGDSPYSGRGDSPYSGRGDSPYSGRGDSPWSGR | |
| GDSPYSGRGDSPYSGRGDSPYSGRGDSPWSGRGDSPYSGRGDSPYSGRGDSPYSGRGDSPWSGRGDSPYS | |
| GRGDSPYSGRGDSPYSGRGDSPWSGRGDSPYSGRGDSPYSGRGDSPYSGRGDSPWSGRGDSPYSGRGDSP | |
| YSGRGDSPYSGRGDSPWSGRGDSPYSGRGDSPYSGRGDSPYSGRGDSPWSGRGDSPYSGRGDSPYSGRGD | |
| SPYSGRGDSPWSGRGDSPYSGRGDSPYSGRGDSPYSGRGDSPWSGY | |
| SKGP-[GRGDSPYSGRGDSPWS]20-GY | |
| SKGPGRGDSFYSGRGDSPWSGRGDSPYSGRGDSPWSGRGDSPYSGRGDSPWSGRGDSPYSGRGDSPWSGR | |
| GDSPYSGRGDSPWSGRGDSPYSGRGDSPWSGRGDSPYSGRGDSPWSGRGDSPYSGRGDSPWSGRGDSPYS | |
| GRGDSPWSGRGDSPYSGRGDSPWSGRGDSPYSGRGDSPWSGRGDSPYSGRGDSPWSGRGDSPYSGRGDSP | 274 |
| WSGRGDSPYSGRGDSPWSGRGDSPYSGRGDSPWSGRGDSPYSGRGDSPWSGRGDSPYSGRGDSPWSGRGD | |
| SPYSGRGDSPWSGRGDSPYSGRGDSPWSGRGDSPYSGRGDSPWSGY | |
| SKGP-[GRPDSPYSGRGDSPYSGRGDSPYSGRGDSPYSPRGDSPYSGRGDSPYSGRGDSPY | 275 |
| SGRGDSPYSGRPDSPYSGRGDSPYSGRGDSPYSGRGDSPYSPRGDSPYSGRGDSPYSGR | |
| GDSPYSGRGDSPYSGRPDSPYSGRGDSPYSGRGDSPYSGRGDSPYS]2-GY | |
| SKGPGRPDSPYSGRGDSPYSGRGDSPYSGRGDSPYSPRGDSPYSGRGDSPYSGRGDSPYSGRGDSPYSGR | |
| PDSPYSGRGDSPYSGRGDSPYSGRGDSPYSPRGDSPYSGRGDSPYSGRGDSPYSGRGDSPYSGRPDSPYS | |
| GRGDSPYSGRGDSPYSGRGDSPYSGRPDSPYSGRGDSPYSGRGDSPYSGRGDSPYSPRGDSPYSGRGDSP | |
| YSGRGDSPYSGRGDSPYSGRPDSPYSGRGDSPYSGRGDSPYSGRGDSPYSPRGDSPYSGRGDSPYSGRGD | |
| SPYSGRGDSPYSGRPDSPYSGRGDSPYSGRGDSPYSGRGDSPYSGY | |
| SKGP-[GRPDSPYSGRGDSPYSPRGDSPYSGRGDSPYS]10-GY | |
| SKGPGRPDSPYSGRGDSPYSPRGDSPYSGRGDSPYSGRPDSPYSGRGDSPYSPRGDSPYSGRGDSPYSGR | |
| PDSPYSGRGDSPYSPRGDSPYSGRGDSPYSGRPDSPYSGRGDSPYSPRGDSPYSGRGDSPYSGRPDSPYS | |
| GRGDSPYSPRGDSPYSGRGDSPYSGRPDSPYSGRGDSPYSPRGDSPYSGRGDSPYSGRPDSPYSGRGDSP | 276 |
| YSPRGDSPYSGRGDSPYSGRPDSPYSGRGDSPYSPRGDSPYSGRGDSPYSGRPDSPYSGRGDSPYSPRGD | |
| SPYSGRGDSPYSGRPDSPYSGRGDSPYSPRGDSPYSGRGDSPYSGY | |
| SKGP- | 277 |
| [GRPDSPYSPRGDSPYSGRPDSPYSGRGDSPYSPRGDSPYSGRPDSPYSPRGDSPYSGRG | |
| DSPYSGRPDSPYSPRGDSPYSGRPDSPYSGRGDSPYSPRGDSPYSGRPDSPYSPRGDSPY | |
| SGRGDSPYSGRPDSPYSPRGDSPYSGRPDSPYSGRGDSPYS]2-GY | |
| SKGPGRPDSPYSPRGDSPYSGRPDSPYSGRGDSPYSPRGDSPYSGRPDSPYSPRGDSPYSGRGDSPYSGR | |
| PDSPYSPRGDSPYSGRPDSPYSGRGDSPYSPRGDSPYSGRPDSPYSPRGDSPYSGRGDSPYSGRPDSPYS | |
| PRGDSPYSGRPDSPYSGRGDSPYSGRPDSPYSPRGDSPYSGRPDSPYSGRGDSPYSPRGDSPYSGRPDSP | |
| YSPRGDSFYSGRGDSPYSGRPDSPYSPRGDSPYSGRPDSPYSGRGDSPYSPRGDSPYSGRPDSPYSPRGD | |
| SPYSGRGDSPYSGRPDSPYSPRGDSPYSGRPDSPYSGRGDSPYSGY | |
| SKGP-[GRGDSPYSGRGDSPYSGRGDSPYSGRGDSPVS]10-SfGFP | 278 |
| SKGPGRGDSPYSGRGDSPYSGRGDSPYSGRGDSPVSGRGDSPYSGRGDSPYSGRGDSPYSGRGDSPVSGR | |
| GDSPYSGRGDSPYSGRGDSPYSGRGDSPVSGRGDSPYSGRGDSPYSGRGDSPYSGRGDSPVSGRGDSPYS | |
| GRGDSPYSGRGDSPYSGRGDSPVSGRGDSPYSGRGDSPYSGRGDSPYSGRGDSPVSGRGDSPYSGRGDSP | |
| YSGRGDSPYSGRGDSPVSGRGDSPYSGRGDSPYSGRGDSPYSGRGDSPVSGRGDSPYSGRGDSPYSGRGD | |
| SPYSGRGDSFVSGRGDSPYSGRGDSPYSGRGDSPYSGRGDSPVSGKGEELFTGVVPILVELDGDVNGHKE | |
| SVRGEGEGDATNGKLTLKFICTTGKLPVPWPTLVTTLTYGVQCFSRYPDHMKRHDFFKSAMPEGYVQERT | |
| ISFKDDGNYKTRAEVKFEGDTLVNRIELKGIDFKEDGNILGHKLEYNYNSHNVYITADKQKNGIKANFKI | |
| RHNIEDGSVQLADHYQQNTPIGDGPVLLPDNHYLSTQSVLSKDPNEKRDHMVLLEFVTAAGITHGMDELY | |
| KELHHHHHHG | |
| AzF-([GRGDSPYSGRGDSPYSGRGDSPYSGRGDSPVS]5-AZF)2 | |
| XGRGDSPYSGRGDSPYSGRGDSPYSGRGDSPVSGRGDSPYSGRGDSPYSGRGDSPYSGRGDSPVSGRGDS | 279 |
| PYSGRGDSPYSGRGDSPYSGRGDSPVSGRGDSPYSGRGDSPYSGRGDSPYSGRGDSPVSGRGDSPYSGRG | |
| DSPYSGRGDSPYSGRGDSPVSXGRGDSPYSGRGDSPYSGRGDSPYSGRGDSPVSGRGDSPYSGRGDSPYS | |
| GRGDSPYSGRGDSPVSGRGDSPYSGRGDSPYSGRGDSPYSGRGDSPVSGRGDSPYSGRGDSPYSGRGDSP | |
| YSGRGDSPVSGRGDSPYSGRGDSPYSGRGDSPYSGRGDSPVSX | |

X:AzF

### Binding Polypeptide

The binding polypeptide (or “targeting polypeptide”) may comprise any polypeptide that is capable of binding at least one target. The binding polypeptide may bind at least one target. “Target” may be an entity capable of being bound by the binding polypeptide. Targets may include, for example, another polypeptide, a cell surface receptor, a carbohydrate, an antibody, a small molecule, or a combination thereof. The target may be a biomarker. The target may be activated through agonism or blocked through antagonism. The binding polypeptide may specifically bind the target. By binding target, the binding polypeptide may act as a targeting moiety, an agonist, an antagonist, or a combination thereof. In some embodiments, the binding polypeptide domain binds

The binding polypeptide may be a monomer that binds to a target. The monomer may bind one or more targets. The binding polypeptide may form an oligomer. The binding polypeptide may form an oligomer with the same or different binding polypeptides. The oligomer may bind to a target. The oligomer may bind one or more targets. One or more monomers within an oligomer may bind one or more targets. In some embodiments, the fusion protein is multivalent. In some embodiments, the fusion protein binds multiple targets. In some embodiments, the activity of the binding polypeptide alone is the same as the activity of the binding protein when part of a fusion protein.

In one aspect, the wherein the binding polypeptide comprises one or more of an antibody binding domain derived from Staphylococcus protein A (ZD) (SEQ ID NO:159), an antimicrobial peptide selected from LL37 (SEQ ID NO: 161), Ib-M1 (SEQ ID NO: 163), Ib-M2 (SEQ ID NO: 165), Ib-M5 (SEQ ID NO: 167), Cathelecidin-1 (SEQ ID NO: 169), A(A1R, A8R, I17K) (SEQ ID NO: 171), H5 (SEQ ID NO: 173), H5-61-90 (SEQ ID NO: 175); RGD peptide (RGDSPAS, SEQ ID NO: 39); protein drugs, GLP-1 (SEQ ID NO: 177); fluorescent reporters (sfGFP (SEQ ID NO: 179), mRuby3 (SEQ ID NO: 181); RNA binding proteins (PUM-HD (SEQ ID NO: 183), eIF4E (SEQ ID NO: 185), PABP (SEQ ID NO: 187), Tis11D (SEQ ID NO: 189)); KH domains (Yifan or FMRP (SEQ ID NO: 191)); or AAV binding peptides PKD1 (SEQ ID NO: 193) or PKD2 (SEQ ID NO: 195).

### Linker

In some embodiments, the fusion protein further includes at least one linker. In some embodiments, the fusion protein includes more than one linker. In such embodiments, the linkers may be the same or different from one another. The fusion protein may include, none, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 linkers. The fusion protein may include less than 500, less than 400, less than 300, or less than 200 linkers. The fusion protein may include between 1 and 1000, between 10 and 900, between 10 and 800, or between 5 and 500 linkers.

The linker may be positioned in between a binding polypeptide and a polypeptide with controlled reversible phase separation, in between binding polypeptides, in between polypeptides with controlled reversible phase separation, or a combination thereof. Multiple linkers may be positioned adjacent to one another. Multiple linkers may be positioned adjacent to one another and in between the binding polypeptide and the polypeptide with controlled reversible phase separation.

The linker may be a polypeptide of any amino acid sequence and length. The linker may act as a spacer peptide. The linker may occur between polypeptide domains. The linker may sufficiently separate the binding domains of the binding polypeptide while preserving the activity of the binding domains. In some embodiments, the linker comprises charged amino acids. In some embodiments, the linker is flexible. In some embodiments, the linker comprises at least one glycine and at least one serine. In some embodiments, the linker comprises at least one proline.

### Polynucleotides

Further provided are polynucleotides encoding the fusion proteins detailed herein. A vector may include the polynucleotide encoding the fusion proteins detailed herein. To obtain expression of a polypeptide, one typically subclones the polynucleotide encoding the polypeptide into an expression vector that contains a promoter to direct transcription, a transcription/translation terminator, and if for a nucleic acid encoding a protein, a ribosome binding site for translational initiation. An example of a vector is pET24. Suitable bacterial promoters are well known in the art. Further provided is a host cell transformed or transfected with an expression vector comprising a polynucleotide encoding a fusion protein as detailed herein. Bacterial expression systems for expressing the protein are available in, e.g., E. coli, Bacillus sp., and Salmonella (Paiva et al., Gene 1983, 22, 229-235; Mosbach et al., Nature 1983, 302, 543-545). Kits for such expression systems are commercially available. Eukaryotic expression systems for mammalian cells, yeast, and insect cells are well known in the art and are commercially available. Retroviral expression systems can be used in the present invention. In some embodiments, the fusion protein comprises repeats or single sequences of one or more of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, or 197-279. In some embodiments, the fusion protein comprises repeats or single sequences of one or more of a polypeptide encoded by a polynucleotide sequence of any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, or 158. In some embodiments, the fusion protein comprises a polypeptide comprising an amino acid sequence of any one of SEQ ID NO: 284, 286, 288, 290, 292, 294, 296, 298, 300, 302, 304, 306, 308, 310, 312, 314, or 316.

### Administration

The peptide biopolymers comprising one or more fusion proteins as detailed herein can be formulated in accordance with standard techniques well known to those skilled in the pharmaceutical art to form a therapeutic agent or targeted delivery agent. Such compositions comprising peptide biopolymers comprising one or more fusion proteins can be administered in dosages and by techniques well known to those skilled in the medical arts taking into consideration such factors as the age, sex, weight, and condition of the particular subject, and the route of administration.

The peptide biopolymers comprising one or more fusion proteins can be administered prophylactically or therapeutically. In prophylactic administration, the peptide biopolymer can be administered in an amount sufficient to induce a response. In therapeutic applications, the peptide biopolymers are administered to a subject in need thereof in an amount sufficient to elicit a therapeutic effect. An amount adequate to accomplish this is defined as “therapeutically effective dose.” Amounts effective for this use will depend on, e.g., the particular composition of the peptide biopolymer regimen administered, the manner of administration, the stage, and severity of the disease, the general state of health of the patient, and the judgment of the prescribing physician.

The peptide biopolymer can be administered by methods well known in the art as described in Donnelly et al. Ann. Rev. Immunol. 1997, 75, 617-648; Feigner et al., U.S. Pat. No. 5,580,859; Feigner, U.S. Pat. No. 5,703,055; and Carson et al., U.S. Pat. No. 5,679,647, the contents of each of which are incorporated herein by reference in their entirety. The peptide biopolymer can be complexed to particles or beads that can be administered to an individual, for example, using a vaccine gun. One skilled in the art would know that the choice of a pharmaceutically acceptable carrier, including a physiologically acceptable compound, depends, for example, on the route of administration.

The peptide biopolymers can be delivered via a variety of routes. Typical delivery routes include parenteral administration, e.g., intradermal, intramuscular, or subcutaneous delivery. Other routes include oral administration, intranasal, intravaginal, transdermal, intravenous, intraarterial, intratumoral, intraperitoneal, and epidermal routes. In some embodiments, the peptide biopolymer is administered intravenously, intraarterially, or intraperitoneally to the subject.

The peptide biopolymer can be a liquid preparation such as a suspension, syrup, or elixir. The peptide biopolymer can be incorporated into liposomes, microspheres, or other polymer matrices (such as by a method described in Feigner et al., U.S. Pat. No. 5,703,055; Gregoriadis, Liposome Technology, Vols. I to III (2^{nd} ed. 1993), the contents of which are incorporated herein by reference in their entirety). Liposomes can consist of phospholipids or other lipids, and can be nontoxic, physiologically acceptable, and metabolizable carriers that are relatively simple to make and administer.

In some embodiments, the peptide biopolymer is administered in a controlled release formulation. In some embodiments, the peptide biopolymer comprises one or more thermally responsive polypeptides, the thermally responsive polypeptide having a transition temperature such that the peptide biopolymer remains soluble prior to administration and such that the peptide biopolymer transitions upon administration to a gel-like depot in the subject. In some embodiments, the peptide biopolymer comprises one or more fusion proteins comprising one or more thermally responsive polypeptides, the thermally responsive polypeptide having a transition temperature such that the fusion protein remains soluble at room temperature and such that the fusion protein transitions upon administration to a gel-like depot in the subject. For example, in some embodiments, the fusion protein comprises one or more thermally responsive polypeptides, the thermally responsive polypeptide having a transition temperature between room temperature (about 25° C.) and body temperature (about 37° C.), whereby the fusion protein can be administered to form a depot. As used herein, “depot” refers to a gel-like composition comprising a fusion protein that releases the fusion protein over time. In some embodiments, the peptide biopolymer can be injected subcutaneously or intratumorally to form a depot (coacervate). The depot may provide controlled (slow) release of the peptide biopolymer. The depot may provide slow release of the peptide biopolymer into the circulation or the tumor, for example. In some embodiments, the peptide biopolymer may be released from the depot over a period of at least about 1 day, at least about 2 days, at least about 3 days, at least about 4 days, at least about 5 days, at least about 6 days, at least about 7 days, at least about 1 week, at least about 1.5 weeks, at least about 2 weeks, at least about 2.5 weeks, at least about 3.5 weeks, at least about 4 weeks, or at least about 1 month.

### Detection

As used herein, the term “detect” or “determine the presence of” refers to the qualitative measurement of undetectable, low, normal, or high concentrations of one or more peptide biopolymers, targets, or peptide biopolymers bound to target. Detection may include in vitro, ex vivo, or in vivo detection. Detection may include detecting the presence of one or more peptide biopolymers comprising one or more peptide biopolymers or targets versus the absence of the one or more peptide biopolymer or targets. Detection may also include quantification of the level of one or more peptide biopolymers or targets. The terms “quantify,” or “quantification” may be used interchangeably, and may refer to a process of determining the quantity or abundance of a substance (e.g., peptide biopolymer or target), whether relative or absolute. Any suitable method of detection falls within the general scope of the present disclosure. In some embodiments, the peptide biopolymer comprises a reporter attached thereto for detection. In some embodiments, the peptide biopolymer is labeled with a reporter. In some embodiments, detection of a peptide biopolymer bound to a target may be determined by methods including but not limited to, band intensity on a Western blot, flow cytometry, radiolabel imaging, cell binding assays, activity assays, SPR, immunoassay, or by various other methods known in the art.

In some embodiments, including those wherein the peptide biopolymer is an antibody mimic for binding and/or detecting a target, any immunoassay may be utilized. The immunoassay may be an enzyme-linked immunoassay (ELISA), radioimmunoassay (RIA), a competitive inhibition assay, such as forward or reverse competitive inhibition assays, a fluorescence polarization assay, or a competitive binding assay, for example. The ELISA may be a sandwich ELISA. Specific immunological binding of the f peptide biopolymer to the target can be detected via direct labels, attached to the peptide biopolymer or via indirect labels, such as alkaline phosphatase or horseradish peroxidase. The use of an immobilized peptide biopolymer may be incorporated into the immunoassay. The peptide biopolymers may be immobilized onto a variety of supports, such as magnetic or chromatographic matrix particles, the surface of an assay plate (such as microtiter wells), pieces of a solid substrate material, and the like. An assay strip can be prepared by coating the peptide biopolymer or plurality of peptide biopolymers in an array on a solid support. This strip can then be dipped into the test biological sample and then processed quickly through washes and detection steps to generate a measurable signal, such as a colored spot.

### Methods of Treating a Disease

The present invention is directed to a method of treating a disease in a subject in need thereof. The method may comprise administering to the subject an effective amount of the peptide biopolymer comprising one or more peptide biopolymers as described herein. The disease may be selected from cancer, metabolic disease, autoimmune disease, cardiovascular disease, and orthopedic disorders. In some embodiments, the disease is a disease associated with a target of the at least one binding polypeptide.

Metabolic disease may occur when abnormal chemical reactions in the body alter the normal metabolic process. Metabolic diseases may include, for example, insulin resistance, non-alcoholic fatty liver diseases, type 2 diabetes, insulin resistance diseases, cardiovascular diseases, arteriosclerosis, lipid-related metabolic disorders, hyperglycemia, hyperinsulinemia, hyperlipidemia, and glucose metabolic disorders.

Autoimmune diseases arise from an abnormal immune response of the body against substances and tissues normally present in the body. Autoimmune diseases may include, but are not limited to, lupus, rheumatoid arthritis, multiple sclerosis, insulin dependent diabetes mellitis, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, polyarteritis nodosa, myocarditis, psoriasis, Celiac disease, Crohn's disease, ulcerative colitis, and fibromyalgia.

Cardiovascular disease is a class of diseases that involve the heart or blood vessels. Cardiovascular diseases may include, for example, coronary artery diseases (CAD) such as angina and myocardial infarction (heart attack), stroke, hypertensive heart disease, rheumatic heart disease, cardiomyopathy, heart arrhythmia, congenital heart disease, valvular heart disease, carditis, aortic aneurysms, peripheral artery disease, and venous thrombosis.

Orthopedic disorders or musculoskeletal disorders are injuries or pain in the body's joints, ligaments, muscles, nerves, tendons, and structures that support limbs, neck, and back. Orthopedic disorders may include degenerative diseases and inflammatory conditions that cause pain and impair normal activities. Orthopedic disorders may include, for example, carpal tunnel syndrome, epicondylitis, and tendinitis. Cancers may include, but are not limited to, breast cancer, colorectal cancer, colon cancer, lung cancer, prostate cancer, testicular cancer, brain cancer, skin cancer, rectal cancer, gastric cancer, esophageal cancer, sarcomas, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers. In some embodiments, the cancer is colorectal cancer. In some embodiments, the cancer is colorectal adenocarcinoma.

One application of protein therapeutics is cancer treatment. In specific embodiments, the present invention provides a method for using scaffold proteins in developing antibody mimetics for oncological targets of interest. With the emergence of scaffold protein engineering come the possibilities for designing potent protein drugs that are unhindered by steric and architectural limitations. Although potent protein drugs can be invaluable for diagnostics or treatments, successful delivery to the target region can pose a great challenge.

### Methods of Diagnosing a Disease

Provided herein are methods of diagnosing a disease. The methods may include administering to the subject a peptide biopolymer comprising one or more fusion proteins as described herein and detecting binding of the peptide biopolymer to a target to determine presence of the target in the subject. The presence of the target may indicate the disease in the subject. In other embodiments, the methods may include contacting a sample from the subject with a peptide biopolymer as described herein, determining the level of a target in the sample, and comparing the level of the target in the sample to a control level of the target, wherein a level of the target different from the control level indicates disease in the subject. In some embodiments, the disease is selected from cancer, metabolic disease, autoimmune disease, cardiovascular disease, and orthopedic disorders, as detailed above. In some embodiments, the target comprises a disease marker or biomarker. In some embodiments, the fusion protein may act as an antibody mimic for binding or detecting a target.

### Methods of Determining the Presences of a Target

Provided herein are methods of determining the presence of a target in a sample. The methods may include contacting the sample with a peptide biopolymer comprising one or more fusion proteins as described herein under conditions to allow a complex to form between the peptide biopolymer and the target in the sample and detecting the presence of the complex. Presence of the complex may be indicative of the target in the sample. In some embodiments, the peptide biopolymer is labeled with a reporter for detection.

In some embodiments, the sample is obtained from a subject and the method further includes diagnosing, prognosticating, or assessing the efficacy of a treatment of the subject. When the method includes assessing the efficacy of a treatment of the subject, then the method may further include modifying the treatment of the subject as needed to improve efficacy.

### Methods of Determining the Effectiveness of a Treatment

Provided herein are methods of determining the effectiveness of a treatment for a disease in a subject in need thereof. The methods may include contacting a sample from the subject with a peptide biopolymer comprising a fusion protein as detailed herein under conditions to allow a complex to form between the peptide biopolymer and a target in the sample, determining the level of the complex in the sample, wherein the level of the complex is indicative of the level of the target in the sample, and comparing the level of the target in the sample to a control level of the target, wherein if the level of the target is different from the control level, then the treatment is determined to be effective or ineffective in treating the disease.

Time points may include prior to onset of disease, prior to administration of a therapy, various time points during administration of a therapy, and after a therapy has concluded, or a combination thereof. Upon administration of the peptide biopolymer comprising one or more fusion proteins to the subject, the peptide biopolymer may bind a target, wherein the presence of the target indicates the presence of the disease in the subject at the various time points. In some embodiments, the target comprises a disease marker or biomarker. In some embodiments, the peptide biopolymer may act as an antibody mimic for binding and/or detecting a target. Comparison of the binding of the peptide biopolymer to the target at various time points may indicate whether the disease has progressed, whether the diseased has advanced, whether a therapy is working to treat or prevent the disease, or a combination thereof.

In some embodiments, the control level corresponds to the level in the subject at a time point before or during the period when the subject has begun treatment, and the sample is taken from the subject at a later time point. In some embodiments, the sample is taken from the subject at a time point during the period when the subject is undergoing treatment, and the control level corresponds to a disease-free level or to the level at a time point before the period when the subject has begun treatment. In some embodiments, the method further includes modifying the treatment or administering a different treatment to the subject when the treatment is determined to be ineffective in treating the disease.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A polypeptide with controlled reversible phase separation comprising ten or more repeats of an amino acid sequence comprising:

(X-Z₁-X-Z₂-Z₃-X-Z₄-Z₃)ₙ,
where:
X is proline (P) or glycine (G) and the ratio of P:G is any number;
Z₁ is arginine (R), aspartic acid (D), or lysine (K) and the ratio of R:D is any number and the ratio of K:R can be any number;
Z₂ is Asp (D), Arg (R), Glu (E), where the ratio of R:D can be any number and D:E can be any number;
Z₃ is asparagine (N), glutamine (Q), serine (S), or threonine (T) were the ratio among N:Q:S:T can be any number; and
Z₄ is tyrosine (Y), histidine (H), tryptophan (W), phenylalanine (F), methionine (M), valine (V), isoleucine (I), alanine (A), or leucine (L) and the ratio among Y:H:W:F:M:V:I:A:L can be any number.
Clause 2. The polypeptide of clause 1, wherein X is proline (P) or glycine (G) and the ratio of P:G is between 1:3 and 3:1.
Clause 3. The polypeptide of clause 1 or 2, wherein Z₁ is arginine (R), aspartic acid (D), or lysine (K) and the ratio of R:D does not exceed 1:5 and the ratio of K:R can be any number.
Clause 4. The polypeptide of any one of clauses 1-3, wherein the phase separation is dependent on temperature, molecular weight, hydrophobicity, aromatic:aliphatic ratio, and concentration.
Clause 5. The polypeptide of any one of clauses 1-4, wherein n is 10 to 200.
Clause 6. The polypeptide of any one of clauses 1-5, wherein the molecular weight is at least 5 kDa to 500 kDa.
Clause 7. The polypeptide of any one of clauses 1-6, wherein the molecular weight is about 5 kDa to about 100 kDa.
Clause 8. The polypeptide of any one of clauses 1-7, wherein the phase separation temperature is 0 to 100° C.
Clause 9. The polypeptide of any one of clauses 1-8, wherein the phase separation temperature is 4 to 25° C.; ˜25° C.; 25 to 37° C.; ˜37° C.; 35 to 38° C.; or >38° C.
Clause 10. The polypeptide of any one of clauses 1-9, wherein the polypeptide comprises modified amino acids, a reporter protein, or an enzyme.
Clause 11. The polypeptide of any one of clauses 1-10, wherein the sequence comprises:

(G-R-G-D-S-P-Y-S)ₘ,
where m is 20 to 80.
Clause 12. The polypeptide of any one of clauses 1-11, wherein the polypeptide comprises a sequence selected from one or more of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, or 197-279, or combinations thereof.
Clause 13. A pharmaceutically acceptable composition comprising a polypeptide with controlled reversible phase separation comprising ten or more repeats of an amino acid sequence comprising:

(X-Z₁-X-Z₂-Z₃-X-Z₄-Z₃)ₙ,
where:
X is proline (P) or glycine (G) and the ratio of P:G is any number;
Z₁ is arginine (R), aspartic acid (D), or lysine (K) and the ratio of R:D is any number and the ratio of K:R can be any number;
Z₂ is Asp (D), Arg (R), Glu (E), where the ratio of R:D can be any number and D:E can be any number;
Z₃ is asparagine (N), glutamine (Q), serine (S), or threonine (T) were the ratio among N:Q:S:T can be any number; and
Z₄ is tyrosine (Y), histidine (H), tryptophan (W), phenylalanine (F), methionine (M), valine (V), isoleucine (I), alanine (A), or leucine (L) and the ratio among Y:H:W:F:M:V:I:A:L can be any number.
Clause 14. The composition of clause 13, wherein X is proline (P) or glycine (G) and the ratio of P:G is between 1:3 and 3:1.
Clause 15. The composition of clause 13 or 14, wherein Z₁ is arginine (R), aspartic acid (D), or lysine (K) and the ratio of R:D does not exceed 1:5 and the ratio of K:R can be any number.
Clause 16. The composition of any one of clauses 13-15, further comprising an attached molecule comprising one or more of an antibody binding domain derived from Staphylococcus protein A (ZD) (SEQ ID NO:159), an antimicrobial peptide selected from LL37 (SEQ ID NO: 161), Ib-M1 (SEQ ID NO: 163), Ib-M2 (SEQ ID NO: 165), Ib-M5 (SEQ ID NO: 167), Cathelecidin-1 (SEQ ID NO: 169), A(A1R, A8R, I17K) (SEQ ID NO: 171), H5 (SEQ ID NO: 173), H5-61-90 (SEQ ID NO: 175); RGD peptide (RGDSPAS, SEQ ID NO: 39); protein drugs, GLP-1 (SEQ ID NO: 177); fluorescent reporters (sfGFP (SEQ ID NO: 179), mRuby3 (SEQ ID NO: 181); RNA binding proteins (PUM-HD (SEQ ID NO: 183), eIF4E (SEQ ID NO: 185), PABP (SEQ ID NO: 187), Tis11D (SEQ ID NO: 189)); KH domains (Yifan or FMRP (SEQ ID NO: 191)); or AAV binding peptides PKD1 (SEQ ID NO: 193) or PKD2 (SEQ ID NO: 195).
Clause 17. The composition of any one of clauses 13-16, wherein the composition enhances bioavailability of the attached molecule as compared to the free form of the attached molecule.
Clause 18. The composition of any one of clauses 13-17, wherein the composition enhances expression of the attached molecule as compared to the free form of the attached molecule.
Clause 19. The composition of any one of clauses 13-18, wherein the composition enhances the stability of the attached molecule as compared to the free form of the attached molecule.
Clause 20. The composition of clause 19, wherein the composition enhances stability of the attached molecule during prokaryotic and eukaryotic expression as compared to the free form of the attached molecule.
Clause 21. The composition of clause 19 or 20, wherein the enhanced stability includes resistance to denaturation during freezing, thawing, or lyophilization.
Clause 22. The composition of any one of clauses clause 13-21, wherein the composition modulates enzymatic, metabolic, or physiological functions within cells or organisms.
Clause 23. The composition of clause 22, wherein the modulation reduces bioavailability of the attached molecules.
Clause 24. The composition of clause 23, wherein the attached molecules comprise therapeutic or cytotoxic proteins or peptides.
Clause 25. A method for enhancing the bioavailability or stability of a protein, the method comprising creating a fusion protein of one or more proteins and a polypeptide with controlled reversible phase separation comprising ten or more repeats of an amino acid sequence comprising:

(X-Z₁-X-Z₂-Z₃-X-Z₄-Z₃)ₙ,
where:
X is proline (P) or glycine (G) and the ratio of P:G is any number,
Z₁ is arginine (R), aspartic acid (D), or lysine (K) and the ratio of R:D is any number and the ratio of K:R can be any number;
Z₂ is Asp (D), Arg (R), Glu (E), where the ratio of R:D can be any number and D:E can be any number;
Z₃ is asparagine (N), glutamine (Q), serine (S), or threonine (T) were the ratio among N:Q:S:T can be any number; and
Z₄ is tyrosine (Y), histidine (H), tryptophan (W), phenylalanine (F), methionine (M), valine (V), isoleucine (I), alanine (A), or leucine (L) and the ratio among Y:H:W:F:M:V:I:A:L can be any number.
Clause 26. The method of clause 25, wherein X is proline (P) or glycine (G) and the ratio of P:G is between 1:3 and 3:1.
Clause 27. The method of clause 25 or 26, wherein Z₁ is arginine (R), aspartic acid (D), or lysine (K) and the ratio of R:D does not exceed 1:5 and the ratio of K:R can be any number.
Clause 28. The method of any one of clauses 25-27, wherein the protein comprises one or more of an antibody binding domain derived from Staphylococcus protein A (ZD) (SEQ ID NO:159), an antimicrobial peptide selected from LL37 (SEQ ID NO: 161). Ib-M1 (SEQ ID NO: 163), Ib-M2 (SEQ ID NO: 165), Ib-M5 (SEQ ID NO: 167), Cathelecidin-1 (SEQ ID NO: 169), A(A1R, A8R, I17K) (SEQ ID NO: 171), H5 (SEQ ID NO: 173), H5-61-90 (SEQ ID NO: 175); RGD peptide (RGDSPAS, SEQ ID NO: 39); protein drugs, GLP-1 (SEQ ID NO: 177); fluorescent reporters (sfGFP (SEQ ID NO: 179), mRuby3 (SEQ ID NO: 181); RNA binding proteins (PUM-HD (SEQ ID NO: 183), eIF4E (SEQ ID NO: 185), PABP (SEQ ID NO: 187), Tis11D (SEQ ID NO: 189)); KH domains (Yifan or FMRP (SEQ ID NO: 191)); or AAV binding peptides PKD1 (SEQ ID NO: 193) or PKD2 (SEQ ID NO: 195).
Clause 29. The method of any one of clauses 25-28, where the enhanced bioavailability of the fusion protein can be used for isolation or separation of a biologic molecule.
Clause 30. The method of any one of clauses 25-26, wherein the biologic molecule comprises one or more of a lipid, a cell, a protein, a nucleic acid, a carbohydrate, or a viral particle.
Clause 31. The method of clause 30, wherein the nucleic acid is single stranded or double stranded DNA or RNA.
Clause 32. The method of clause 30, wherein the viral particle is an adenovirus particle, an adeno-associated virus particle, a lentivirus particle, a retrovirus particle, a poxvirus particle, a measle virus particle, or herpesvirus particle.
Clause 33. The method of clause 30, wherein the protein comprises albumin, monoclonal IgG antibodies, or Fc fusion antibodies.
Clause 34. The method of clause 29, wherein the isolation or separation is accomplished via reversible phase separation.

### EXAMPLES

### Example 1

### De Novo Engineering of Intracellular Condensates Using Artificial Disordered Proteins

We have taken a different and complementary approach to understand how phase behavior is encoded in polypeptides. Analogous to—and inspired by—synthetic polymers that exhibit lower and upper critical solution temperature (LCST/UCST) phase behavior, we began by systematically scanning the sequence space of native IDPs to identify minimal peptide motifs that will confer LCST or UCST phase behavior when polymerized into a macromolecule that consists of many repeats of the peptide motif. With the greatly reduced sequence complexity of these repetitive polypeptides—compared to native IDPs that exhibit LCST/UST phase behavior—we then made rational changes in the amino acid repeat motif that systematically propagate along the sequence. These repetitive polypeptides can be rationally designed to exhibit both LCST and UCST phase behavior, and their phase behavior can be systematically modulated by amino acid mutations of the repeat motif. These artificial polypeptides also exhibit the same basic principles of phase separation inside cells as native IDPs.

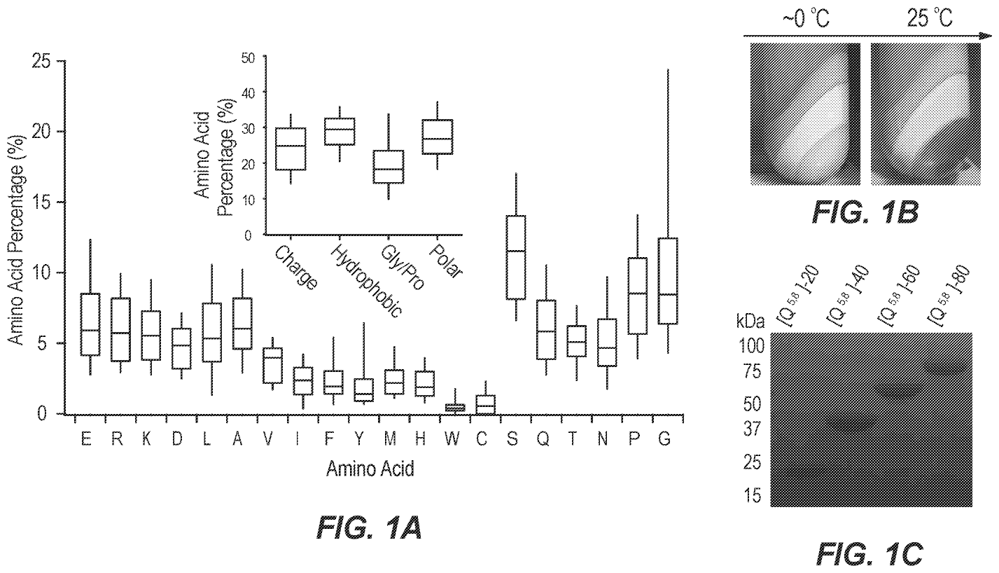

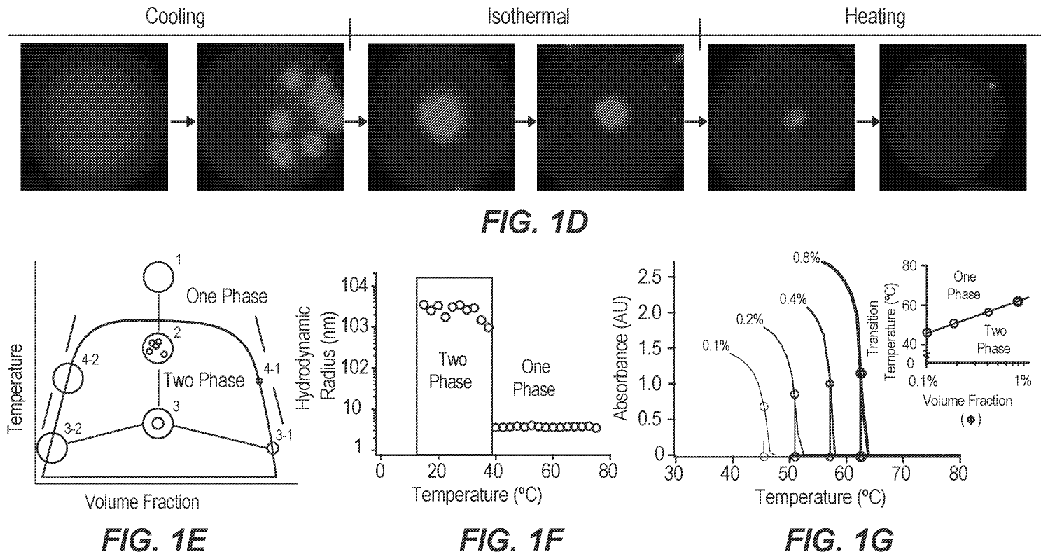

Informed by a heuristic knowledge of factors that drive phase separation in repetitive polypeptides from these studies as well as the natural composition of membrane-less organelle IDPs, we set out to create artificial IDPs (A-IDPs) that exhibit phase separation in living cells to impart new functionality to the cell. Our design began with (G₁-R₂-G₃-D₄-S₅-P₆-Y₇-S₈)ₓₓ (where xx is the number of repeats between 20 and 80) a sequence inspired by Drosophila melanogaster Rec-1 Resilin, known to exhibit UCST phase behavior, that is chemically similar to IDPs that are critical constituents of membrane-less organelles (FIG. 1A). We chose this sequence precisely because it exhibits UCST phase behavior, which appears to be far more common among native IDPs than LCST phase behavior. Thus, we created a set of 63 A-IDPs consisting of repeats of the parent (G₁-R₂-G₃-D₄-S₅-P₆-Y₇-S₈)ₓₓ motif and variants with rational amino acid mutations of this motif. We characterized the UCST phase behavior for this set of 63 IDPs from which we were able to quantify the effect of various amino acid mutations and modifications to the chain architecture on homotypic liquid-liquid phase separation.

We then used a subset of A-IDPs from this library to engineer intracellular condensates in living cells. The behavior of intracellular condensates for these A-IDPs proved to be surprisingly predictable and tunable, and enabled dynamic control over their cytoplasmic solubility and their interaction with the surrounding environment. Capitalizing on these observations, we created intracellular droplets capable of sequestering an enzyme whose catalytic efficiency within the engineered condensates can be genetically encoded by modulating the MW of the A-IDP.

### Materials and Methods

pET24+ vectors were purchased from Novagen (Madison, Wis.). gBlock fragments encoding repetitive IDP (A-IDP) sequences of interest, superfolder GFP (sfGFP), mRuby3 and primers for pcDNA5 vector were purchased from Integrated DNA Technologies (Coralville, Iowa). Ligation enzymes, restriction enzymes, DNA ladders were purchased from New England Biolabs (Ipswich, Mass.). BL21(DE3) chemically competent Escherichia coli (E. coli) cells were purchased from Bioline (Taunton, Mass.). All E. coli cultures were grown in Terrific Broth media purchased from VWR Intemational (Radnor, Pa.). Kanamycin sulfate was purchased from EMD Millipore (Billerica, Mass.). Protein expression was induced with isopropyl β-D-1-thiogalactopyranoside (IPTG) from Gold Biotechnology (St. Louis, Mo.). All salts, 10/40 kDa fluorescein labeled dextran molecules, L-(+)-Arabinose, L-Rhamnose and Fluorescein di(β-D-galactopyranoside) were purchased from Sigma-Aldrich (St. Louis, Mo.). 1× phosphate buffered saline (PBS) tablets (10 mM phosphate buffer, 140 mM NaCl, 3 mM KCl, pH 7.4 at 25° C.) were purchased from EMD Millipore (Billerica, Mass.). KRX E. coli cell line that endogenously expresses mutated LacZ were purchased from Promega (Madison, Wis.). NHS Ester reactive fluorophores (NHS-Alexa Fluor® 350 and NHS-Alexa Fluor® 647) were purchased from Life Technologies (Grand Island, N.Y.). DNA extraction kits, DNA gel purification kits were purchased from Qiagen Inc. (Germantown, Md.). Expi293 Eurkaryotic Expression System for HEK293 expression was purchased from Thermo Fischer Scientific (Waltham, Mass.). Whatman Anotop sterile syringe filters (0.02 μm) were purchased from GE Healthcare Life Sciences (Pittsburgh, Pa.). ABIL® EM 90 and TEGOSOFT® DEC surfactants were purchased from Evonik Industries (Essen, Germany). A single emulsion droplet-generating chip was purchased from Dolomite Microfluidics (Royston, United Kingdom). Syringe pumps were acquired from Chemyx Inc. (Stafford, Tex.).

### Proteomic Analysis

A search of the literature provided an excellent list of intrinsically disordered proteins or protein regions are present in genes known to form membrane-less organelles. Each gene was divided into disordered regions and ordered regions according to the Predictor of Natural Disordered Regions (PONDR) VSL2 algorithm which is a meta-predictor of protein disorder of various lengths. Amino acid quantity was normalized to total protein length.

### Gene Synthesis

Each octapeptide amino acid motif inspired by our proteomic analysis was propagated twenty times in silico. This repetitive amino acid sequence was fed into an algorithm that creates an optimally non-repetitive DNA template from a repetitive protein gene. This 20-mer repeat gene was then ordered from IDT with Gibson assembly overhangs for easy insertion into modified pET24+ vector. To increase the number of total repeats of the gene, we performed iterative cloning steps of Recursive Directional Ligation by Plasmid Reconstruction adding an addition twenty repeats during each step. Transformations were performed into the desired E. coli cell line—BL21(DE3) for recombinant expression and single plasmid confocal experiments and a modified BL21(DE3) cell line termed KRX by Promega that contains a mutated LacZ gene for enzymatic experimentation.

In experiments with dual expression, genes were inserted into the pBAD33.1 vector by cutting custom pET24+ vector and pBAD33.1 cut with Hind III and Xba I. Gel purification was used to isolate the gene of interest from the housing pET24+ vector, which was then ligated into the similarly cut pBAD33.1 vector. Co-transformation was performed with ˜1 ng final concentration of each plasmid on kanamycin/chloramphenicol dual selection plates.

### Protein Expression, Purification and Characterization

Individual liquid cultures of BL21 E. coli strains each harboring our gene of interest from Table 2 or Table 3 were inoculated into 5 mL of Terrific Broth (TB) medium from frozen glycerol stocks and grown to confluence overnight (16-18 hours). Cultures were then inoculated at a 1:200 dilution in 1 L TB media supplemented with 45 μg mL⁻¹ kanamycin. Cells were grown at 37° C. in a shaking incubator (˜200 RPM) for 9 h, at which time protein expression was induced by the addition of 500 μM IPTG (final concentration). Cells were then incubated at 37° C. (shaking at ˜200 RPM.) for an additional 18 h. Protein was then purified from the insoluble cell suspension fraction. In brief, cell pellets were isolated by centrifuging cultures at 3500 RCF and resuspending in 20 mL of milli-Q water. Cells were then lysed by sonicating the cell solutions for 2 minutes, with 10 seconds of pulsing followed by 40 seconds of rest on ice (Misonix; Farmingdale, N.Y.).

Centrifuging each lysate suspension at 20,000 RCF for 20 minutes results in a soluble and insoluble fraction. The supernatant was discarded with the insoluble fraction resuspended in an approximately equal volume of 8 M urea+150 mM PBS (˜6-8 mL). For proteins with a fluorescent fusion tag, the insoluble fraction was resuspended in 3× insoluble volume at a final concentration of urea of ˜2 M to prevent protein misfolding. This suspension was heated for 10 min in a 37° C. water bath and then centrifuged at 20,000 RCF for 20 minutes. The supernatant was collected from this suspension and dialyzed in a 10 kDa membrane (SnakeSkin™, Thermo Fischer Scientific) against a 1:200 milli-Q water solution at 4° C. The dialysis water was changed twice over a 48-hour period. From inside the dialysis bag, both insoluble and soluble components were collected and centrifuged at 3500 RCF for 10 minutes and 4° C. The supernatant was removed and the remaining insoluble pellet containing the protein of interest was lyophilized for a minimum of three days to remove all water from the pellet.

Protein purity was characterized by 4-20% gradient tris-HCl (Biorad, Hercules, Calif.) sodium dodecyl sulfate polyacrylamide gel electrophoresis (SDS-PAGE) and staining with either 0.5 M copper chloride or SimplyBlue™ SafeStain (Thermo Fischer Scientific). Protein yield was determined by weight after lyophilization.

Creation of Water in Oil Droplets with Chip Microfluidics

To create water-in-oil emulsion droplets, two liquid phases—a dispersed, aqueous phase containing protein of interest in 150 mM PBS and an organic, continuous phase comprised of 75%/5%/20% vol/vol TEGOSOFT® DEC/ABIL® EM 90/mineral oil—were injected into the microfluidic droplet generators at constant flow rates using precision syringe pumps. The flow rates of the dispersed and continuous fluids were tuned to ensure droplet formation in the dripping regime; in these experiments, the dripping regime was achieved using a constant flow rate of 500 μL hr⁻¹ for the organic continuous phase and 50-75 μL hr⁻¹ for the aqueous, dispersed phase. The production of droplets within the microfluidic device was monitored using a 5× objective on an inverted microscope (Leica) equipped with a digital microscopy camera (Lumenera Infinity 3-1 CCD).

### Circular Dichroism Spectroscopy

Circular Dichroism (CD) spectroscopy was performed using an Aviv Model 202 instrument and a 1 mm quartz sample cell (Hellma). A-IDPs were prepared by dissolving the purified lyophilized product in 5 mM PBS, pH 7.4 at a final concentration of 10 μM. The CD spectra were obtained at 50° C. from 260 nm to 180 nm in 1 nm steps at a 0.5 second average time. Data points with a dynode voltage above 500 V were ignored in the analysis. The CD spectra were corrected for the 5 mM PBS buffer signal at 50° C. This data collection was repeated in triplicate, and the average of the three measurements was represented as molar ellipticity.

### Light Scattering

Dynamic light scattering (DLS) measurements were performed over a temperature range of 10-80° C. using a Wyatt DynaPro temperature-controlled microsampler (Wyatt Technology, Santa Barbara, Calif.). Samples for the DLS system were prepared in 1×PBS and filtered through 0.02 μm Whatman Anotop sterile syringe filters (GE Healthcare Life Sciences, Pittsburgh, Pa.) into a 12 μL quartz crystal cuvette (Wyatt Technology, Santa Barbara, Calif.). 5 acquisitions were taken at each temperature for a 5 second duration, and the results presented represent the mean Rₕ of the sample at each temperature.

### Temperature-Controlled UV-Vis Spectrophotometry

Cloud point transition temperatures (Tₜ) were determined via temperature-controlled spectrophotometry using a Cary 300 (Agilent Technologies). Samples containing various concentrations of protein in 150 mM PBS were cooled at 1° C./min while the absorbance at λ=350 nm was recorded every 1° C. Absorbance was normalized to the absorbance at the highest temperature point collected, corresponding to the more soluble point during a given experiment. The cloud point was determined as the maximum in the first derivative of the absorbance as a function of temperature. Transition temperature was calculated by the point of minimum slope. Saturation concentration was defined by the natural logarithm fit line created from a minimum of three volume fractions. Error bars are standard error of the mean from three repeats of a minimum of three transition temperatures.

### Dextran Uptake Experiments

The uptake of dextran molecules into the phase separated space of [WT]-20 and [Q_{5,8}]-20 was performed to quantify the isolation of A-IDPs from their surroundings. Fluorescein isothiocyanate labeled dextrans (10 kDa, 40 kDa, Sigma-Aldrich, St. Louis, Mo.) were added to 4 mg mL⁻¹ solutions of unlabelled [WT]-20 and [Q_{5,8}]-20 at final concentrations of 4 mg mL⁻¹ and 1 mg mL⁻¹, respectively at 60° C. Soluble samples were then transferred to room temperature glass slides and mounted with #1.5 cover slip. Samples were imaged on an upright Zeiss Axio Imager D2 microscope with a 20× objective and the appropriate filter set (ex 470/40, em 525/50) after 1-hour incubation below the transition temperature. Fluorescent intensity was calculated from background corrected fluorescent intensities inside/outside droplets in ImageJ portioned using bright field images of the phase separated space.

Sample Preparation for Temperature Gradient Experiments High concentration A-IDP stock solutions (60 wt %) were prepared by resuspending a mass of lyophilized A-IDP pellets with an appropriate volume of phosphate buffer saline solution (PBS) at a solution pH of 7.0. The concentration was converted to mg mL⁻¹ by assuming that the density of the A-IDP was 1 g mL⁻¹. The RLP stock solution was heated in a water bath at 85° C. for 60 minutes and mixed periodically along with sonication to ensure homogeneity. Lower concentration samples were made by mixing the initial stock solution volumetrically with PBS at a pH of 7. To prepare for temperature gradient microfluidics (TGM) measurements, the solutions were loaded into 12 mm×1 mm×0.1 mm rectangular borosilicate glass capillary tubes (VitroCom, Inc.), by capillary action, and sealed with wax to avoid sample evaporation and convection. The capillary tubes were held in contact with a hot plate at 85° C. housed within an incubator at 65° C. during the loading process. The high temperature environment ensured that the RLP solutions were held above the critical phase transition temperature (˜85° C. for [WT]-20). Capillary arrays were prepared by taping several capillaries together. The arrays were stored at 85° C. in an oven for 10 minutes prior to subjecting them to the temperature gradient experimentation.

### Measuring Phase Transition Temperatures on a Temperature Gradient Device

The temperature gradient device imposed a linear temperature gradient across the A-IDP solutions. This was accomplished by placing the glass capillary array into thermal contact with a heat source on one side and a cold sink on the other. The sample was then bathed in white light. This light was scattered by phase separated A-IDP droplets at cold temperature and was imaged via dark-field microscopy. The temperature gradient was calibrated for each experiment using two reference solutions placed alongside the A-IDP samples of interest. The cold temperature calibration reference contained 10 mg mL⁻¹ poly(N-isopropyl acrylamide) (PNIPAM) with MW=1.868×10⁵ g mol⁻¹ in H₂O (Polymer Source, Inc.). The hot temperature calibration reference contained 10 mg mL⁻¹ poly(ethylene oxide) (PEO) with MW=9×10⁵ g mol⁻¹ in a 1 M NaCl aqueous solution (Sigma-Aldrich). The LCST of each reference solution was obtained with a melting point apparatus that measured the light scattering intensity as the temperature was increased at a rate of 0.5 K min⁻¹. When placed onto the temperature gradient device, the reference solutions became cloudy at temperatures above the LCST. The pixel position of the LCST was obtained by the onset of light scattering intensity relative to the low intensity baseline on the cold side of the capillary. The temperature gradient was calculated using the pixel positions and the LCSTs of the two samples, assuming a linear relationship between position and temperature.

### Fitting of Phase Diagram Binodal

Fits for the roughly dilute, overlap and semi-dilute regions of our obtained phase diagrams were calculated using fitting methods for lower critical solution transition polypeptides adopted for upper critical solution transition polypeptides as described previously.

Briefly, for low volume fractions (ϕ<0.1), A-IDPs exhibit roughly a log-normal dependence on UCST cloud point with respect to volume fraction as seen with other repeat polypeptides. For the high density regime (ϕ>˜0.4) using surface tension scaling methods previously described for elastin-like polypeptides, we determined the coefficients of proportionality (A) and estimated theta temperature (θ) of [WT]-20 and [Q_{5,8}]-20 to be A=−0.00092, θ=389 K and A=−0.00092, θ=392 K respectively. In a poor solvent, the surface tension of a dilute phase globule γ can be written in the form γ≈C kTb2 ϕ″2 where b is the polypeptide Kuhn length (b=2.2 nm as measured by Fluegel and co-workers for other repetitive polypeptides) and C an adjustable coefficient. Replacing the surface tension γ by kTϕ₂\hat{ }2*A*(T−θ) we obtain an equation for the temperature dependence of the coacervate volume fraction

$${ϕ}_{2} = {[ \frac{A}{C} {b}^{2} ( T - θ ) ]}^{\frac{3}{4}}$$

Using a least-squares fit in Igor (WaveMetrics Inc. Portland, Oreg.), we adjust the coefficient C for this temperature dependence to match the measured [WT]-20 binodal and the [Q_{5,8}]-20 binodal points to determine C=0.62 and 1.05, respectively.

Closer to the critical point in the so-called Ginzburg zone one needs to use the critical Ising model to describe the phase behavior of polymer solutions. The phase boundary in the critical zone varies more gradually than predicted by mean field theory:

$${ϕ}_{2} - {ϕ}_{1} = {{C}_{c} ( \frac{T}{{T}_{c}} - 1 )}^{0.3}$$

where T_{c}=351.5 K for [WT]-20 and T_{c}=332.3 K for [Q_{5,8}]-20, 0.3 is the critical Ising exponent (Flory-Huggins mean field value is 0.5) for both [WT]-20 and [Q_{5,8}]-20, and C_{c} is the fitting coefficient. We calculated fitting coefficients in Igor (WaveMetrics Inc. Portland, Oreg.) equal to 1.29 and 1.27 for [WT]-20 and [Q_{5,8}]-20 respectively. Note that we calculated ϕ₁ explicitly using data collected with UV-Vis spectrophotometry and according to natural logarithm fits described in Table 1.

### Whole Cell Fluorescent Intensity Measurements

Cells were grown overnight in 5 mL of TB media from glycerol stocks. In conjunction to fluorescent or confocal imaging, cells were analyzed for total sfGFP fluorescence and OD₆₀₀. Briefly, 50 ul of cell culture at various time points was resuspended in 1 mL of 150 mM PBS. Using a combination of a UV-Vis spectrophotometry signal from a NanoDrop 1000 (Thermo Fisher Scientific, Waltham, Mass.) and fluorescent spectra from a NanoDrop 3300 (Thermo Fisher Scientific, Waltham, Mass.), we calculated the relative ratio of sfGFP fluorescence normalized to cell density. Using this information in conjunction with imaging analysis, we were able to determine the intracellular saturation concentration normalized to cell density.

Temperature Controlled Fluorescent Microscopy of Protocell Droplets and E. coli Bacteria

Water-in-oil droplets were collected on a glass microscope slide and cooled using a precision Peltier heating and cooling stage (Linkam LTS120) equipped with a temperature control unit (Linkam PE95). The spatial distribution of Alexa Fluor 350-labeled (25% molar fraction N-terminal labeled) [Q_{5,8}]-20 and Alexa Fluor 594-labeled +4 Net was characterized via fluorescence microscopy using an upright Zeiss Axio Imager D2 microscope with a 20× objective and the appropriate filter set. Similarly, intracellular pattering of A-IDP-superfolder GFP over time was characterized via fluorescence microscopy using an upright Zeiss Axio Imager D2 microscope with a 20× objective and the appropriate filter set (ex 470/40, em 525/50). Cell fluorescent was calculated using ImageJ software. Temperature ramps began at various temperatures but always were set to a constant speed of 5° C./min.

### Transient Transfection of [WT]-20-sfGFP in HEK293 Cells

[WT]-20-sfGFP was extracted from the pET24(+) vector using polymerase chain reaction (PCR). Briefly, the forward and reverse primers were resuspended with 1 ng of pET24(+) plasmid containing [WT]-20-sfGFP gene fusion. Using a PCR cycle of [98° C., 1 min; 65° C., 30 sec; 72° C., 2 min]×30 cycles, followed by gel purification, the gene was finally constructed with Gibson assembly. pcDNA5 vector containing [WT]-20-sfGFP was transfected into HEK293 cells according to manufacturer instructions (Expi293 Expression System, Thermo Fischer Scientific, Waltham, Mass.). Cells were spun down at 500 RCF for 10 min at room temperature on day 5 of transient transfection and resuspended in 150 mM PBS for imaging.

### Confocal Imaging of A-IDP-sfGFP Fusions for Puncta Formation and Colocalization

Cells were prepared as follows. A tube containing 5 mL of TB media was inoculated overnight with protein of choice from bacterial glycerol stock. After 16 hr of growth, induction with 1 mM IPTG and 2% L-rhamnose (Sigma-Aldrich, St. Louis, Mo.) was added each flask of interest. Samples were collected at the indicated time points and prepared for imaging as follows: 50 μL of cell suspension was pelleted under 20,000 RCF for 1 min at room temperature. Cells were resuspended to OD₆₀₀=0.15 at 1 cm path length. 50 μL of resuspended bacterial cells were transferred to a 384-well plated with #1.5 glass bottom (Cellvis). There was a 10 min equilibration period to the incubation chamber prior to each time point data collection.

Images were collected at different time points with a 63× oil-immersion objective on a Zeiss 710 inverted confocal with temperature-controlled incubation (Car Zeiss AG, Oberkochen, Germany). sfGFP fluorescent was detected with a 488 nm excitation laser and 488/594 emission filter. Data was primarily taken at 25° C. unless otherwise noted. All fluorescent quantification and cell portioning analysis was performed in ImageJ.

In colocalization experiments, cells were grown overnight from glycerol stock in dual antibiotic media containing 45 ug/mL kanamycin and 25 μg/mL chloramphenicol (final concentration). After 16-18 hours, pET24(+) expression was induced with 1 mM concentration of IPTG (final concentration). After 24 hours of IPTG induction, media was replaced with 5 mL of TB supplemented with 1 mM IPTG and 2% arabinose (final concentration) (Sigma Aldrich, St. Louis, Mo.). After 9 hours of induction with both, cells were prepared for confocal imaging by spinning down 50 μL of culture at room temperature and resuspended in 150 mM PBS to OD₆₀₀=0.15 at 1 cm path length. All imaging details remain the same except that mNeonGreen/sfGFP detection was performed with 488 nm excitation laser and 488/594 emission filter and mRuby3 detection with 561 nm excitation laser and 488/561 emission filter.

### Spinning Disc Confocal Imaging of Lac Z Alpha-Peptide-A-IDP Gene Fusions for Localization and Quantification of Enzymatic Activity

Cells were prepared as follows. A tube containing 5 mL of TB media was inoculated overnight with protein of choice from bacterial glycerol stock. After 16 hr of growth, induction with 1 mM IPTG and 2% L-rhamnose (Sigma-Aldrich, St. Louis, Mo.) was added each flask of interest. ˜24 hours later, 50 μL of cell suspension was pelleted under 20,000 RCF for 1 min at room temperature. Cells were resuspended in 150 mM PBS to OD₆₀₀=0.15 at 1 cm path length. Fifty microliters of sample were added to Culture-Insert 4 Well (1.5 coverslip, Ibidi, Madison, Wis.) petri dishes and allowed to incubate at room temperature for 10 minutes. After incubation, 2 μL of 1 mg mL⁻¹ FDG resuspended in 98% water, 1% DMSO and 1% EtOH was added. Imaging began immediately (within 20 seconds) and images were captured every minute for 30 minutes total. Imaging was performed on an Andor Dragonfly Spinning Disk 500 series confocal on a LeicaDMi8 microscope stand (Oxford Instruments, Abingdon, UK) with a 63× water immersion objective and equipped with a Zyla 4.2 series camera. Converted FDG was detected with a 488 nm excitation laser and 525/50 nm emission filter and mRuby3 fluorescence with a 561 nm excitation laser and 600/50 nm emission filter.

Fluorescent Spectroscopy for Determining Kₘ, Vₘₐₓ and k_{cat}

Liquid cultures of KRX E. coli containing plasmid of interest were grown from glycerol stocks overnight (16-18 hours). Cells were then induced with 1 mM IPTG and 2% L-rhamnose (Sigma-Aldrich, St. Louis, Mo.) for 24 hours. Cells were pelleted and resuspended at OD₆₀₀=˜0.15 in 140 mM PBS. Various concentrations of FDG were added while monitoring fluorescent intensity at 520 nm using NanoDrop 3300 (Thermo Fisher Scientific, Waltham, Mass.). The same instrument was used to also calculate the fluorescent intensity of mRuby3 as a relativistic measure of expression level of the various alpha peptide fusions. Plotting the observed fluorescent intensity at different times provides a surrogate measure of the rate of hydrolysis at various concentrations of the substrate (Vₒ). These rates were then converted into typical Lineweaver-Burk conventions to determine Vₘₐₓ and Kₘ. For consistency in units, [FDG] was converted into fluorescent intensity using a fluorescein standard curve of y=185919*[FDG in mg]+1045. This conversion assumes that converted FDG into fluorescein has similar fluorescent intensity profile to free fluorescein dye.

### Image Quantification and Statistical Analysis

For experiments performed with regard to determining the intracellular fluorescent intensity of A-IDP-sfGFP at various points post-IPTG induction the following statistical analysis was performed. For determining the saturation concentration intracellular, whole cell fluorescence normalized to cellular density (OD₆₀₀) on three independent samples was calculated while imaging of their intracellular architecture. Upon first observation of phase separation in E. coli in more than 50% of cells within a microscopic field of view, this normalized cell density was recorded as the saturation concentration. Data is normalized to data collected for [WT]-40 as a reference point. Error bars represent propagated standard error of the mean of three separate samples from the same original cell suspension.

With the microscope images collected with confocal microscopy at various time points, we isolated the soluble and puncta fractions within the cells at various points in time via analysis in ImageJ. Puncta consistently create pixels dense enough to saturate the detector while simultaneously observing the rest of the cell. Thus, by thresholding around the upper 2% of total pixel intensities, one can easily partition this section from the remaining cell cytoplasm. Using this constant thresholding between timepoints in each experimental group, we were able to track the total size of these puncta over time with regard to the total size of the cell (puncta+soluble fraction). Error bars of these data are standard errors of the mean of normalized puncta (two-phase) area of three images of different fields of view of the sample overall cell samples. These two channels are combined and split differently in FIG. 2 but have the same thresholding process applied to each image.

Given the lack of automated tools for the detection of intracellular phase separation between two images, we calculated the intracellular transition temperatures manually. Similar to the detection of phase separation with UV-Vis spectrophotometry, the intracellular transition temperature was determined as the midpoint between a frame that was certainly homogenous and a second frame that was certainly two phases. All transition temperatures were determined in this way, going from a point of solubility to insolubility whether the solution was being heated or cooled. Due to the level of subjectivity of this assessment, sample identifiers were blinded to the analyst and a high number of cells were analyzed in each experiment (n=30). Data was normalized to the initial mean fluorescence of the homogeneous cells at a consistent temperature (often 60° C. unless otherwise noted). Error bars indicate standard error of the mean.

The error bars of dextran fluorescence indicate the standard error of the mean fluorescence inside and outside of the phase separated space from three separate fields of view.

For quantification of Fluorescein Di-β-D-Galactopyranoside (FDG) relative to the different expression levels of alpha peptide, channels were split between fluorescence from FDG and mRuby3 respectively. Using the particle analysis tool from ImageJ, areas of green fluorescence were isolated from the background. If the mean fluorescence of this area was 5% greater than the background fluorescence (mean fluorescent of the area excluded by the previous particle mask), then this particular particle's background subtracted green fluorescence was included in the analysis. Particles were excluded if their area was below 0.1 um². Using the same particle mask, the background subtracted mean fluorescence of mRuby3 was calculated on the other fluorescent channel. We report the ratio of these two channels as a surrogate for enzymatic efficiency. Error bars are standard errors of the mean at each timepoint.

For quantification of Fluorescein Di-β-D-Galactopyranoside (FDG) inside the cellular space versus outside the cellular space, channels were first split between fluorescence from FDG and mRuby3 respectively. Using the same particle analysis tool from ImageJ, areas of green fluorescence were isolated from the background. If the mean fluorescence of this area was 5% greater than the background fluorescence (mean fluorescent of the area excluded by the previous particle mask), then this particular particle's background subtracted green fluorescence was included in the analysis. Particles were excluded if their area was below 0.1 um². Ratio of fluorescent intensity inside of cells versus the extracellular space is the background corrected mean fluorescence of FDG divided by the background fluorescence. Error bars are standard errors of the mean at each timepoint.

To quantify the amount of colocalization we used the Coloc2 plug-in available through ImageJ software. Using automated thresholding, we report the Mander's colocalization coefficient which accounts for the intensity to the two channels of interest as described previously.

Identification of a Minimal IDP Repeat from Proteomic Analysis and Sequence Heuristics

We conducted a proteomic analysis of 63 IDPs that form membrane-less organelles to investigate their sequence composition. We were particularly interested in categories of amino acids suspected to drive phase behavior via intrachain interactions, such as charge-charge, cation-π and hydrogen bonding via non-charged polar residues (FIG. 1A). The composition of these 63 proteins is remarkably similar to previously identified repetitive protein polypeptides that exhibit UCST phase behavior and their side-chains groups are chemically similar to synthetic UCST polymers. Using a combination of our previously developed sequence heuristics and insights from this proteomic analysis, we designed an octa-peptide motif that we expected would exhibit robust phase behavior when polymerized into a macromolecule, under physiologically relevant solution conditions.

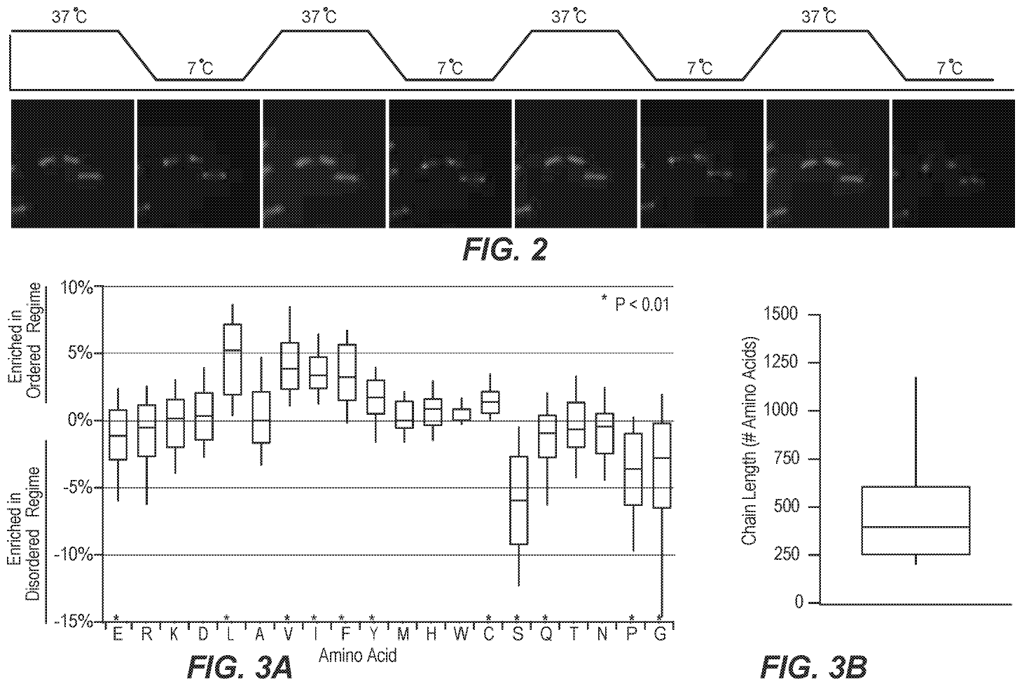

In order to manage the vast sequence space of all possible mutations of the octapeptide repeat, we classify each amino acid into categories of intrachain interactions that could contribute to UCST phase behavior. N, Q, S, T are classified as polar, uncharged amino acids. R-K and D-E are pairs of positively charged and negatively charged amino acids. G and P are placed into a separate category given their unusual structure and importance in promoting a disordered polypeptide backbone (FIG. 3A). The remaining amino acids are classified as “hydrophobic.” To ensure that we modulate the UCST phase behavior via mutagenesis of the WT repeat, but do not abolish it completely, we only make mutations wherein the mutant maintains the type of interactions and simply modulates the strength of that interaction. For example, R and K are both positively charged under normal physiological pH. Thus, by substituting K for R we maintain the charge neutral state of the polymeric backbone—a parameter known to dramatically affect the observed phase behavior. Similarly, N, Q, S and T are all capable of creating hydrogen bonds with water and one another more readily than an aliphatic amino acid such as V. Thus, substituting these four amino acids for one another maintains an equal number of residues per chain capable for forming this particular type of bond.

The wild-type (WT) repeat unit is (G-R₂-G₃-D₄-S₅-P₆-Y₇-S₈)₄₀ where 40 refers to the number of repeats. The MW of the A-IDPs was varied between ˜15 and ˜70 kDa—by varying the number of repeat motifs from 20 to 80—to account for observed differences in MW in the intrinsically disordered regions (IDRs) of naturally occurring IDPs (FIG. 3B). The parent sequence is referred to as WT in this paper, and we use a short-hand notation to refer to sequences throughout the text where the bracketed letter refers to a specific point—substitution—mutant. For example, a mutant with a complete substitution of Y₇ in the WT repeat unit with V would result in a notation of “[V₇]-XX”. When a residue is only partially substituted in the A-IDP, we use the notation “[DYₒ:ZVₒ]” where the B to Z ratio represents the ratio of Y to V ratio in the variant and the subscript o is the position of that residue along the repeat unit. For example, [Y₇:V₇]-40 would hence represent 50% of all Y replaced with V, whereas [3Y₇:V₇]-40 would represent a 25% substitution of V for Y. A double mutant, such as 100% substitution of residues at the 5^{th} and 8^{th} position in the octapeptide repeat with Q, would be denoted as [Q_{5,8}]-XX with and fractional substitution at these positions with S and Q would be denoted as [BS_{5,8}:ZQ_{5,8}]-XX where B and Z represent the ratio of S to Q. Full sequence descriptions of common sequences used throughout the paper can be found in Table 1. A full description of all architectures of A-IDPs wherein mutant and WT repeats are mixed along the A-IDP chain can be found in Table 2 and Table 3.

TABLE 1
Amino Acid Sequences of A-IDPs

| | | | Mol. | SEQ |
|---|---|---|---|---|
| Protein | Full Amino Acid | Aa | Wt. | ID |
| Name | Sequence | (N) | (Da) | NO: |
| [WT]-20 | SKGP-[GRGDSPYS]₂₀-GY | 166 | 17004 | 197 |
| [WT]-40 | SKGP-[GRGDSPYS]₄₀-GY | 326 | 33400 | 198 |
| [WT]-60 | SKGP-[GRGDSPYS]₆₀-GY | 486 | 49797 | 199 |
| [WT]-80 | SKGP-[GRGDSPYS]₈₀-GY | 646 | 66193 | 200 |
| [Q_{5,8}]-20 | SKGP-[GRGDQPYQ]₂₀-GY | 166 | 18646 | 201 |
| [Q_{5,8}]-40 | SKGP-[GRGDQPYQ]₁₀-GY | 326 | 36685 | 202 |
| [3S_{5,8}:Q_{5,8}]- | SKGP-[GRGDSPYSGRGD | 326 | 34221 | 203 |
| 40 | SFYSGRGDSP | | | |
| | YSGRGDQPYQ]₁₀-GY | | | |
| [S_{5,8}:Q_{5,8}]- | SKGP-[GRGDSPYSGR | 326 | 35042 | 204 |
| 40 | GDQPYQ]₂₀-GY | | | |
| [S_{5,8}:3Q_{5,8}]- | SKGP-[GRGDQ | 326 | 35863 | 205 |
| 40 | PYQGRGDQPYQGR | | | |
| | GDQPYQGRGDSFYS]₁₀-GY | | | |
| [3Y₇:V₇]- | SKGP-[GRGDSP | 326 | 32760 | 206 |
| 40 | YSGRGDSPYSGRG | | | |
| | DSPYSGRGDSPVS]₁₀GY | | | |
| [Y₇:V₇]-40 | SKGP-[GRGDSPYSGRG | 326 | 32119 | 207 |
| | DSPVS]₂₀-GY | | | |
| [V₇]-40 | SKGP-[GRGDSPVS]₄₀- | 326 | 30839 | 208 |
| | GY | | | |
| [3R₂:K₂]- | SKGP-[GRGDSPY | 326 | 33120 | 209 |
| 40 | SGRGDSPYSGRGD | | | |
| | SPYSYGKGDSPS]₁₀-GY | | | |
| [R₂:K₂]- | SKGP-[GRGDSPYSGKG | 326 | 32840 | 210 |
| 40 | DSPYS]₂₀-GY | | | |

TABLE 2
Amino Acid Sequence of A-IDPs with
a Single Repeat Motif

| | | | Mol. | μM, | | SEQ |
|---|---|---|---|---|---|---|
| Protein | Full Amino Acid | AA | Wt. | 37° | image | ID |
| Name | Sequence | No. | (Da) | C. | index | NO: |
| [WT]-20 | SKGP-[GRGDSPYS]₂₀-GY | 166 | 17004 | 44.18 | 1 | 197 |
| [WT]-40 | SKGP-[GRGDSPYS]₄₀-GY | 326 | 33400 | 0.755 | 2 | 198 |
| [WT]-60 | SKGP-[GRGDSPYS]₆₀-GY | 486 | 49797 | 0.044 | 3 | 199 |
| [WT]-80 | SKGP-[GRGDSPYS]₈₀-GY | 646 | 66193 | 0.0002 | 4 | 200 |
| [Q_{5,8}]-20 | SKGP-[GRGDQPYQ]₂₀-GY | 166 | 18646 | 247.9 | 8 | 201 |
| [Q_{5,8}]-40 | SKGP-[GRGDQPYQ]₄₀-GY | 326 | 36685 | 6.593 | 9 | 202 |
| [Q_{5,8}]-60 | SKGP-[GRGDQPYQ]₆₀-GY | 486 | 54723 | 1.034 | 10 | 211 |
| [Q_{5,8}]-80 | SKGP-[GRGDQPYQ]₈₀-GY | 646 | 72762 | 0.241 | 11 | 212 |
| [T_{5,8}]-40 | SKGP-[GRGDTPYT]₄₀-GY | 326 | 34522 | 0.856 | 60 | 213 |
| [N_{5,8}]-20 | SKGP-[GRGDNPYN]₂₀-GY | 166 | 18085 | 337.2 | 12 | 214 |
| [N_{5,8}]-40 | SKGP-[GRGDNPYN]₄₀-GY | 326 | 35562 | 39.18 | 13 | 215 |
| [N_{5,8}]-60 | SKGP-[GKGDNPYN]₆₀-GY | 486 | 53040 | 4.827 | 14 | 216 |
| [H₇]-20 | SKGP-[GRGDSPHS]₂₀-GY | 166 | | | | 217 |
| [H₇]-40 | SKGP-[GRGDSPHS]₄₀-GY | 326 | 32359 | 8376.0 | 61 | 218 |
| [H₇]-60 | SKGP-[GRGDSPHS]₆₀-GY | 486 | 48235 | 286.8 | 24 | 219 |
| [H₇]-80 | SKGP-[GRGDSPHS]₈₀-GY | 646 | 64111 | 327.0 | 25 | 220 |
| [F₇]-20 | SKGP-[GRGDSPFS]₂₀-GY | 166 | | | | 221 |
| [F₇]40 | SKGP-[GRGDSPFS]₄₀-GY | 326 | 32760 | 1.923 | 62 | 222 |
| [Q₈]-20 | SKGP-[GRGDSPYQ]₂₀-GY | 166 | 17825 | 0.029 | 38 | 223 |
| [Q₈]-40 | SKGP-[GRGDSPYQ]₄₀-GY | 326 | 35042 | 0.135 | 39 | 224 |
| [Q₈]-60 | SKGP-[GRGDSPYQ]₆₀-GY | 486 | 52260 | 3.179 | 40 | 225 |
| [Q₈]-80 | SKGP-[GRGDSPYQ]₈₀-GY | 646 | 69478 | 15.72 | 41 | 226 |
| [Q₅]-20 | SKGP-[GRGDQPYS]₂₀-GY | 166 | 17825 | 149.0 | 29 | 227 |
| [Q₅]-40 | SKGP-[GRGDQPYS]₄₀-GY | 326 | 35042 | 7.941 | 30 | 228 |
| [Q₅]-60 | SKGP-[GRGDQPYS]₆₀-GY | 486 | 52260 | 0.723 | 31 | 229 |
| [Q₅]-80 | SKGP-[GRGDQPYS]₈₀-GY | 646 | 69478 | 0.086 | 32 | 230 |
| [N₅]-20 | SKGP-[GRGDNPYS]₄₀-GY | 166 | | | | 231 |
| [N₅]-40 | SKGP-[GRGDNPYS]₄₀-GY | 326 | 34481 | 0.670 | 21 | 232 |
| [N₅]-60 | SKGP-[GRGDNPYS]₆₀-GY | 486 | 51418 | 0.028 | 22 | 233 |
| [N₅]-80 | SKGP-[GRGDNPYS]₈₀-GY | 646 | 68356 | 0.004 | 23 | 234 |
| [N₈]-20 | SKGP-[GRGDSPYN]₂₀-GY | 166 | 17544 | 484.1 | 18 | 235 |
| [N₈]-40 | SKGP-[GRGDSPYN]₄₀-GY | 326 | 34481 | 16.17 | 19 | 236 |
| [N₈]-60 | SKGP-[GRGDSPYN]₆₀-GY | 486 | 51418 | 3.748 | 20 | 237 |
| [N₅, Q₈]-20 | SKGP-[GRGDNPYQ]₂₀-GY | 166 | | | | 238 |
| [N₅, Q₈]-40 | SKGP-[GKDNPYQ]₄₀-GY | 326 | 36123 | 2.449 | 5 | 239 |
| [N₅, Q₈]-60 | SKGP-[GRGDNPYQ]₆₀-GY | 486 | 53882 | 0.176 | 6 | 240 |
| [N₅, Q₈]-₈0 | SKGP-[GRGDNPYQ]₈₀-GY | 646 | 71640 | 0.108 | 7 | 241 |
| [Q₅, N₈]-20 | SKGP-[GRGDQPYN]₂₀-GY | 166 | 18365 | 637.5 | 15 | 242 |
| [Q₅, N₈]-40 | SKGP-[GRGDQPYN]₄₀-GY | 326 | 36123 | 52.62 | 16 | 243 |
| [Q₅, N₈]-60 | SKGP-[GRGDQPYN]₆₀-GY | 486 | 53882 | 13.47 | 17 | 244 |
| [WT]20-sfGFP | SKGP-[GRGDSPYS]₂₀-sfGFP | 410 | 44551 | 216.1 | 26 | 245 |
| [WT]40-sfGFP | SKGP-[GRGDSPYS]₄₀-sfGFP | 570 | 60947 | 3.448 | 27 | 246 |

TABLE 3
Amino Acid Sequence of A-IDPs with Multiple Repeat Motifs

| | | | Mol. Wt. | pM, | Image | SEQ ID |
|---|---|---|---|---|---|---|
| Protein Name | Full Amino Acid Sequence | AA No. | (Da) | 37 °C | Index | NO: |
| [3S_{5,8}:Q_{5,8}]-40 | SKGP- | 326 | 34221 | 1.600 | 33 | 254 |
| | [GRGDSPYSGRGDSFYSGRGDSPYS | | | | | |
| | GRGDQPYQ]₁₀-GY | | | | | |
| [S_{5,8}:Q_{5,8}]-40 | SKGP-[GRGDSPYSGRGDQPYQ]₂₀- | 326 | 35042 | 1.907 | 34 | 255 |
| | GY | | | | | |
| [S_{5,8}:3Q_{5,8}]-40 | SKGP- | 326 | 35863 | 5.062 | 35 | 256 |
| | [GRGDQPYQGRGDQPYQGRGDQPYQ | | | | | |
| | GRGDSPYS]₁₀-GY | | | | | |
| [3Y₇:V₇]-40 | SKGP- | 326 | 32760 | 24.54 | 36 | 257 |
| | [GRGDSPYSGRGDSFYSGRGDSPYS | | | | | |
| | GRGDSPYS]₁₀-GY | | | | | |
| [Y₇:V₇]-40 | SKGP-[GRGDSPYSGRGDSPVS]₂₀- | 326 | 32119 | 815.5 | 37 | 258 |
| | GY | | | | | |
| [Y₇:3V₇]-40 | SKGP-[GRGDSPYSGRGDSPVS]₂₀ | 326 | 31479 | Unk | N/A | 259 |
| | GY | | | | | |
| [V₇]-40 | SKGP-[GRGDSPVS]₄₀-GY | 326 | 30839 | Unk | N/A | 260 |
| [3Y₇:A₇]-40 | SKGP- | 326 | 32479 | 28.46 | 45 | 261 |
| | [GRGDSPYSGRGDSPYSGRGDSPYS | | | | | |
| | GRGDSPAS]₁₀-GY | | | | | |
| [Y₇:A₇]-40 | SKGP-[GRGDSPYSGRGDSPAS]₂₀ | 326 | 31558 | 1816 | 46 | 262 |
| | GY | | | | | |
| [3Y₇:H₇]-40 | SKGP- | 326 | 32900 | 12.88 | 47 | 263 |
| | [GRGDSPYSGRGDSPYSGRGDSPYS | | | | | |
| | GRGDSPIS]₁₀-GY | | | | | |
| [Y₇:I₇]-40 | SKGP-[GRGDSPYSGRGDSPIS]₂₀- | 326 | 32400 | 214.8 | 48 | 264 |
| | GY | | | 506 | | |
| [3Y₇:M₇]-40 | [GRGDSPYSGRGDSPYSGRGDSPYS | 326 | 33080 | 7.914 | 49 | 265 |
| | GRGDSPMS]₁₀-GY | | | | | |
| [Y₇:M₇]-40 | SKGP-[GRGDSPYSGRGDSPMS]₂₀- | 326 | 32761 | 110.5 | 50 | 266 |
| | GY | | | | | |
| [3Y₇:H₇]-40 | SKGP- | 326 | 33140 | 7.125 | 42 | 267 |
| | [GRGDSPYSGRGDSPYSGRGDSPYS | | | | | |
| | GRGDSPHS]₁₀-GY | | | | | |
| [Y₇:H₇]-40 | SKGP-[GRGDSPYSGRGDSPHS]₂₀- | 326 | 32880 | 70.86 | 43 | 268 |
| | GY | | | | | |
| [Y₇:3H₇]-40 | SKGP- | 326 | 32619 | 508.5 | 44 | 269 |
| | [GRGDSPHSGRGDSPHSGRGDSPHS | | | | | |
| | GRGDSPYS]₁₀-GY | | | | | |
| [3R₂:K₂]-40 | SKGP- | 326 | 33120 | 8.537 | 51 | 270 |
| | [GRGDSPYSGRGDSPYSGRGDSPYS | | | | | |
| | GKGDSPYS]₁₀-GY | | | | | |
| [R₂:K₂]-40 | SKGP-[GRGDSPYSGKGDSPYS]₂₀- | 326 | 32840 | 79.88 | 52 | 271 |
| | GY | | | | | |
| [D₄:E₄]-40 | SKGP-[GRGDSPYSGRGESPYS]₂₀- | 326 | 33681 | 1.779 | 53 | 272 |
| | GY | | | | | |
| [3Y₇:W₇]-40 | SKGP- | 326 | 33671 | 0.066 | | 273 |
| | [GRGDSPYSGRGDSPYSGRGDSPYS | | | | | |
| | GRGDSPWS]₂₀-GY | | | | | |
| [Y₇:W₇]-40 | SKGP-[GRGDSPYSGRGDSPHS]₂₀- | 326 | 33901 | 0.023 | | 274 |
| | SKGP[GRPDSPYSGRGDSPYSGRGD | | | | | |
| | SPYSGRGDSPYSPRGDSPYSGRGDS | | | | | |
| | PYSGRGDSPYSGRGDSPYSGRPDSP | | | | | |
| | YSGRGDSPYSGRGDSPYSGRGDSPY | | | | | |
| [5G:7P]-40 | SPRGDSPYSGRGDSPYSGRGDSPYS | 326 | 33801 | 0.742 | 54 | 275 |
| | GRGDSPYS | | | | | |
| | GRPDSPYSGRGDSPYSGRGDSPYSG | | | | | |
| | RGDSPYS]₂-GY | | | | | |
| [G:P]-40 | SKGP- | 326 | 34202 | 0.728 | 55 | 276 |
| | [GRPDSPYSGRGDSPYSPRGDSPYS | | | | | |
| | GRGDSPYS]₁₀-GY | | | | | |
| [7G:5P]-40 | SKGP- | 326 | 34602 | 0.910 | 56 | 277 |
| | [GRPDSPYSPRGDSPYSGRPDSPYS | | | | | |
| | GRGDSPYSPRGDSPYSGRPDSPYSP | | | | | |
| | RGDSPYSGRGDSPYSGRPDSPYSPR | | | | | |
| | GDSPYSGRPDSPYSGRGDSPYSPRG | | | | | |
| | DSPYSGRPDSPYSPRGDSPYSGRGD | | | | | |
| | SPYSGRPDSPYSPRGDSPYSGRPDS | | | | | |
| | PYSGRGDSPYS]₂GY | | | | | |
| [3Y₇:V₇]-40- | SKGP- | 570 | 60307 | 42.46 | 28 | 278 |
| sfGFP | [GRGDSPYSGRGDSPYSGRGDSPYS | | | | | |
| | GRGDSPVS]10-sfGFP | | | | | |
| [3Y₇:V₇]-40- | ([GRGDSPYSGRGDSPYSGRGDSPY | 323 | 32660 | Unk | N/A | 279 |
| UAA | SGRGDSPVS]-5-AzF)2 | | | | | |

TABLE 4
Amino Acid Sequence of Fluorescent
Protein Reporters

| | | | | SEQ ID |
|---|---|---|---|---|
| | | | Mol. | NO: |
| | Full Amino Acid | AA | Wt. | (AA, |
| Protein | Sequence | No. | (Da) | DNA) |
| sfGFP | GKGEELFTGVVPILVELDGD | 246 | 27785 | 280, |
| | VNGHKFSVRGEGEGDATNGK | | | 281 |
| | LTLKFICTTGKLPVPWPTLV | | | |
| | TTLTYGVQCFSRYPDHMKRH | | | |
| | DFFKSAMPEGYVQERTISFK | | | |
| | DDGNYKTRAEVKFEGDTLVN | | | |
| | RIELKGIDFKEDGNILGHKL | | | |
| | EYNYNSHNVYITADKQKNGI | | | |
| | KANE | | | |
| mRuby3 | KIRHNIEDGSVQLADHYQQN | 237 | 26486 | 282, |
| | TPIGDGPVLLPDNHYLSTQS | | | 283 |
| | VLSKDPNEKRDHMVLLEFVT | | | |
| | AAGITHGMDELYKELHHHHH | | | |
| | HGGVSKGEELIKENMRMKVV | | | |
| | MEGSVNGHQFKCTGEGEGRP | | | |
| | YEGVQTMRIKVIEGGPLPFA | | | |
| | FDILATSFMYGSRTFIKYPA | | | |
| | DIPDFFKQSFPEGFTWERVT | | | |
| | RYEDGGVVTVTQDTSLEDGE | | | |
| | LVYNVKVRGVNFPSNGPVMQ | | | |
| | KKTKGWEPNTEMMYPADGGL | | | |
| | RGYTDIALKVDGGGHLHCNF | | | |
| | VTTYRSKKTVGNIKMPGVHA | | | |
| | VDHRLERIEESDNETYWQRE | | | |
| | VAVAKYSNLGGGMDELYK | | | |

TABLE 5
Primers for pcDNA5 Cloning of [WT]-20-sfGFP

| | | |
|---|---|---|
| PCDNA5-Fwd- | CTCACTATAGGGAGACCCAAG | SEQ ID NO: |
| [WT]-20- | CTGGCTAGCATGAGCAAAGGG | 282 |
| sfGFP | CCGGGACGCGGCGATAGT | |
| PcDNA5-Rev- | TTAGCCGTGATGGTGATGGTG | SEQ ID NO: |
| [WT]-20- | ATGGAGCTCGTTGATTGTCGA | 283 |
| sfGFP | GGGCCCTCTAGACTCGAG | |

### A-IDPs Exhibit Robust and Reversible UCST Phase Behavior in an Aqueous Environment

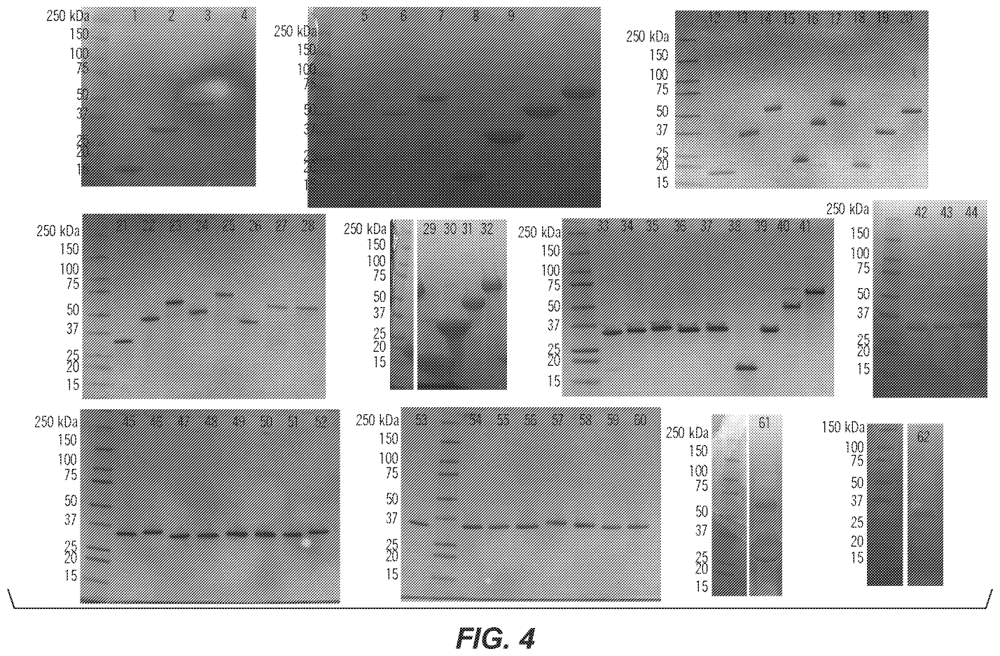

One advantage of A-IDPs is their minimal interaction with other proteins or biomolecules stemming from their repetitive nature. This feature of A-IDPs combined with their reversible aqueous two-phase separation enables simple column-free purification by UCST phase transition cycling between the one- and two-phase regime of the phase diagram. An example of this purification process is shown in FIG. 1B, where the highly expressing A-IDP, [Q_{5,8}]-20, completely phase separates from the soluble fraction of the cell lysate and can be isolated by centrifugation. Subsequent removal of the protein-poor supernatant, dissolution of the protein-rich pellet with urea, and dialysis of the soluble fraction in milli-Q water results in 95-99% pure protein as observed by sodium dodecyl sulfate polyacrylamide gel electrophoresis (SDS-PAGE) (FIG. 1C and FIG. 4). The yield of purified A-IDP ranges from 25-300 mg per liter of culture media in shaker flask culture.

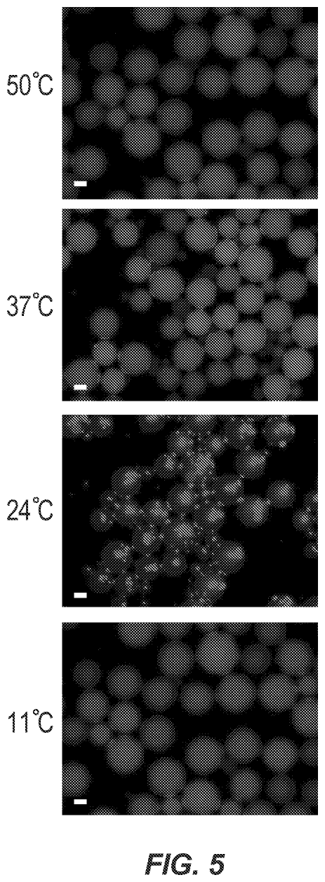

A-IDPs [WT]-20 and [Q_{5,8}]-20 exhibit UCST phase behavior in vitro. To characterize their phase transition behavior, we employed three different techniques. First, we utilized droplet microfluidics, where monodisperse water droplets are formed in oil containing the A-IDP of interest (FIG. 1D). Phase separation can be directly visualized in the spatially limited compartment of water-in-oil microdroplets to observe the types of structures formed as one cools the surrounding medium. These A-IDPs exhibit classic liquid-liquid phase separation where, upon crossing the phase boundary upon cooling from 50° C. to 10° C., multiple nucleation sites of coacervate condensates are observed (FIG. 1D Panel 2). These nucleation sites wet one another and quickly coalesce into a single, spherical A-IDP-dense phase that is in equilibrium with the surrounding A-IDP-poor phase (FIG. 1D panel 3 and 3-1/3-2). Upon reheating to 50° C., the A-IDP-rich phase shrinks in size, as the A-IDP re-solubilizes, re-establishing equilibrium rapidly (FIG. 1D Panel 4-1/4-2). A wider field of view of this transition can be found in FIG. 5. These data clearly show that these A-IDPs exhibit reversible UCST phase separation via coalescence and growth kinetics (FIG. 1E).

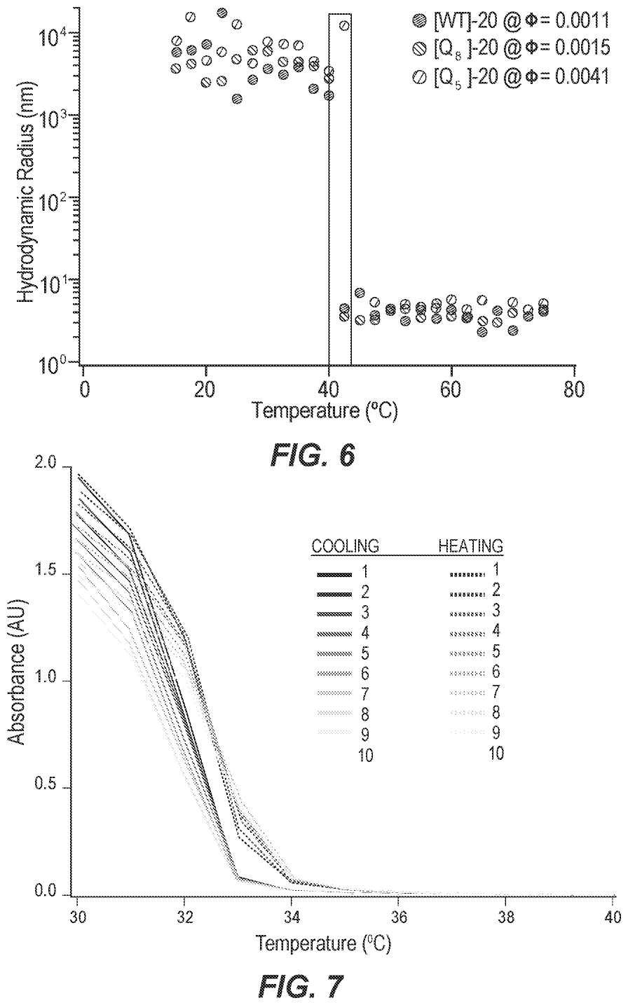

Second, we employed temperature dependent dynamic light scattering (DLS) to observe the two-phase separation in bulk. A solution of [Q_{5,8}]-20 is heated to 80° C. and DLS data was collected as the solution is cooled to 10° C. We observe a transition from soluble A-IDP molecules with a hydrodynamic radius (Rₕ) of 4 nm to aggregates larger than 1 μm as a function of temperature (FIG. 1F and FIG. 6). This transition is quite sharp, as it occurs within a 2° C. window at ˜38° C.

Third, we employed temperature-dependent turbidity measurements at a fixed wavelength of 350 nm to characterize the UCST phase separation while heating and cooling a solution of an A-IDP at a rate of 1° C. min⁻¹ (FIG. 1G). Using this technique, we can capture partial phase diagrams of each A-IDP of interest as a function of many different sequence and solution parameters. At dilute volume fractions of [Q_{5,8}]-40 we observed different UCST cloud points that increase as a function of the natural logarithm of A-IDP concentration (Tₜ=m*ln([A-IDP])+b). We also confirmed the complete reversibility of the UCST phase behavior of these A-IDPs, with a <1° C. difference in UCST Tₜ after ten successive heating and cooling ramps (FIG. 7).

### Arginine Composition, Aromatic to Aliphatic Ratio, Charge Balance and Molecular Weight Define UCST Cloud Point

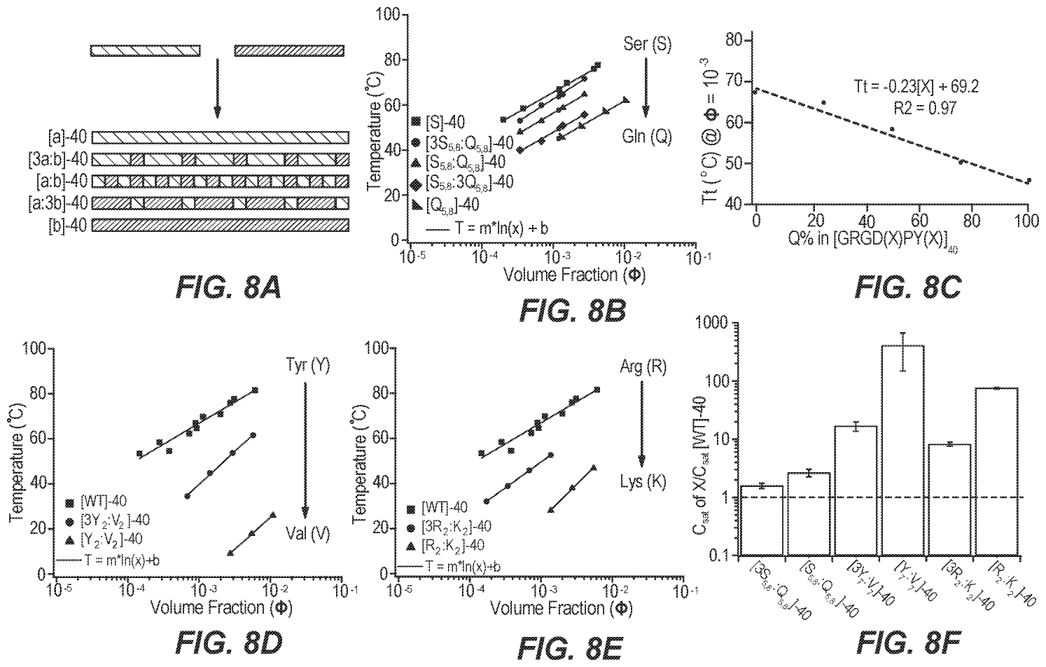

To understand the effects of a particular residue substitution in the octapeptide repeat on the phase separation for the A-IDP, we created a set of “mutant” A-IDPs ranging from 100% of a to 100% of b where a is the WT repeat unit. The doping scheme wherein the mutant repeat unit b is periodically inserted into the WT sequence is visually illustrated by the color-coded schematic in FIG. 8A. The mutant repeat is well mixed-distributed along the WT sequence to reduce “blockiness” of the co-polypeptide, which has been shown in LCST polypeptides to lead to nanoscale self-assembly instead of the desired liquid-liquid coacervation. Measuring the UCST phase behavior of these copolymers is analogous to a loss-of-function or gain-of-function screen for UCST cloud point upon substitution of motif b for a (FIG. 8A). Due to experimental limitations, loss of function—phase separation that cannot be detected—is operationally defined as a Tₜ<4° C. at volume fractions less than or equal to 0.1 in a 140 mM salinity aqueous buffer. A total length of 40 repeats was chosen for these A-IDPs to approximate the median length (˜320 amino acids) of IDRs found in the proteomic analysis of naturally occurring IDPs.

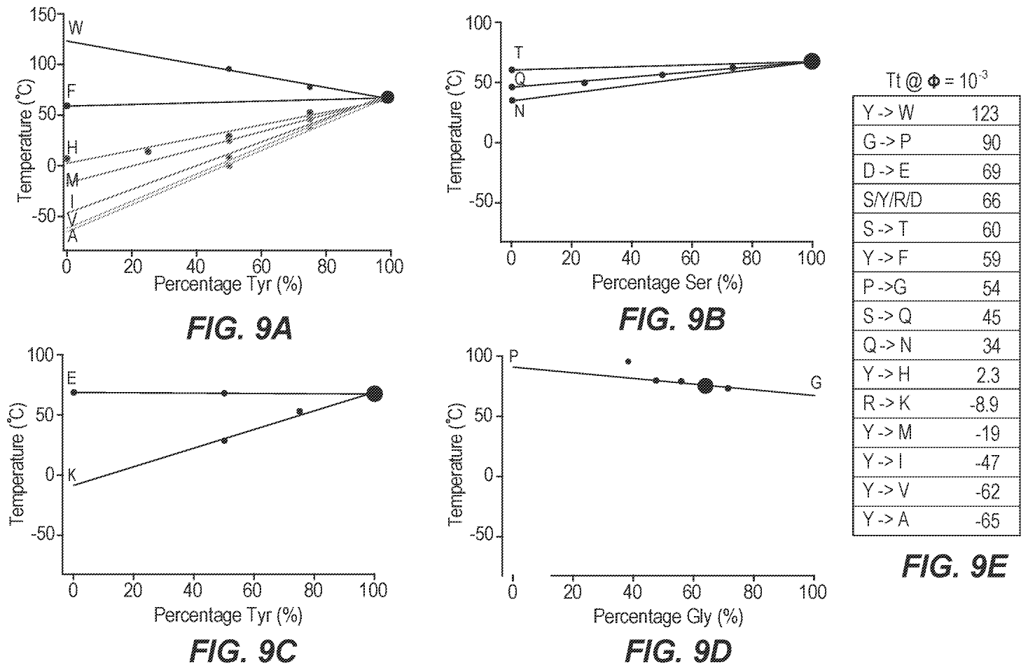

The Tₜ of the WT and each A-IDP is a linear function of its volume fraction (ϕ) (FIGS. 8B and C). At a specified ϕ₁ the Tₜ is a function of composition (R²=0.97), which demonstrates that the behavior of the mutant A-IDPs-block co-polypeptides of a and b—can be linearly interpolated between that of pure polypeptides of a and b. The linear behavior of the Tₜ of these mutant A-IDPs also allows extrapolation of the UCST phase behavior for homopolymers that exhibit a UCST cloud point beyond the experimentally observable range of detection thus putting each point mutation on a single relative scale (FIG. 9).

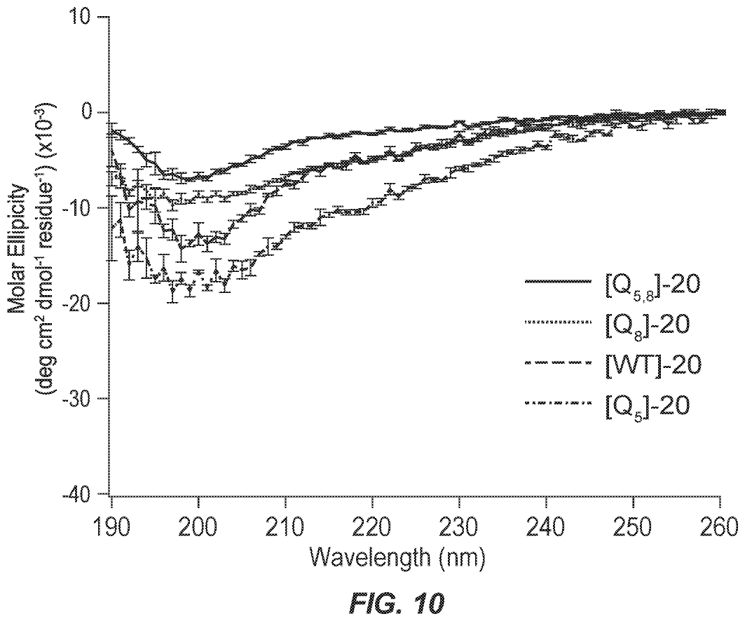

We next tested the effect of fifteen different site-specific substitution mutations of the reference—WT—repeat motif on the saturation concentration (Cₛₐₜ)—defined as is the concentration at which the Tₜ is 37° C.—of the A-IDPs. We found that single residue changes in the octapeptide repeat are capable of changing the Cₛₐₜ—normalized to the degree of substitution defined by the percent change in amino acid composition—of the repeat polypeptide by over by two orders of magnitude at constant molecular weight that ranged from 1-800 μM (FIGS. 8D, E and F). This can be visualized by normalizing to the saturation concentration of [WT]-40 which is conveniently ˜1 μM and is shown by the dashed horizontal line in FIG. 8F. We do not believe that changes in chain conformation as a result of these mutations is responsible for these effects on the Tₜ and Cₛₐₜ Indeed, circular dichroism spectrophotometry shows that the mutant A-IDPs are structurally disordered, consistent with their G- and P-rich composition (FIG. 10).

These substitutions present quantitative evidence for the importance of interactions between R and aromatic residues in the repeat motif of the A-IDP. When Y₇ is substituted, we observe dramatic shifts in the UCST cloud point at ϕ=10⁻³, from 66° C. to 123° C., 59° C. and ˜2° C. for W, F or H respectively (FIG. 9A). These data indicate that interactions between the cationic side-chain of R and the aromatic side-chain of W, F, Y and H are important driving forces for phase separation, although the strength of these interactions is side-group dependent with W>>>Y>F>>>H. Likewise, replacing R for K lowers the UCST cloud point temperature and hence the phase boundary (FIG. 8E) and increases the Cₛₐₜ (FIG. 8F).

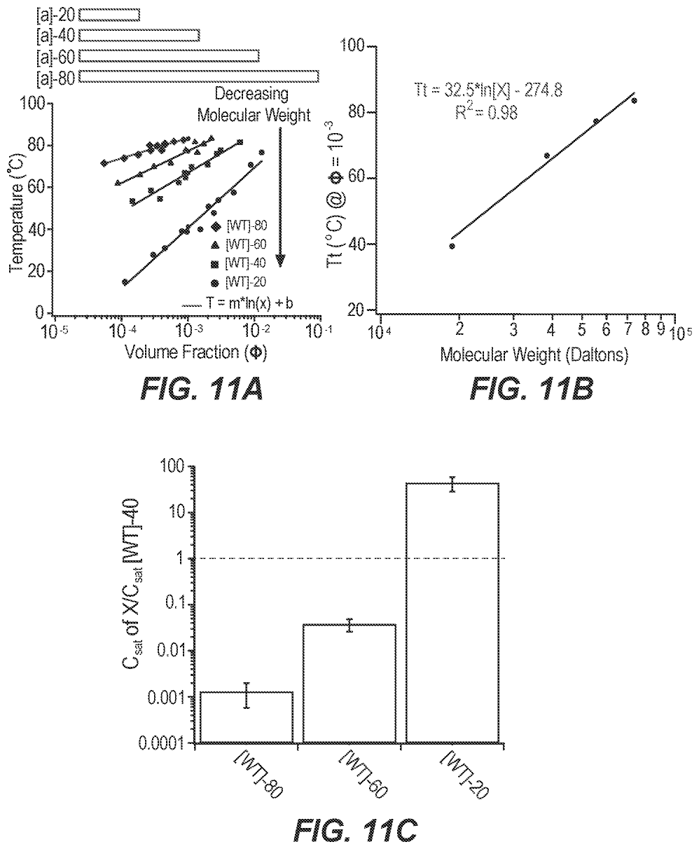

We next looked at the effect of A-IDP MW on phase behavior, we chose A-IDPs with MWs between ˜17 kDa and ˜70 kDa, as this MW range covers 75% of the IDRs in our proteomic analysis of native IDPs (FIG. 8B). Our results indicate that MW exhibits at least as large an effect on UCST cloud point as amino acid substitution (FIG. 11A). We observed that the effect of MW on Tₜ—in the ˜17-70 kDa range that we studied—can be approximated with a linear fit to the natural logarithm of MW (FIG. 11B). By simply doubling the MW of [WT]-40, we were able to create A-IDPs with predicted Cₛₐₜ in the nanomolar regime (FIG. 11C), similar to the Cₛₐₜ exhibited by some native IDPs. Notably, by varying both the MW and composition we can vary the Cₛₐₜ of A-IDPs by over seven orders of magnitude, ranging from 10⁻⁴ to 10² μM.

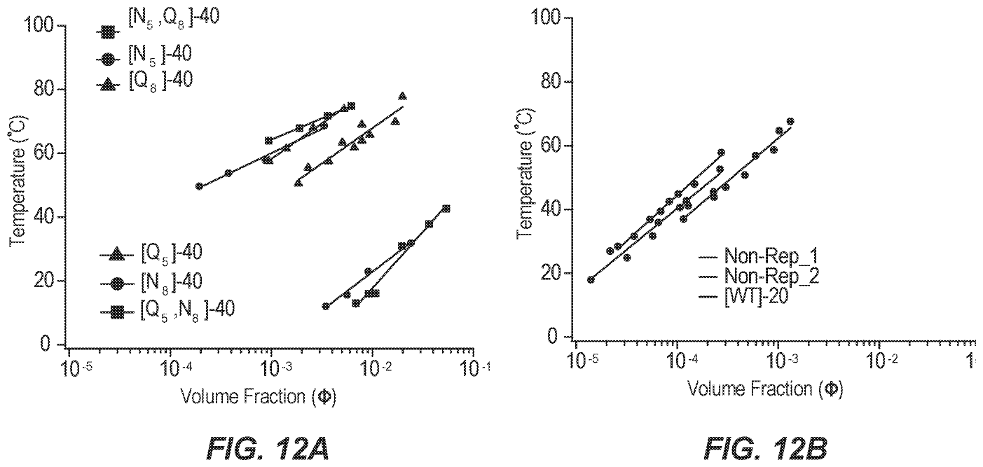

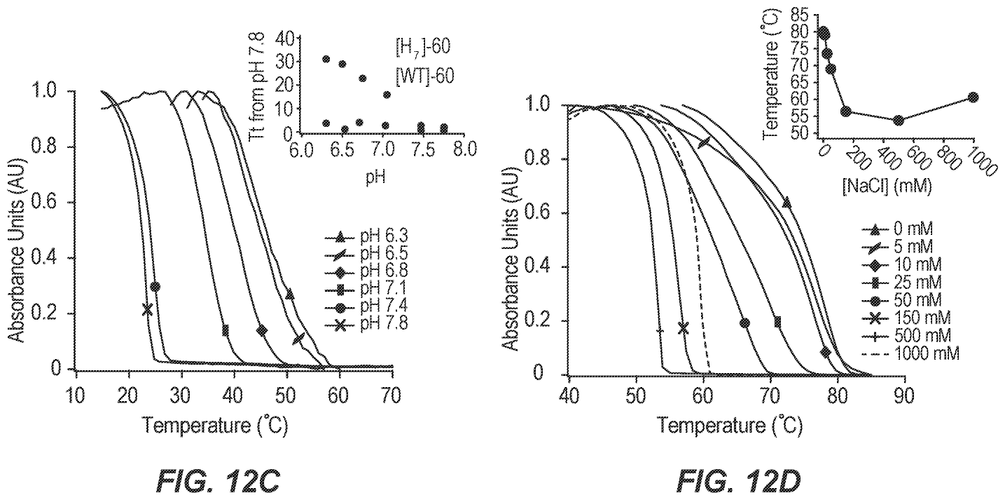

In addition to composition, concentration (ϕ) and MW on Tₜ, there are several other parameters that have a measurable effect on UCST phase behavior but that do not eliminate UCST phase behavior under physiologically relevant conditions. Uncharged polar substitutions, the ratio of G/P, the syntax of the repeating polypeptide, solution salt content, pH (in the absence of H) and identity of the negatively charged amino acid (E vs. D) all result in smaller changes to the UCST binodal phase boundary than MW, volume fraction, aromatic:aliphatic amino acid ratio and R content (FIG. 9B-C). The residue N-terminal to P₆ appears to have a unique impact on the UCST binodal boundary where compositionally identical A-IDPs shifted the UCST binodal lines depending on which polar non-charged reside is located at position 5 of the octapeptide repeat (FIG. 12A). We also produced and tested non-repetitive, but compositionally identical versions of [WT]-20 and observed minimal effects of scrambling the amino acid sequence on the UCST binodal (FIG. 12B). Collectively, these results indicate that three parameters—the aromatic:aliphatic ratio, the volume fraction (ϕ), and the MW are the most important for controlling UCST phase boundaries or Cₛₐₜ in vitro.

### A-IDPs Create Dense Phase Separated Condensates at Saturation Concentrations Mediated by Amino Acid Composition

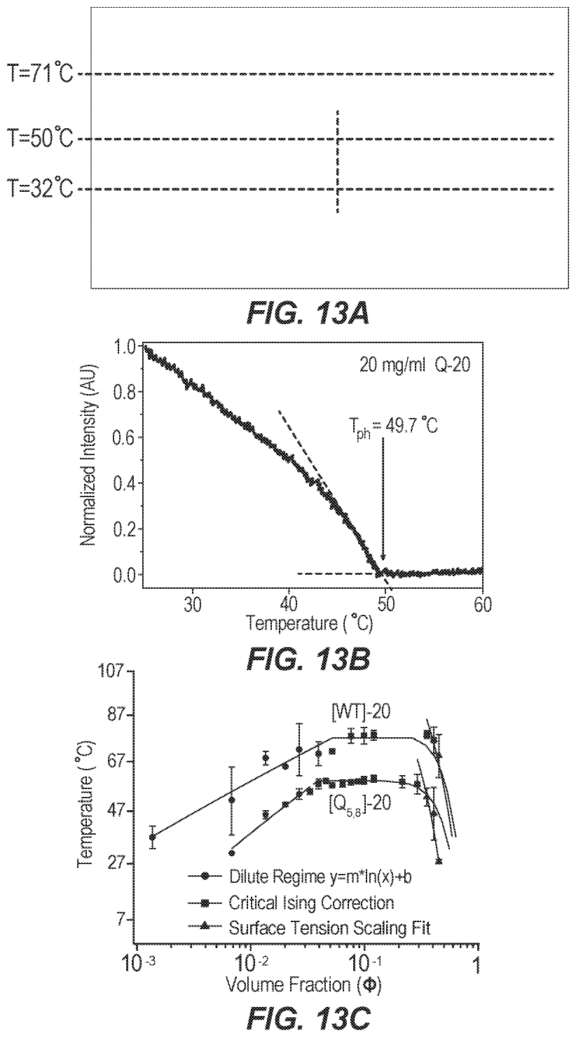

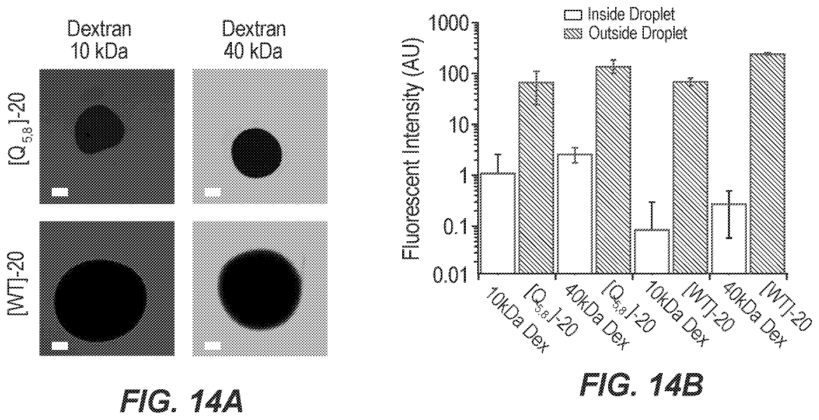

Having observed that Cₛₐₜ and the binodal phase boundary in the dilute regime of the UCST phase diagram of A-IDPs can be modified drastically by amino acid substitution, we were interested in the factors that modulate the high concentration regime of the phase diagram of A-IDPs. Polypeptides [WT]-20 and [Q_{5,8}]-20 express extraordinarily well for recombinant proteins, with yields of ˜500 mg L⁻¹ in shaker flask culture, which made it easy to purify over one gram of material to measure the UCST cloud point behavior at high-volume fractions of these A-IDPs directly (>0.1). To minimize the amount of material required, these experiments were performed in a multiplexed linear temperature gradient microfluidic device mounted on an upright light microscope, wherein the Tₜ could be quantified by the temperature at which phase separation occurs by a visible increase in light scattering intensity. These experiments produce binodal phase boundaries similar to optical turbidity measurements that are typically carried out in a UV-vis spectrophotometer (FIG. 13) and demonstrate that a ˜25° C. difference between the two binodal lines of [WT]-20 and [Q_{5,8}]-20 is maintained over the entire range of volume fractions tested. This corresponds to an increase in A-IDP volume fraction in the dense phase (ϕ₂) from ϕ₂=0.4 for [Q_{5,8}]-20 to ϕ₂=0.55 for [WT]-20 at an isotherm of 37° C. In addition to these phase diagram descriptions, phase separation in the presence of low (10 kDa) and high (40 kDa) MW fluorescently labeled dextran indicate that both [WT]-20 and [Q_{5,8}]-20 droplets are highly exclusionary, as we observed no fluorescence partitioning of dextran into the dense phase (FIG. 14A-B). These data in combination with our ability to easily purify A-IDPs from bacterial cell lysate with phase separation indicate that A-IDPs form highly exclusionary droplets in vitro at physiological solution, temperature, and pH conditions (ϕ₂>0.4).

A-IDPs have Controlled Cₛₐₜ in Eukaryotic and Prokaryotic Cell Lines

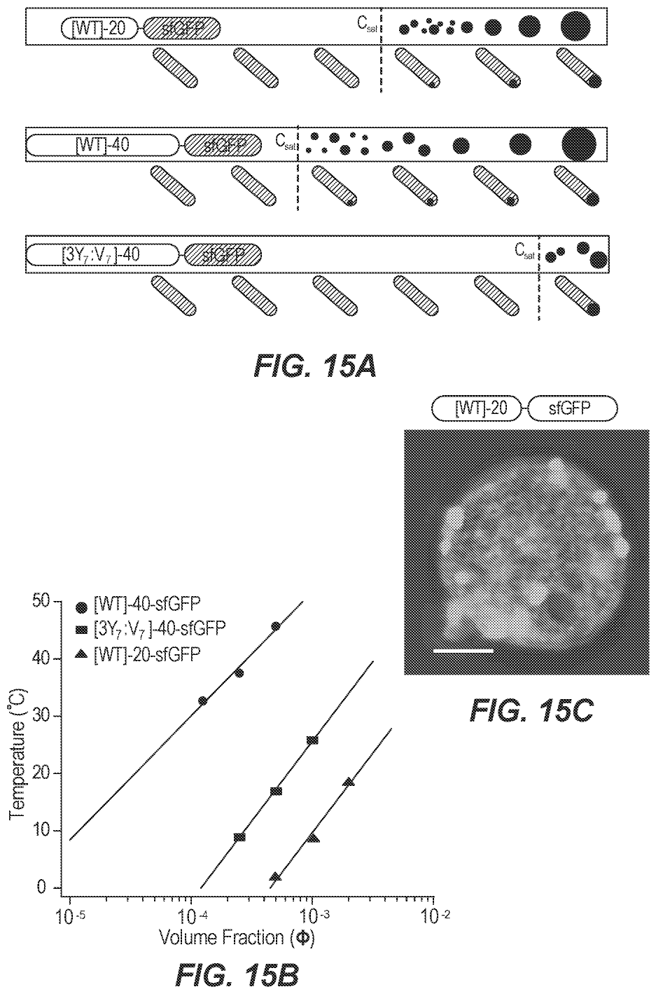

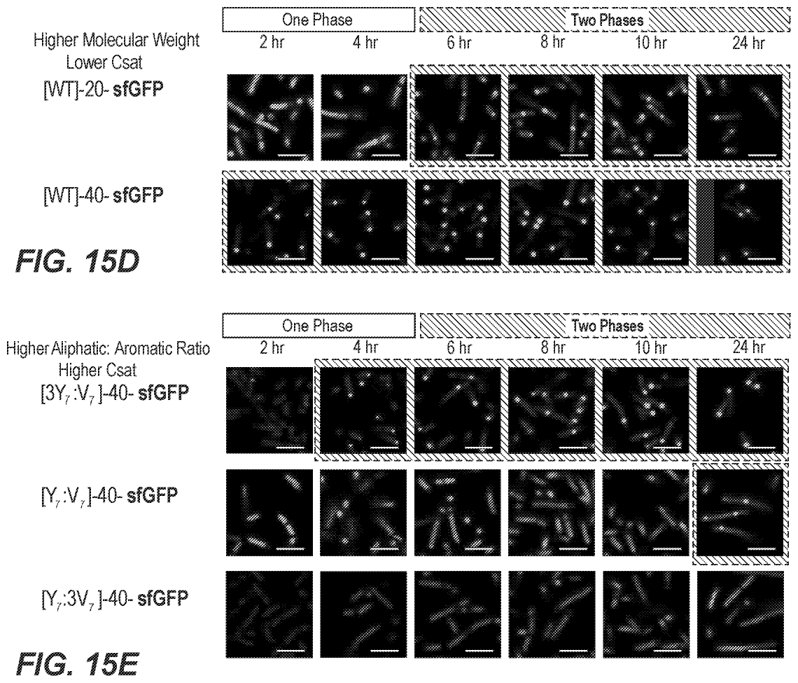

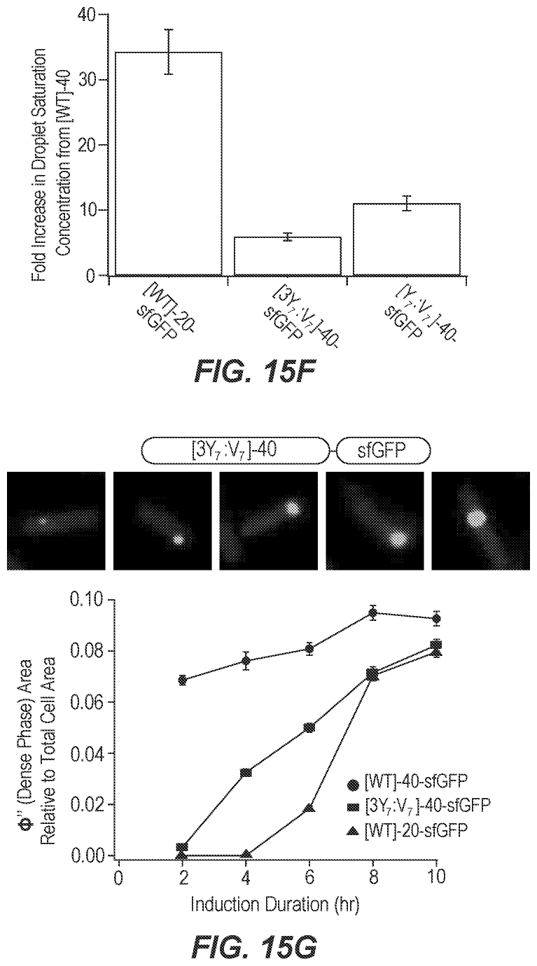

With a set of A-IDPs that exhibit a range of Tₜ as a function of concentration, and Cₛₐₜ that vary over seven orders of magnitude we sought to understand: (1) the dynamics of droplet assembly in living cells, and (2) to elucidate if it proceeds in vivo similarly to in vitro. To explore these two issues, we chose a set of IDPs that have a range of Cₛₐₜ from 1 to 815 μM with MWs of either ˜17 kDa or ˜32 kDa. To visualize localization of the A-IDPs within bacterial cells, each A-IDP was genetically fused to a super folder version of green fluorescent protein (sfGFP) (FIG. 15A).

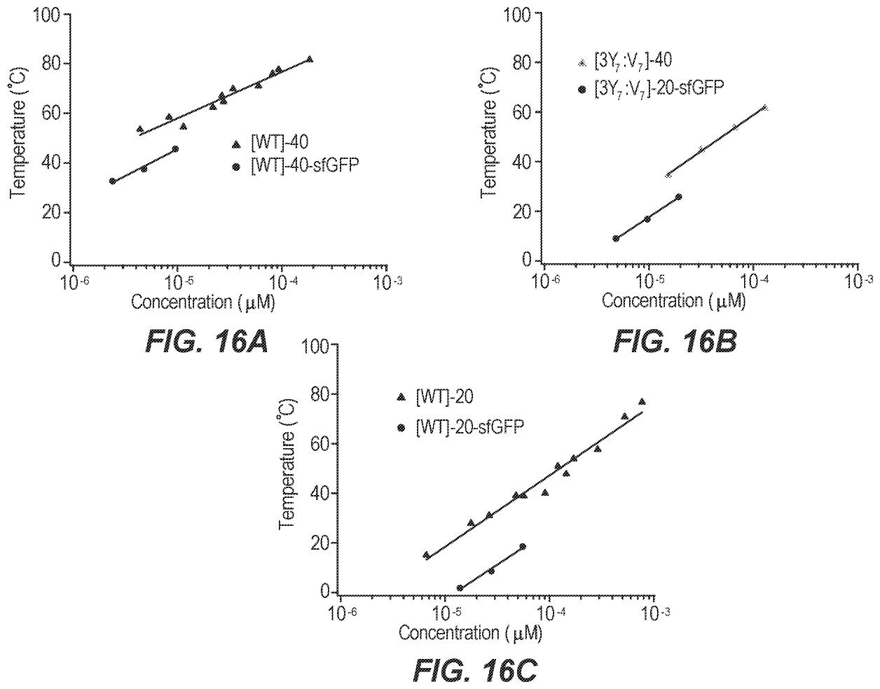

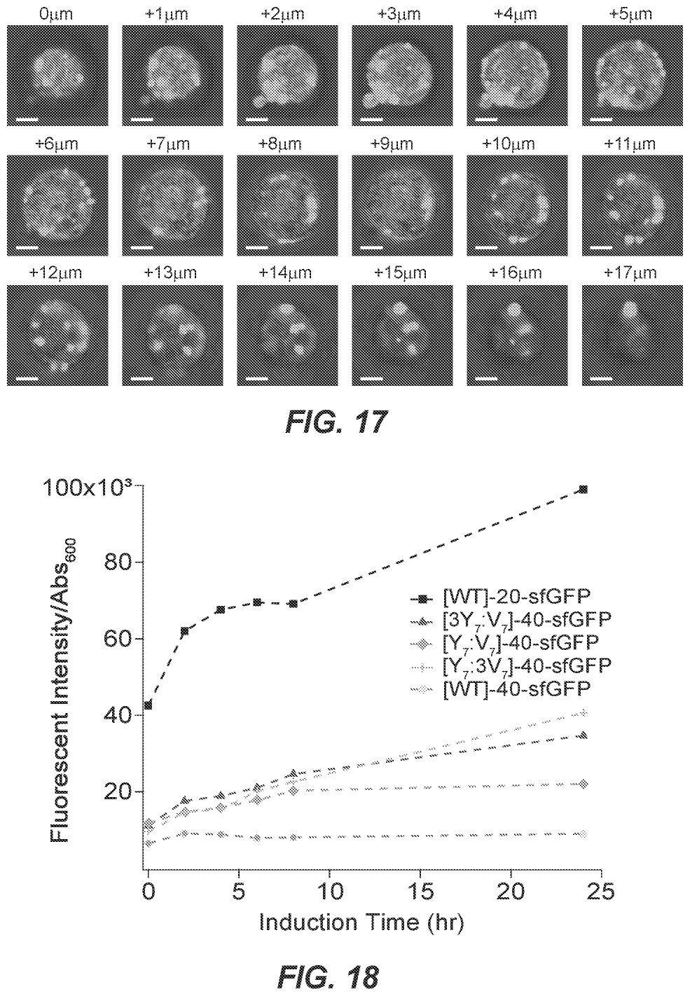

Fusion of sfGFP to A-IDPs to [WT]-20, [WT]-40, [3Y₇:V₇]-40, [Y₇:V₇]-40 did not eliminate the phase behavior but shifted the phase diagram (FIG. 15B and FIG. 16). Despite this shift, using confocal fluorescence microscopy, we were able to observe the formation of intracellular droplets of [WT]-20-sfGFP in both transfected human embryonic kidney (HEK) cells and E. coli (FIG. 15C and FIG. 15D, respectively). Interestingly, in the in vitro environment of an aqueous droplet in oil, we observed that nucleation occurs at multiple points in the aqueous compartment, but with time all the individual coacervate puncta coalesce into a single, large coacervate puncta. This indicated a lack of any significant energetic barriers to diffusion or coalescence. In HEK cells nucleation of coacervate puncta also occurred at multiple locations throughout the cell. However, unlike the in vitro situation, these puncta never coalesced into a single coacervate droplet so that individual coacervate puncta in the 2-4 μm diameter range remained dispersed throughout the cytosol of the HEK cell (FIG. 17).

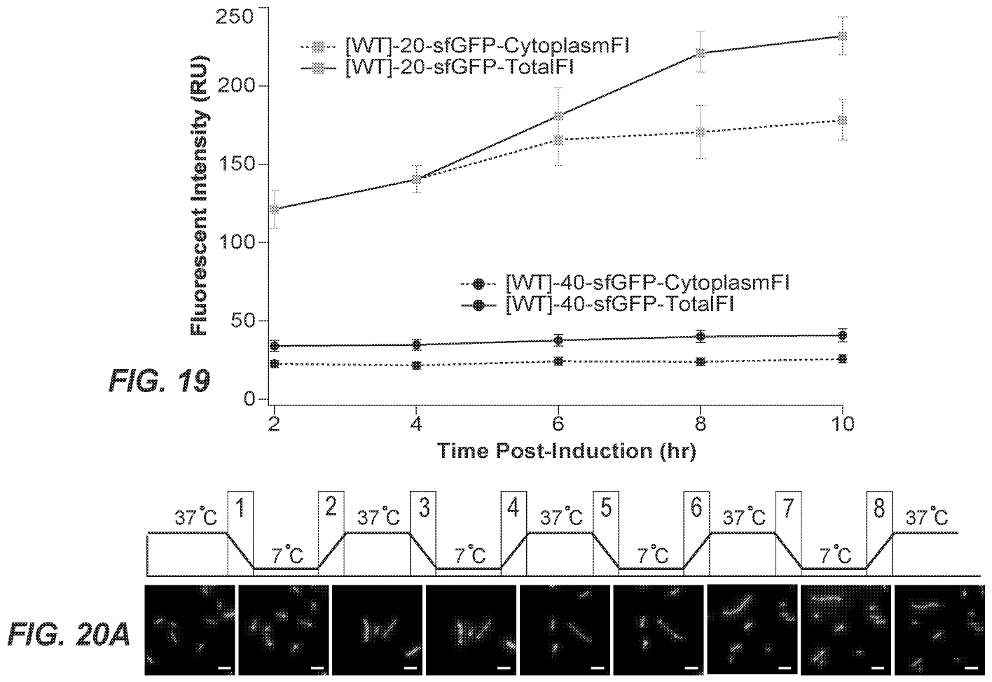

In contrast, phase separation in E. coli is significantly different from eukaryotic cells. The initiation of the UCST phase transition in E. coli is similar to HEK cells—and in vitro—where small densely fluorescent puncta form after the A-IDP concentration in the cell exceeds Cm, that then grow in size over time (FIG. 15G). The growth in the size of these puncta, as more A-IDP is expressed with time, is consistent with measurements of sfGFP fluorescence from the bulk E. coli population normalized to the absorbance at 600 nm (OD₆₀₀). The increase in fluorescence with time indicates that the intracellular concentration of the A-IDP-sfGFP fusion increases with increased protein induction time (FIG. 18). Unlike HEK cells however, but similar to in vitro experiments, these puncta in E. coli coalesce to form a single coacervate droplet per cell (Table 6). This result suggests differences in the diffusivity of the A-IDP between the prokaryotic and eukaryotic cytoplasm and suggest that the barriers to diffusion and coalescence of coacervate droplets in E. coli are far lower than in HEK cells. Simultaneously, the residual dilute regime remains at a relatively constant concentration (FIG. 19). Together, these results suggest that as the global concentration of the A-IDP within the cell increases with time, the cytoplastic concentration of the protein is buffered—remains constant—but that the volume of the coacervate increases relative to the size of the cell.

TABLE 6
Number of phase-separated domains per E. coli
as a function of induction time (n = 3 images)

| Time Post- | [WT]-20- | [WT]-40- |
|---|---|---|
| Induction (hr) | sfGFP | sfGFP |
| 2 | N/A | 1.01 ± 0.02 |
| 6 | 1.03 ± 0.01 | 1.03 ± 0.03 |
| 10 | 1.13 ± 0.01 | 1.10 ± 0.09 |

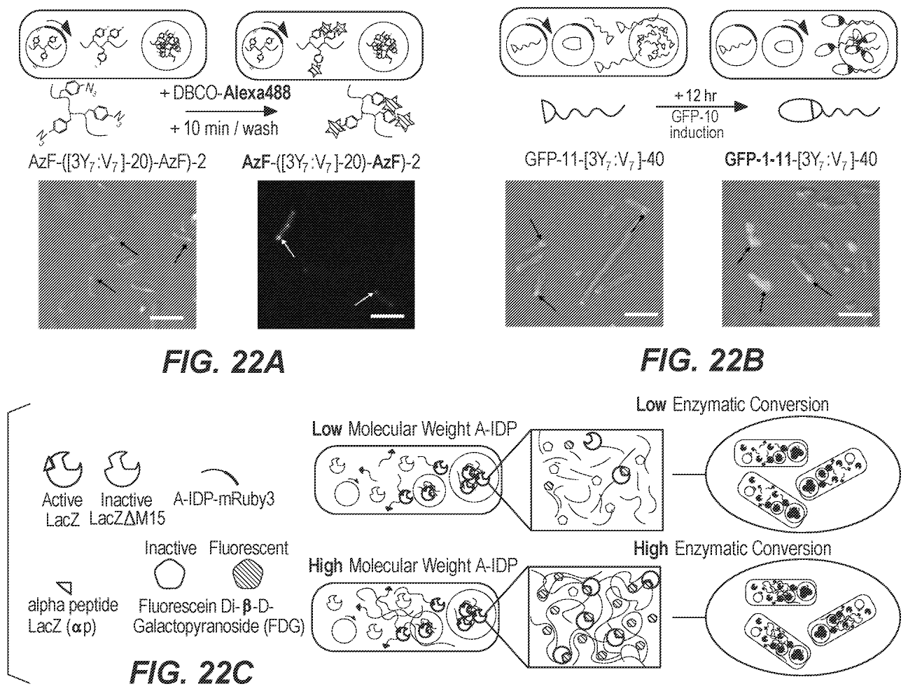

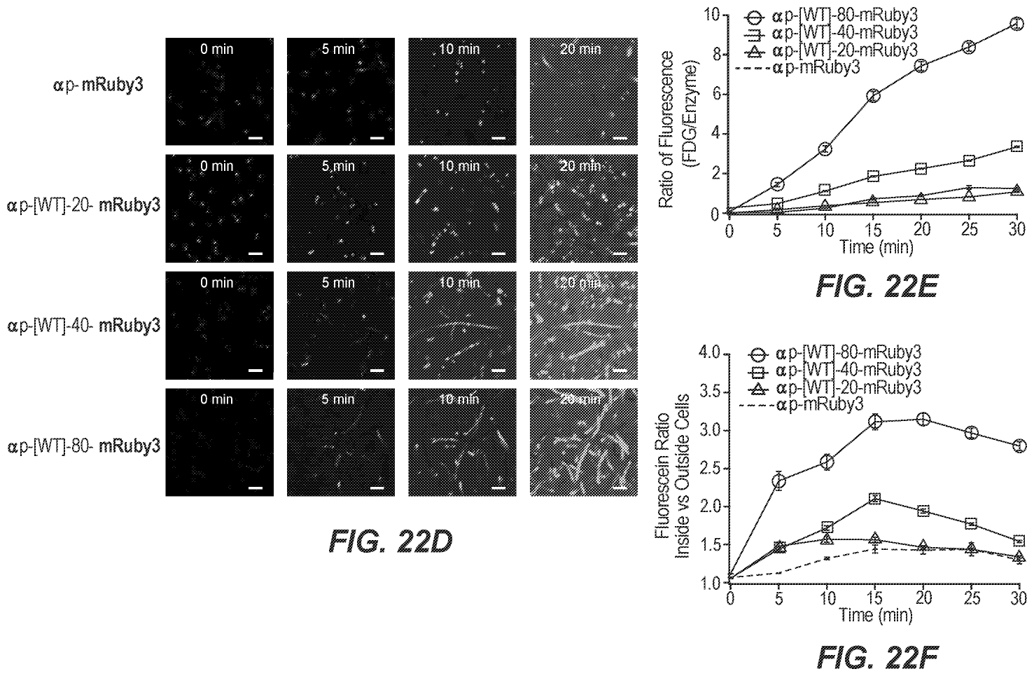

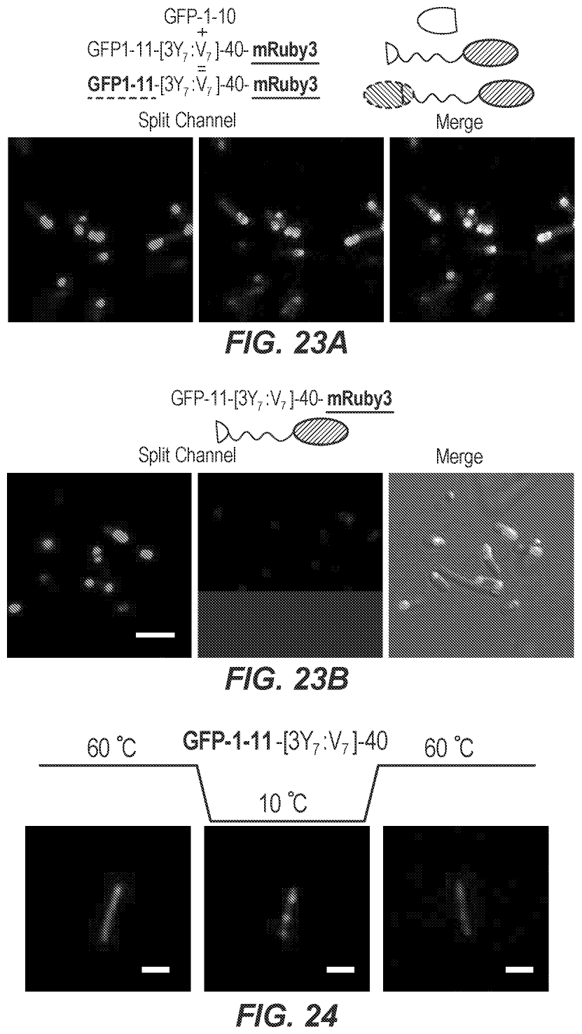

Similar to in vitro, the MW and aromatic:aliphatic content affect droplet formation in E. coli. Doubling the MW of [WT]-20-sfGFP to [WT]-40-sfGFP decreases Cₛₐₜ enough to cause droplet formation even prior to A-IDP induction, presumably because of leaky transcriptional regulation (FIG. 15D). Similarly, increasing the aliphatic content with V at the expense of Y increases the Cₛₐₜ to a concentration that is not measurable in the time course of these experiments (FIG. 15E). Although differences in Cₛₐₜ in vivo are not as dramatic as predicted by in vitro experiments with A-IDP-sfGFP fusions (FIG. 22F), perhaps because of the effect of intramolecular crowding within the cell, we can modulate the intracellular Cₛₐₜ by at least an order of magnitude using both MW and aromatic:aliphatic ratio of the A-IDP.

A-IDPs Exhibit Reversible UCST Droplet Formation in E. coli

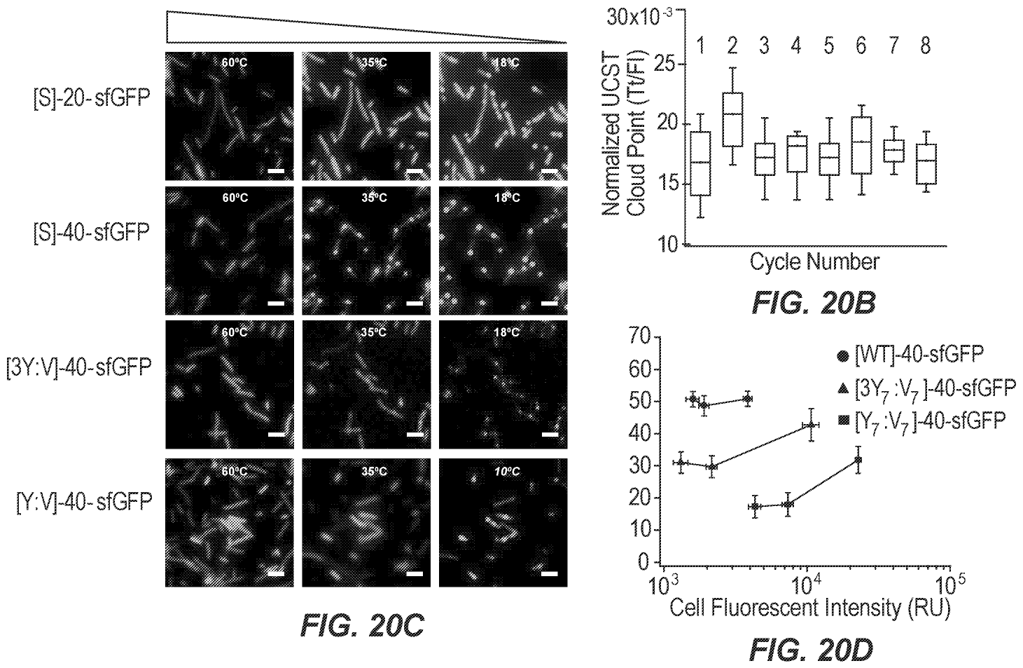

Just as one can cross a binodal line into the two-phase regime under isothermal conditions by increasing polypeptide volume fraction, this line may be crossed under constant volume fractions by decreasing solvent quality or the chi parameter (X). Experimentally this is most easily accomplished by reducing the temperature of the bulk solution. Similar to the UCST phase behavior of A-IDPs in vitro, A-IDPs exhibit reversible UCST phase separation inside cells that is reversible by repeated four cooling and heating cycles (FIG. 20A). The phase separation exhibits minimal hysteresis as the difference in the transition temperature of cooling (T_{t,C}) and the transition temperature of heating (T_{t,H}) varies by less than 2° C. (FIG. 20B).

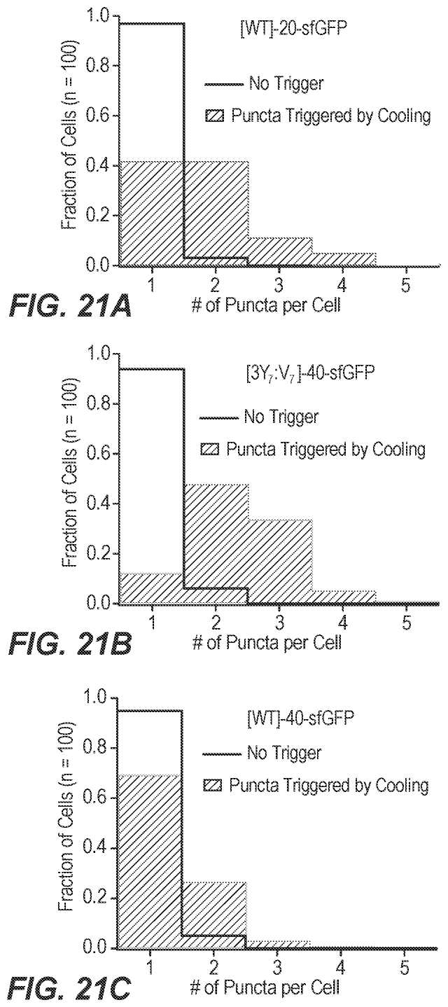

Interestingly, upon multiple heating and cooling cycles, we observed that E. coli exhibit spatial phase separation memory, with puncta forming in the same location as the first cycle (FIG. 2). Additionally, we observed cooling-triggered phase separation results in a higher number of puncta per cell (FIG. 21). The greater number of puncta observed with higher MW species indicates that the number of puncta formed per cell is a function of their diffusion coefficient, consistent with prior studies.

Increasing the MW of the A-IDP increases the observed Tₜ in E. coli (FIG. 20C). By manipulating the aromatic:aliphatic ratio while keeping MW constant and observing the formation of puncta within individual bacterium at various times post-induction (varying intracellular concentration), we were able to create partial intracellular phase diagrams as a function of Tₜ and intracellular fluorescence (FIG. 20D). This result is important because it ties the observed behavior upon cooling to a specific concentration for a given construct, essentially normalizing the observed cloud point for differing overall levels of protein expression throughout the cell population. Again, with increasing concentration we see an increase in UCST cloud point, although the rate of increase upon increasing concentration does not appear to follow a log-normal dependence.

### De Novo Design of Functional A-IDP Droplets in Cells

In order to understand the potential of using spatially confined intracellular coacervate droplets to carry out new functions, we asked the following questions: (1) Can coacervate droplets in cells recruit other molecules, and if so, what, if any, are the size limitations of such molecules?(2) Can these molecules interact with the A-IDP to impart a new function to the droplet?

To answer these questions, we first examined whether a small molecule could diffuse into and react with the A-IDP in a coacervate droplet located within an E. coli cell. We designed and expressed an A-IDP—[3Y₇:V]-40-UAA—that carries three copies of a unique biorthogonal reactive group—an azide; its primary amino acid sequence is listed in Table 3. After reaching intracellular concentrations greater than the Cₛₐₜ of [3Y₇:V₇]-40-UAA, we incubated live E. coli with 1 mg mL⁻¹ dibenzocyclooctyne-dye (DBCO-Alexa488) for 10 min (FIG. 22A). After a single wash step to remove excess dye, we observed fluorescent condensates in the cells. These experiments also demonstrate that the ϕ₁ fraction is also labeled in addition to the condensates. These data clearly show that a small molecule can diffuse from the extracellular environment into preformed condensates created by A-IDP phase separation and react with the A-IDP.

Next, we asked if larger molecules such as proteins are also capable of interacting with an A-IDP puncta. To answer this question, we designed a droplet capture experiment based on split green fluorescent protein (GFP). We first verified if the two components of a split GFP can interact with each other to create a functional GFP molecule if one of the components is fused to an A-IDP. GFP-11-[3Y₇:V₇]-40-mRuby3 was co-expressed in the presence of GFP-1-10; because the IDP is fused to mRuby3, the A-IDP condensates fluoresce red and can be visualized by fluorescence microscopy within the cell. We see fluorescently active GFP only in the interior of the condensates as seen by the co-localization of green fluorescence with the red fluorescence from the A-IDP condensates, indicating that the fragments GFP bind to each other to create an intact and functional GFP molecules that fluoresces green (FIG. 237A). In contrast, in the absence of GFP-1-10 induction, there is minimal green fluorescent inside the red fluorescent condensates (FIG. 237B).

These results show that two protein fragments of GFP can find and bind to each other in the cell despite the steric hindrance imposed by an A-IDP and a fluorescent reporter fused to one fragment of the protein. It does not however, prove that a protein can be recruited after an A-IDP condensate has formed, as the protein partners in the previous experiment are co-expressed and could bind in the cytoplasm prior to phase separation that occurs once the intracellular concentration of GFP-11-[3Y₇:V₇]-40-mRuby3 exceeds its Cₛₐₜ. To directly answer this question, we co-transformed E. coli with two plasmids—a Lac operon regulated plasmid that encodes one fragment of GFP (GFP-11) that is fused to [3Y₇:V₇]-40 and a second plasmid regulated by araBAD operon that encodes the other fragment of GFP (GFP-1-10). Once expression of GFP-11-[3Y₇:V₇]-40 at 37° C. proceeds long enough that its intracellular concentrations is greater than its Cₛₐₜ. we removed the IPTG induction media, and replaced it with arabinose containing media that induce the expression of the larger GFP fragment (GFP-1-10). We observed that subsequent to arabinose induction, both the ϕ₁ and ϕ₂ fractions of the E. coli contained fluorescently active GFP (FIG. 22B). This result suggested that the large GFP fragment is capable of penetrating the preformed condensate in the cell, find its binding partner and form a fully functional molecule, despite the fusion to the A-IDP. Once a fully functional GFP molecule is recruited into the intracellular droplets, it is then possible to dynamically modify the intracellular solubility of the reconstituted GFP-A-IDP by changing the temperature of the bulk (FIG. 24).

These experiments clearly show that small molecules and proteins can be recruited into intracellular coacervate droplets in E. coli and that a protein can be reconstituted within a coacervate droplet. These results suggested a path for the de novo design of intracellular coacervate droplets with new enzymatic function. We chose biocatalysis as the function of interest, because one of the proposed reasons for the evolutionary development of biomolecular condensates is to modulate the kinetics of various biological functions, including enzymatic reactions. However, there is little experimental evidence demonstrating how the function of enzymes is modulated by phase separation.

To investigate this, we created an A-IDP fusion that can recruit an enzyme into intracellular droplets to modulate its catalytic activity. We chose β-galactosidase for two reasons: (1) it has a range of small molecule substrates, one of which, Fluorescein Di β-Galactopyranoside (FDG), is colorless but when cleaved by ß-galactosidase, will fluoresce green. Thus, using a combination of a red fluorescent protein tagged to our enzyme-A-IDP fusion and fluorescein florescence we can track the colocalization of enzymatic reactions with A-IDPs in real time. (2) We had concerns that a large enzyme fused to a large A-IDP would not express at high enough concentrations in E. coli and thus not phase separate in vivo. To alleviate this concern, we took advantage of the widely used β-galactosidase (LacZ) blue-white screening system, where the alpha peptide (αp) complements the mutated enzyme LacZΔM15 to create a functional β-galactosidase enzyme. In our system, the αp is fused to a A-IDP-mRuby3 construct, such that enzyme activity is physically linked to the A-IDP which in turn is physically linked to red fluorescence.

Our studies with the DBCO-Alexa488 and split GFP provided the basis for this more complicated experiment. The DBCO-Alexa488 experiment suggested that a small molecule such as an enzyme substrate can penetrate puncta, even if delivered extracellularly (FIG. 22A). The split GFP experiment suggested that relatively large proteins can be recruited to A-IDP condensates to form functional proteins, suggesting that the same should be possible with the split β-galactosidase system (FIG. 22B). This peptide binding system also represents a more ubiquitous, engineered puncta platform as there are a number of split enzyme systems or small protein motifs that have been engineered to bind various intracellular targets.

Thus, we genetically fused the α-peptide (αp) from LacZ β-galactosidase to a A-IDP-mRuby3 construct. Our hypothesis is that the αp-A-IDP-mRuby3 protein can bind and recruit the other fragment of the enzyme—LacZΔM15 that has an α-peptide deletion—that is expressed endogenously in genetically modified E. coli (KRX, Promega) into intracellular droplets. After protein induction and resulting condensate formation, we deliver the substrate Fluorescein Di β-Galactopyranoside (FDG) to the cell medium where it is trafficked intracellularly, hydrolyzed into fluorescein at the sites of active β-galactosidase, and eventually exported outside the cell (FIG. 22C). By tracking the onset of the green fluorescence of fluorescein with confocal microscopy we can specifically observe where and when enzymatic activity is occurring within the cell and quantitatively track enzyme activity.

In our control experiment—αp-mRuby3—we observe limited persistence of fluorescence within the cells. It is important to note that the α-peptide itself is known to form inclusion bodies, and therefore, even in this control experiment, we observe some puncta inside the bacterial cells. However, upon fusion with [WT]-20, we observe that the fluorescence localizes long enough with the A-IDP condensates to be observed with confocal microscopy (FIG. 22D). Despite this increased colocalization, the total fluorescent production over time is not statistically significant from the αp-mRuby3 control (FIG. 22E).

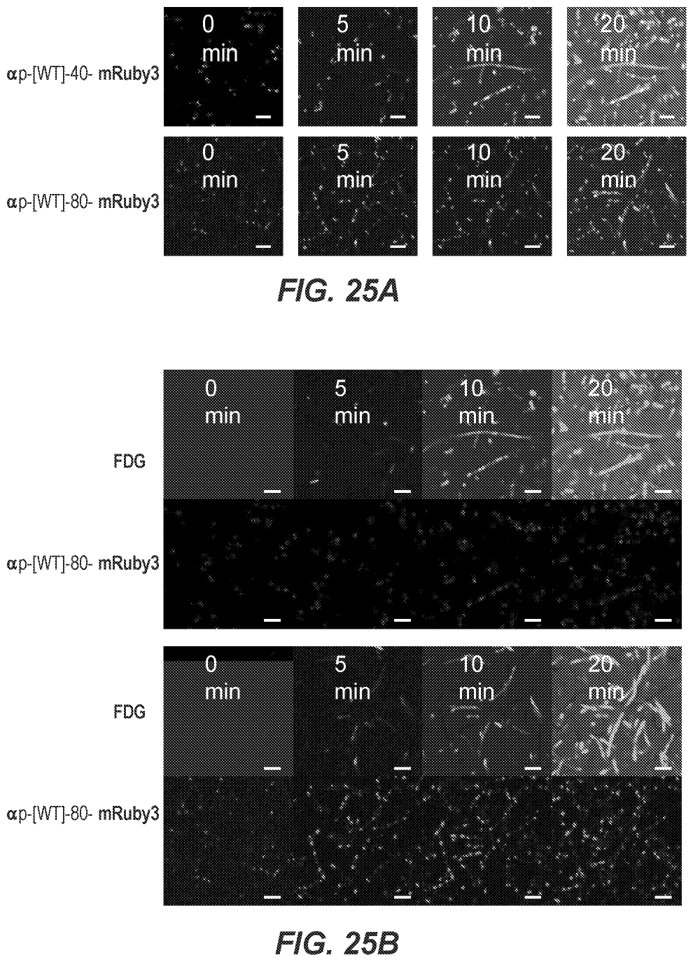

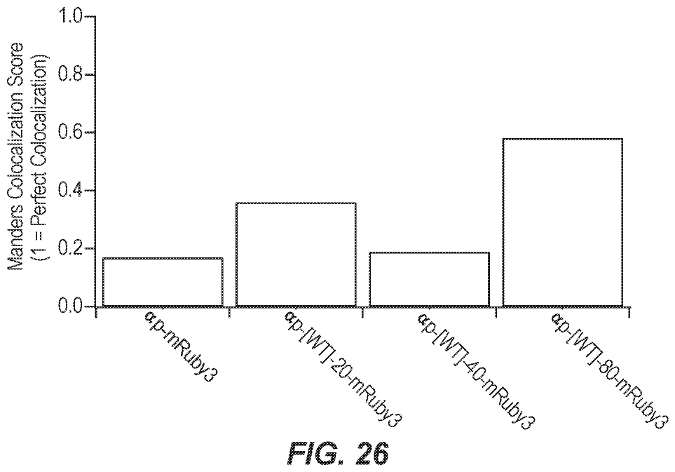

When we increase the MW of the A-IDP, and thus decrease Cₛₐₜ, we observe a dose-response effect in the total FDG fluorescence intensity as well as colocalization with the αp-A-IDP-mRuby3 fusion (FIG. 22D). αp-[WT]-40-mRuby3 and αp-[WT]-80-mRuby3 have 2.5× and 7.5× greater FDG converted at 20 minutes compared with the αp-mRuby3 control (FIG. 22D and FIG. 25). Quantification of the colocalization of green and red fluorescence with Mander's overlap coefficient indicates increased colocalization when the α-peptide is fused to A-IDPs compared to the fluorescent reporter alone (FIG. 26). To quantify the observed colocalization, we analyzed individual cells within the image frame with green fluorescence that were above the background threshold at each timepoint. Higher MW A-IDPs exhibit higher fluorescence inside the cell normalized to the background at each point in time (FIG. 22F). This dose-response effect of MW emphasizes a mechanism of increased persistence of the substrate molecule inside the droplets leading to more efficient green fluorescence conversion.

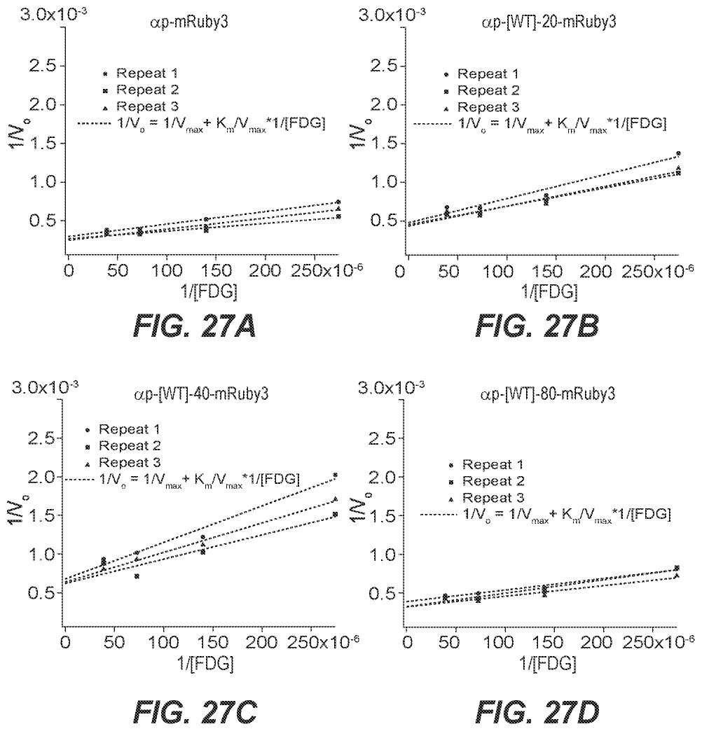

Quantification of fluorescence production at various substrate concentrations in vitro suggests that the mechanism of this effect is a statistically significant increase in the catalytic constant (K_{cat}) of the enzyme with increasing MW. This constant can be interpreted as the “turnover efficiency” of the enzyme or the number of catalytic events that occur per unit time. We observed 1.4×, 1.6× and 4.2× increase in the Kw for αp-[WT]-20-mRuby3, αp-[WT]-40-mRuby3, αp-[WT]-80-mRuby3 compared to the αp-mRuby3 control (FIG. 27 and Table 7). Considering our previous observation of increased colocalization of product (fluorescein) and the labeled A-IDP as a function of MW, we propose that the observed increase in fluorescence is caused by increased colocalization of the enzyme and substrate in the condensates, leading to a higher measured K_{cat}. We observed non-significant changes to the Michaelis-Menten constant (Kₘ) which describes the affinity of the enzyme for the substrate, suggesting that fusion of the A-IDP does not change the binding constant of the enzyme-substrate complex. Using K_{cat} and Kₘ, we can define a catalytic efficiency which also supports our hypothesis of an increase in the enzymes' efficiency within condensates with increasing MW of the A-IDP. This enhancement in enzymatic efficiency is on the order of magnitude of change observed by various protein engineering techniques used primarily to improve K_{cat}.

TABLE 7
Michaelis-Menten enzyme kinetic
parameters (standard error of the mean, n = 3)

| | Vₘₐₓ | | | k_{cat}/Kₘ |
|---|---|---|---|---|
| | (FI_{FDG}* | | k_{cat} | (FI_{FDG}⁻¹* |
| Protein | min⁻¹) | Kₘ (FI_{FDG}) | (min⁻¹) | min⁻¹) |
| ap-mRuby3 | 3708 ± 183.2 | 4972 ± 636.3 | 3.56 ± 0.10 | 7.43E−04 ± |
| | | | | 1.02 × 10⁻⁴ |
| ap-[WT]-20- | 2165 ± 35.02 | 5896 ± 380.5 | 5.10 ± 0.43 | 8.79E−04 ± |
| mRuby3 | | | | 1.16 × 10⁻⁴ |
| ap-[WT]-40- | 1549 ± 34.75 | 5988 ± 475.1 | 5.75 ± 0.13 | 9.81E−04 ± |
| mRuby3 | | | | 9.96 × 10⁻⁵ |
| ap-[WT]-80- | 2922 ± 174.9 | 4504 ± 439.7 | 15.2 ± 0.68 | 3.45E−03 ± |
| mRuby3 | | | | 4.58 × 10⁻⁴ |

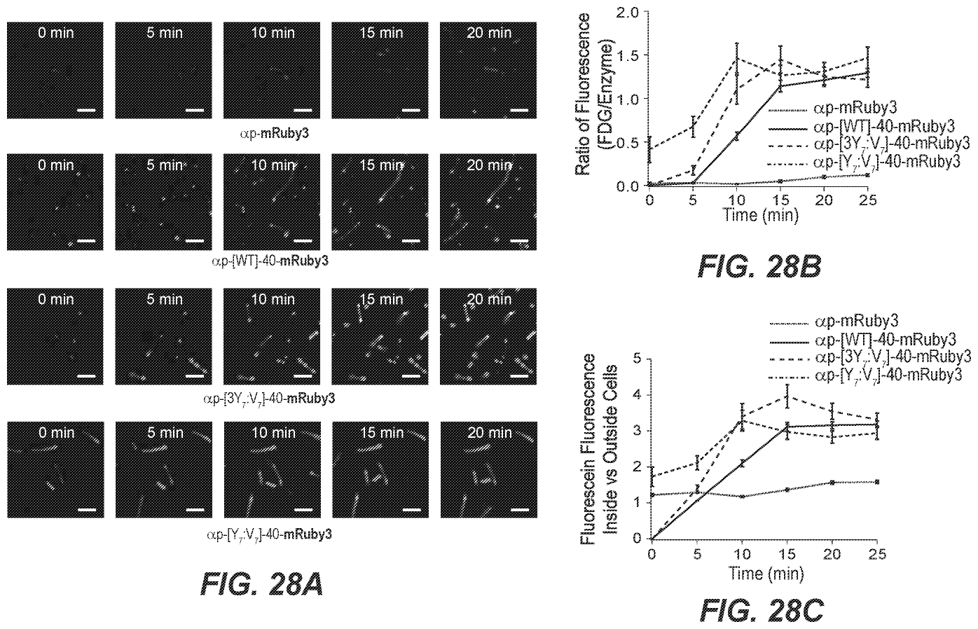

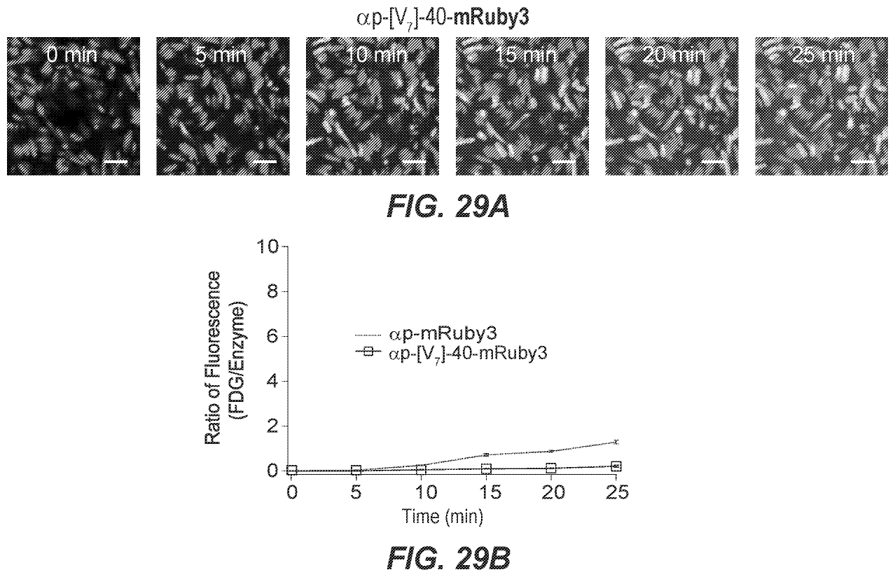

We also fused the LacZ alpha peptide to A-IDPs with differing levels of aromatic content at a constant MW (FIG. 28A). We hypothesized that differing levels of aromatic content would affect FDG uptake into the droplets and therefore affect overall enzymatic activity. Surprisingly, we observed similar overall levels of fluorescence between αp-[WT]-40-mRuby3, αp-[3Y₇:V₇]-40-mRuby3 and αp-[Y₇:V₇]-40-mRuby3. However, the dynamics of enzymatic activity are different, with A-IDPs of greater aliphatic content allowing for faster uptake into the condensates (FIG. 28B). The differences between the ratio of FDG fluorescence inside the cell and outside the cell between αp-A-IDP-mRuby3 fusions with different aliphatic content were insignificant, indicating that MW is the primary driving force for fluorescein and/or FDG persistence inside intracellular droplets (FIG. 28C). Completely deleting the aromatic residues from the repeat unit of the A-IDP results in apparently soluble enzymes that do not form intracellular condensates, and whose activity is lower than the enzyme formed by complementation of LacZΔM15 with the αp-mRuby3 fusion that has no A-IDP tag (FIG. 29A-B).

We show herein A-IDPs that consist of repeats of an octapeptide motif inspired by native IDP exhibit reversible UCST phase separation in aqueous solution. Despite the simplicity of their sequence, they recapitulate many of the features seen in more complex, native IDPs. The formation and dynamics of their phase separation into coacervate droplets are controlled by two simple design parameters that are genetically encodable at the sequence level—MW of the A-IDP and the ratio of aromatic:aliphatic residues in the octapeptide repeat. Using these two parameters—aromatic:aliphatic ratio and MW—we were able to produce A-IDPs with Cₛₐₜₛ ranging from nanomolar to millimolar concentrations. This work supports the growing evidence of R-aromatic interactions that drive phase behavior and also adds additional evidence of the molecular hierarchy that exists between the aromatic groups W, Y, F and H in modulating UCST phase behavior. Although the IDP literature often ignores the importance of MW, our results suggest that MW may be more critical than composition for defining the UCST binodal. We anticipate that these results will inform and dramatically shift the strategy for mutating native IDPs and designing de novo IDPs.

These design parameters faithfully translate from in vitro to intracellular environments. The A-IDPs phase separate inside cells by the same principles that drive their UCST phase separation in vitro indicating that the same thermodynamic driving forces embedded in the sequence and molecular weight also modulate droplet formation dynamics in isolation. Due to the simplicity of their design, A-IDPs behave in vivo as their phase diagrams in vitro suggest—as their intracellular concentration increases to a Cₛₐₜ, small phase separating droplets form at individual points in space that continue to grow in size with increasing overall A-IDP concentration inside the cell. This predictable observation has been theorized by previous studies but has not been conclusively demonstrated until now.

Finally, these proteins can be used for the de novo design of functional intracellular droplets. We rationally designed intracellular puncta capable of binding and recruiting a β-galactosidase deletion mutant, which could modify the catalytic efficiency of the enzyme-substrate complex—a complex which has not evolved to form intracellular condensates. The catalytic efficiency of the reconstituted enzyme in phase separated coacervate droplets is MW dependent and increases with the MW of the A-IDP. Higher MW A-IDPs more efficiently sequester the substrate in the enzymatically active, intracellular phase separated puncta, which results in a higher catalytic efficiency as measured by K_{cat}. These proof-of-concept experiments demonstrate that intracellular droplets can be engineered to have non-canonical functions in live cells and provide a new platform for intracellular material manipulation. In summary, with over 60 IDPs synthesized for this study that span a range of Cₛₐₜ, and proof of concept experiments recruiting proteins into coacervate droplets within a cell and thereby modulating protein function, these studies lay the groundwork for the de novo design of functional intracellular condensates. We expect that these A-IDPs will be useful as building blocks from which new biological condensates with emergent behaviors can be built within living cells to better study the functional significance of phase separation in living cells and to encode new functions for droplets within cells. We also anticipate that these IDPs will prove useful in other biomedical applications beyond the design of intracellular droplets that can profit from the their tunable UCST phase behavior. This marriage of soft material science with biophysical characterization of subcellular materials will continue to be an exciting space for to engineer cells with new or improved function and new biomaterials.

### Example 2

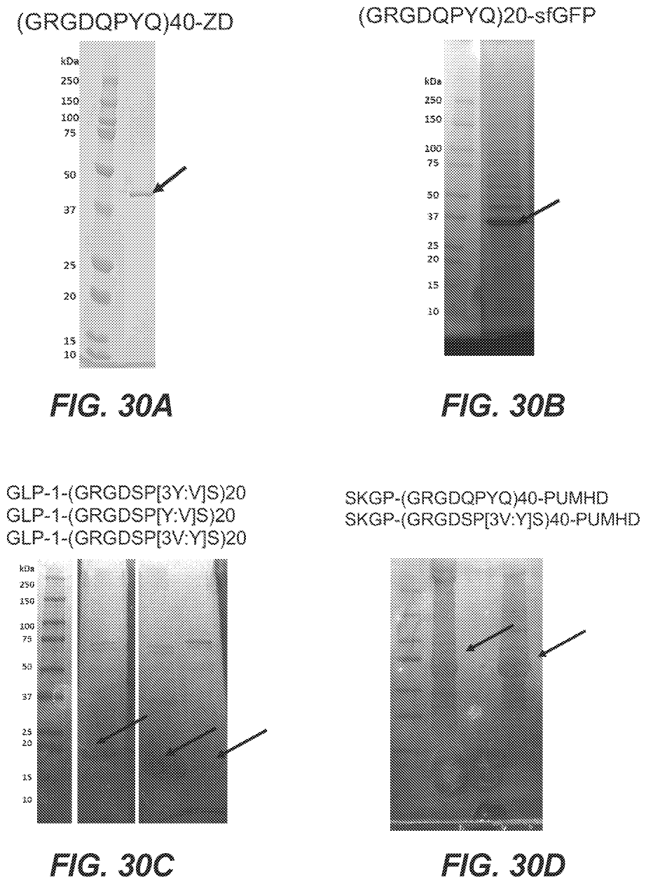

FIG. 30A-D shows examples of various fusion proteins that express at low levels in prokaryotic expression systems that when fused to disordered biopolymers rescue expression levels and using the phase separation behavior of the biopolymers allow for recovery into soluble fractions. This can be performed with mAb binding proteins that have a nanobody folded structure that bind to mAb (ZD), fluorescent fusion proteins that have beta-barrel structures (sfGFP), therapeutic protein peptides (GLP-1) with strong alpha-helical tendencies, RNA binding proteins (PUMHD) that have tandem repeat structures and antimicrobial peptides that exhibit cytotoxic tendencies in E. coli.

Table 8 shows the expression levels of various fusion proteins.

TABLE 8
Expression levels of various fusion proteins

| | Protein | | SEQ ID NO: |
|---|---|---|---|
| Sequence | Function | Titer | (AA, DNA) |
| SKGP-(GRGDQPYQ)₄₀-ZD | mAb binding | ~300 mg/L | 284, 285 |
| SKGP-(GRGDQPYQ)₂₀-ZD | mAb binding | ~150 mg/L | 286, 287 |
| SKGP-(GRGDSPYS)₄₀-PKD2 | AAV binding | ~100 mg/L | 288, 289 |
| SKGP-(GRGDSPYS)₄₀- | Fluorescent | ~50 mg/L | 290, 291 |
| SfGFP | reporter | | |
| GLP-1-(GRGDSP[3Y:V]S)₂₀ | Therapeutic | ~200 mg/L | 292, 293 |
| GLP-1-(GRGDSP[Y:V]S)₂₀ | Therapeutic | ~50 mg/L | 294, 295 |
| GLP-1-(GRGDSP[3V:Y]S)₂₀ | Therapeutic | ~25 mg/L | 296, 297 |
| GLP-1-(GRGDSPYS)₄₀ | Therapeutic | ~50 mg/L | 298, 299 |
| GLP-1-(GRGDSPYS)₂₀ | Therapeutic | ~100 mg/L | 300, 301 |
| GLP-1-(GRGDSP[3Y:V]S)₄₀ | Therapeutic | ~150 mg/L | 302, 303 |
| GLP-1-(GRGDSP[Y:V]S)₄₀ | Therapeutic | ~50 mg/L | 304, 305 |
| GLP-1-(GRGDSP[3V:Y]S)₂₀ | Therapeutic | ~50 mg/L | 306, 307 |
| AMP 1 | Therapeutic | ~25 mg/L | 308, 309 |
| AMP 2 | Therapeutic | ~20 mg/L | 310, 311 |
| AMP 3 | Therapeutic | ~50 mg/L | 312, 313 |
| SKGP-(GRGDSP[3Y:V]S)₄₀- | RNA binding | ~25 mg/L | 314, 315 |
| PUMHD | | | |
| SKGP-(GRGDQPYQ)₄₀- | RNA binding | ~20 mg/L | 316, 317 |
| PUMHD | | | |

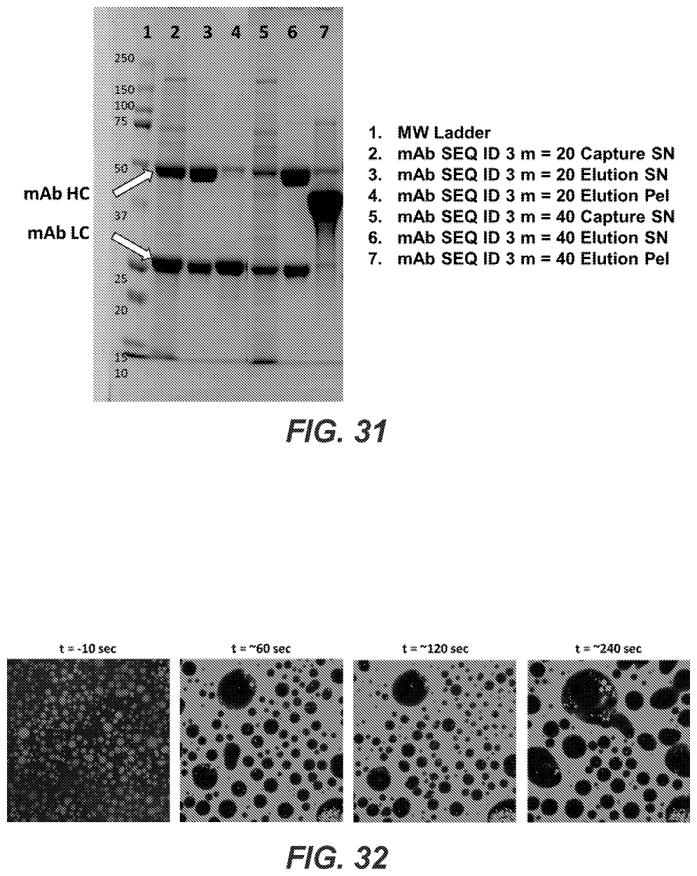

FIG. 31 shows the in incubating mAb with a phase separating biopolymer fused to a domain from protein A that binds mAbs. First, the biopolymer is bound to the mAb and centrifuged to capture the mAb heavy (HC) and light chain (LC). Then, the supernatant was removed, and the pellet was resuspended in an elution buffer that is a lower pH which causes dissociation between the biopolymer-ZD fusion and the mAb. The solution was spun again creating an elution supernatant which contains pure mAb HC and LC that had few other protein contaminants. The elution pellet contained the biopolymer and no mAb.

FIG. 32 shows microscopic images of fluorescently labeled mAb (red/white in grey-scale) is visualized in the presence of phase separated protein ((GRGDQPYQ)40, SEQ ID NO: 3 with m=40) fused to the Z-domain of Protein A (ZD). The first frame shows colocalization of the droplets with a fluorescent signal. When the buffer pH is dropped at t=0, there is an inversion in the fluorescent signal suggesting the mAb has completely dissociated from the phase separated protein-ZD fusion protein (white arrow) and entered the surrounding solution (red). These biopolymer-fusion proteins retain their liquid-like behavior as droplet fusion occurs between 60-240 sec.

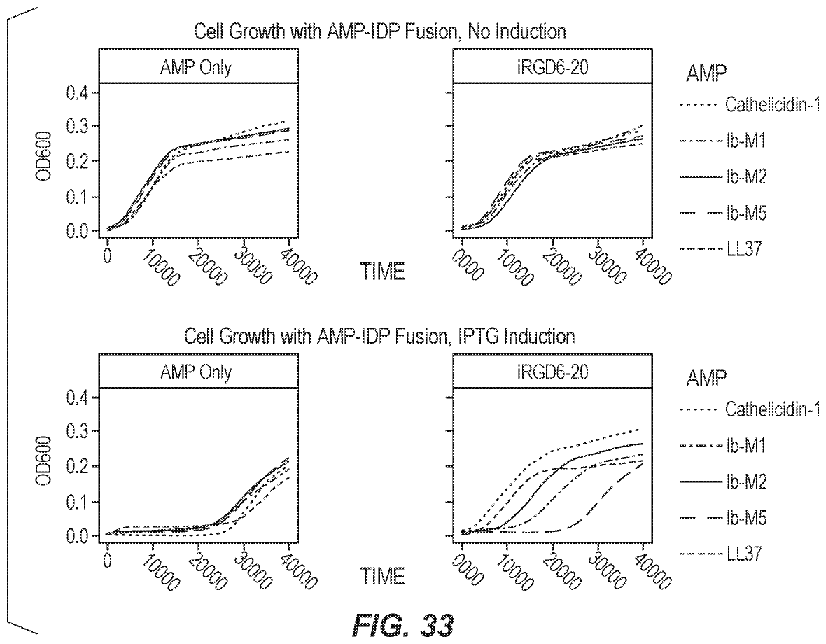

FIG. 33 shows fusion proteins containing various AMPs fused to biopolymers. When the fusion protein is not expressed cell growth proceeds as normal measured by an increasing absorbance at OD₆₀₀. When the AMP alone is expressed, cell growth is stunted. When AMP-biopolymer fusion protein is expressed, normal growth is recovered, suggesting reduced availability of the AMP.

### Example 3

Effect of Repetitive Polypeptide Design on In Vivo Release of Glucagon Like Peptide 1 (GLP-1) from Sub-Cutaneous Depots

We utilized the phase behavior of the repeat polypeptide, chemically inspired from naturally occurring IDPs, to control the bio-availability of resources when confined by a lipid bi-layer in bacteria and to control the assembly of micelles when sterically confined by a relatively hydrophilic ELP or RLP molecule exerting a surfactant like effect. We were motivated to test the efficacy of controlling bio-availability in a system where the dilute phase is attached to an infinite sink—where the dilute phase is in equilibrium with a biological system capable of protein clearance. The Chilkoti lab has extensive experience with subcutaneous delivery of peptide molecules in vertebrae animals in this exact set up, the primary focus on delivering therapeutic molecules for diabetes and various cancers, albeit exclusively with LCST polypeptides.

The delivery of peptides remains an outstanding challenge for drug delivery. Despite protein engineering improvements focused on improving half-life, their effective window lasts from minutes to a few hours rendering them unsuitable for therapeutic use. Interestingly, nature utilizes peptides in various biological applications, but regulation of their activity is often tightly controlled by a cellular population that can react to a phenotype change. Thus, man-made peptide drugs require a delivery solution, one that can improve the pharmacokinetics of these valuable macromolecules.

The most common approaches to improve a peptide's half-life include protein engineering and changing the formulation to prolong release and/or reducing renal clearance. Sequence engineering—such as the incorporation of D-amino acids or other chemically esoteric amino acid derivatives—can limit proteolytic degradation of the protein, but severely limit manufacturing choices. Encapsulation methods produce inconsistent effects on bioavailability or require harsh production conditions that limit the type of peptide drug that can be delivered via these methods. Strategies to decrease renal clearance revolve around increasing the size of the molecule and reducing opsonization including attachment to synthetic or biological polymers that extend half-life, fusion to a large protein, and conjugating chemical moieties that allow the peptide to piggyback on endogenous biomolecules with slow turnover rates like albumin or antibody fragments. These strategies are not without limitations as they dramatically reduce potency and rely on a patient population to express these piggybacking biomarkers consistently between individuals.

These engineered polypeptides offer an elegant solution-through their primary amino acid sequence and molecular weight they control the dense and local dilute phase of the bioactive molecule effectively controlling the bioavailability of the drug. Unless proteases possess the unique ability to diffuse into the dense phase of the polypeptide-peptide drug depot, the availability of peptide drug is mediated exclusively by the primary amino acid sequence of the polypeptide. This local dilute phase can then diffuse from the subcutaneous space into circulation and exert a therapeutic effect. Using the principles of polypeptide design described herein we can rationally design drug release depots that can prolong the half-life of the peptide drug in vivo.

For this study, the relevant peptide drug is GLP-1, which is a 31 amino acid peptide produced in the L cells of the intestines, capable of exerting blood glucose control over a large therapeutic window. Previous experience with GLP-1-polypeptide depots revealed much about the design of sub-cutaneous depots for drug release. (1) Fusion of a macromolecule such as the polypeptide ELPs reduce potency of the GLP-1 molecule by about ˜30 fold but this does not preclude in vivo activity. (2) Zero order release can be achieved for up to 10 days in mice and 17 days in monkeys under optimal conditions. (3) Optimal conditions are an injectable transition temperature 5-7° C. below the body temperature of the animal and a molecular weight of 35 kDa or greater to avoid renal clearance. This optimal 5-7° C. below body temperature corresponds to a dilute phase concentration of approximately 1-100 μM where non-optimal depots exhibited Cₛₐₜₛ an order of magnitude above and below this optimal range.

The peptide drug of choice for these experiments is GLP-1 for several reasons. (1) GLP-1 can rapidly exert a therapeutic effect in vivo. (2) GLP-1 is a prime candidate for improved pharmacokinetics with a half-life of ˜5 min in vivo. (3) GLP-1 can be easily studied in established mice models of diet induced obesity where a high fat diet increases the blood glucose. 4) GLP-1 is a stable peptide drug which will eliminate confounding variables associated with genetic fusion to various polypeptide partners and myriad delivery strategies.

Previous studies suggested that transition temperature at injection concentration is the important parameter for determining efficacy. However, this misjudges the important isotherm on the phase diagram to be room temperature instead of the operating temperature of the depot which is defined by the animals resting body temperature. In subcutaneous mice models, this temperature is approximately 35° C. Thus, we are looking proteins with variable dilute phase concentrations, at an isotherm of 35° C. (similar to a saturation concentration at 35° C.) and different molecular weights to test to observe how these two variables affect the bioavailability of GLP-1.

A potentially confounding effect of this study is the method of delivery. With polypeptides that exhibit a UCST, to achieve Cₛₐₜₛ that are equivalent to the range of Cₛₐₜₛ previously tested with LCST polypeptides, the solution cloud point of the polypeptide will often be dramatically higher than the body temperature of the animal. Thus, one of our first tests will be establishing depots using either solubilizing small molecules (urea) that can diffuse from the injection site more rapidly than the polymer, thus enabling the polypeptide to be injected in a soluble solvent that rapidly exchanges with the environment to become a poor solvent, forming a depot (FIG. 34). Secondly, we physically implanted a desiccated depot of a prescribed shape, size and dose that will rehydrate and begin to release active polypeptide after an initial delay (FIG. 34). We searched for consistent dosing that rapidly produces depots of similar size in the sub-cutaneous space.

Next, we designed polypeptides of similar molecular weight that exhibit different Cₛₐₜ. We will achieve this feat by utilizing the same parameter as before, the aromatic:aliphatic ratio to rationally tune the binodal of the GLP-1-polypeptide fusion. This allowed us to observe the effect to Cₛₐₜ on the therapeutic efficacy of the fusion. However, Cₛₐₜ is just one parameter that affects bioavailability. The overall size of the molecule is also critical to the diffusion/convection in the subcutaneous space. Thus, using similar ranges of Cₛₐₜ we will test depots for delivery as we increase the molecular weight of the molecule overall. Previous studies by previous lab members have demonstrated that there diminishing returns of molecular weight beyond ˜35 kDa.

### Gene Synthesis

Each octapeptide amino acid motif with the desired saturation concentration were genetically fused to the C terminus of GLP-1. To increase the number of total repeats of the gene, we performed iterative cloning steps of Recursive Directional Ligation by Plasmid Reconstruction adding an addition twenty repeats during each step. Transformations were performed into the desired E. coli cell line—BL21 (DE3) for recombinant expression.

### Protein Purification

Individual liquid cultures of BL21 E. coli strains each harboring our gene of interest were inoculated into 5 mL of Terrific Broth (TB) medium from frozen glycerol stocks and grown to confluence overnight (16-18 hours). Cultures were then inoculated at a 1:200 dilution in 1 L TB media supplemented with 45 μg·mL⁻¹ kanamycin. Cells were grown at 37° C. in a shaking incubator (˜200 RPM.) for 9 hr, at which time protein expression was induced by the addition of 500 μM Isopropyl-β-D-thiogalactoside (IPTG). Cells were then incubated at 37° C. (shaking at ˜200 RPM) for an additional 18 hr. Protein was then purified from the insoluble cell suspension fraction. In brief, cell pellets were isolated by centrifuging cultures at 3500 RCF and resuspending in 20 mL of milli-Q water. Cells were then lysed by sonicating the cell solutions for 2 minutes, with 10 seconds of pulsing followed by 40 seconds of rest on ice (Misonix; Farmingdale, N.Y.)

Centrifuging each lysate suspension at 20,000 RCF for 20 minutes results in a soluble and insoluble fraction. The supernatant was discarded with the insoluble fraction resuspended in an approximately equal volume of 8 M urea+140 mM PBS (˜6-8 mL). This suspension was heated for 10 min in a 37° C. water bath and then centrifuged at 20,000 RCF for 20 minutes. The supernatant was collected from this suspension and dialyzed in a 10 kDa membrane (SnakeSkin™, Thermo Fischer Scientific) against a 1:200 milli-Q water solution at 4° C. The dialysis water was changed twice over a 48-hour period. From inside the dialysis bag, both insoluble and soluble components were collected and centrifuged at 3500 RCF for 10 minutes and 4° C. The supernatant was removed and the remaining insoluble pellet containing the protein of interest was lyophilized for a minimum of three days to remove all water from the pellet.

Protein purity was characterized by 4-20% gradient tris-HCl (Biorad, Hercules, Calif.) sodium dodecyl sulfate polyacrylamide gel electrophoresis (SDS-PAGE) and staining with either 0.5 M copper chloride or SimplyBlue™ SafeStain (Thermo Fischer Scientific). Protein yield was determined by weight after lyophilization.

### Temperature Dependent UV-Vis Spectrophotometry

Turbidity profiles were obtained for each of the constructs by recording the optical density as a function of temperature (1° C. min⁻¹ ramp) on a temperature-controlled UV-vis spectrophotometer (Cary 300 Bio; Varian Instruments; Palo Alto, Calif.). The transition temperature (T₁) was defined as the inflection point of the turbidity profile. Samples were measured in PBS at 10 μM.

### Animal Husbandry

All experimental procedures were conducted under protocol A053-15-02 approved by the Duke Institutional Animal Care and Use Committee (IACUC). 6-week old, male C57Bl/6J mice were purchased from Jackson Labs (strain 000664) and group housed in a room with a controlled photoperiod (12 hr light/12 hr dark cycle) and allowed at least 1 week to acclimate to the facilities prior to that start of procedures. Animals had unlimited access to water and food and were observed daily for signs and symptoms of distress. The diet-induced obese (DIO) phenotype was achieved by maintaining the mice on a high-fat (60 kcal % fat) diet upon arrival to the facility.

### Endotoxin Removal

Constructs were endotoxin purified prior to injection by passing the solution through a sterile 0.22 μm Acrodisc filter comprised of a positively charged and hydrophilic Mustang® E membrane (Pall Corporation). Constructs were filtered in 2 M urea+140 mM PBS at 37° C. and then dialyzed against milli-Q H₂O at 4° C., changing the water three separate times over the course of 72 hours. Aggregated material was removed from the dialysis bag and pelleted with centrifugation (4° C., 3500 rpm). Samples were frozen and lyophilized for a minimum of 48 hrs.

### Method of Establishing GLP-1 Releasing Sub-Cutaneous Depots

In one method, the polypeptide is resuspended at 175 μM in 2 M urea+140 mM PBS. A total volume of 200 μL is injected into the right hind flank after shaving and removing all hair with chemical dissolution at the site of injection. Mice were weighed to determine injection volume required for ˜2100 nmole of GLP-1 per kg of animal weight. Injection volume did not exceed 200 μL. In a second method, a small incision is made on the right hind flank with surgical scissors after animals were anesthetized with isoflurane. Incision site was pre-sterilized according to Duke husbandry guidelines. Pre-weighed, dehydrated polypeptide pellets are then inserted under the skin. The pellets rapidly rehydrate and become adherent to the skin tissue and thus for sealing the incision site, only a small amount of surgical glue was used to secure the skin flap.

### Blood Glucose Measurement and Weight Measurements

Mice were put into a clear restraining tube. Their tails were wiped with 50% ethanol in sterile water and then dried. A small incision was made adjacent to the tail vein using a small lancet. The first drop of blood was blotted away. Blood glucose was quantified by applying the second drop of blood to the test strip of an AlphaTRAK 2 blood glucose meter (Abbott Laboratories). Weight was measured on a scale zeroed with a container into which the mice were briefly placed.

### Statistical Analysis

Experimental numbers for both in vitro and in vivo studies were selected based on knowledge gleaned from previous experiments or other published data. Because of the small sample size (n s 6), normality of groups was not tested. Variance across groups was similar except in untreated versus treated in vivo groups, which is not unexpected given the lack of glucose control in the mouse models tested. Blood glucose and percent change in weight studies were analyzed using repeated measures ANOVA, followed by lower order ANOVAs and Dunnett's Test for multiple comparisons. For comparing two groups, two-tailed Student's t-tests were used. No blinding was performed. Analysis and data processing were performed using Igor and R software.

### Results

We envisioned two strategies with the potential to establish depots subcutaneously for the UCST polypeptides which have transition temperatures far above safe biological temperature ranges, precluding a soluble to insoluble transition upon cooling to body temperature. The two strategies are to 1) employ urea to lower the solution cloud point for injection and 2) injection of a concentrated, dehydrated GLP-1-IDP fusion that will rehydrate, releasing peptide fusion. We decided to directly visualize this effect via fluorescent tomography. We chose a model IDP, (Gly-Arg-Gly-Asp-Ser-Pro-Tyr-Gln)-40 which has a predicted transition temperature of >70° C. at the injection concentration necessary (175 μM or 1.2 mg which corresponds to equivalent doses of GLP-1-ELP fusions used in previous studies, 1000 nmole kg⁻¹). Using a near infrared fluorescent tag (CW800) attached via NHS-ester chemistry at free amines, we visualized the localization of the polypeptide in the hind flank.

We know that inclusion of 1 M urea in solution with the polypeptide will reduce is observed cloud by point by ˜25° C. Thus, by resuspending the model polypeptide in 2 M urea+PBS at 175 μM we can inject in a soluble state under ambient conditions. We hypothesize that considering the two order of magnitude difference in molecular weight between urea and the model polypeptide, urea will rapidly diffuse out of the sub-cutaneous space, leaving the remaining polypeptide in a poor solvent and thus will transition in situ.

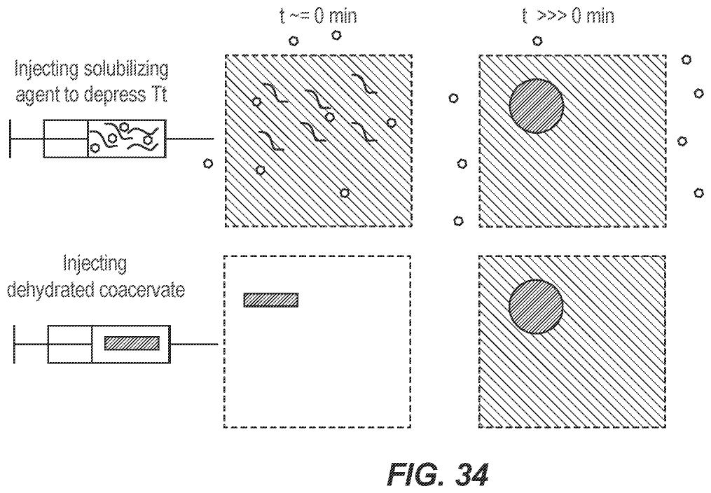

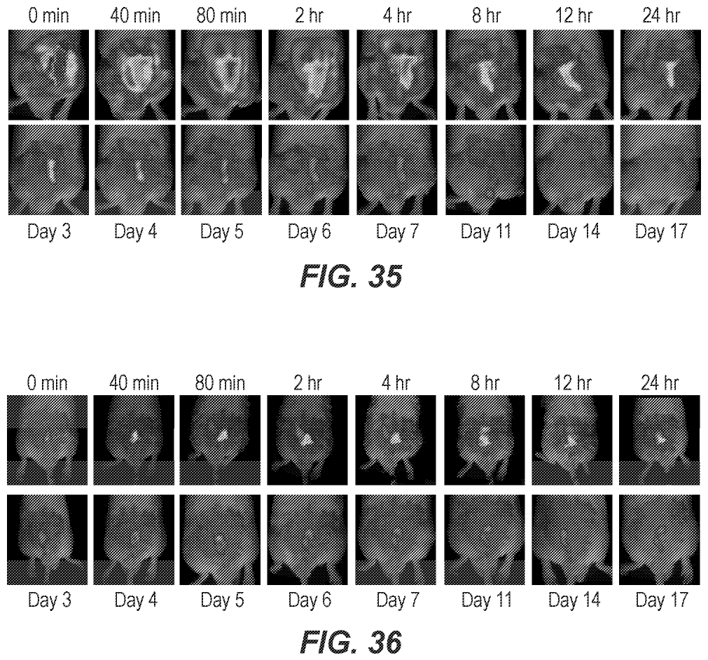

Upon injection we observe something akin to a “burst” release of the polypeptide (FIG. 35). This is characterized by a large area of fluorescence with a center of mass along the axis of the injection path of the needle. Over the first 8 hours this center of mass reduces in intensity, slowly reaching an equilibrium shape over the first two days. This center of mass disappeared by day 14.

We also injected a dehydrated coacervate that had the same total protein content as the urea experiments. Here we observe a completely different behavior. Although our initial intention was to use convective flow from the syringe to push the dehydrated depot from the needle point into the subcutaneous space, upon contact with water, the dehydrated depot becomes extremely adhesive to the hydrophobic needle (FIG. 36). Thus, we implanted the material with forceps, placing the dehydrated pellet underneath the skin through a small incision. Upon implantation, we observe an extremely small area of fluorescent that slowly expands over the first 90 minutes. The center of mass of the implant does not move noticeably over the course of two weeks. However, the mass of the depot is decreasing over this time, slowly releasing material surrounding the depot primarily during the first three days of implantation. Comparing these two injection strategies, we decided to move forward with the dehydrated depot strategy due to the lack of burst release and the increased persistence of the depot.

Fusion of GLP-1 to the N terminus of polypeptides was generally well tolerated. We observed minimal loss in yield from recombinant expression with most constructs expressing between 25-50 mg L⁻¹. As mentioned previously, we wanted to design peptide-polypeptide fusions that have Cₛₐₜ in the general ranges of ˜0.1, 10, >100 μM corresponding to slow release, optimal release and near soluble release from the depot. This roughly corresponds to the Cₛₐₜₛ predicted for [3Y:V]-20, [Y:V]-20 and [3V:Y]-20. [3V:Y]-20 was not expected to exhibit phase behavior under physiologic conditions and thus six His residues were fused to the C terminus of the polypeptide and purified from the soluble fraction with chromatography.

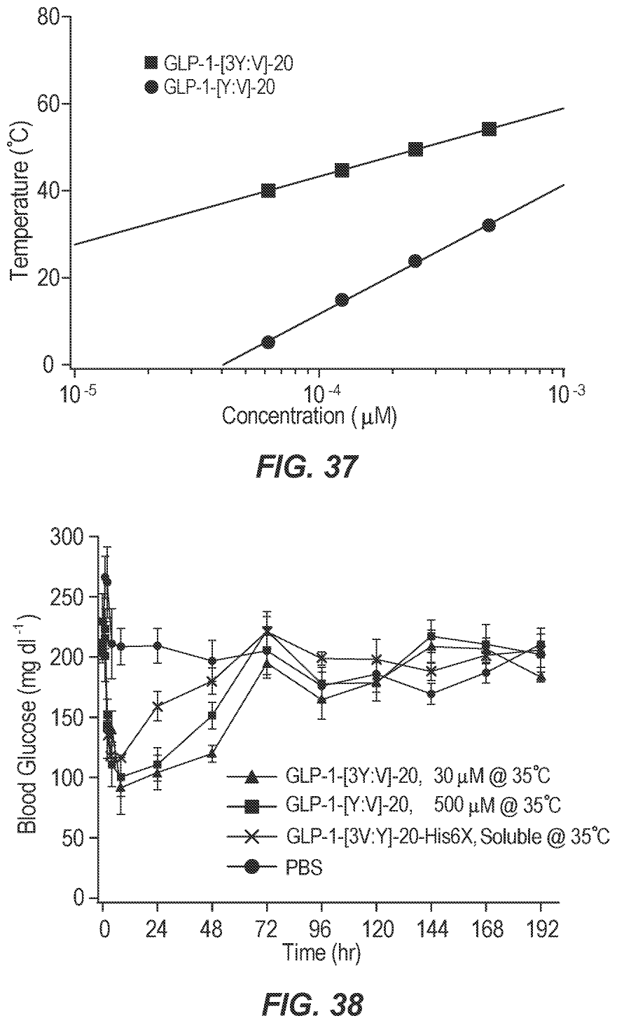

The phase behavior of these polypeptide fusions was measured as before with temperature dependent UV-vis spectrophotometry. In determining the UCST binodal line, we identify these two proteins indeed have the desired phase behavior with GLP-1-[3Y:V]-20 exhibiting a Cₛₐₜ of ˜30 μM and GLP-1-[Y:V]-20 exhibiting a Cₛₐₜ of ˜500 μM (FIG. 37). These roughly correspond to the values predicted by the RIDP of choice alone. As predicted GLP-1-[3V:Y]-20-His6× did not exhibit any phase behavior under physiologic conditions.

After endotoxin purification, 1.2 mg of GLP-1-[3Y:V]-20, GLP-1-[Y:V]-20 and GLP-1-[3V:Y]-20-His6× were weighed and implanted in the hind flank of C57Bl/6J mice that have been fed 60% fat diet. In addition to these 3 groups, there is an additional group that received a saline injection. Over the course of the study, we measured blood glucose via tail vein blood draws at 0, 1, 2, 4, 8, 24 hrs and then each day thereafter for a total of 8 days.

The blood glucose data can be visualized in FIG. 38. Overall, our strategy of implanting dehydrated depots was successful at controlling blood glucose. It is also positive that we are observing an effect of aromatic:aliphatic ratio, even in non-optimal molecular weight polypeptides. First, it is notable that in the early time points blood glucose drops at approximately same speed suggesting that even in the soluble control, there are limitations regarding the minimal time to observe an effect on blood glucose. Second, each experimental group exhibits elements of burst release, with the largest change observed for those constructs that form subcutaneous depots. This result suggests that upon solubilization there is a larger bolus dose that reaches the blood stream, which is reduced upon reaching an equilibrium state between depot release and protein clearance. Third, our depot forming formulations (GLP-1-[3Y:V]-20 and GLP-1-[Y:V]-20) each control blood glucose at least one additional day compared to the soluble RIDP control.

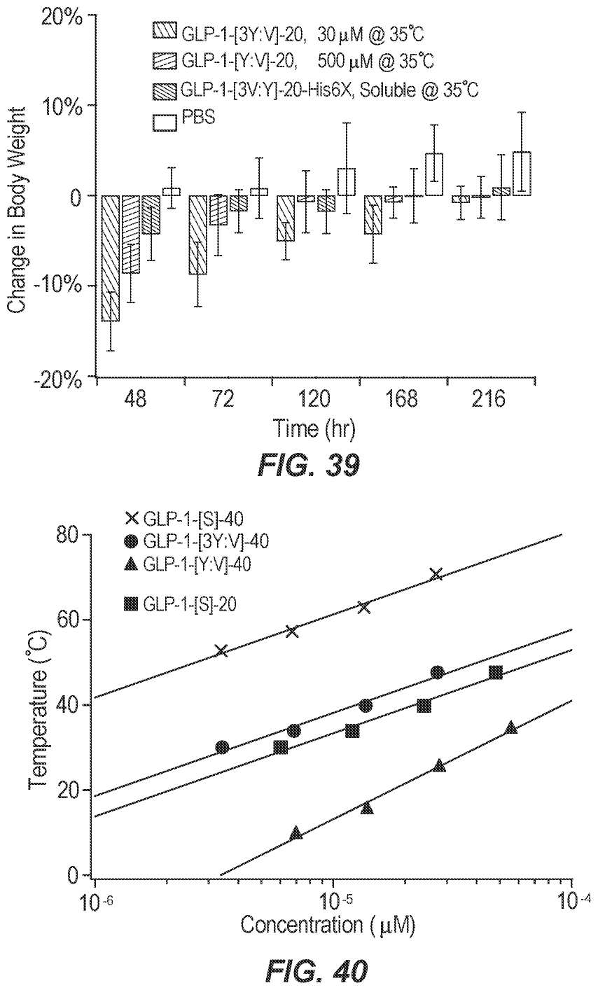

Measurements of the body weight of the mice provide supplementary information on the efficacy of our sub-cutaneous depots (FIG. 39). Again, we observe that our depot-forming proteins exhibit the greatest level of a burst release effect, resulting in the largest change in body weight in the first 2 days. This effect appears to be somewhat depot-dose dependent where the lower Cₛₐₜ construct exhibits the largest depression in appetite. As expected, the saline injection does not affect body weight.

Body weight measurements also differentiate our two depot forming fusions from one another.

The high Cₛₐₜ construct body weight measurements suggest that their efficacy has waned by day 5 whereas the low Cₛₐₜ appears to be exerting a phenotypic effect until the end of the study (day 8).

These experiments mirror similar results of optimization experiments with GLP-1-ELP depots. There were diminishing returns of polypeptides with molecular weights exceeding 35 kDa but improvements to glucose control between 20-35 kDa. Thus, we explored creating higher molecular weight variants of GLP-1-RIDP fusions.

Increasing the molecular weight of polypeptide fusions produced a series that have Cₛₐₜₛ of 0.5, 7, and 60 μM by progressively reducing the aromatic content with aliphatic substitutions. (FIG. 40). Another GLP-1 protein fusion was also made with 75% aliphatic content that did not exhibit UCST phase behavior under physiologic conditions. I also synthesized a molecular weight control (GLP-1-[S]-20) that has similar Cₛₐₜ GLP-1-[3Y:V]-40 (7 μM compared to 12 μM) but is half the molecular weight.

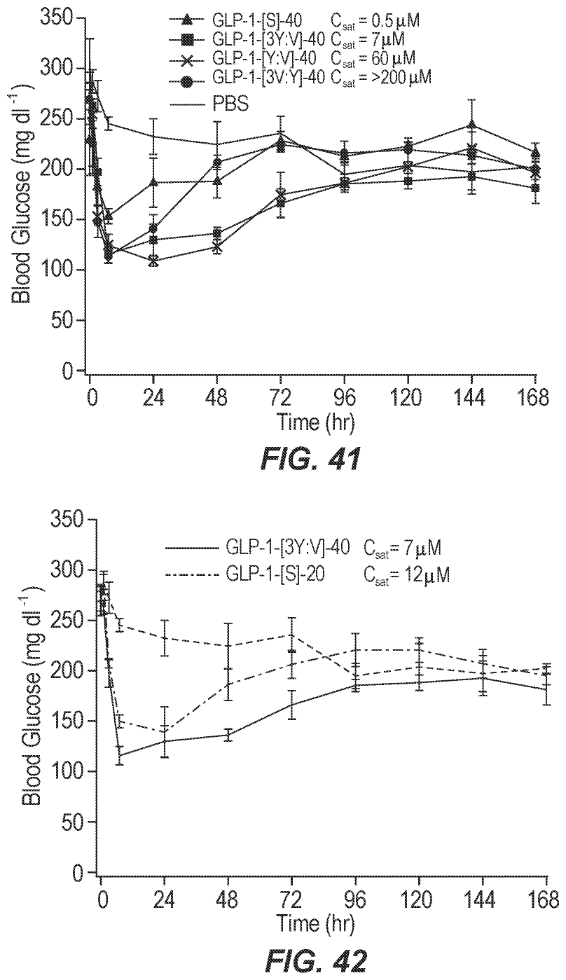

The blood glucose measurements of mice with 2.0 mg depots implanted in their subcutaneous space can be visualized in FIG. 41. These proteins that exhibit variable Cₛₐₜ also exhibit variable release from the depot in the subcutaneous space. The most hydrophobic depot, GLP-1-S-[40], appears to release the least amount of material suggesting that the depot biophysical properties is retarding a phenotypic effect of the peptide drug. The middle hydrophobic depots, GLP-1-[3Y:V]-40 and GLP-1-[Y:V]-40, with Cₛₐₜ between 7 and 60 μM, exert similar levels of glucose control that is nearly a full 24 hr improvement over the low molecular weight versions. The most hydrophilic “depot,” predicted to be soluble can only manage glucose control over the first 24 hr. This is still an improvement from the soluble control at a lower molecular weight. These experiments support previous conclusions of optimal depot design, identifying that optimal release kinetics can be achieved with polypeptides that exhibit a Cₛₐₜ between 7 and 60 μM.

The effect of molecular weight, independent of Cₛₐₜ, on blood glucose can be visualized in FIG. 42. Increasing the molecular weight but retaining a Cₛₐₜ that is within an optimal release range can prolong glucose control by an additional two days. This effect is likely a result of delayed diffusion into the blood stream and prolonged drug half-life from delayed renal clearance.

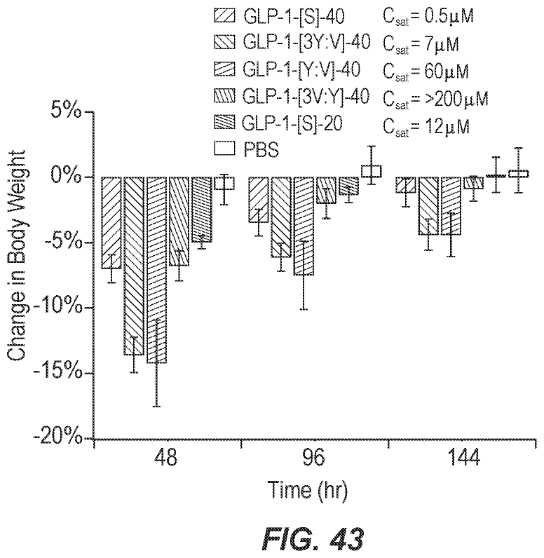

Tracking the body weight of the mice supports the conclusions inferred from the blood glucose measurements (FIG. 43). Here the parabolic effect of depot hydrophobicity is clear—at the extremes of hydrophobicity and hydrophilicity there is a lesser burst release and shorter duration of weight control. The “optimal” constructs, GLP-1-[3Y:V]-40 and GLP-1-[Y:V]-40, are still exhibiting weight control at 7 days suggesting that there must be small amounts of material releasing from the depot even at 144 hours after implantation. The molecular weight control, GLP-1-[S]-20, exhibits lesser burst released and shorter duration of efficacy than its larger molecular weight analogue, GLP-1-[3Y:V]-40, again supporting the conclusion of delayed entry and prolonged persistence in the blood stream from higher molecular weight depots.

In summary, we designed mimetic fusions with the GLP-1-ELP system. We also discovered multiple new routes of establishing sub-cutaneous depots with unfavorable working transition temperatures. Using these two innovations we were able to create depots that performed with similar efficacy to previous GLP-1-ELP depots, controlling blood glucose for up to 5 days in vivo in a DIO mice model.

What is claimed:

1. A polypeptide with controlled reversible phase separation comprising ten or more repeats of an amino acid sequence comprising:
(X-Z₁-X-Z-Z₃-X-Z₄-Z₃)ₙ,
where:
X is proline (P) or glycine (G) and the ratio of P:G is any number;
Z₁ is arginine (R), aspartic acid (D), or lysine (K) and the ratio of R:D is any number and the ratio of K:R can be any number;
Z₂ is Asp (D), Arg (R), Glu (E), where the ratio of R:D can be any number and D:E can be any number;
Z₃ is asparagine (N), glutamine (Q), serine (S), or threonine (T) were the ratio among N:Q:S:T can be any number; and
Z₄ is tyrosine (Y), histidine (H), tryptophan (W), phenylalanine (F), methionine (M), valine (V), isoleucine (I), alanine (A), or leucine (L) and the ratio among Y:H:W:F:M:V:I:A:L can be any number.

2. The polypeptide of claim 1, wherein X is proline (P) or glycine (G) and the ratio of P:G is between 1:3 and 3:1.

3. The polypeptide of claim 1, wherein Z₁ is arginine (R), aspartic acid (D), or lysine (K) and the ratio of R:D does not exceed 1:5 and the ratio of K:R can be any number.

4. The polypeptide of claim 1, wherein the phase separation is dependent on temperature, molecular weight, hydrophobicity, aromatic:aliphatic ratio, and concentration.

5. The polypeptide of claim 1, wherein n is 10 to 200.

6. The polypeptide of claim 1, wherein the molecular weight is at least 5 kDa to 500 kDa.

7. The polypeptide of claim 1, wherein the molecular weight is about 5 kDa to about 100 kDa.

8. The polypeptide of claim 1, wherein the phase separation temperature is 0 to 100° C.

9. The polypeptide of claim 1, wherein the phase separation temperature is 4 to 25° C.; ˜25° C.; 25 to 37° C.; ˜37° C.; 35 to 38° C.; or >38° C.

10. The polypeptide of claim 1, wherein the polypeptide comprises modified amino acids, a reporter protein, or an enzyme.

11. The polypeptide of claim 10, wherein the sequence comprises:
(G-R-G-D-S-P-Y-S)ₘ,
where m is 20 to 80.

12. The polypeptide of claim 1, wherein the polypeptide comprises a sequence selected from one or more of SEQ ID NO: 1-1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, or 197-279, or combinations thereof.

13. A pharmaceutically acceptable composition comprising a polypeptide with controlled reversible phase separation comprising ten or more repeats of an amino acid sequence comprising:
(X-Z₁-X-Z₂-Z₃-X-Z₄-Z₃)ₙ,
where:
X is proline (P) or glycine (G) and the ratio of P:G is any number;
Z₁ is arginine (R), aspartic acid (D), or lysine (K) and the ratio of R:D is any number and the ratio of K:R can be any number;
Z₂ is Asp (D), Arg (R), Glu (E), where the ratio of R:D can be any number and D:E can be any number;
Z₃ is asparagine (N), glutamine (Q), serine (S), or threonine (T) were the ratio among N:Q:S:T can be any number; and
Z₄ is tyrosine (Y), histidine (H), tryptophan (W), phenylalanine (F), methionine (M), valine (V), isoleucine (I), alanine (A), or leucine (L) and the ratio among Y:H:W:F:M:V:I:A:L can be any number.

14. The composition of claim 13, wherein X is proline (P) or glycine (G) and the ratio of P:G is between 1:3 and 3:1.

15. The composition of claim 13, wherein Z₁ is arginine (R), aspartic acid (D), or lysine (K) and the ratio of R:D does not exceed 1:5 and the ratio of K:R can be any number.

16. The composition of claim 13, further comprising an attached molecule comprising one or more of an antibody binding domain derived from Staphylococcus protein A (ZD) (SEQ ID NO:159), an antimicrobial peptide selected from LL37 (SEQ ID NO: 161), Ib-M1 (SEQ ID NO: 163), Ib-M2 (SEQ ID NO: 165), Ib-M5 (SEQ ID NO: 167), Cathelecidin-1 (SEQ ID NO: 169), A(A1R, A8R, I17K) (SEQ ID NO: 171), H5 (SEQ ID NO: 173), H5-61-90 (SEQ ID NO: 175); RGD peptide (RGDSPAS, SEQ ID NO: 39); protein drugs, GLP-1 (SEQ ID NO: 177); fluorescent reporters (sfGFP (SEQ ID NO: 179), mRuby3 (SEQ ID NO: 181); RNA binding proteins (PUM-HD (SEQ ID NO: 183), eIF4E (SEQ ID NO: 185), PABP (SEQ ID NO: 187), Tis11D (SEQ ID NO: 189)); KH domains (Yifan or FMRP (SEQ ID NO: 191)); or AAV binding peptides PKD1 (SEQ ID NO: 193) or PKD2 (SEQ ID NO: 195).

17. The composition of claim 13, wherein the composition enhances bioavailability of the attached molecule as compared to the free form of the attached molecule.

18. The composition of claim 13, wherein the composition enhances recombinant expression of the attached molecule as compared to the free form of the attached molecule.

19. The composition of claim 13, wherein the composition enhances the stability of the attached molecule as compared to the free form of the attached molecule.

20. The composition of claim 19, wherein the composition enhances stability of the attached molecule during prokaryotic or eukaryotic expression as compared to the free form of the attached molecule.

21. The composition of claim 19, wherein the enhanced stability includes resistance to denaturation during freezing, thawing, lyophilization or prolonged storage at temperatures greater than 4° C.

22. The composition of claim 13, wherein the composition modulates enzymatic, metabolic, or physiological functions within cells or organisms.

23. The composition of claim 22, wherein the modulation reduces bioavailability of the attached molecules.

24. The composition of claim 23, wherein the attached molecules comprise therapeutic or cytotoxic proteins or peptides.

25. A method for enhancing the bioavailability or stability of a protein, the method comprising creating a fusion protein of one or more proteins and a polypeptide with controlled reversible phase separation comprising ten or more repeats of an amino acid sequence comprising:
(X-Z₁-X-Z₂-Z₃-X-Z₄-Z₃)ₙ,
where:
X is proline (P) or glycine (G) and the ratio of P:G is any number;
Z₁ is arginine (R), aspartic acid (D), or lysine (K) and the ratio of R:D is any number and the ratio of K:R can be any number;
Z₂ is Asp (D), Arg (R), Glu (E), where the ratio of R:D can be any number and D:E can be any number;
Z₃ is asparagine (N), glutamine (Q), serine (S), or threonine (T) were the ratio among N:Q:S:T can be any number; and
Z₄ is tyrosine (Y), histidine (H), tryptophan (W), phenylalanine (F), methionine (M), valine (V), isoleucine (I), alanine (A), or leucine (L) and the ratio among Y:H:W:F:M:V:I:A:L can be any number.

26. The method of claim 25, wherein X is proline (P) or glycine (G) and the ratio of P:G is between 1:3 and 3:1.

27. The method of claim 25, wherein Z₁ is arginine (R), aspartic acid (D), or lysine (K) and the ratio of R:D does not exceed 1:5 and the ratio of K:R can be any number.

28. The method of claim 25, wherein the protein comprises one or more of an antibody binding domain derived from Staphylococcus protein A (ZD) (SEQ ID NO:159), an antimicrobial peptide selected from LL37 (SEQ ID NO: 161), Ib-M1 (SEQ ID NO: 163), Ib-M2 (SEQ ID NO: 165), Ib-M5 (SEQ ID NO: 167), Cathelecidin-1 (SEQ ID NO: 169), A(A1R, A8R, I17K) (SEQ ID NO: 171), H5 (SEQ ID NO: 173), H5-61-90 (SEQ ID NO: 175); RGD peptide (RGDSPAS, SEQ ID NO: 39); protein drugs, GLP-1 (SEQ ID NO: 177); fluorescent reporters (sfGFP (SEQ ID NO: 179), mRuby3 (SEQ ID NO: 181); RNA binding proteins (PUM-HD (SEQ ID NO: 183), eIF4E (SEQ ID NO: 185), PABP (SEQ ID NO: 187), Tis11D (SEQ ID NO: 189)); KH domains (Yifan or FMRP (SEQ ID NO: 191)); or AAV binding peptides PKD1 (SEQ ID NO: 193) or PKD2 (SEQ ID NO: 195).

29. The method of claim 25, where the enhanced bioavailability of the fusion protein can be used for isolation or separation of a biologic molecule.

30. The method of claim 25, wherein the biologic molecule comprises one or more of a lipid, a cell, a protein, a nucleic acid, a carbohydrate, or a viral particle.

31. The method of claim 30, wherein the nucleic acid is single stranded or double stranded DNA or RNA.

32. The method of claim 30, wherein the viral particle is an adenovirus particle, an adeno-associated virus particle, a lentivirus particle, a retrovirus particle, a poxvirus particle, a measle virus particle, or herpesvirus particle.

33. The method of claim 30, wherein the protein comprises albumin, monoclonal IgG antibodies, or Fc fusion proteins.

34. The method of claim 30, wherein the isolation or separation is accomplished via reversible phase separation.
